SEQUENCE LISTING (287 sequences)

SEQ ID NO: 1 - protein, 2261 aa, Homo sapiens [sequence omitted]

SEQ ID NO: 2 - DNA, 7860 bp, Homo sapiens
gtccctgctgtgagctctggccgctgccttccagggctcccgagccacacgctgggggtgctggctgagggaacatggcttgttggcctcagctgaggttgctgctgtggaagaacctcactttcagaagaagacaaacatgtcagctgttactggaagtggcctggcctctatttatcttcctgatcctgatctctgttcggctgagctacccaccctatgaacaacatgaatgccattttccaaataaagccatgccctctgcaggaacacttccttgggttcaggggattatctgtaatgccaacaacccctgtttccgttacccgactcctggggaggctcccggagttgttggaaactttaacaaatccattgtggctcgcctgttctcagatgctcggaggcttcttttatacagccagaaagacaccagcatgaaggacatgcgcaaagttctgagaacattacagcagatcaagaaatccagctcaaacttgaagcttcaagatttcctggtggacaatgaaaccttctctgggttcctgtatcacaacctctctctcccaaagtctactgtggacaagatgctgagggctgatgtcattctccacaaggtatttttgcaaggctaccagttacatttgacaagtctgtgcaatggatcaaaatcagaagagatgattcaacttggtgaccaagaagtttctgagctttgtggcctaccaagggagaaactggctgcagcagagcgagtacttcgttccaacatggacatcctgaagccaatcctgagaacactaaactctacatctcccttcccgagcaaggagctggctgaagccacaaaaacattgctgcatagtcttgggactctggcccaggagctgttcagcatgagaagctggagtgacatgcgacaggaggtgatgtttctgaccaatgtgaacagctccagctcctccacccaaatctaccaggctgtgtctcgtattgtctgcgggcatcccgagggaggggggctgaagatcaagtctctcaactggtatgaggacaacaactacaaagccctctttggaggcaatggcactgaggaagatgctgaaaccttctatgacaactctacaactccttactgcaatgatttgatgaagaatttggagtctagtcctctttcccgcattatctggaaagctctgaagccgctgctcgttgggaagatcctgtatacacctgacactccagccacaaggcaggtcatggctgaggtgaacaagaccttccaggaactggctgtgttccatgatctggaaggcatgtgggaggaactcagccccaagatctggaccttcatggagaacagccaagaaatggaccttgtccggatgctgttggacagcagggacaatgaccacttttgggaacagcagttggatggcttagattggacagcccaagacatcgtggcgtttttggccaagcacccagaggatgtccagtccagtaatggttctgtgtacacctggagagaagctttcaacgagactaaccaggcaatccggaccatatctcgcttcatggagtgtgtcaacctgaacaagctagaacccatagcaacagaagtctggctcatcaacaagtccatggagctgctggatgagaggaagttctgggctggtattgtgttcactggaattactccaggcagcattgagctgccccatcatgtcaagtacaagatccgaatggacattgacaatgtggagaggacaaataaaatcaaggatgggtactgggaccctggtcctcgagctgacccctttgaggacatgcggtacgtctgggggggcttcgcctacttgcaggatgtggtggagcaggcaatcatcagggtgctgacgggcaccgagaagaaaactggtgtctatatgcaacagatgccctatccctgttacgttgatgacatctttctgcgggtgatgagccggtcaatgcccctcttcatgacgctggcctggatttactcagtggctgtgatcatcaagggcatcgtgtatgagaaggaggcacggctgaaagagaccatgcggatcatgggcctggacaacagcatcctctggtttagctggttcattagtagcctcattcctcttcttgtgagcgctggcctgctagtggtcatcctgaagttaggaaacctgctgccctacagtgatcccagcgtggtgtttgtcttcctgtccgtgtttgctgtggtgacaatcctgcagtgcttcctgattagcacactcttctccagagccaacctggcagcagcctgtgggggcatcatctacttcacgctgtacctgccctacgtcctgtgtgtggcatggcaggactacgtgggcttcacactcaagatcttcgctagcctgctgtctcctgtggcttttgggtttggctgtgagtactttgccctttttgaggagcagggcattggagtgcagtgggacaacctgtttgagagtcctgtggaggaagatggcttcaatctcaccacttcggtctccatgatgctgtttgacaccttcctctatggggtgatgacctggtacattgaggctgtctttccaggccagtacggaattcccaggccctggtattttccttgcaccaagtcctactggtttggcgaggaaagtgatgagaagagccaccctggttccaaccagaagagaatatcagaaatctgcatggaggaggaacccacccacttgaagctgggcgtgtccattcagaacctggtaaaagtctaccgagatgggatgaaggtggctgtcgatggcctggcactgaatttttatgagggccagatcacctccttcctgggccacaatggagcggggaagacgaccaccatgtcaatcctgaccgggttgttccccccgacctcgggcaccgcctacatcctgggaaaagacattcgctctgagatgagcaccatccggcagaacctgggggtctgtccccagcataacgtgctgtttgacatgctgactgtcgaagaacacatctggttctatgcccgcttgaaagggctctctgagaagcacgtgaaggcggagatggagcagatggccctggatgttggtttgccatcaagcaagctgaaaagcaaaacaagccagctgtcaggtggaatgcagagaaagctatctgtggccttggcctttgtcgggggatctaaggttgtcattctggatgaacccacagctggtgtggacccttactcccgcaggggaatatgggagctgctgctgaaataccgacaaggccgcaccattattctctctacacaccacatggatgaagcggacgtcctgggggacaggattgccatcatctcccatgggaagctgtgctgtgtgggctcctccctgtttctgaagaaccagctgggaacaggctactacctgaccttggtcaagaaagatgtggaatcctccctcagttcctgcagaaacagtagtagcactgtgtcatacctgaaaaaggaggacagtgtttctcagagcagttctgatgctggcctgggcagcgaccatgagagtgacacgctgaccatcgatgtctctgctatctccaacctcatcaggaagcatgtgtctgaagcccggctggtggaagacatagggcatgagctgacctatgtgctgccatatgaagctgctaaggagggagcctttgtggaactctttcatgagattgatgaccggctctcagacctgggcatttctagttatggcatctcagagacgaccctggaagaaatattcctcaaggtggccgaagagagtggggtggatgctgagacctcagatggtaccttgccagcaagacgaaacaggcgggccttcggggacaagcagagctgtcttcgcccgttcactgaagatgatgctgctgatccaaatgattctgacatagacccagaatccagagagacagacttgctcagtgggatggatggcaaagggtcctaccaggtgaaaggctggaaacttacacagcaacagtttgtggcccttttgtggaagagactgctaattgccagacggagtcggaaaggattttttgctcagattgtcttgccagctgtgtttgtctgcattgcccttgtgttcagcctgatcgtgccaccctttggcaagtaccccagcctggaacttcagccctggatgtacaacgaacagtacacatttgtcagcaatgatgctcctgaggacacgggaaccctggaactcttaaacgccctcaccaaagaccctggcttcgggacccgctgtatggaaggaaacccaatcccagacacgccctgccaggcaggggaggaagagtggaccactgccccagttccccagaccatcatggacctcttccagaatgggaactggacaatgcagaacccttcacctgcatgccagtgtagcagcgacaaaatcaagaagatgctgcctgtgtgtcccccaggggcaggggggctgcctcctccacaaagaaaacaaaacactgcagatatccttcaggacctgacaggaagaaacatttcggattatctggtgaagacgtatgtgcagatcatagccaaaagcttaaagaacaagatctgggtgaatgagtttaggtatggcggcttttccctgggtgtcagtaatactcaagcacttcctccgagtcaagaagttaatgatgccatcaaacaaatgaagaaacacctaaagctggccaaggacagttctgcagatcgatttctcaacagcttgggaagatttatgacaggactggacaccagaaataatgtcaaggtgtggttcaataacaagggctggcatgcaatcagctctttcctgaatgtcatcaacaatgccattctccgggccaacctgcaaaagggagagaaccctagccattatggaattactgctttcaatcatcccctgaatctcaccaagcagcagctctcagaggtggctctgatgaccacatcagtggatgtccttgtgtccatctgtgtcatctttgcaatgtccttcgtcccagccagctttgtcgtattcctgatccaggagcgggtcagcaaagcaaaacacctgcagttcatcagtggagtgaagcctgtcatctactggctctctaattttgtctgggatatgtgcaattacgttgtccctgccacactggtcattatcatcttcatctgcttccagcagaagtcctatgtgtcctccaccaatctgcctgtgctagcccttctacttttgctgtatgggtggtcaatcacacctctcatgtacccagcctcctttgtgttcaagatccccagcacagcctatgtggtgctcaccagcgtgaacctcttcattggcattaatggcagcgtggccacctttgtgctggagctgttcaccgacaataagctgaataatatcaatgatatcctgaagtccgtgttcttgatcttcccacatttttgcctgggacgagggctcatcgacatggtgaaaaaccaggcaatggctgatgccctggaaaggtttggggagaatcgctttgtgtcaccattatcttgggacttggtgggacgaaacctcttcgccatggccgtggaaggggtggtgttcttcctcattactgttctgatccagtacagattcttcatcaggcccagacctgtaaatgcaaagctatctcctctgaatgatgaagatgaagatgtgaggcgggaaagacagagaattcttgatggtggaggccagaatgacatcttagaaatcaaggagttgacgaagatatatagaaggaagcggaagcctgctgttgacaggatttgcgtgggcattcctcctggtgagtgctttgggctcctgggagttaatggggctggaaaatcatcaactttcaagatgttaacaggagataccactgttaccagaggagatgctttccttaacaaaaatagtatcttatcaaacatccatgaagtacatcagaacatgggctactgccctcagtttgatgccatcacagagctgttgactgggagagaacacgtggagttctttgcccttttgagaggagtcccagagaaagaagttggcaaggttggtgagtgggcgattcggaaactgggcctcgtgaagtatggagaaaaatatgctggtaactatagtggaggcaacaaacgcaagctctctacagccatggctttgatcggcgggcctcctgtggtgtttctggatgaacccaccacaggcatggatcccaaagcccggcggttcttgtggaattgtgccctaagtgttgtcaaggaggggagatcagtagtgcttacatctcatagtatggaagaatgtgaagctctttgcactaggatggcaatcatggtcaatggaaggttcaggtgccttggcagtgtccagcatctaaaaaataggtttggagatggttatacaatagttgtacgaatagcagggtccaacccggacctgaagcctgtccaggatttctttggacttgcatttcctggaagtgttctaaaagagaaacaccggaacatgctacaataccagcttccatcttcattatcttctctggccaggatattcagcatcctctcccagagcaaaaagcgactccacatagaagactactctgtttctcagacaacacttgaccaagtatttgtgaactttgccaaggaccaaagtgatgatgaccacttaaaagacctctcattacacaaaaaccagacagtagtggacgttgcagttctcacatcttttctacaggatgagaaagtgaaagaaagctatgtatgaagaatcctgttcatacggggtggctgaaagtaaagaggaactagactttcctttgcaccatgtgaagtgttgtggagaaaagagccagaagttgatgtgggaagaagtaaactggatactgtactgatactattcaatgcaatgcaattcaatgcaatgaaaacaaaattccattacaggggcagtgcctttgtagcctatgtcttgtatggctctcaagtgaaagacttgaatttagttttttacctatacctatgtgaaactctattatggaacccaatggacatatgggtttgaactcacacttttttttttttttttgttcctgtgtattctcattggggttgcaacaataattcatcaagtaatcatggccagcgattattgatcaaaatcaaaaggtaatgcacatcctcattcactaagccatgccatgcccaggagactggtttcccggtgacacatccattgctggcaatgagtgtgccagagttattagtgccaagtttttcagaaagtttgaagcaccatggtgtgtcatgctcacttttgtgaaagctgctctgctcagagtctatcaacattgaatatcagttgacagaatggtgccatgcgtggctaacatcctgctttgattccctctgataagctgttctggtggcagtaacatgcaacaaaaatgtgggtgtctccaggcacgggaaacttggttccattgttatattgtcctatgcttcgagccatgggtctacagggtcatccttatgagactcttaaatatacttagatcctggtaagaggcaaagaatcaacagccaaactgctggggctgcaactgctgaagccagggcatgggattaaagagattgtgcgttcaaacctagggaagcctgtgcccatttgtcctgactgtctgctaacatggtacactgcatctcaagatgtttatctgacacaagtgtattatttctggctttttgaattaatctagaaaatgaaa

SEQ ID NO: 3 - DNA, 22 bp, Homo sapiens
gcagagggcatggctttatttg

SEQ ID NO: 4 - DNA, 24 bp, Homo sapiens
ctgccaggcaggggaggaagagtg

SEQ ID NO: 5 - DNA, 23 bp, Homo sapiens
gaaagtgactcacttgtggagga

SEQ ID NO: 6 - DNA, 20 bp, Homo sapiens
aaaggggcttggtaagggta

SEQ ID NO: 7 - DNA, 20 bp, Homo sapiens
catgcacatgcacacacata

SEQ ID NO: 8 - DNA, 27 bp, Homo sapiens
ctttctgcgggtgatgagccggtcaat

SEQ ID NO: 9 - DNA, 20 bp, Homo sapiens
ccttagcccgtgttgagcta

SEQ ID NO: 10 - DNA, 26 bp, Homo sapiens
cctgtaaatgcaaagctatctcctct

SEQ ID NO: 11 - DNA, 26 bp, Homo sapiens
cgtcaactccttgatttctaagatgt

SEQ ID NO: 12 - DNA, 20 bp, Homo sapiens
gggttcccagggttcagtat

SEQ ID NO: 13 - DNA, 21 bp, Homo sapiens
gatcaggaattcaagcaccaa

SEQ ID NO: 14 - DNA, 10545 bp, Homo sapiens
  misc_feature (1)...(10545): n = a, t, c, or g
acctcttatagaatgatagaattcctctggaatgattggataacttcatttcatccttgacttttaccttggaggatttcttaccccttttggcttctcaaatttgactattaaaatgttgcctttaaaaataggaacacagtttcaggggggagtaccagcccatgacccttctgcaaggccccctaactcaaggtagtttccctggaactgtggtttatggaatgtttcaggagtgtgaggaggtataatttaaggctgtcctagcaaggatacccttaaggatagagggcccagtagcatctggaggccagaaaagttaaactgaggcagtcagattagcttcaggctcaattaagctgatgggtcagcctgggagaaattgcaggatgactctcaatatcccctcccacccccacagcagccacgatctgtctgtctttaatcatgggtgcagtgaacctgttctttccaggtgtcttggccttcagtaaccttgttaggcttgtccctgaacgtggctaccgatccaaagacacatgatcagagaggcaattagagaacagaccttttccaaagcaagcatgttctgttgggcttagaagtttcatgtcctaatattataggaccctgtgcatctctctggagatgaggcacatgagtcatatctgtgattcttgcttttgtgtcaacatctcatgaataggcaatcagagctttggcaccaatgtattttcagttcatatctgatgtagttaaatccacctcctgctttgtagtttactggcaagctgtttttgatataagacatctagaacactgtaaatatataacatttttatttgtctattatacctcaattacgaaaaagacatctagaagcaacctcatcaagagagatactgaggccgggcatggtagctcacacttgcaatcccattactttgggaggctgaggcaggtagatcacttgaggtcaagagtttgaaaccagcctggccaacatgttgaaaccctgtctctattaaaaatacaaaaaagttagctgggcttggtggtgggcacctgtaatcccagctactccggaggctgaggcaggagaatcacttgaacctgggaggcagaggttgcagtgagctgagatcacaccactgcactccaacctgggcaccagagtgagattacatctaaaaaataaaataaagtaataaaaaagagagatattgatagctgttgttggaaatttcaacttccatctcacttctggtaactttttggaagtttgttgaacaaagtggaatacacgcacatacacacacacacatactctcttgtttgtttaaggtttaatgaaatagctgtcatataatcactgtttttgaaagaggagaattagttgctatctgtacattttgggtatgtgaactatttggatagaactctgagaaatgcattcagaacaacaaacaaaatcataggagaaatagctaagtgggaaggggcatataagagttgttgaaaaagttatttcttgagaaaccagctctaatgctaggcaagtcacttgctttgggggaggcctcagcttctctgtctataagattgcagcaggggtgtagtgggaatgagtcttcaacattccaagagattttatctactaatacgacagtcaaatggagcatgactttgtggaagcctctcctcttccacccagaggggccaatttctctgtcccagtgagatgttgacacttgtatgatccctgcttggagacttccctcttctggaacctgccctggctcaggcatgagggctgactgtcacccttcgataggagcccagcactaaagctcatgtgttggcagtgttcttgcgggaaggaaaaagaccagccagcccatttgttactgcacaagcaaacagcttctggtagctgtacagatacatgcactttctttcctcactgtgtttccatagacagatttagtgctgtagaagagtagagggcagtcacgggaaggagttcctgtttttcttttggctatgccaaatggggaaaaatcctcctatcttgtctttttagtgtcatcctctctccccttttcttcttctttataattctcatctctcatctctcctggaaatgtgcatgtcaagttcaaaagggcacaatgttttggtgaggaagaggtgggagaacacgtgccaggtgctaactagggtcatcatttcccccttcacagccagcttcctgtgaatgtgtgtgtgtgtgtgtgtgtgtgtgtgtgtgtgtgtgtgtgtgtgtatttcttttgccagcatcactgaatctgtctgctgtctggtattccaggttttggtttagggaaaagtaaaagtaattttataatcccagctgtcatttaagccacccctttgtgggtagcatatggtccactctctcagttcattgtcctaaagatgcttcatcagaaaggaataacttccaccccgttactctctgtccccttactctgctttatttttcttcgtcaatcctaccaccaccacccactgtttgaacaacccactattatttgtctgtttcccatccctggtagaataggagccccatgaatgaaggaactttgcttctgttgttcaccactgaatctctaaggtatggaacacacctggcatgtgataggcactcgataaatatttgttgtggctcatgggcaccttgcagagttaaggctgcagttgtttgtggaatttataagtggtaatgaatatttatctactattcctcttccaaggcgatcacacaataatcaggctttacactatccagttcttaggtcttccaagttatgacttgtgaggtatgttaattatgataatagaaggcagtttatttggttcagatttattgatgtgtaatttaccacagtaagacttcccctttacaaaagtatgatgagttttgacaaatggatacacatgtgtatctaccactgccatgctccttttcagtctgtcgtcccctccacccatgaccactggtcaccactgcagtgatttctgtccccttcatttcaccttttccagaatgtcatataaatggaatcatgcagtatgtagttttttgtgtctggcttatttttcttagcattaggcttttgggattcatccaggttgtcgcatgtaacagtagcttattcctttttatggctgagtaagtgtcccagttttatttatatatttatttatgaggaggtgtctcactctgtcacccaggctggagtgcggtagcgcgatctcagctcactgcaacctccgcctcccaggttcaagcaattctcctgcctcctgagtagctgggattacaggcacccaccgccacgcccaactaatttttatatttttagtagagatggggtttcaccatgttggccaggctgatctcaaactcttgacctcaggtgatccgcccacctctggctcccaaagtgctaggattacaggcatgagccactgtgcccagccccagttttatttattcaccagttgatggtcttttcgacaactaattgtttccagtttttggctattctgtataaggcttctataaatattcacaaatacctaggatgggatgactgggtcatataatagtactgtataaccttagcagaaactgtcaaactattttccaaagtggctcttccattttacaattccacagtgtattgagtcccagtgtctccatacacatgctagcacttttaatatttaatttagtgggtatgtaatgatatctcattgtggttttaatttgcatttctctgcagctaatgatgagtgtttctgcttatttgggaaggttttaatttagcagtctgttgtattctgtagatattaataacttcaaaatatcagtggcatttgcagttaaaatttccttaaaaaattggccaaaggtttccagcagtcacttctgccatgcccaaactgtatgaaacaaggctgaggtgtggagattgtcacattttggcaaggagtgatccacttgggtgactgatgagacccagagagcgtacgcctcgggcttgagggtgaggacgggcgggaagtcgactgcatggccctgctggccttgggaggctgcccagtccttagctaaagctggcagttatgggaaacagacttagattctattacgtttttcaggatgtcccaggagtcacctgggaagctcagcagtcctttgtgactttcaagcatatggtagaagctgctgaacacagagctccctctttggggataatttgcccaaatcatttaatcaggcttgagaaatgagttaccacaggtccaggagtgctgccacccttgaattctgacaccctatttctcctatccgtctcttaattaattaagcagacatccccaagtgcttacgacaagccaggacccttttgcatactaaggaaaacagggatgaaggaaacagaaatggtctctgctctgactcagaaggtagaaatcctctttcccagccaagtcttcctagggagcacgtaggaagggctctgaacccacgtgtcagttgcaggggaggatatcaggaaaggacattgaagaagtggagacctaagtttgagacctaggcattagccaggctagcagtgcttgaaaaagtgtcttaggacaagagaactcaccagtgaagtcccagtggtaggagagcgtgcagcatattctgagcctgtatacacatctccagggcattgcttagcaggtggggagtggcaagagagtaggctggagtcacagaagggaggccaggtagaccttggtgagcactggactctatgttcaggtgctgaggagctggcaaaaggttttaagtcggggagaggcatgttcagatatttggtctagctgagtaactttgggtgctctgtgacaaatggttgggagaccagtgaggtggcagttgcggtcatctaggagcaggatcagagtggcctattgactgggatgactgtgaagtgggatcctttccagccagtaactggaaatgtgtatgagggcagaagtgagtgtactgcatttgaaacattgagaaatctagtacatagtactgtctcttttatatcttttttttttttttttttgattttggtttgtttgttcactaacttggaaaactgatgtggaaatgtccctttggcttcagttacctgagcagaaggggccgggcattgccaaactctcctcttaggacagaattgctcccagtattgatcattgtgttctgagttgggggagcaaattgtgcaggaggccaggtcagtgccaaggtgggtgggaggaattggagcaggaagcttgcctaagtgtgcccagcaaagccacggtagaactttctactgtggctctatgctacttcttagcaaccttctccatgtgcttcctggagagtccttggagtcagaacctttttcttgaaacccagacactttacttccaagaaaatgctgtccaagaaaactcatccttcccttcttctcatgaacgttgtgtagaggtgtgtcttctcttcctttgagcttttccactcagggtttaggggaggtgatattctatatttgggtttggctctgggtactgcaacactaggctattaagatttcatccttactgctttgcccctcctatctttccagaaacccacaatggatttgctagaaataatggaacgtcctgtttggacaggatataaccatttctcagctagaggatattgttggaatgaagaaagataaatggggagaagggaactcacattgctttggcacttaaattaagccatgtactgtgttgggaaattatttatattatctcgttgaatccacagtagaacacagttgaacaccatacaaggtaagtattgtcatccttattttaccatgaggaaattgatgcttagagagcataaagccttggccaggggcacatagttgggaagccggggctaattcatgcctgggctctttctgatagttttccttttttaattgtcccctcctcattgttaccttggggatttcaagagattcatgtagcttctaaatcaacgaactgattcctggagagcagcttctgtatgagaaaaatctagctaattatttatttcagtgtctctggaatgcaagctctgtcctgagccacttagaaaacaatttgggatgacaagcatgtgtctcacaatgctgctctggttgccagtgctgtgctgccagttgtcatctttgaacaaactgatgcagtgctggtttaactcttcctctttttggagtaagaaactttggaggcctgtgtccttctagaagtttgctgagcaaatggtaaggaaaagaaataggtcctaaggcttgactatttcagagaatttcttgatttattggactgtcaatgaatgaattggaatacatagtggtaggctgtcttttcttctcagacactgcaatttcctccaatctcttgacttttctagaagttttaatccaagtccttgttgggtggtagataaaagggtattgttctactagagactgaccttggcatggagatctcatttggactcacagatttctagtctagcgcttggttttgtatccatacctcgctactgcattcttagttccttctgctccttgttcctcatgcccagtgtcccaccctacccttgcccctactcctctagaggccacagtgattcactgagccatttcataagcacagctaggagagttcatggctaccaagtgccagcagggccgaattttcacctgtgtgtcctcccttccatttttcatcttctgccccctccccagctttaactttaatataactacttgggactattccagcattaaataagggtaactgctggatgggtggctgggatacacagaatgtagtatcccttgttcacgagaagaccttcttgccctagcatggcaaacagtcctccaaggaggcacctgtgacacccaacggagtaggggggcggtgtgttcaggtgcaggtggaacaaggccagaagtgtgcatatgtgctgaccatgggagcttgtttgtcggtttcacagttgatgccctgagcctgccatagcagacttgtttctccatgggatgctgttttctttccagagacacagcgctagggttgtcctcattacctgagagccaggtgtcggtagcattttcttggtgtttactcacactcatctaaggcacgttgtggttttccagattaggaaactgctttattgatggtgctttttttttttttttttgagacagagtctcgctctgtcgccatgctggagtgtagtggcacaatcttggctcactgcacctccgcctgccaggttcagcgattctcctgcctcagcctcccaagtagctgggactacaggtgcctgccaccatgcccagctaatttttgtatttttagtagagacggggtttcaccgtattggctaggatggtctcgatttcttgacctcgtgatccgcctgcctcggcctcccaaagtgctgggattataggcttgagccaccacgcctggccgatggtgctttttatcatttgaaggactcagttgtataacccactgaaaattagtatgtaaggaagttcagggaatagtataagtcactccaggcttgaggcaaaatttacaaatgctgctgactttgtatgtaaggggaggcattttcttagaaaagagaggtaggtctctgggattccagtatgccatttccatcctcagtgtttttggccacctgagagaggtctattttcagaaatgcattcttcattcccagatgataacatctatagaactaaaatgattaggaccataacacgtagctcctagcctgctgtcggaacacctcccgagtccctctttgtgggtgaacccagaggctgggagctggtgactcatgatccattgagaagcagtcatgatgcagagctgtgtgttggaggtctcagctgagagggctggattagcagtcctcattggtgtatggctttgcagcaataactgatggctgtttcccctcctgctttatctttcagttaatgaccagccacggcgtccctgctgtgagctctggccgctgccttccagggctcccgagccacacgctgggggtgctggctgagggaacatggcttgttggcctcagctgaggttgctgctgtggaagaacctcactttcagaagaagacaaacagtaagcttgggtttttcagcagcggggggttctctcattttttctttgtggttttgagttggggattggaggagggagggagggaaggaagctgtgttggttttcacacagggattgatggaatctggctcttatggacacagaactgtgtggtccggatatggcatgtggcttatcatagagggcagatttgcagccaggtagaaatagtagctttggtttgtgctactgcccaggcatgagttctgatccctaggacctggctccgaatcgcccctgagcaccccactttttccttttgctgcagccctgggaccacctggctctccaaaagcccctaatgggcccctgtatttctggaagctgtgggtgaagtgagttagtggccccactcttagagatcaatactgggtatcttggtgtcaatctggattctttccttcaggcctggaggaatataataactgagacttgttttatttctgcagagggttctaagccattcacttcccagatgggccaataatgctttgagtaatctggagatcatctttaatgcgcaggtgaatggaactcttccacagagggatgtgagggctgtagagcagagtgaactccctgaaactcagacgtcagctctttgtctctctatctctgaacacccttccttagagatcccatctctaggatgcatttctctgtagttagtttctaagtctcttgttcctgttctgcctttatttttttttcctggattctaagccagtatccccacttggctgtcttaatgtagcttaacatgtctgtaatcaaaatgatcatctttctgagattcaaagggctataagggactttggagagaatttcattcagttttcctcaaactagaataatgcttgcactgtctgtaaaagaacaaaagtgtcaaagcatccttttgttcactaaatttccttttttattatagtgttacttaaatattaggaagttaaaagtaggtataaacttcttataggctgttattatacaactatatgacccatacatatttacaaattaagtgcagccaaaattgcaaaatcaataccattcaaattaataccttaaatgtggtgaggcagctgttgttcaactgaaaccaaattataagttgcatggcagtaaatgctatcatgctgatcattttgagtttggccagtctatattatcatgtgctaatgattgaattctccacccatttttctacttgtatgaccttaatttgatggcacctgttccatcctcatgagtttgctacaattatactggtgccaacacaatcataaacacaaatataaacttgggctttgaaatcttgtgccagaacttggctttaaagtaagcatttaaaaaatccatatgtgtttattagactttgtttagatgactgttgaaatgaaaacaaagtgtttaaaatcctcttagagaacttaaatataatccctcagcaatatgtatacagatcttcctttgagaaaaactgattgtgttcagcctctcatgttacaaatggggaacctgaattctgaggtctctagtgagagaacagggactggaatctgtggatcctatctgttttaataataattgtaaagtataatagataatattatattaaaaagagagnnnnnnacacttagaatgagcttccatgtgtgaggcactaactgattaggcattattaactagatttattccttttaaggccccgcgatgtactgttatttccacatgttgtagctggggaacgtgctactcagagaggttaagtaacttgtctgaggtccacaccactaacaaggagcacaggtagggttcaaatccagataatctgactttggagctggcactctaactcaatgtgcctaatcgcttttcagtggtgtcattattttgcctattctccatctgagaatattgaagtttctgactccttccttgcctttctccctgcctcccgtggttatccccaggtcttggtgttccagtcctctatgtccgtccttactcttattcctttgctacagtgtgatccagggctcctgcccttcttatcctggtagagggggcccacttgctgggaaattgtctccgccatggtttatccatgttgtgtgtccattagtgagtagtgggaagaatcatatcatgttggcaatgaaaggggggctatggctctggggtagtctagtctgaactcttatttt

SEQ ID NO: 15 - DNA, 4736 bp, Homo sapiens
cttttttttttttttttttttttttttttttgaggtgaagtctcactctgttgcccaggctggagtgcaatggagcgatcttggctcaccccaacctctgtctcctgggttcaaacagttctcctgcctcagcctcccgagtagctgggattacaggctcccgccaccatgcccagctatttttttgtattttcagtagagatggggtttcacccttttgaccaggctggtcttgaactcctgacctcatgatcaacccacctcagcctcccaaagtgctgggattacaggtgtgagccaccacgcccggcctcataagtattttctaaatttatttacagtcatgccatttaaaaggaaagttgtattcctgtctttgttaatatttataagtgattttattcagctacaagcttggaatggcatataattttgtattctgcttttttcacttaatattacatggctaatgatttctgtgtttcataaacattattctgatgatggcatgatatattgttgagtacatgtaccataattgaatcatttccctattgctatgcaattaagttgtttccaatattttgcaattataatgtttcaatgaatgaataactttatgcatatagctttttgatatcttaagttcagtttcctaggatgaatttccaggaatagtaattgggcaaatgggataaacatgactcttgaatacgtattgttaacattgctttcccaaagggctcaactgatttatatttccgtgttcattatcttttaaaccagctcatttactcaccaaacatttttaaagccattatcatgtggtaggcttagtaagaagaaagtgaccctaagggagaagcttatatataaatagggtccctggtgtaccaagtgctgatacagacacaaagtacctggggaaattgagatgagggagtcctggctcagctgggagaaaagttcattttcatagagtcatggttttgttctttggcagaaagaaaattgctttcttccccacccccacccccagctttattgaggtataattgacaaataaaaattgtatatctttaagatatgcaatgtgatatatatgtatatctcaacttaaaaaataagctacagaataaaaaggtgtttgctattaaaaaaaaagaaaaggctgaatgtcattcccaagcttggaaatttgagtatgttgcctctttgggattatttacagaaatattagcaagaccagccccatctttggtcttgagtactccactgtcagcatgctttcttccagagagggatccatttgcctttatttttcattctgttgtgccgtctatgcaaactattcttgatagttttatggtaacagtgtttttttgttccatgagataaatttatacatgctcattgtggaaaatttagaaaagacaggaaagtattaaaaacatcmcytttttttttttttttttttttttttttttamgcagacagagtcttgctctgtcgcccaggccggagtgcagtggcgtgatctcagctcacagcaacctccgcttcccaggtttaagtgattctcctgcctcagcctcccaagtagctgggagtacaggcatgcaccaccacgcccggctaattttgtatttttagtagagatggggtttcaccatgttggccaggctggtctcaaactcctgacctcaggtgatccgcctgccttggcctcgcaaagttctgggattataggcaggagccactgcgccagccacacctacgttcttatcatcctagtacatccactgtcattatcttgctgtatttccttctgcccagtctcactctgatcatgcagtggcgtgatcatgcagtgatctcggctcactgcaacctaggccttctgggttcgagtgattctcctgccttagcctcctgggttcaagtgattctcttgccttggcctcccaagtagctgggattacaggcatacacccccatgcccatctaatttttgtatttttagtagacacagcgtttcactaaaattttgtatttttagtagagatggggtttcaccatgttggccaggctggtctccaactcctgacctcaggtgatccgcctgccttggcctcacaaagtgattacaggcatgagccactgcatccatcgccaaaaagattttttaaaagagtttaatgtagaaccatatcaaaggtctttggaaataaaaaacagttttttaaaaatatcagaaataaaacaacaaataaataaataaataaaaacacccaaaacaatctgaagcacgagcacctagcagaaaggttcaattatgatctattcatagagtggaatatcaagtagacattacaggacatgttttaagattatattttatgtcatgggaaatgctctcccagtatgatgttaaatgaaaaaacagaatacaaaagtatatatgctgcatagtctcaatattgtagagaaaaaatattatttatgtatgcatgaaaaaagacaaaagatgttaacagagatccattgttacttcagtttactagggattgtctctgggaggtaggattaaggtgatttatatttacctttttaaacttttctgtatttttttattttcaaattttccataaaaatataaggacttgaagatcaagaaaaaatttctgctttggctcagtgcagtcgtcacgcctgtaatcccagcagtttgggagccctaggggagaggatcacttgaacccaagagtttgacgttccagtgagctatgatctccggatcgtaccgcctggacgatggagcaagaccctgtctcaaaaaaaaaaatctttgctttttttttttgtttgtttttgagacggagtctctctctgttgccccagctggagtacagtggcacaatctcagctcaccgcaacctctgcctcctgggttcaagcgattctcttgcctcagcctcccaagtacctgggattccatgcacccaccactatgcccagctacttttttgtattttcagtagagacagggtttcaccatgttggccaggctggtctcgaattcctgacctcagctgatccaccggccttggcctcccaaagtgctgggattacaggcatgagccactgtgcccagcccaatcttttgctttttttaaaaaaagaagacaaaaagggattttataccagtattatcttggctgtgtgactctgaagccacagttgtaagttataattactctgaaacacaaggccctgtgactcttttgggctctttggtgtttatcttgattacaacgttggaatatagaaatgaaaggaatgggagaggtgatagacttcaggcagtgtaactagttgtctgaacactactggctcaattatattgtgtctagtgatttccatcttgtccgtctgctaatttatcgcctggtaactcactgaggcagggttttcctttggagaaacctcattgttttaaccagtgtatcatgcttgtttagaagttcaatgatctttttaactcatcggagaagatgatgaccagacctggacagatggggaaggactttgcactctctctttacagtcctgagtgcacacaggtcaatatggaactatgtgtgaattttcattgtctttgagagccctcttctctgccccatagggagcagctttgtgtgcaattagaggagcaagggttgtgtgtatttagcacagcaggttggcctggtcctctcctctcaacatagtcaccacatacctggcactatgctaaggctgggaatgcagacagatgggtgcctgctttcagagtgctcaatgtgctgaggaagccagcaacagaaacagatgatttcaggagctccaggaaaatgctacaggaggagtgtgcctgggttactggagtagcacaggaggagggcttctagctcaggctgagattttagtaaaggaaattatgccacgatgaatcctgaagaatgaatagaagtgaaccagataaagcacgataggaagcatcttcccttacctaagggaagacacagaggtatatggaatggtatgttaaaaggttgggactccaaacagttctgttaaagcttagagagtggtgggagagactggagaagttgattaattagtaaatgaagttgtctgtggatttcccagatcccagtggcattggatatccatattatttttaaatttacagtgttctatcttatttcccactcagtgtcagctgctgctggaagtggcctggcctctatttatcttcctgatcctgatctctgttcggctgagctacccaccctatgaacaacatgaatgtaagtaactgtggatgttgcctgagactcaccaatggcagggaaaatccaggcaattaacgtgggctaaattggacttttccaaagatgctgtctttgggaaacatcacacatgctttggatcagaaaacctaggcttctaatttgttgataaggcatgaactcaggagactgttttcagtcctagtgaatggtgataattgtaattataacagtagacaacatctcttttacacattttaaatcatgaaaatagaataaccttactgataattttagaaagtggtgattaaaagcacatttaagataatgccttaacacctagtcttttccatatgcatgatgtcttaatcacacattgcaaatcatggaacacagaatttt

SEQ ID NO: 16 - DNA, 4768 bp, Homo sapiens
atcttacaatcacagtctttctcttagggctgggctcagtgggtggattgacactgcagaaatggccagatctaaaggatcaacatttacgtagctgggaaatgtagctgggacttcagtttcactgccctagtgatttttcctaccactaagcagctcagtccatacccctacgagacccacaagcttatgagatactgttcttccaggaaagcagtggggccagggccaccttttaattgtgtttcttggcctggtcccatctttctcacaatatatagcaacagttatttacttgctgattttctaatgcacatcacacatagtcatattaaacacacacacacacacacacacacacacacacccctcaagaaacattttctgagacgtgatttcctgatttcatcaaaaaagaaaagagcgggccaggcacagtgggaagtcaaggtgggtggatcacttgaggtcaggagtttgaaaccagcctggccaacacggtggaacctcgtctctactaaaaatacaaaaattagccaggcgtggtggcgcacacctgtaatcccagctactggggaggctgaggcaggagaattgcttcaacctgcgaggctgaggttgcagtgagccgagattgcgccattgcactccagcctgggcaacagagtgagactctgtctcaaaaaaaaaaaaaaaaaaaaaagcataaactgaaatttatatgcaatttatatgcctgtgagataattctgttttctcttttggaaccccaaagagatttttttgattgatgagcaaatacattttagattttatttaagcattatgccaagcaccactgaagtataagtttcaagggcaaactcagttttttcatctactagacgaatgattttctggaatgattacaagcaggcaagatggtgtagtggaaatagcaaatgtcttcggcatcagacaagttggggtttgtttgtatcctgcctctgcccttcaccgaggttgtgatcttgggcagattgttgagttttaacctagattcctctgactccagatcataaattttcagaaaagttctgaaattcttgtatatactgatggtaaatgagacttttccttacatctatgcacttctttgtttgtttgttttgagatggtcttgctctgttgcccagactggagtgcagtagtgcaatctccgctcactacaatgtctgcctcccaggttccagtgagcctcctgcctcagcctcccaaatagctgagactacaggcatgtgccaccacgtccggctaatttttgtatttttagtagagacagggttttgccatgttgaccacactggtctcgaactcctggcctcaggtgattcgcccgcctcagcctcccaaagtgctgggattacaggcatgagccaccatgcccggccatatccatgcacttcttgcaaccttaccttcttttctcatcaccctccagggacctagttggaagagcagagttaaaagttaaggtgaaacttggagaggtgtcttgtccctaggaacaaaggactggtttgaaattctctgtaaatcttccccagttcaaaccagagttatcaaggtcttaaaaacttccctgggtcctgagagcccattatattatttacttgtcttcctgtacacccactgcctagtcctgatcctacttttgtttgcaaataggatggggcacaacgtacaaggaagggcctttgccacccctgctaagggataacctgaaataccttcaccatcactgccctgtgctgcttttcacctatgccagtctgtctacagtgccagtgtctcctggcattgaaaggggagaatcttttggtcctttgagtatttggttgggttacataaatctccctgaatgaagagcagctgacttaggcaaggggccttgtttggttttccttgaactattaacaggaagatagggagattaactgtgtaaatgttcaataggccagagtccctgcagagggtggccacagtgatcagatcttatcacatccttgctttgggtgttgcctctctggttggagtatggatagaaaagaaagaaagaccctatattgaaatgcaaagtgcagcaagtcctgactttggattaacttctcagcccatttgcatgaaaataaaaagatgaataaaacaaggttcccactttggagggaggtggtagctgtgagatggaaggagtgttcctgctgggcaacagcagagtaagtgctggggtagattcactcccacagtgcctggaaaatcctcataggctcatttgttgagtctttgtcctacaccaggcactctgcaaaaacgctttgcctgcaaggtctcatgcgatgctcaccacagctctgtgaagttaattgtacttttatcaccattttacagatgagaaaactgagggtatggggtcaatgacttggctaaagtcactgcttagcaagctgcagggactggatgtgaattccaattggtttgactccaaagcctgtgaagctacttgttcttcaccacctagagctgtggttcttgataactgtgaactcttttggggtcacaaatagccctgagaatatgatagaagcaggagctctggcctttctgtccatacctgaacaggtccttgggttaagagcccctcgtccagggcctattaatcttgatcctcataagcagcatccatgtattacggccgcaaaccaaactgtgccagaccgaatcctaggaccaagcccaaatatgtcccatcatccttttggtaagaagctcattgtaagaaagaaagaggagagcaagaggatgacctagtgcatggggcctcattgttttaattagtgacaaaacaacaataataacaacaaaacccccgaagcttcacagatgacatcagaccccaagcctgtgtgtttttcaggtgcccttgaggagctttgtagctggcagaggaggtgaaactgacaaatgtttggcagatggaggagagtaccagaggggtttgagatgagctaaattccaatctaaccgcagtgttgaggaagaggcttggattgggaccatggagatgggggttctactcccagtcacgccagctgactttgcgagtgttctttgtcagtcactttatcttattttatttatttttatttttttgaaatggagtttcgctcttgtcgcccaggctggagtgaaatggcgcgatcttggctcactgcaacctccccctcctgagttcaagcgattctcctgcctcagcctccagagtacctgggattacaggcgcctgccaccaagcccatcgaatttttgtatgcttagtagagacagggtttcgccatgttggccagggtggtcttgaactcctgacctcaggtgatccgcccaccttggcctcccaaagtgctgggattacaggcgcgagccactgtgcccagcccacttcatcttaccgtagttacctccttagagtatgaaaaaataggcttagggcatccccaagtcccctctatgtctgagagctgaggctggctgtcaaagaggaactaaggatgccagggactttctgcttaggacccctctcatcacttctccaacgctggtatcatgaaccccattctacagatgatgtccactagattaagaatggcatgtgaggccaagtttccacctgagagtcagttttattcagaagagacaggtctctgggatgtggggaatgggacggacagacttggcatgaagcattgtataaatggagcctcaaaatcgcttcagggaattaatgtttctccctgtgtttttctactcctcgatttcaacaggccattttccaaataaagccatgccctctgcaggaacacttccttgggttcaggggattatctgtaatgccaacaacccctgtttccgttacccgactcctggggaggctcccggagttgttggaaactttaacaaatccatgtaagtatcagatcaggttttctttccaaacttgtcagttaatccttttccttcctttcttgtcctctggagaattttgaatggctggatttaagtgaagttgtttttgtaaatgcttgtgtgatagagtctgcagaatgagggaagggagaattttggagaatttggggtatttggggtatccatcacctcgagtatttatcatttctgtatgttgtgaacatttcaagtcctgtctgctagctattttggaatatactatatgttgttaatgatatcatgcagcagacgtgcatctgaatgggctggctctaggagctagagggtaggggctggcacaaagatgcatgctggaagggtccttgcccataagaagcttacagccaaggctaggggagttctgtcttctctgcatcaggtcacctctctcacctctgtcactgccccatcagactacaatgtctgcaggtctttctcccctgagtgtgagctccctgagcaaagcaggatgctgccccttccctttgtattccttgctccttgcttcagtgcctgtacataagtatgggcataataagtgtcccccaaatgagacattgaggattcttcaaatgcacaggaccgtgatgtgagttaggacggagtaaggacgatgggatgtggctcatgacaatcctgaggaagctgcagctgcggcacgcagggccacactgtcatgttcatggaccctagactggctttgtagcctccatgggccccttccatacac

SEQ ID NO: 17 - DNA, 1295 bp, Homo sapiens
tcatgactgccattggtataaagatgaatataatccagaccagattcatgattattcatacatttttagtgtattaacttttaattctgcttttaaaataaattaaaacattctaatatgcccttaagagtatcccagcccaggccactgagcctactgtggttcatggataagtttgcccctgggggcatgtgtgtgcatgcatgtgtgtgcacatgcatgatgagccgggccttgaagggtggtaagatttgggtgtgtagaccaatggagaaaggcatttggggcagtgatgatgggtgggggagggaacatggtgatgaatggagctgggtgtggggagccatgggagtgggttagggccagcctgtggaggacctgggagccaggctgagttctatgcacttggcagtcacttctgtaaagcagcagaggcagttggcctagctaaagcctttcgccttttcttgcaccctttacagtgtggctcgcctgttctcagatgctcggaggcttcttttatacagccagaaagacaccagcatgaaggacatgcgcaaagttctgagaacattacagcagatcaagaaatccagctcaagtaagtaaaaaccttctctgcatccgtttataattggaaattgacctgcaccagggaaaagagtagcccaggtgtctggggcttgttcccattagatcttccccaaggggtttttctccttggtggctggcctgtggggcccctctccaggaggcattggtgaagaaactaggggagctggttgccacagacagtgatgtactaatcttctctgggaagacagaagaaaagtccccagggaagaatactacagacttggccttagggacagctaggggtgcagattgctgccaactgcattttttctgaagttggccatatggttgcagtgaatggatttatagacagagtatttctgtgcatataagagcaattacagttgtaagttgatatggataagtgaaagttaagcacttctttctaaaaagagaatgcaattcattttcccctaatcatttcaattagtctgatgggcatttgaacttgttgtctttaaaaagtgaaatctttacctctgatctggtaagtatccaggcaatttcttgtgtgccacccaggaggtatctggggagtgggcattttctgactgaggcattggctgccatagcatcagagcagccttccaggcagtggcctggcaaggggacagaggctggtgggagcagctggctgagtgcagccagtaatggcatgt

SEQ ID NO: 18 - DNA, 2188 bp, Homo sapiens
agctctccaggtgattctgatgcatacttaagtttgagaaccattgcttgttttgcattaaacaggagattagtctctgcagcttgtgggaataaagctttaaatctctccaattttagctctgtgaaaaggcagtggggagacaggaatgaacggactagtgccacaaagctcaggtggggtgggtgagatcatttagaagagaaagaccgggcatggtggctcacgcctgtactgtcagcactttgggaggccaaggcaggttggatcacaaggtcaggagtttgagaccagcctgcctatcatggtgaaaccctgtctgtactaaagataaaaaaaaaaaaatttgccagtcatggtgatgcatacctgtaatcccagctactcgggaggctgaggcaggagaatctcttgaacccgggaggcgggggttgcagtgagctgagattccaccattgcactccaacctaggtgacagggtgagactccgtctcaaaataaaaaaaaaaaaagaaaaggaaaggctgtgtgtgtgtgtatgtgtgtgtgtgtgtgtgtgtgtgtgtgtaacagcaccatcacactgtttgagttgaggagcacatgctgagtgtggctcaacatgttaccagaaagcaatattttcatgcctctcctgatatggcgatgctcccctatctcattcctgtgtgtgtttagccaggcaactgttgatcatcaatattatgataacgtttctccactgtcccattgtgcccactttttttttttttttgagttacttactaaataaaaataaaacactatttctcaatagacttgaagcttcaagatttcctggtggacaatgaaaccttctctgggttcctgtatcacaacctctctctcccaaagtctactgtggacaagatgctgagggctgatgtcattctccacaaggtaagctgatgcctccagcttcctcagtagggctgatggcaattacgttgtgcagctactggaaagaaatgaataaacccttgtccttgtaatggtggtgaaggggagggaggtagtttgaatacaacttcacttaattttacttccctattcaggcaggaattgccaaaccatccaggagtggaatatgcaacctggcgtcatgggccagctggttaaaataaaattgatttctggcttatcacttggcatttgtgatgatttcctcctacaagggatacattttaagttgagttaaacttaaaaaatattcacagttctgaggcaataaccgtggttaagggttattgatctggaggagctctgtctaaaaaattgaggacaggagactttagacaagggtgtatttggagacttttaagaattttataaaataagggctggacgcagtggcactgagttgagaactgttgcttgctttgcattaaataggagatcagtccctgcagcttgtgggaataaggctttaaatctctccaattttagctctgtgagatggcactggggaaacagaaatgaacggactagtgtcacaaagctcaggtgggatggacgagatcacttcaaaggtctgtaatcccacgtctataatcccagcactttgggaggccaaggcgggaaaatcacttgaggtcaggagttcgagaccatcctggccaacaatgcaaagcctgtctctactaaaaatatgaaaattagctcagcgtggtggcatgctcctgtagtcccagctactcgtgaggctgagacaggagaatcgtttgaacctgggaggcggaggttgcagtgagccaatatcacgccattgcactccagcctggctgacagagtgagactccatctcaaaaaaaaaaaaaaaaaaagaattttataaaatcaggaaataatattagtgtttatgttgaattttaactttagaatcatagaaaacttcctctggcatcattattagacagctcttgtgcagtgggtagcaccagacccagcttgcatggttattgatttttcagagacactttttgagcttattctctggcagaaaggggaactgcttcctcccctatctcgtgtctgcatactagcttgtctttacaagaagcagaagtagtggaaatgtttattcttgaaaataagctttttgcttcacatgatctagaatttttaaaattagaaaaatgtgcttactgcg

SEQ ID NO: 19 - DNA, 1183 bp, Homo sapiens
  misc_feature (1)...(1183): n = a, t, c, or g
agtaaaatggagaattccaaattctgaaattgttagaacatagttctgtgtcttagttaaatatcgacacttacagataaatagcataaatgctttctccccatatttcagcccagtcctacttaaagacaacataaattgcaaaatagtgaggatgttgttcatctaataaaagtggttccaggaattcagactctggattcctgtttgccaaatcatgtgtcccactcttaagaaaacgagttggactntggatttttctttgcaagagggacaagagtgtgggagatactgagttaatgcaacttgcaggttttaagtgtcctgtcattgtgccttgtgctttgatacattctgagtttcagtaaagagacctgatgcattggactgttgcaatggaacctgttttaagatcttcaaagctgtattgatatgaagttctccaaaagacttcaaggacccagcttccaatcttcataatcctcttgtgcttgtctctctttgcatgaaatgcttccaggtatttttgcaaggctaccagttacatttgacaagtctgtgcaatggatcaaaatcagaagagatgattcaacttggtgaccaagaagtttctgagctttgtggcctaccaagggagaaactggctgcagcagagcgagtacttcgttccaacatggacatcctgaagccaatcctggtgagtagacttgctcactggagaaacttcaagcactaatgctttcggaatgtgaggcttttccttggacagcatgactttgttttgtagaaaagtacggctggctgggagtttgtgatataatttagttcagtggtattctaagtgttcttagtgttctttcagacttttgggccatctcccaaagggtgaatgggaagaataagctgggtgtggctgagtttaagccaaaagttttttgtgcttgtttcaatcagagaagacctgctttttcatgtttttactattataatactaagcaagagctcatttgaaaacagagttcttcatatttaaaaaaaaaaagtcttgaaaccattgatgggaagatggatatctatttatgtttaaaaacccatcataaagatgacattgtgggctgtcacagttggaaggccctggaattagatgagaccacactatttagcttacttagtaataacattg

SEQ ID NO: 20 - DNA, 8981 bp, Homo sapiens
ccgtttggcaaatgctcagtaaaagaaaagggttagaaggggagaaaggcattttatcccaagccttcaggaatcaggatgaggatgtcttcaccttgtggtggggagtaattatacaattagagacagcacattggagtgtggctgatatgctgtgtgatgatagctctagctctctgcctagcagaggaaggacatttcaatagaagaaaaagtttaagaccttgccgagaaacagagaaaggatgtttgtctttttaagaagttgaaaaccctgtttgcagacaaaagccctccagttttggcagtaaactttcatgcaagggaagaaaaaggcaggggatgacattgttgacaattgtgaggaattaccatgtgccaggcactgtgcgaggggctttgtacatatcctctagttttagtgcttataaaaactctgtgatatgtgcacagcattttaaactttgctgcatagtcgagaaaatggaaggatggggaatttgagtcatttgcccagggttctatagctaccccaggttcccatgactggagaattggggcacagggtggcgggggagagtgagtgacaagaatcctaacaatcttatttccattgagtccttataaaagaagtggattaactaccacgtttttaagtttttcttaaatttaggttatgtggatctggcgtttcttgttttgtcctgggtttgttttgtttttgctatgctgtcttgaacatctgtcatcttgtaggcctaacggtaaacacaaaaacactttacctcctatagctttcaattaagatctctcagtttgtgtttgtaatagttttccaggcaagttctccctaggttcggcttctagtgtgttaacctttagttataaagtgaacccaaagagagaaagtagaaacaaaacacctcacctgtttttgctcatgaattactctctatggaaggaacaatcatgaacacctctgcgtatcacagaggcctatctgagtctgacgtttaagggagaccgcgtaggtccctttgaggactgtgaatgtgggagtcctgggactctggtgaagaacccgttccagaagagatgaatgagctggacaagttctttcatagaacctttaggcaggttttcttagaaatgcacattgaggattatgcttggatattgtgatgatcagaatgatactcaatcccttctgcatttggaattctctttgaaagaaaacatcccaggcagctatttctcagagatagtgagtcccagccacttctagacattttcttgtgtagtctacattataatttcacagcagtctctgatatgacaaatgtcaaaatagcccaaccttctctaaacttcagagatgtctgatatgatattgaataaaacaatgctcatagaaacatcaagaaaggtggattttccctggatacttttttcctgcttgacaaataacagtgaagaaactgatctcacgtctttttctctttggaagcctgaacactcagaacccaacttgaggctcctcagctatagcaattctgacttcacagtctgtaaattattgttcttttttttctttagcttatgctttctgccctaatttatcttttccctgttctaatgaattattgtcctatatctgctgtgcagttaggtgacatataacagcaattaaatatatgaattggtacatataaagatttgactaaaactcgatgtaaaaataagtgttctacattcaatttccagtgttagaaacagtgctgacttgaacagagtgacagaattccatctttccctatttttgacagctttaaactttatattttcttcctttcttgtgagccgtcattaacttgtttctcaaagccattcccgtattacccatcttgcagacgcagacagatttgggaatttgcggtcagagttgtattggacacatccccccagcccacatgagatccttttaatctattgcatattaactagttttaagtacaatattcctacttcatttaaaaccattaatcaaagaatgagtttgaaaatgaacaaaatgcaaacttacagttagaaataattgtagtgtctttagttttggttaggagtcggtttcttgtttgttaaactcaagattgtgaacagttttaattcacttgtttatttccaatagagatttcaggtttacatttgaattcagaaacaaagttttctttctcattacagagaacactaaactctacatctcccttcccgagcaaggagctggccgaagccacaaaaacattgctgcatagtcttgggactctggcccaggaggtaagttgtgtctttccagtaccaggaagcggatcatccactgtatcagtattttcattcctgagtctggcaagaggtccttttgagttgaatatcacatgggatgtaatatcaattttcaaagtataagtgatgtaaacaataatgttttgatttccttattttagaaatgaagaaacctaaaactcatagatgtctcagagctaattggttagtggctaacagctggatatctagtttagaaccttctccattttttctttttgcccctaggtaatcatacatttgtaaagaggagaattatctctgccactgcccatgcactgcttttgtctgaccagcaatttctccatattgcttcttcagtagcaaggccaatcattttaccaacacacatgcttgctaactaacaggaataacgtggtacccctaattcagccctttcccttgaaagcatctggcttctgaggttcaactatgggaatatggtctcttaatgaacattaagttgagtttgccttttaggtccacatgttgacaaatgtatcagagtaatctctgtcctaggatcagagggcctgtaggcacttgcaaaagcagttagctctgactcccagccagtgcacactccacctttctgactcccagccttgtctcaaattaggcttggaagcgaggaactgtctggtgtcccccagcataggaagctgagccagggggcagtgctcacaaacaatacagactttaacgtgtaggatattggaaaataataatttgtggggaaattgtctcagacttggtccacccttatttttagctgcttctctaatccgtttttctttttttggtgcttgtatctaacctacccattttttggtgcttgcatcattttttcaaatatcaaaaacgaactttatgttttctaacaatgaaagtattgcatgttcattgtggaaaatgctgaagacttggaaaatacaaaaatgctgagatcaaacactattgatacgttagtgtatttcttcctgtcctgttctactttctttctttgaattctgctcacgtgtttctgactgatgaggtctgacttttgggttccttttccagaggagaagccttctttcagcttgccatttgttaccctggttatgaaggctggtaaccttttttactaggtagagaagctggaccaactggggttcttccagggggagaatgagaaagagaaactgttttgcaagtccgtagctatttctctagggccctgttagctgacattgacatgccttgcattgctctgcagatcccctcgcagccctctgtcccttgttcatttctggccttagagaaagcaaagcagggtctgtaacaggggaggctgcctctaaactcagggtttggttacagctgttttcacttacatcactggccctggttttttttttttttctggcattaaaaaaaaaaattggaagcaggtgatgttcccattgctgatgtggtggaaactctccaagtgaacaatatacgtttttcttggcagctgtttcttgtgccctgcttgctcctggtccaggacaagcaaggaccatctgcctctttcaatagaacacctccagatccctttgatcaaaagttactcattgtctgacttgctatttctgtgagataaatgggagaagatcaataaatgcacttgtttgtccagtcagcgtgtggaaagttgataattttgaccaaagcacaaccctgaaaggaaaagaaaaagggagtgaatgtcttctgagaagctgcctaggttcagacagtgtcacccatttccctgtatgctccacatgacaaacctgagtgggtctcatcatgtccattttgcagatggcaccaaggctcagaaaggttaggcaacttttccagtcacccaatgagttaattgacaaaactgggattcaaacccagaactgttggattccaaagcctgtgttgttgcctgcttcgtgaaaaactccagtagcgactggaatagaaaggagaaccttccaagaaagaaaatacgcactagcagaacctggaaattgggaggaaatgaggacttgaggaataagatgaatgaaagctgacctgagtttcacatctgggtgatgggaagggaggacagggaggcagcatctcagatgtccacccagcaccgaccagctgcctggcattgctaggtgttgaggactcagcagtgaacacgctaacttctctgctttcttggggcacgtatagggtgagagacagaaacaaacaggtcagtgtacaatgccacaggagggatatatgcagtgaagaaaaagcagggtaaggggcatagagcatgagaaggtgctttttttaaaggggktgattaggaaagctctctctaaggtgacagttggacctgaaggagatgatagcatgtctgtggtgagggaaggaaactccgaacaggaagaatggcagatacaaagacattgatgctagagcatgcctaaggaatgtgtttaaggaccagggaaagtgagcaagtggtggggggaggagaggagctcagagcaggaggaggtgagtgccatacaggcctggcaagactttggattcctgctgggtgagatgagaatccagcggagggcttgagggaggggacatgatgtgatctagagtttagactgtttacactctggttgttgggttgagaagagactgggatgggggaaagggaggacaaaggacattgtgctggattgagaaagcagtaagtcagtttcattcattcactcaaccgatgatgttcaaataccaccatcatccgtgggctaaaggatgaagagccatccctccctgagagtcaggaagcacttcccagataaagtttggagtgtgagctgaggtgtaggagaaagagtaagagtttacccctgaaacgggtgctgggaagagtcaatagtttggaataactcaataatttatggtgcttctttagaaagatttgctggctttatgtgggaagaaatttktttttttgattggggagtggtgggttggtggtgaggctgcctgtggaaagagaagtgagtgttttgactcactgttatttaaaaatctctagggctgttccaataagcaacaaaaggcaaaatggcctggttctctgtcccctttctgtctgtatgcctcgtacaggttatgaaaagaaaaagttgggaaaagctgtccacctcacctaattgtgttcttgtggagtgtgctagatgccccctctctggagaaaaaaaatccttgtggcctctgacccacctctggagagcctagttcccttctggaggcagaaggcaaagcttaggacctagagagtgctggaccacgccactcacaggaaccagcaggctgtgaggttgaaagctaggcatatggagctttccaggctgggtgcagggcctcgtggcccttcccctcccctctgtgctctatagctcagtcttcccaggcggtgtgaacacgcagtgacatttccaggaatacagggatttattaatgatttcttgtgaaatgtttggaaatacaaagtactctataaatatttcataatagcattggggctgagaactccacaaagtgccggaatacatttgcatgtaagacagaacgctgcctgggtcattgatgcctgttgagtggcagtcacagacactgcctagggtttctgactcacgctgttgggactgttctatgcagggcaccctcttgtgtggcataggatttgtgcctcaccacacactgttgtagctttgctgtcttgatgatgagtagagggcagtgtccaggccatggtataagcatctactgccccccagggttaccaaaaccaagccaagttgtgtctcagcgagctccgtgaagcatggagaagttgagtactcagagacatgacgtgacttttcaaaggctgtaagctgacgagggacatagctagggttcagacttgagtttttctttttctttttctttttcttttttttttaagactgagtcttgcttttgtcgcccaggctggattgcagtggtgcttggctcactgcaacctctgcctcccgggttcaagcaattctcctgcctcagcctccccagtagctgggattacaggcacctgccaccatgcctggccaacatttttgtatttttttagtagagatggggtttcaccatgttggccaggctggtcttgaactcctgacctcaggtgatccacccgcctcgacctcccaaagtactgggattacaggtgtgagccactgcacccggcccagactcgagtttttcatcttaatgctttttcattgcctgacactttactgagaccaagatagggaacttcacatacagtaccttttctcccaaggcggaagagggctgttcaatttctacactagagttcggggagttttagaaatgagtcagttatcgaggatgagagcagttcctgataggctcaaccacaatgagatgtagctgttcagagaaagcattcttttatctataaactggaagataatcccggtgaaacgaagcccagccccaggggcttcactaactccaggctgtgcttctcaaactttagtgagcataggaatcacctgggcatcttgtgaagctgtagatttgaattctgcaggtcggcagaggggtctcagaatccgcatttccaacaatgtctccagtaatgctgatgctgctcgtccctggaccacagattgggtagccaggttctggcaagctcatcccaaggctttgagatgacatcagacaaaatatgttctgggacatggcttttgagaggtcaagaaaataagatgtttctttctcttctcatccccaacccttgcactgcccttttctcccttcccctaccctcctttctgtccccatccctgacgccagctgttcagcatgagaagctggagtgacatgcgacaggaggtgatgtttctgaccaatgtgaacagctccagctcctccacccaaatctaccaggctgtgtctcgtattgtctgcgggcatcccgagggaggggggctgaagatcaagtctctcaactggtatgaggacaacaactacaaagccctctttggaggcaatggcactgaggaagatgctgaaaccttctatgacaactctacaagtgagtgtccatgcagaccccagccctgtccccaaccccatccctcccttagttctggccttggcctgtgtcatctcctccctctgtagcagcgttagatgtctacatgcccatttgcccaccagactgagctcttcctagaggagagaggcttctcttgaatagctacctgtccccagttctctgaatgcagcctggcacatctcaggtgcacagtagtgtttatcaatggaatgaatgattgacagccaaccttctggttttctgggggatgtggaagggtggcttccagggtgatcaagaatgagataatggcagaaggacaaatcctgcaagatctcacttatatatggaatatatgtaaggtagaaagtgtcagtttcacatgatgaataagttcctgggatcttgatgtacatcgtgatgactatagttagtaacactgtatagtatacttgaaatttgctaagagagtagatccgaagtgttcacactacacaaaaaaggcaactatgaggtgatggatttattaacagcttgattgtggtgatccttttacaaagtatacatatattaaaacatcacattgtataccttaaatatatacaatttttatttgtcagttgtaactcaaaaaagctagaaaagcatttttaaaaaggatgatgtactggtcttaatattaccattgagataagctttataataacataaaaagaaataacagtaatgataatagcaacaacaacaacaacaaagaactaacatttaagtagaatttcttgtgcactgtgcattctgtttaagttatctcattttaccctcatgataacctgcagggaagattctttaaccccacatttcataggctcagagaggttaagtgccttggttagagccacatcagagttaatccacaagagccaggattcaagcccaaatctgcctggatctgtgctctctaagataactgttagtggtggcgtgtgtgttctcacactcagacatttgatctgccctttgtttcccattcttagctgcaaggcagtgttaaagaaccctgtgtctccatatccactccccacacttaagcacttttgtgggcccgtgtgccgtatgcctcgtggcagcagggatccaatgtcacagttttaggcagtggcatccttttccttgaaaacttgatgcaggggaacctttctccatttccaaccacaggtgtgtctttcagacactgagtgaggcaggttttgtactttattgtaacacaagaaccttttcttctctggagtaaagcactccagacattcgcaagttgctttacaagccttaaaaggatggtattgtaggcaactttaattaaatcccatctcctcctctcccccagcttgcaagttgacccaaggaagccttcatttccatgacagacttaattgtgagggcatcctca

SEQ ID NO: 21 - DNA, 20284 bp, Homo sapiens
  misc_feature (1)...(20284): n = a, t, c, or g
actgtgttagcaaggatggtctcgatctcctgacctcgtgatccgcctgtatcggcctcccaaagtgctgggattacaggcgtgaaccactgcgccctgttgagaatttttttttttttttttgggagaaagagtttcgctcttgttgcccgggctagagtgcagtgacacaatctcggctcactgcaacctctgcctcctgggttcaagcaattctcctgcctcagcctcatgcgtcaccacgcccagctaattttgtatttttagtagagacagggtttctccatgttggtcaggctggtctcgaactcccaacctcaggtggttcgcccgccttggcctcccaaagtgctgggattgcaggcatgagccactgcgcccagccccaaattttggtttttgcttgaaaactgaggtctgaattcagccttctggttgcccctcaagagtcagtttaaatgttggtcatgttagttgtcagtgaaaacaatggtgaggctggcatgagagtgtgaatctggatgggagggcttgtgcttcatgaaaacatttttccagatcagctcagtcgtgagttatccgtcattgacgttataataagctctgattatttatcaagcatcattctttatagatatctcagtttaatctgagataatcttctccacatctctccacatagatgttatgaattttacttttacagaggagccaactgaggctcagataagttacttattatatgactagtagtggtagagctggggtttcaactaagaactctctggctccaaagcccttgtaagtttctatcagtatatgaccatgcatatgagcatttgtctctcctcttcttcatagctccttactgcaatgatttgatgaagaatttggagtctagtcctctttcccgcattatctggaaagctctgaagccgctgctcgttgggaagatcctgtatacacctgacactccagccacaaggcaggtcatggctgaggtaagctgcccccagcccaagactccctccccagaatctccccagaactgggggcaaaaaactcaaggtagcttcagaggtgtgcgctaagtatactcacggctcttctggaattcccagagtgaaaacctcaagtctgatgcagaccagagctgggccagctccccagtcgtgggtatagaatcatagttacaagcaggcatttcttggggatggggaggactggcacagggctgctgtgatggggtatcttttcagggaggagccaaacgctcattgtctgtgcttctcctcctttttctgcggtccctggctccccacctgactccaggtgaacaagaccttccaggaactggctgtgttccatgatctggaaggcatgtgggaggaactcagccccaagatctggaccttcatggagaacagccaagaaatggaccttgtccgggtgagtgtccctcccattattaccatgtgcctgcttgatactggagaggtgagtttctggtcactttcccaggtgtgagtgaggtgagaattctttcagtttatctagctgggggaatgtagtgagcatagctaaagtcacagggcaccacctctccagaagtacaggccatggtgcagagataacgctgtgcatatcagcatccatgccactcacggtcaaatagcagttttctgcaaaacttagtgagggctggtgtttggaagtggagttgagtaattgcagtaccctattttcctttttgctgcagcctctcagccagccacagcatctccctgtgtcttggtaggttttggaaagaagtgtgggagcaaaagcatgatgttacatgtagactggcctgagatactcattctcagggcactgtgtgaatgatgagctgctgttactgtgtggaggggaaatgcacttagtgcttcagagccacttgaaagggataagtgctctagagacaattgggttcaaatgtggagcaggctgagcaagaacagaatgtctcctttgcctgagcctgagtgctgttaatcacatcttcctgccttgggctgagttagagaatcattagactatttcctgtttccatggtgagggaggcctcttccttttgtctctgctccccttaagaagcaggtgaggattttgccaggtttcttgttttgaaccttattgactttaagggcggctgggttttagagactgtacctacctagggggaacacttccgaagtttaggactattccctgatccgctgggaggcaggttactgaggaagtccctttaaaaacaaaggagtttatactgagaaaagcataaacagtgatttgtatggattcacactgactaatatagctcatgccattaaagtggggtctcttctctaaaggagggttatatgatctagccccgtagacctaagtgtggtttcagacctgttcttcctggtcctctccttggaatccatatttctactagttggactttttctgtttgtctggctctcagaggattataggaggccctgtgaagtgactcagtgaattttgatttgtgggcaagtagatggttccctagtctgaaattgactttgccttaggtgcttcaattcttcataagctcccagttcttaaaggacaagatccttgtaaacatggcaatggcattcattaggaatctagctgggaaaatccagtgtgtatgcttggaaatgagggatctggggctggagagaaaggcatgggcatgccttggagggacttgtgtgtcaagctgaggacctttactttaagctctaggggaccaggcaaggggagatgtagatacgttactctgatggggtggatgaattgaagaaggatgaggcaagaatgaaggcagagaccagggaggaggctctccaagtggccaaggcataaagcaagaaatgaggcctggtgactgcttagtggcagagcagtgaaagagagggaggcatcaaagtgagtctcgatttctagctgggtgggtggtagcgatgtccagtaggccagtggctactgaggtctgcagtggaggagggtggttgggctggagacagatgatgagggagtcatcagcctgtgggtggaagaaaagggaacctcttccaactgttttctttgcttcttccctctctttctctttttttttttttttggacagagtcttgctctgtcacccaggctgaaatgcagtggcatgatcttggctcaccacagcctccgcctcctgggttcaagcaattctcctgtctcagcctccagagtagctgggattacaggcacatatcactgtgcccggctaatttttgtattttcagtggagatgggatttcaccatgttggtcgggctggaatgaactcctgacctcaagtgatccacctgcctcagcctcccaaagtgttgggattacaggcatgagccaccgcgcccggcctttcttccctctcttaaagagtgtttatttaattccacaaacatgagcttgtcaccccctgtagcctggcatctcctacacgaggtgatggctgaggcttctgcttctgctggggtagctctgatctttctgctttctctggcactgtctacccatgttgcctcaccccacaggtcccagggcacctctctcgggcaagtcttggaaccctctgacactgatttgctctcttttctgagctgcttttagccacccatcctcgggacctgttttctctctgcctccacccctgcgggcagtcttaggtctcctgcccctcacgagcaccccagagaggccacgtgctcagtgatctcagtgggcgcatctttctagtcttgctattctttttggccatgttgttcagaaaccatactgggcagggccgacttcaccctaaaggctgcgtctcttcactctgcttttgtttgttccaaataaagtggcttcagaattgctaaccctagcctctgtgaacttgtgaggtacaattttgtgtctgttatgttaacaaaaatacatacataccttcctggtgatggtataaattgctattctctattggaaagcaatttggaatgaaaatttaaagaaccattttaaaatatgctatcctgcgtacctccattccacccacccccagggatgtagcctactgaaataattttaaagaagtcaccatatgagagaaaatgttattgctatattgttattgtgagaaattggaaatagactaaatgttcagcactataggaataattaatgaaattacatatactctatacaatcattatgctgccattgaaataataaatacaaaggcgcaaggggggaaaagcttataatgttagtgaaactaagactgatttttttataaagcagcagttttcagacccttggagactccaattcggtagaaccagagcttcatcttctctgtcgaagctgtgacaggagttgcaaatgcctctcctttttgctgagtttgcagctgctgtttttccggcagcacatctgtgcaggcctctgcctcggcccctctggatctgctgattgagcagcggattgatctgtccttctctttcgtgttgacccatgtgaggaaccaactggcaagggaacaagaaatggaaataggcctcctttgcatcatgacctgtacatcctgcaattggaaaagattgtactttagttggtttaaccagcagcattatttttctaaactaagcagtaagaaggaattaggttttatgtgggatcaacagactgggtctcaaaagaggaaggtgatagaacacagtggggagggggaggtgcactagaaacagagggcctatgctttcattctggctttgctacttaatagctgtgtgacccaatcttagagacttaacctctctgaacttccattttctcatgtataaaatgggaaatattaaaggatactcactgggctggtggcttgtgcctgtaatcccagcacttggggaggttgaggtgggaggatcacttgagcccaggtgttcaagaccagcccaggcaacatggcaagactctgtctctatgaaaaaattaaaaattagccaggtgtggtggtgtgcacctgtagtcttagctacttggtaggctgagatgggaggatcacttgggcttgggaggtcaaggctgcggtgagctgtgattccatcactgcactccagcccgggcggcagagcgagacactgaatccaaacgacaacaacaacaaaaggcaaaaaaataaaagtgccctctttatggagttgtgtaaggtgaagcatatacactattcaacatagtaactatataaaggaagtattgttgttgttactgtagttaataccattaagtgagatgtttcgtatagtggaaagcacatggactctgaattcagactggtctgactttgagtctcagctccacatctagtaatactatgaccaagccctggttaaaatcatgtttttttttcttcagcctcagtcttctcacatataaaatagggacactgtcatttacctcagttttctgtgaggataaaacaacgacagtgtatatgcaagtattttgtaaattttgtagtgctcctcaagatttagttggtgtttactacttgtactttctcactggaatggcagatgctgttggacagcagggacaatgaccacttttgggaacagcagttggatggcttagattggacagcccaagacatcgtggcgtttttggccaagcacccagaggatgtccagtccagtaatggttctgtgtacacctggagagaagctttcaacgagactaaccaggcaatccggaccatatctcgcttcatggaggtgaatctgttgctgggatcatttagaaaagacttaacggcttctttctctgagacgttacaataaggttcaggcaggaggcaagtttagaaataatgtatagtctcatttacaaaactatccctcaagcctaacacaggatttgataacaaaaggcacttaataaatgttagttgagtggttgaatgagtaaataaactctagctttagtaaattaactctagcttattctatataggctcaagagaatatttctacccattttcttctaggttttcctatctcagtgactaatggtagcaaagcattcccttaaaaaggcattatttgtgaaacttayctaaaatcgaattcgggtccaattaaatttttgaaattttatattaaaaattatattagtagggatgggtaagaggtgttttggtctggttggttggttagttgctatgactcagaattgctaagaaaacagaaaagtaagataagatcattgttttaacctcttttcctccacaaaatcaataaataacatatccctaaattactcttagaatttctcttaaattgcagtgaaaaaccaaaatccttcattcttggttgaaggttggaaaactacgttagagaggattagagagagaggatgagcaatcgtgtagtcagcccttgcctcctagtgtaggatttgtctcagccactgcttgttgtcctggctgccaacgttctcatgaaggctgttcttctatcagtgtgtcaacctgaacaagctagaacccatagcaacagaagtctggctcatcaacaagtccatggagctgctggatgagaggaagttctgggctggtattgtgttcactggaattactccmrgcagcattgagctgccccatcatgtcaagtacaagatccgaatggacattgacaatgtggagaggacaaataaaatcaaggatgggtaagtggaatcccatcacaccagcctggtcttggggaggtccagagcacctattatattaggacaagaggtactttattttaactaaaaatttggtagaaatttcaacaacaacaaaaaaactcaacttggtgtcatgattttggtgaaattggtacatgacttgctggaaggtttttcataggtcataaaataacagtatcttttgatttagcatttctactcaagggaattaattccaggaattttggtggcaggcacctgtaatcccagctactcgggaggctgaggcaggagaattgcttgaacccaggaggcagaggttgcagtgagctaagatcgcatcattgcactcccgcctgggcaataagagtgaaactccatctcaaaaaaaaaaaagatacaaaaatagaaaaaggggcttggtaagggtagtagggttttgggcaatttttttttttttttttttttttattgtatggttctaaaggaatggttgattacctgtggtttggttttaggtactgggaccctggtcctcgagctgacccctttgaggacatgcggtacgtctgggggggcttcgcctacttgcaggatgtggtggagcaggcaatcatcagggtgctgacgggcaccgagaagaaaactggtgtctatatgcaacagatgccctatccctgttacgttgatgacatgtaagttacctgcaagccactgtttttaaccagtttatactgtgccagatgggggtgtatatatgtgtgtgcatgtgcatgcatgtgtgaatgatctggaaataagatgccagatgtaagttgtcaacagttgcagccacatgacagacatagatatatgtgcacacactagtaaacctctttccttctcatccatggttgccacttttatctttttatttttatttttttttttgagatggagtctcgctctgacgcccaggctggagtgcagtggctcgatctcggctcactgcaacctttgcctcccgggttcaagctattctcctgcctcagcctccacagtagctgggactacaggctcatgctgccacgcccggctgactttttgtattttagtagagacgaggtttcaccatgttacccaggctagacttcaactcctgagctcaggcaatccaccctccttggcctcccaaagtgctgggattacaggtgtgagccactgcacccagcccaccactttaattttttacactctacccttttggtcaaaatttgctcaatctgcaagcttaaaatgtgtcatgacaaacacatgcaagcacatactcacacatagatgcagaaacagcgtctaaacttataaaagcacagtttatgtaaatgtgtgcacttcttctccctaggtggtaaaccacatttcaaaacaacccaaataaaactgaacaaagcttcttcctcttagactttttagaaaatctttcagtgctgagtcactaagctgccaagttctcattgtgggaactatgcctttggatgtaatgatttcttctaagacaatgggcggaggtgtagttattgcagacatctgaaatatgtaatgtttcttccagattctggaaattctcttattctctgtggttggtggtggtggtgggatgtgtgtgtgtgtgtgtgtgtgtgtgtgtgtgtgtgtgtagggatcaggatgcgggaggagctgggttctgcttgtattggttctctgttttgcattgaatagtgtgtttccttgtatggctatctatagcttttcaaggtcaccagaaattatcctgtttttcaccttctaaacaattagctggaatttttcaaaggaagacttttacaaagacccctaagctaaggtttactctagaaaggatgtcttaagacagggcacaggagttcagaggcattaagagctggtgcctgttgtcatgtagtgagtatgtgcctacatggtaaagctttgacgtgaacctcaagttcagggtccaaaatctgtgtgcctttttactttgcacatctgcattttctattctagcttggaatctgaaacattgacaagagctgcctgaaatgtatgtctgtggtgtgattagagttacgataagcaagtcaatagtgagatgaccttggagatgttgaacttttgtgagagaatgagttgtttttttgttttggtttttagtactttaacataatctacctttagtttaagtatcgctcacagttacctagttactgaagcaagcccccaaagaaatttggtttggcaacactttgttagcctcgtttttctctctacattgcattgctcgtgaagcattggatcatacgtacatttcagagtctagagggcctgtccttctgtggcccagatgtggtgctccctctagcatgcaggctcagaggccttggcccatcaccctggctcacgtgtgtctttctttctccccttgtccttccttggggcctccagctttctgcgggtgatgagccggtcaatgcccctcttcatgacgctggcctggatttactcagtggctgtgatcatcaagggcatcgtgtatgagaaggaggcacggctgaaagagaccatgcggatcatgggcctggacaacagcatcctctggtttagctggttcattagtagcctcattcctcttcttgtgagcgctggcctgctagtggtcatcctgaaggtaaggcagcctcactcgctcttccctgccaggaaactccgaaatagctcaacacgggctaagggaggagaagaagaaaaaaaatccaagcctctggtagagaaggggtcatacctgtcatttcctgcaatttcatccatttatagttggggaaagtgaggcccagagaggggcagtgacttgcccaaggtcaacccagccgggtagcagctaagtaggatgagagtgcagggttcatgctttccagataaccacatgctcaactgtgccatgctgtctcattggtagtggttcatggcagcatctgaaagctatttattttcttagatatattgggtggcgattcttcctaagtttctaagaacaataatcagaaggatatatattgttgcaggttagactgtctggaagcagaggctgaaatagagtttgatgtatgggtatttatgagggctcaatacctatggaagagatatggaagatgcaggattgggcagagggaggagttgaactgtgatatagggccaaccccgtggggcactctagagaatatgcagcttgttggagttgttcttcatcgagctgaaacatccagccctttgtgctcccccaaggcctccctcctgacaccacctacctcagccctctcaatcaatcactggatgtgggctgccctgggaaggtcgtgccccagggcctacatggctctctgctgctgtgacaaacccagagttgctgatgcctgaggccgtctactgacagctgggcaacaaggcttccctgaatggggactctgggcagtgcagttttgtgtctgaaccatacattaatatatttatatccgaattttctttctctgcaagcatttcatataaagacacatcaggtaaaaataaatgtttttgaagcaaaaggagtacaaagagataagaactaactaatttaatactagttaccatctgttacaaatagttcctactgattgccaaggactgtttaaacacatcacatgggcttcttcttctatcctcactaacccttttaacagacaaggaaatgaggctcaggaaggtcaaggactttattgaggttccacagtaggatacagttcttgctaaaagcaacccctccctcatgctctgttatctaactgcaaggggaaggtcagtggcagaggtagtggtcccatggttggtgcataagagctgctctgagacaactgcatgctggtgggtcctgcagacatgtacccatcagccggagataggctcaaaatatccacaagagtttggatgattgtgggaatgcagaatccatggtgatcaagagggaaagtcaagttgcctggccattttccttggcttttagacagaaaagttacgtgggatattatctcccacagctcttctgtggtgccaccagtcatagtccttatataaggagaaaccagttgaaattacctattgaagaaacaaagagcaaactcgcccactgaaatgcgtagaaagccctggactctgttgtattcataactctgccattatttttctgcgtagttttgggtaagtcacttatcttctttaggatggtaatgatcagttgcctcatcagaaagatgaacagcattacgcctctgcattgtctctaacatgagtaggaataaaccctgtcttttttctgtagatcatacaagtgagtgcttgggattgttgaggcagcacatttgatgtgtctcttccttcccagttaggaaacctgctgccctacagtgatcccagcgtggtgtttgtcttcctgtccgtgtttgctgtggtgacaatcctgcagtgcttcctgattagcacactcttctccagagccaacctggcagcagcctgtgggggcatcatctacttcacgctgtacctgccctacgtcctgtgtgtggcatggcaggactacgtgggcttcacactcaagatcttcgctgtgagtacctctggcctttcttcagtggctgtaggcatttgaccttcctttggagtccctgaataaaagcagcaagttgagaacagaagatgattgtcttttccaatgggacatgaaccttagctctagattctaagctctttaagggtaagggcaagcattgtgttttattaaattgtttacctttagtcttctcagtgaatcctggttgaattgaattgaatggaatttttccgagagccagactgcatcttgaactgggctggggataaatggcattgaggaatggcttcaggcaacagatgccatctctgccctttatctcccagctctgttggctatgttaagctcatgacaaaccaaggccacaaatagaactgaaaactcttgatgtcagagatgacctctcttgtcttccttgtgtccagtatggtgttttgcttgagtaatgttttctgaactaagcacaactgaggagcaggtgcctcatcccacaaattcctgacttggacacttccttccctcgtacagagcagggggatatcttggagagtgtgtgagcccctacaagtgcaagttgtcagatgtccccaggtcacttatcaggaaagctaagagtgactcataggatgctcctgttgcctcagtctgggcttcataggcatcagcagccccaaacaggcacctctgatcctgagccatccttggctgagcagggagcctcagaagactgtgggtatgcgcatgtgtgtgggggaacaggattgctgagccttggggcatctttggaaacataaagttttaaaagttttatgcttcactgtatatgcatttctgaaatgtttgtatataatgagtggttacaaatggaatcattttatatgttacttggtagcccaccactccctaaagggactctataggtaaatactacttctgcaccttatgattgatccattttgcaaattcaaatttctccaggtataatttacactagaagagatagaaaaatgagactgaccaggaaatggataggtgactttgcctgtttctcacagagcctgctgtctcctgtggcttttgggtttggctgtgagtactttgccctttttgaggagcagggcattggagtgcagtgggacaacctgtttgagagtcctgtggaggaagatggcttcaatctcaccacttcggtctccatgatgctgtttgacaccttcctctatggggtgatgacctggtacattgaggctgtctttccaggtacactgctttgggcatctgtttggaaaatatgacttctagctgatgtcctttctttgtgctagaatctctgcagtgcatgggcttccctgggaagtggtttgggctatagatctatagtaaacagatagtccaaggacaggcagctgatgctgaaagtacaattgtcactacttgtacagcacttgtttcttgaaaactgtgtgccaggcagcatgcaaaatgttttatacacattgcttcatttaattctcacaaggctactctgaagtagttactataataaccagcaattttcaaatgagagaactgtgactcaaagacgttaagtaaccagctttggtcacacaactgttaaatgttggtacgtggaggtgaatccacttcggttacactgggtcaataagcccaggcgaatcctcccaatgctcacccaattctgtatttctgtgtcctcagagggggtacaactaggagaggttctgtttcctgagtacaggttgttaataattaaatatactagctctaaggcctgcctgtgatttaattagcattcaataaaaattcatgttgaatttttctttagtacttctttcttaatataatacatcttcttgaccaagtccaagaggaacctgcgttggacagttttcatatgagatcaaattctgagagagcaagatttaaccctttttggttcaccttctgatcctcccctaaggaggtatacatgaaatatttattactcctgcctgaacttctttcattgaatatgcaattttgcagcatgcagattctggatttaaattctgagtcttaacttactggctgagggaccttggataggctccttatccctcagtttcctcatctctaaaatggggatggcacctgccccgtgggttgttggaaggacttacagaggtgcagaatgtacgttgtacatagcaggtttcagcaaatgttagctccctctttccccacatccattcaaatctgttccttctccaaaggatgtgtcaaggaggaaatggacctggctgggaaaccctcagaatactgggatgatgctgagcttggctcatacctgtgctttgctttcaggccagtacggaattcccaggccctggtattttccttgcaccaagtcctactggtttggcgaggaaagtgatgagaagagccaccctggttccaaccagaagagaatgtcagaaagtaagtgctgttgacctcctgctctttctttaacctagtgctgctgcctctgctaactgttgggggcaagcgatgtctcctgcctttctaaaagactgtgaaaccactccaggggcagagaaatcacatgcagtgtccctttccaaatcctcccatgccatttatgtccaatgctgttgacctattgggagttcacggtctcgatccctgagggacattttctttgttgtcttggcttctagaagagtatcttttacttgccccctcccaaacacacatttcatggtctcctaacaagctagaagaaagaggtaaagacaagcgtgattgtggaaccatagcctcgctgcctgcctgtgacatggtgacctgtgtatcagcctgtgtgggctgagaccaagtggctaccacagagctcagcctatgcttcataatgtaatcattacccagatccctaatcctctcttggctcttaactgcagacagagatgtccacagctcatcaaaggctctgcttctgggttctttgtgcttagagtggcttcctaaatatttaataggtcccttttctgccagtctcttctgtgcccatcccctgattgcccttggtaaaagtatgatgccccttagtgtagcacgcttgcctgctgttcctaatcatcttctcctacctcctctttacacctagctcctgtttcagtcacctagaaatgctcacagtcgctggaatatgtcatgttcttccacacctccatgcctttgtaggtactgtttgctctcacaggagaactttctctctaacttgcctatcttctcaactcctcctttctctccaagatctagttccggatcccctcccctgagcatccctccttggttctcaggtagtcagtcactctctgccctgaacttccatggcacgtgaaagaaaatctttttattttaaaacaattacagactcacaagaagtaatacaaattacatgagggggttcccttaaacctttcatccagtttccccaatggtagcagcatgtgtaactgtagaatagtatcaaaaccatgaaattgacataggtacaattcacaaaccttcttcagatttcactagctttatgtgcgctcatttgtgtgtgtgtgtgcgtatttagttctatgcaattttatcatgtgtgaattcatgtaattactagctcagtcaagctgcagaaatatctcattgtcacaaagctccttcatgctaccccttaatggccacagccacctcccttcttcctcagttcctgacacctgtcaaccactaatgcgttcctcgtttttacagttttattatttctagaatgttacataaatggaaccatacagtaggtatccttttgatactggcttttttttttttttcactcagcagtattcccttagatctatccaagttgtgtgtgtcaacagttcattcctcttcactgctgagtagtgttccctgggaggggtgtatcacagttccatggcatttttagatgtattttttaaacagctttcagcatcctctattttaattgttcatcaagtcctttttcccaatagactctgaatgctcctttatcatcgtattcccatcaccaacatcagtacccaaataggccctaaataaacatttatagcctcctgcctgcctgagaaaccagggtggacatggagagaaggcacttctgaaagttcaagcgcagtgcsctgtgtccttacactccactcctcagtgctttctgtgggttcatttctgtcttctctcctgtcacagtctgcatggaggaggaacccacccacttgaagctgggcgtgtccattcagaacctggtaaaagtctaccgagatgggatgaaggtggctgtcgatggcctggcactgaatttttatgagggccagatcacctccttcctgggccacaatggagcggggaagacgaccaccatgtaagaagagggtgtggttcccgcagaatcagccacaggagggttctgcagtagagttagaaatttataccttaggaaaccatgctgatccctgggccaagggaaggagcacatgaggagttgccgaatgtgaacatgttatctaatcatgagtgtctttccacgtgctagtttgctagatgttatttcttcagcctaaaacaagctggggcctcagatgacctttcccatgtagttcacagaattctgcagtggtcttggaacctgcagccacgaaaagatagattacatatgttggagggagttggtaattcccaggaactctgtctctaagcagatgtgagaagcacctgtgagacgcaatcaagctgggcagctggcttgattgccttccctgcgacctcaaggaccttacagtgggtagtatcaggaggggtcaggggctgtaaagcaccagcgttagcctcagtggcttccagcacgattcctcaaccattctaaccattccaaagggtatatctttggggggtgacattcttttcctgttttctttttaatctttttttaaaacatagaattaatatattatgagcttttcagaagatttttaaaaggcagtcagaaatcctactacctaacacaaaaattgtttttatctttgaataatatgttcttgtttgtccattttccatgcatgcgatgttaggcatacaaaatacattttttaaagaatactttcattgcaaattggaaacttcgtttaaaaaatgctcatactaaaattggcatttctaacccataggcccacttgtagttatttaccgaagcaaaaggacagctttgctttgtgtgggtctggtagggttcattagaaaggaatgggggcggtgggagggttggtgttctgttctctctgcagactgaatggagcatctagagttaagggtaggtcaaccctgacttctgtacttctaaatttttgtcctcaggtcaatcctgaccgggttgttccccccgacctcgggcaccgcctacatcctgggaaaagacattcgctctgagatgagcaccatccggcagaacctgggggtctgtccccagcataacgtgctgtttgacatgtgagtaccagcagcacgttaagaataggccttttctggatgtgtgtgtgtcatgccatcatgggaggagtgggacttaagcattttactttgctgtgtttttgttttttctttttttcttttttatttttttgagatggagtctcgctctgtagccaggctggactgtagtggcgcgatctcggctcactgcaaccttggcctcccaggttcaagcgattctcctgcctcagcctcccgagtagctgggactctaggcacacaccaccatgcccagctaatttttgtgtttttagtagagacggggtttcaccatgttggccaggatggtctcaatgtcttgacctcgtgatccgcccacctcggtctcccaaagtgctgggaacacaggcatgagccactgtgtctggccacattttactttctttgaatatggcaggctcacctccgtgaacaccttgagacctagttgttctttgattttaggagaagtgggaggtgaatggttgagctgtagaggtgacatcagcccagccagtggatgggggcttgggaaacattgcttcccattattgtcatgctggagggccctttagcccatcctctccccccgccaccctccttattgaggcctggagcagacttcccagacctggtagtgcttcagggccctggtatgatggacctatatttgctgcttaagacatttgctcccactcaggttgtcccatcagccataaggcccccagggagcccgtgtgatggagcagagagagacctgagctctgcaatcttgggcaaggcttttcccttatgtttcttcttatctaaagtgaacagctggggctcatgtgctccctcctcatctaaagtgaacacatggggctcatgtgcagggtcctccccgctttcagagcctgaggtcccctgaggctcaggaaggctgctccaggtgagtgccgagctgacttcttggtggacgtgctgtggggacagcccattaaagaccacatcttggggccctgaaattgaaagttgtaactgcctggtgcatggtggccaggcctgctggaaacaggttggaagcgatctgtcacctttcactttgatttcctgagcagctcatgtggttgctcactgttgttctaccttgaatcttgaagattatttttcagaaattgataaagttattttaaaaagcacggggagagaaaaatatgcccattctcatctgttctgggccaggggacactgtattctggggtatccagtagggcccagagctgacctgcctccctgtccccaggctgactgtcgaagaacacatctggttctatgcccgcttgaaagggctctctgagaagcacgtgaaggcggagatggagcagatggccctggatgttggtttgccatcaagcaagctgaaaagcaaaacaagccagctgtcaggtgcggcccagagctaccttccctatccctctcccctcctcctccggctacacacatgcggaggaaaatcagcactgccccagggtcccaggctgggtgcggttggtaacagaaacttgtccctggctgtgcccctaggtcctctgccttcactcactgtctggggctggtcctggagtttgtcttgctctgtttttttgtaggtggaatgcagagaaagctatctgtggccttggcctttgtcgggggatctaaggttgtcattctggatgaacccacagctggtgtggacccttactcccgcaggggaatatgggagctgctgctgaaataccgacaaggtgcctgatgtgtatttattctgagtaaatggactgagagagagcggggggcttttgagaagtgtggctgtatctcatggctaggcttctgtgaagccatgggatactcttctgttakcacagaagagataaagggcattgagactgagattcctgagaggagatgctgtgtctttattcatctttttgtccccaacatggtgcactaaatttatggttagttgaaagggtggatgcttaaatgaatggaagcggagaggggcaggaagacgattgggctctctggttagagatctgatgtggtacagtatgaggagcacaggcaggcttggagccaactctggcttggccctgagacattgggaaagtcacaacttgcctcaccttctttgccgataataatagtggtgcgttacctcatagaggattaaattaaatgagaatgcacacaaaccacctagcacaatgcctggcatatagcaagttcccaaataaaatgcgtactgttcttacctctgtgaggatgtggtacctatatatacaaagctttgccattctaggggtcatagccatacagggtgaaaggtggcttccaggtctcttccagtgcttacccctgctaatatctctctagtccctgtcactgtgacaaatcagaactgagaggcctcacctgtcccacatccttgtgtttgtgcctggcaggccgcaccattattctctctacacaccacatggatgaagcggacgtcctgggggacaggattgccatcatctcccatgggaagctgtgctgtgtgggctcctccctgtttctgaagaaccagctgggaacaggctactacctgaccttggtcaagaaagatgtggaatcctccctcagttcctgcagaaacagtagtagcactgtgtcatacctgaaaaaggtgagctgcagtcttggagctgggctggtgttgggtctgggcagccaggacttgctggctgtgaatgatttctccatctccaccccttttgccatgttgaaaccaccatctccctgctctgttgcccctttgaaatcatatcatacttaaggcatggaaagctaaggggccctctgctcccattgtgctagttctgttgaatcccgttttccttttcctatgaggcacanagagtgatggagaaggtccttagaggacattattatgtcaaagaaaagagacttgtcaagaggtaagagccttggctacaaatgacctggtcgttcctgctcattacttttcaatctcattgaccttaacttttaaactataaaacagccaatatttattaggcactgatttcatgccagagacactctgggcattgaaagaaagtaatgataatagttaattttatatagcgttgttaccatttcaacctttttttttttttaacctctatcatctcaattaaag

SEQ ID NO: 22 - DNA, 7052 bp, Homo sapiens
gtgaacacacattaaagcatgagaagcatgaactagacatgtagccaggtaaaggccttgctgagatggttggcaaaggcctcattgcagcattcattggcaggccacagttcttttggcagctctgcttcctgacctttcaccctcaggaagcgaggctgttcacacggcacacacatgccagacagggtcctctgaagccacggctgccagtgcatgtgtcccagggaaagctttttcctttagttctcacacaacagagcttcttggaagccctccccggcgaaggtgctggtggctctgccttgctccgtccctgacccgttctcacctccttctttgccatcaggaggacagtgtttctcagagcagttctgatgctggcctgggcagcgaccatgagagtgacacgctgaccatcggtaaggactctggggtttcttattcaggtggtgcctgagcttcccccagctgggcagagtggaggcagaggaggagaggtgcagaggctggtggcgctgactcaaggtttgctgctgggctggggctgggtggctgcgggggtgggagcagcttggtggcgggttggcctaatgcttgctggggtgcctggggctcggtttgggagctagcagggcagtgtcccagagagctgagatgattggggtttggggaatcccttaggggagtggacactgaataccagggatgaggagctgagggccaagccaggagggtgggatttgagcttagtacataagaagagtgagagcccaggagatgaggaacagccttccagatttttcttgggtagcgtgtgtaggaggccagtgtcaccagtagcatatgtggaacagaagtcttgacccttgctatctctgcctagtcctaatggctggcttttcccaggaaggcttctgcttccatggactgttagattaaccctttatttaggtaaatgagggaacctactttataagcataggaaagggtgaagaatcttttaagattcctttactcaagttttcttttgaagaatcccagagcttaggcaatagacaccagactttgagcctcagttatccattcacccatccacccacccacccacccatccttccatcctcccatcctcccattcacccatccacccatccagctgtccacccattctacactgagtacctataatgtgcctggctttggtgatacaaaggtgaataagacatagtcctttcctttgcccccaaccctcagaccagagatgaacatgtggaatgacctaaacacctggaacaggtgtggtgtatgagcggcaggcctctgatgagagggtgggggatggccagccctcactccgaagcccctctgagttgattgagccatctttgcattctggtcctgcagatgtctctgctatctccaacctcatcaggaagcatgtgtctgaagcccggctggtggaagacatagggcatgagctgacctatgtgctgccatatgaagctgctaaggagggagcctttgtggaactctttcatgagattgatgaccggctctcagacctgggcatttctagttatggcatctcagagacgaccctggaagaagtaagttaagtggctgactgtcggaatatatagcaaggccaaatgtcctaaggccagaccagtagcctgcattgggagcaggattatcatggagttagtcattgagtttttaggtcatcgacatctgattaatgttggccccagtgagccatttaagatggtagtgggagatagcaggaaagaagtgttttcctctgtaccacagtacatgcctgagatttgtgtgttgaaaccagtggtacctaacacatttacatcccaaccttaaactcctatgcacttatttaccctttaatgagcctctttacttaagtacagtgkgaggaacagcggcatcaggatcacttgggaacttgttagaaattcagcaacttgggcccagctcagacctactgaatcagaatcaggagcaattctctggtgtgactgtgtcacagccaggtatcaactggattctcatacataggaaatgacaaacgtttatggatggatagtctacttgtgccaggtgctgagatttgttttttgttttttgatttttttttaatcactgtgacctcatttaattctcaaaaaaagatgaaaaaatgaacactcaggaatgctgacatgagattcagaatcaggggtttggggcttcaaagtccatcctctctttatccatgtaatgcctccccttagagatacaacatcacagaccttgaaggctgaaggggatataaaagctgtctggccaagtggtctccaagcttgacagtgcagcagaatcacctggggatattattaaaaataaacatactaaggtttggcttcagggcctgtgaatcagaatttctggaggtgaggccttgaagtctgtatttctattgcatactttggacacagtggtctatagactagagtttggaaatgattgcgctcattcagattctcttctgatgtttgaattgctgccatcatatttctagtgctctatttcctcctgctcattctgtcttggataacttatcatagtactagcctactcaaagatttagagccacagtcctgaaagaagccacttgactcattccctgtaggttcagaataaatttcttctgcgcagtgtctgtcatagctttttttaaatttttttttatttttgatgagactggagttttgctcttattgcccaagctggagtgcagtggtgcgattttggctcactgcaacctccacctcccaggttcaagcgattctcctgcctcagcctcccaagtagctgagattacaagcatgtgctaccacgcccagctaattttgtatttttagtagagatgggttttatccatgttggtcaggctggtctcgagctccagacctcaggtgatctgcccgcctcggcctcccaaagtgctgggattataggcctgagccacagcgctcagccataactttaatttgaaaatgattgtctagcttgatagctctcaccactgaggaaatgttctctggcaaaaacggcttctctcccaggtaactctgagaaagtgttattaagaaatgtggcttctactttctctgtcttacggggctaacatgccactcagtaatataataatcgtggcagtggtgactactctcgtaatgttggtgcttataatgttctcatctctctcattttccagatattcctcaaggtggccgaagagagtggggtggatgctgagacctcaggtaactgccttgagggagaatggcacacttaagatagtgccttctgctggctttctcagtgcacgagtattgttcctttccctttgaattgttctattgcattctcatttgtagagtgtaggtttgttgcagatggggaaggtttgttttgttgtaaataaaataaagtatgggattctttccttgtgccttcagatggtaccttgccagcaagacgaaacaggcgggccttcggggacaagcagagctgtcttcgcccgttcactgaagatgatgctgctgatccaaatgattctgacatagacccaggtctgttagggcaagatcaaacagtgtcctactgtttgaatgtgaaattctctctcatgctctcacctgttttctttggatggcctttagccaaggtgatagatccctacagagtccaaagagaagtgaggaaatggtaaaagccacttgttctttgcagcatcgtgcatgtgatcaaacctgaaagagcctatccatatcacttcctttaaagacataaagatggtgcctcaatcctctgaacccatgtatttattatcttttctgcggggtcctagtttcttgtatacattaggtgtttaattgttgaacaaatattcattcgagtagatgagtgattttgaaagagtcagaaaggggaatttgctgttagagttaattgtaccctaagacttagatatttgaggctgggcatggtggctcatgccagtaatcccagcgctttgagaggctgaggtgggtagatcacctgaggtcaggagtttgagaccagtctgaccaacaaggtgaaaccccgtctctactaaatacaaaaaattagccgagtgtggtggcacatgcctgtcatcccagctacttgggaggctgaggcaggagaatcgcttgaacccaggaggcagaggttgcagtcagccacggttgcgccattgcactccagactgggcaacaagagtgaaaactccatctcaaaaaagaaaaaaaaagaattagatattttggatgagtgtgtctttgtgtgtttaactgagatggagaggagagctaagacatcaaacaaatattgttaagatgtaaaagcacatcagttaggtatcattagtttaggacaaggatttctagaaaatttttaggaacagaaaactttccagttctctcacccctgctcaaagagtgtatggctcttacattatatataactgcctgacttcatacagtatcagtacttagatcatttgaaatgtgtccacgttttaccaaaatataatagggtgagaagctgagatgctaattgccattgtgtattctcaaatatgtcaagctacgtacatggcctgtttcatagagtagtctataagaaattgatgacttgattcatccgaatggctggctgtaacacctggttacgcatgaacacctcttttcagttgtctcaagacacctttcttttctgtacttatcagacaaggactgaaaggcagagactgctactgttagacattttgagtcaagcttttccttggacatagctttgtcatgaaagccctttacttctgagaaacttctagcttcagacacatgccttcaagatagttgttgaagacaccagaagaaggagcatggcaatgccgaaaacacctaagataataggtgaccttcagtgttggcttcttgcagaatccagagagacagacttgctcagtgggatggatggcaaagggtcctaccaggtgaaaggctggaaacttacacagcaacagtttgtggcccttttgtggaagagactgctaattgccagacggagtcggaaaggattttttgctcaggtgagacgtgctgttttcgccagagactctggcttcatgggtgggctgcaggctctgtgaccagtgaaggcaggatagcatcctggtcaagatatggatgccggagccagatttatctgtatttcaatcccagttctattccttgccagttgtgtatccgctggcaagttacttctctatgcctcaatctcctcatctgtaaaatggggataataatattacctgcaatacagggttgttacgaaaataaaaatgaataggtgcttagaatggggcctgacattagtaagtgcttagttttgtgtgtgtatatgttatttttattttggaggagaacataaaaaggacaaagtgtagaaaaactggttgggtgtattcagctgtcataacatgagagttgttatgcccagatgcacttgacatgtgaatttattagaaacatgatttttctctgagttgatgtttaactcaaactgatagaaaagataggtcagaatatagttggccaacagagaagacttgttagactattgtctgcatgtcagtgtttgcatgctaacttgcttagttagaaaggttaaattttttcactctataaaatcaagaaatatagagaaaaggtctgcagagagtctttcatttgatgatgtggatattgttaagagcgggagtttggagcatacagagctcaagttgaatcctgactttgctacttattggctatatgaccttgggcaagctgcttagtctctctgatcctcagttacctttgtttgttgatgatgaccattgataacacaaccataaataatgacaacatagagatagttctcattatagtagttgttatacagaattattcactcaatgttaattttctgcattgaaatcccagaacattagaattgggggcattatttgaatctttaaggttataaggaatacatttctcagcaataaatggaaggagttttgggttaacttataaagtatacccaagtcattttttttcagagaagatatggtagaaagtcttaggaggttgaagaaggaattggatatttattctttctgagactatcatgggagataatgactatggttgtccatgattggagccgttgctgtagagttggttttattatagtgtaggatttgaatgggccatgtgttctcagacctcagaataaaaagagaaaactgaggccagtggggagcgtgacttcacatgggtacacttgtgctagagacagaaccaggattcaggacttctggctcctggtcctgggttcatggcccaatgtagtctttctcagtcttcaggaggaggaagggcaggacccagtgttctgagtcaccctgaatgtgagcactatttacttcgtgaacttcttggcttagtgcctctgccaggtggccataacctctggccttgtgttgccagagaaaaggtttagttttcaggctccattgcttcccagctgccaagaatgccttggtgcagcacagtcataggccctgcattcctcattgccgtgctggttggtcggggaggtgggctggactcgtagggatttgccccttggccttgtttctaacacttgccgtttcctgctgtccccctgccccctccactgcctgggtaaagattgtcttgccagctgtgtttgtctgcattgcccttgtgttcagcctgatcgtgccaccctttggcaagtaccccagcctggaacttcagccctggatgtacaacgaacagtacacatttgtcaggtatgtttgtcttctacatcccaggagggggtaagattcgagcagaccaaagatgtttacgagggccaagggaatggacttcagaattacacggtggaat

SEQ ID NO: 23 - DNA, 2534 bp, Homo sapiens
gggaagcatttaaaaaaaaaaaagtatatatatatatatatatatatatatgtaatgtgaattggcctctttttctctaagcccacattttcttcttacatagttcaggtttactttattttttcctttccggctgctgaccctgtattgcccgtagttgtggaacatagcatgtgtttgtgacctgtgcctgttatttttgtgctttctagttgtgcatgcaaagagtacaaagttttcttgccctttcttggaaaatcctgcttgtctgtgccaaagggataattgtgaaagcacttttgaaatacttaatgagttgattttcttcaaattaaaaaaaatatataaatgtatatgtgtatgtacatgtgtgtacacatacacacctttatacatacagcccatttaaaacaagctccactttggagtgctctacgtcaccctgatgccgaatacagggccagagtctgagatccttctgggtggtttctgtgttttgttcatttctgttttaagagcctgtcacagagaaatgcttcctaaaatgtttaatttataaaaacatttttatctctcgattactggttttaatgaattactaagctggctgcctctcatgtacccacagcaatgatgctcctgaggacacgggaaccctggaactcttaaacgccctcaccaaagaccctggcttcgggacccgctgtatggaaggaaacccaatcccgtgagtgccactttagccataagcagggcttcttgtgcttgttgcctggtttgatttctaatatgctgcatttatcaactgcatgccacattgtgaccgccagcatttgccctttgaattattattatgttttatttacaaaaagcgaaggtagtaaccgaactaaattatctaggaacaaacgtttggagagtcttctaacaccgyscaaagcacgtcattacagacatttgtttactgatttagaaccttaatatttaatttaaatacgcactttacacttactgatgaaatgcttttcctttctttctctcccagcccctgtacttaagtgcttcaataggctctcattatatatgatttttaggttttgcttatcagcttcttcgcttttataatctgaaaagatggcatatgaatttttataaaaagggacactttcttcttctcaaattgtatatttttattgtactttccttcaaaacccccttttaaaaagtaagcagtggataaataaattcagtgaagcatccatatgacccttaagtgagtgtaggggaagggaggtcaccagatcactgtgagtgaagatggtggagaggtgaggatcttatgaggccgtgctcaaggctggtagaggtgggttagtgtttccaggtttaggcagaatctcagctgaggtcatgaaacaacagtgatctctgaaaaattatggcaaggtgggaaggtgctggagaattggagagggggcaaacttgactttcaagtttcaatgggaagataggtgactctgcacaccacagaacagtgagcatgataacctgtttatacaaggttctagagcagatttctaaatggatagctactgtgtgcttgtttgttcttaattagtattggatagttactaaatacttgttagtacttagtacataatgggtggtaaatcctagcagctaatattggttcccaaataaccagatgacaaggatagagaaggacacagacacggcctatctggatttcatggtgcctttgattttccacatgaaggttgtgtagggaagatagaagcatgagatgagatgataatatagttatctggattcatcactggccagctgaaccatatgaactcatggattgatgctagcttaggaaggctctgtaggagccagaactgggctgagagccagcccatagagacaaaagaggcccggccctgacatcagagggttcaaacatgatgtctgagccccacctacagtctgccggaggtggttggaaggaagagcctttatccttacaattcttactgaaattcaaatttttaggttttgcaaaaaaatggtggacctgaaggaaatttgacaggagcatgtctcagctgtatttaaatttgtctcagccaatccccttttgaatgttcagagtgtaagcttcaggagggcagcgcgtcttagtgtgacttttctggtcagttcaggtgctttaaggagacaattagagatcaatctggaaaacttcatttgaatttttaatacataagaaaacaataagaaatagttaaaaatatatatttatataatatatatatgtgtgtgtgtgtgtgtgtgtgtgtgtgtgtatatatatatatattttatttatttatttttttttgagatggagtctcgctctgttgcccaggctggagtgcagtggctcaatcttggctcactgccacctctgcctcccaggttcaagtgattctcctacctcagcctcctgagtagctgggattacaagcatgtgccaccacactggctaa

SEQ ID NO: 24 - DNA, 2841 bp, Homo sapiens
tcttgccagtctctactcatttttcagcacatcgagcataagatccagactctttcccaggcctctctcatctggctcctctcctcctcctttatcattactcttcttcgtagcttatcctactccagccatgctgtcttcctattattcctaaaaartagaaatgcatttcttcctagggcctttgtacctgcacttgccatcgcttttgctcagaatgttctttttgccaagcttttgcccagcttgttctccatcattgttatgttttggctgaaatgtcttctcttagtaggttcattctccccagtcactgtctttttattttgctttattttgggccatctaaggttatcttattagtgtatttgttgttcgtctcctccatgggcatacacctccatgaaggcaggtattttcaccttaggccctcgaatatactggacagcatctggcacgtagtagatgctcaacgaatgtttgttgtgtgagcaaatggttggttgattggattgaactgagttcagtatgtaaatatttagggcctctttgcattctattttacttatgtataaaatgatacataatgatgatataaatgatgtcacagtgtacaaggctgttgtgggatcaagcaatcaaatgagatcatgcttgtcttttccaaatggtgagggaatagatgcatgtttgtggttgttacggaatgatcctgtgctcctgaggcaacagaaaggccaggccatctctggtaatcctactcttgctgtcttccctttgcagagacacgccctgccaggcaggggaggaagagtggaccactgccccagttccccagaccatcatggacctcttccagaatgggaactggacaatgcagaacccttcacctgcatgccagtgtagcagcgacaaaatcaagaagatgctgcctgtgtgtcccccaggggcaggggggctgcctcctccacaagtgagtcactttcagggggtgattgggcagaaggggtgcaggatgggctggtagcttccgcttggaagcaggaatgagtgagatatcatgttgggagggtctgtttcagtcttttttgttttttgtttttttttctgaggcggagtcttgctctgtcgcccaggctggagtgctgtggcatgatcttgcctcactgcaacctccacctcccaggttcaagcgattctcctgcctcagcctcctgagtagctgggattacaggcacgcaccaccatgtctggctaatttttgtgtttttagtagagatagggtttcgccgtgttggctaggctggtctggaattcctgacctcaggtgatccacccgcctcggcctcccaaagtgctgggattacaggcgtgagccactacgcccagccctgtttcagtctttaactcgcttcttgtcataagaaaaagcatgtgagttttgaggggagaaggtttggaccacactgtgcccatgcctgtcccacagcagtaaagtcacaggacagactgtggcaggcctggcttccaatcttggctctgcaacaaatgagctggtagcctttgacaggcctgggcctgtttcttcacctctgaattagggaggctggaccagaaaactcctgtggatcttgtcaactctggtattcttagagactctgtttgggaaggagtcctgagccattttttttttcttgagaatttcaggaagaggagtgcttatgatagctctctgctgcttttatcagcaaccaaattgcaggatgaggacaagcaattctaaatgagtacaggaactaaaagaaggcttggttaccactcttgaaaataatagctagtccaggtgcggggtggctcacacctgtaatctcagtattttgggatgccgaggtggactgatcacctaaggtcaggagttcgaaaccagcttggccaatgtggcgaaaccctgtctctactaaaaattcaaaaattagccaggcatggtggcacatgcctgtaatcccagttacttgggaggctgaagcaggagaattgcttgaacctgggaggtggaggtcgcagggagccaaaattgcgccactgtactccagcctgagcaacacagcaaaactccatatcaaaaaataaaatgaataaaataacagctaatctagtcatcagtataactccagtgaacagaagatttattaggcatagtgaatgatggtgcttcctaaaaatctcttgactacaaagaatctcatttcaatgtttattgtttagatgttcagaataaattcttgggaaagaccttggcttggtgtaagtgaattaccagtgccgagggcagggtgaaccaagtctcagtgctggttgactgagggcagtgtctgggacctgtagtcaggtttccggtcacactgtggacatggtcactgttgtccttgatttgttttctgtttcaattcttgtctataaagacccgtatgcttggttttcatgtgatgacagagaaaacaaaacactgcagatatccttcaggacctgacaggaagaaacatttcggattatctggtgaagacgtatgtgcagatcatagccaaaaggtgactttttactaaacttggcccctgccttattattactaattagaggaattaaagacctacaaataacagactgaaacagtgggggaaatgccagattatggcctgattctgtctattggaagtttaggatattatcccaaactagaaaagatgacgagagggactgtgaacattcagttgtcagcttcaaggctgaggcagcctggtctagaatgaaaatagaaatggattcaacgtcaaattttgccac

SEQ ID NO: 25 - DNA, 852 bp, Homo sapiens
gcatgctggagtgatagtgaccatgagtttctaagaaagaagcataatttctccatatgtcatccacaattgaaatattattgttaattgaaaaagcttctaggccaggcacggtggctcatgcctgtaatcccagcactttaggagccaaggcgggtggatcacttgaggtcaggagtttgagaccagcctggccaacatggggaaaccctgtctctactaaaaatacaaaataagctgggcgtggtggtgcgtgcctgtaatcccagctacttgggaggctgaggcaggagaactgcttgaatctgggaggcggaggttgcagtgagctgagttcatgccattgcattccagcctgggcaacaagagcgaaaccatctcccaaaagaaaaaaaaaagaaagaaaaagcttctagtttggttacatcttggtctataaggtggtttgtaaattggtttaacccaaggcctggttctcatataagtaatagggtatttatgatggagagaaggctggaagaggcctgaacacaggcttcttttctctagcacaaccctacaaggccagctgattctagggttatttctgtccgttccttatatcctcaggtggatatttactccttttgcatcattaggaataggctcagtgctttctttgaactgattttttgtttctttgtctctgcagcttaaagaacaagatctgggtgaatgagtttaggtaagttgctgtctttctggcacgtttagctcagggggaggatggtgttgtaggtgtgcttggattgaagaaagccttggggattgtttgtcactcacacacttgtgggtgccatctcactgtgagga

SEQ ID NO: 26 - DNA, 6289 bp, Homo sapiens
gctttatagagtttctgcctagagcatcatggctcagtgcccagcagcccctccagaggcctctgaatatttgatatactgatttccttgaggagaatcagaaatctcctgcaggtgtctagggatttcaagtaagtagtgttgtgaggggaatacctacttgtactttccccccaaaccagattcccgaggcttcttaaggactcaaggacaatttctaggcatttagcacgggactaaaaaggtcttagaggaaataagaagcgccaaaaccatctctttgcactgtatttcaacccatttgtccttctgggttttgaaggaacaggtgggactggggacagaagagttcttgaagccagtttgtccatcatggaaaatgagataggtgatgtggctacgtcagggggcccgaaggctccttgttactgatttccgtcttttctctctgccttttccccaagggccaggacccctggatctctgggcagagcagacgcaggcccctataatagccctcatgctagaaaggagccggagcctgtgtataaggccagcgcagcctactctggacagtgcagggttcccactctcccaactccccatctgcttgcctccagacccacattcacacacgagccactgggttggaggagcatctgtgagatgaaacaccattctttcctcaatgtctcagctatctaactgtgtgtgtaatcaggccaggtcctccctgctgggcagaaaccatgggagttaagagattgccaacatttattagaggaagctgacgtgtaacttctgaggcaaaatttagccctcctttgaacaggaatttgactcagtgaaccttgtacacactcgcactgagtctgctgctgatgatactgtgcaccccactgtctgggttttaatgtcaggctgttcttttaggtatggcggcttttccctgggtgtcagtaatactcaagcacttcctccgagtcaagaagttaatgatgccatcaaacaaatgaagaaacacctaaagctggccaaggtaaaatatctatcgtaagatgtatcagaaaaatgggcatgtagctgctgggatataggagtagttggcaggttaaacggatcacctggcagctcattgttctgaatatgttggcatacagagccgtctttggcatttagcgatttgagccagacaaaactgaattacttagttgtacgtttaaaagtgtaggtcaaaaacaaatccagaggccaggagctgtggctcatgcctgtaatcctagcactttgggaggctgaagcgggtggatcacttgaggtcaggagttcgagaccagcctggcctacatgacaaaaccccgtatctactaaaaatacaaaaaaattagctgggcttggtggcacacacctgtaatcccagctacttgggaggctgaggcaggagaattgcttgaaccctgtaggaagaggttgtagtgagccaagatcgcaccgttgcactccagcctgggcaacaagagcaaaactccatctcaaaaaacaaattaaatccagagatttaaaagctctcagaggctgggcgcggtggcttacacctgttatcccagcattttgggatgccgaggcgggcaaagcacaaggtcaggagtttgagaccagcctggccaacatagtgaaaccctgtctctgctaaaaacatagaaaaattagccgggcatggtggcgtgcgcctgtaatcccagctactcgggaggctgaggtgagagaattrcttgaacccgggaggcggaggttgcagtgagcccagattgcaccactgcactccagcctgggcgacagagcaagactccatctcaaaaaaagctctcagaacaaccaggtttacaaatttggtcagttggtaaataaactgggtttcaaacatactttgctgaaayaatcactgactaaataggaaatgaatctttttttttttttttttaagctggcaagctggtctgtaggacctgataagtactcacttcatttctctgtgtctcaggtttcccatttttaggtgagaattaaggggctctgataaaacagaccctaggattgtggacagcagtgatagtcctagagtccacaagtctgcttttgagtgatgggcccatgtatctggcacatctgcaggcagagcgtggttctggctcttcagatgatgccggtggagcactttgaggagtcctcaccccaccgtgataaccagacattaaaatcttggggctttgcatcccaggatttctctgtgattccttctagacttgtggcatcatggcagcatcactgctgtagatttctagtcacttggttctcaggagccgtttatttaatggcttcacatttaatttcagtgaacaaggtagtggcattgctcttcacagggccgtcctgttgtccacaggttccagattgactgttgccccttatctatgtgaacagtcacaactgaggcaggtttctgttgtttacaggacagttctgcagatcgatttctcaacagcttgggaagatttatgacaggactggacaccagaaataatgtcaaggtaaaccgctgtctttgttctagtagctttttgatgaacaataatccttatgtttcctggagtactttcaactcatggtaaagttggcaggggcattcacaacagaaaagagcaaactattaactttaccagtgaggcagtacggtgtagtgtagtgattcagagaatttgctttgccaccagacataccaggtaaccttgactaagttacttaacctatctaaacctcagttycctcatctgtgaaatggagacagtaatcatagctatttccaaactgttgtgagaattcaatgagttaaaggtataaggtcctcaccacagcgcctgcccacatagtcagtgatcactatgtcctgaacactgtaattacttcgccatattctctgatcatagtgttttgccttggtatgtgactagaatttctttctgaggtttatgggcatggttggtgggtatgcacctgcctgcaggagcccggtttgggggcattaccttgtacctggtatgttttctttcaggtgtggttcaataacaagggctggcatgcaatcagctctttcctgaatgtcatcaacaatgccattctccgggccaacctgcaaaagggagagaaccctagccattatggaattactgctttcaatcatcccctgaatctcaccaagcagcagctctcagaggtggctctgtaagtgtggctgtgtctgtatagatggagtggggcaagggagagggttatggagaaggggagaaaaatgtgaatctcattgtaggggaacagctgcagagaccgttatattatgataaatctggattgatccaggctctgggcagaagtgataagtttacgaattggctggttgggcttcttgaactgcagaagagaaaatgacactgatatgtaaaaatcgtaacatttagtgaattcatataaagtgagttcaaaaattgttaattaaattataatttaattataagtgtttaatcagtttgatttgtttaaaaaccactgttttaaatttggtggaatatgtttttattagcttgtatctttaattcctaaattaagctgtgtgtgtgtgtgtgtgtgtgtgtgtgtgtgtgtgtgtgtgtgtgtgtgaagtttaaagccaggatgagctagtttaaagtatgcagcctttggagtcatacagatctgggtttgaatctggtctctaaactttatagatgtatgatattaaatgaggcagttcatgtaaattgccaagcccagcactcagcacagagttgatatttcacacacattagatacctttcctgtatgtggagcatggcagttcctgtttctgctttactcctacaggatactaatataggacactaggatctttataccaagaccccatgtaatgggcttatgagaccattcttcttataaaaatctgacagaatttttgtatgtgttagatcaataggctgcatactgttattttcaagttgatttacagccagaaatattaatttatttgagtagttacagagtaatatttctgctctcatttagttttcaagccccactagtcctttgtgtgtgaaaatttacaacttactgctcttacaaggtcatgaacagtggaccaaagtgaatgccattaaccactctgacttccttcattagttttattgtgacagtggactcttttgacctcagtaataccagtttggcatttacattgtcatatttttagacttaaaaatgatcatcttaaccctgaataaaatgtgtctggtgaacagatgtttttccttggctgtgcctcagatatctctgtgtgtgtgtacgtgtgtgtttgtctgtgtgtccatgtcctcactgattgagccctaactgcatcaaagacccctcagattttcacacgctttttctctccaggatgaccacatcagtggatgtccttgtgtccatctgtgtcatctttgcaatgtccttcgtcccagccagctttgtcgtattcctgatccaggagcgggtcagcaaagcaaaacacctgcagttcatcagtggagtgaagcctgtcatctactggctctctaattttgtctgggatatggtaaggacacaggcctgctgtatctttctgatgtctgtcagggccatggattgatatggataagaaagaaagagctctggctatcatcaggaaatgttccagctactctaaagatgtatgaaaaagaaatagccagaggcaggtgatcactttcatgacaccaaacacagcattgggtaccagagttcatgtcacaccagagggaaaattctgtacacaatgatgaaaattaataccactaccacttaagttcctatgtgacaactttcccaagaatcagagagatacaagtcaaaactccaagtcaatgcctctaacttctctgatgggttttaacctccagagtcagaatgttctttgccttactaggaaagccatctgtcatttagaaaactctgtacattttatcagcagcttatccatccattgcaaatattgtttttgtgccasccacaatatattgcttctatttggaccaatatgggggatttgaaggaattctgaagttctaattatatttcaactctactttacaatatctccctgaaatatatctccctgtaacttctattaattataagctacacagagcaaatctaattcttctcccaccgaacaagtccctggatatttaaaaataactctcatactctcatttaacctgagtattacccagataagatgatatatgagaatacaccttgtaacctccgaagcactgtacaaatgtgagcaatgatggtggagatgatgatgagatctttgctgtttataccaagccccttagactgtgtcactcttctgatccggttgtccttgtatggccatgctgtatattgtgaatgtcccgttttcaaaagcaaagccaagaattaaccttgtgttcaggctgtggtctgaatggttatgggtccagagggagttgatctttagctcacacttctattactgcagcacaaagattttgcattttggaaggagcaccgtcttactggcaacttagtggtaaaccaaaacctccatttcacacaaatgattgtgaaattcgggtctccttcattctatacaaattcatttgatttttttgaaactaaactttatatttatccatattaaattacatgggttttatttttgttttatcttgattcagtaattactcctttcagtaaacacagactgagtgctgtgtgtctgacttatgccaggcataggtgattcagagatgaaaggtcaagtccctgaacccatctcttgtcttcctgggtattatctgtccctccctgctttagagctcctgaaatttgctagaagcatgtcttcatctaagttgttgataaacacatcaagtaggattggactgaggcagagccctgtagtctgaagctgcagttcttctagcggctgacaagccccactatcacttccctgctggtgctttgctctgccagctgtgaattctcataattgtcctatcgtcaagtctttatttctgcattttactgcttgatacactgtcaggacagactttaaaattattctcagtgcgatgaaacaattctgacattcatgttatgagcagttacctcataaatagattacatg

SEQ ID NO: 27 - DNA, 4244 bp, Homo sapiens
aaattactctgactgggaatccatcgttcagtaagtttactgagtgtgacaccttggcttgactgttggaaagacagaaagggcatgtagtttataaaatcagccaaggggaaaatgcttgtcaaaatgtattgtcgggtattttgattaatagtttatgtggcttcattaattcagagttactctccaatatgtttatctgccctttcttgtctgataatggtgaaaacttgtgtgatgcattgtatatttgatttaggggtgaactggatgtctttgttttcacttttagtgcaattacgttgtccctgccacactggtcattatcatcttcatctgcttccagcagaagtcctatgtgtcctccaccaatctgcctgtgctagcccttctacttttgctgtatgggtaagtcacctctgagtgagggagctgcacagtggataaggcatttggtgcccagtgtcagaaggagggcagggactctcagtagacacttatctttttgtgtctcaacaggtggtcaatcacacctctcatgtacccagcctcctttgtgttcaagatccccagcacagcctatgtggtgctcaccagcgtgaacctcttcattggcattaatggcagcgtggccacctttgtgctggagctgttcaccgacaatgtgagtcatgcagagagaacactcctgctgggatgagcatctctgggagccagaggacagtgtttaattgtgatcttattccacttgtcagtggtattgacactgctgactgccttgtcctgtcttcagagtctgtcttccctgagaaggcaaagcacctttctttcttgctgtgccttacattttgctggtcaagcctttcagtttcttttgacagttttttttacttctttcttttttcaatgttgctcttaccaagagtagctcctctgccttccactttacacatgagagctgggcgacgcattcagtcctaaggcttttaccatcacctctcttggtgtttttattgtcatctctaagatcaatgcctttagccttgatcataaccttgaactctaatctcaaattctcacttgcctagtggattgctccatttagatagtatatagataccccaacctggatatgtcctagttttctttccccttggaacttaatgcttttcttgccatccctgtcacactcagtggcactaccatccactcggttgcccaagctggctcttagagttatcctagatgcttgctttgctgttgcagatttcccacattcaactggttatgttgtcagttcttccaggtatggacctctaaaataaggcttcctctccattccggttgtcattgcctttgtccaaacacagcacacaaggccttttacagttgcacaactcttcctgtccatacccaccacaccctttcccagctgtaagcttcagatgagttgcctccaaccaccatgctcctgtaggcctggcttgaaatgcccttcttctgtcacagggtctggtagtatatcccttgcccttcaagatttagctaaaatgtgaagctttccttacctgctgggaggtgttctctcttttctctgtgctctcagagtccttagtccatgcctccagtacaacgtacatccacttacatggtaatttcctgtttacatacttttcctactcggagtggagtctgtttcttaataattttgcctctcccatgccctagcacagtgcatccagcgtatagccccttattcagttggtagatatttggccactgttgccttgtgggatcataagttctgatgtatttgagaagaatttctaaaattctgacaaaatcctgaaactcaaatattgacccagacatgagcaatttgcttttcaaatgctaagggatttttaatggatttgctttaattaaatctagcctgtttctaagctttattcattatttctccatactcagagcatttctccagattttctaaagaatagaattttattgctacatatcatcagctatgcctgctgctatttaattggtatctgaattaaaaggtctggtttgtccctagagaatcaaattttttcttcactcccatatttcagaacttgatacatttttaggataaaccatgaatgacacccgtttcttctccctcaccctcccttccctcccatttttttttttttttttttttagaagctgaataatatcaatgatatcctgaagtccgtgttcttgatcttcccacatttttgcctgggacgagggctcatcgacatggtgaaaaaccaggcaatggctgatgccctggaaaggtttggtgagtgaagcagtggctgtaggatgctttaatggagatggcactctgcataggccttggtaccctgaactttgttttggaaagaagcaggtgactaagcacaggatgttcccccacccccatgcccagtgacagggctcatgccaacacagctggttgtggcatgggttttgtgacacaaccatttgtctgtgtctctgatagcattgagaaaagtgaaagggcagttttgaaggtaaggaaaatagtgttatttgcttggatccactggctcatgccactgtctgggttggttagaagcactggaaaagtcaaaccataactttgagaattaggtgatcagggaatcagaaggaaagatgcaaactttggctcttttaggcgaatcatgtgcctgcagatgaggtcatttattatcttttacacagtctataaaattataatgtattacatctttttctacctttagaatggttaaaaatatttctccggtagccatatgattattattcatccattagataatatagtcaaatgggccatgttatttactgttcatagaagaggggctttttgcaacttgggctacaaaggagatatgtaaggaatttaaggaatggttacatggaactagatttaattgaatctagtggtttaattgattcactaggatatatgctactgaaaggggaatctgcttaaagtgctttctgatatttattattactaaaacttagaatttattaaaaatactgactgtgaaaattacttgggtcgtttgcctttttaaaaggatttttggcatgtctcattaaaaaaagaaatactagatatcttcagtgaagttacaaatcgaatacacattggctctgaaattctgattgatactgggtcataaaaagttttcccaaatcagacttggaaagtgatcactctcttgttactcttttttccttgtcatgggtgatagccatttgtgtttattggaagatcggtgaattttaaggaacataggcccaaatttgaggaagggccatggtttttgatccctccattctgaccggatctctgcattgtgtctactaggggagaatcgctttgtgtcaccattatcttgggacttggtgggacgaaacctcttcgccatggccgtggaaggggtggtgttcttcctcattactgttctgatccagtacagattcttcatcaggcccaggtgagctttttcttagaacccgtggagcacctggttgagggtcacagaggaggcgcacagggaaacactcaccaatgggggttgcattgaactgaactcaaaatatgtgataaaactgattttcctgatgtgggcatcccgcagccccctccctgcccatcctggagactgtggcaagtaggttttataatactacgttagagactgaatctttgtcctgaaaaatagtttgaaaggttcatttttcttgttttttcccccaagacctgtaaatgcaaagctatctcctctgaatgatgaagatgaagatgtgaggcgggaaagacagagaattcttgatggtggaggccagaatgacatcttagaaatcaaggagttgacgaaggtgagagagtacaggttacaatagctcatcttcagtttttttcagctttatgtgctgtaacccagcagtttgctgacttgcttaataaaagggcatgtgttcccaaaatgtacatctataccaaggttctgtcaattttattttaaaaacaccatggagacttcttaaagaattcttactgagaattcttttgtgatatgaattcccattctcgaatactttggttttatatgcttacatttatgtgttagttattaaaacatactaatattgtatatctagtcaaactgagtagagagataatggtgatt

SEQ ID NO: 28 - DNA, 5023 bp, Homo sapiens
ttttaaaatacctgcaatacatatatatgttgaatagatgaaaaattatgtagatgataatgaatgatacggttctaaaaagacaggttaaaaagtaagttcacttttattttgagcttcagaatcattcagaagccagtcgccacaaacgcagaccaaggctcttggcacatcaaatatgcctatggcttagggttattgacaagtcttatgttgcagtgtatgtggtttatagtcctgccttccacagttgcttgggagagctgtgagtcactgaggcttatgaatgtttacattttgtttgttgcagatatatagaaggaagcggaagcctgctgttgacaggatttgcgtgggcattcctcctggtgaggtaaagacactttgtctatattgcgtttgtccctattagttcagactatctctacccaatcaagcaacgatgctcgttaagaggtaaaagtggattttaaaggcttctgtatttatgccaggatggagcaattagtcatcgagaagagagggaccctgtatgtcaagagaatgatttcagagaatccaatacaatttaagaaaaagcatggggctgggcgcagtgattcactcctgtaatcccagcactttgggaggccgaggtgggcggactcacgaggtcaggagattgagaccatcctggccaacatggtgaaaccccatctctactataaatacaaaaattagctgggcatagtagtgcattcctgtagtcccagctactcgggaggctgaggcaggagaattgcttgaacctaggagggggaggttgcccagattgcgctgctgcactccagcctggtgacagagtgagactcatgtcaacaacaaaaacagaaaaagcacgcacatctaaaacatgcttttgtgatccatttgggatggtgatgacattcaaatagttttttaaaaatagattttctcctttctggtttccgtttgtgttcttttatgcccttttgccagagtaggtggtgcaatttggctagctggctttcattactgtttttcacacattaactttggcctcaacttgacaactcaaataatatttataaatacagccacacttaaaatggtcccattatgaaatacatatttaaatatctatacgatgtgttaaaaccaagaaaatatttgattcttctctgatatttaagaattgaaggtttgaggtagttacgtgttaggggcatttatattcatgtttttagagtttgcttatacaacttaatctttccttttcagtgctttgggctcctgggagttaatggggctggaaaatcatcaactttcaagatgttaacaggagataccactgttaccagaggagatgctttccttaacaaaaataggtgagaaaagaagtggcttgtattttgctgcaaagactttgtttttaatttatttaaagaaataggttgttatttttgattacagtggtatttttagagttcataaaaatgttgaaatatagtaaagggtaaagaagcacataaaatcatccatgatttcaatatctagagataatcacaatttacatttcctttcagtctcattctcttcttttaacagctttattcaggtataatttacatacaatataatttgcttgttttttaagagtataatttagtgatttttggtaaattgagagttttgcaaccatcaccacaatccagttttagaacttttccatcaccccacatctgtcttatatacacatataaatgtgccatacaattgagatcatactgtatgtagaatttaaaattagtttttattgttaatgagtgtattatgaatatttcccagtgggttacatttcctaagatgtggaattttacattgctacataaaatccccctatgtacatgtacctataatttatttaataaattccttataaatgttggacacattagtttccatttttcactatgtaaatatgtccctgtatacatcttttattatttcctcaggaacaattcctacaaagtaaattgccctctctaaagagcatacaaattgactgagccaccgttaggccattttctgagactgcacaggtcacaaagcaatctgatctttgggaatacagctacattttataggcttcttagataatgttactctaagtactttaaatatgtggggcttctctgggcttttttttttttgagacggagtttcactcttactgcccaggctggagagcaatggcgcgaccttggctcactgcaacctccgcctcccaggttcaagcgattctcctgcctcagcctcctgagtagctgagattacaggtgcccgccacaatgcctgcctaatttttttgtattttcagtagagatggggtttcaccatgttggccagactggtctcgagctcctgacctcaggtgatccacctgcctcagcctcccaaagttctgggattacaggcatgagccactgcgcccggcttctctggacttattatgtggagagatagtacaaggcagtggctttcagagttttttgaccatgaccgttgtgggaaatacattttatatctcaacctagtatgtacacacagacatgtagacacatgtataacctaaagtttcataaagcagtacctactgttactaattgtagtgcactctgctatttcttattctaccttatactgcgtcattaaaaaagtgctggtcatgacccactaaatttatttcccaaaccactaatgaacaatgactcacaatttgaacacactggacagggggatagccaataaaattgaaaagagcaaggaaattaatgtattcatgatctcctctcctgtctcttacatttttgcagtagcaatgtaaaggaatcctaagagaacagacattctgggaatagcaggcctagcgctgcacaactgctttcctaggcttgctcctagtaccaagctcctgacgcatatagcagtggcagtaataaccagcccatagtaaggtttgtcacagggactggttgtaagaactgatttgrttggtatagctgtgagggcctggcacggtgtccacgtgtgcctcaatcctaattctgaaaaaggctgaccctgggggtgctaattagatacacagagaggaatgaatgctgccagaaggccaagttcatggcaatgccgctgtggctgaggtgcagtcatcagtctggaacgtgaacactgaacttctctcacatgtgattcttcacttgactggcttcatagaaccccaaagccaccccaccaccacataaattgtgtctctaggttctgtgttgctcacactcaaaatttctgggccttctcatttggtgcatgtgaatggtgcatatgagtgaagtctaggatggggccttagcgttaaagccctggggtagtgtgactgagattgttggtaaagaatgtgcagtggttggcatgacctcagaaattctgaaatgggactgcacctgcagactgaagtgttcagagagccagggaggtgcaaggactggggagggtagaggcaggaaccctgcctgccaggaagagctagcatcctgggggcagaaaggctgtgctttcaagtagcagcagatgtattggtatctttgtaatggagaagcatactttacaggaacattaggccagattgtctaaccagagtatctctacctgcttaaaatctaagtagttttcttgtcctttgcagtatcttatcaaacatccatgaagtacatcagaacatgggctactgccctcagtttgatgccatcacagagctgttgactgggagagaacacgtggagttctttgcccttttgagaggagtcccagagaaagaagttggcaaggtactgtgggcacctgaaagccagcctgtctcctttggcatcctgacaatatataccttatggcttttccacacgcattgacttcaggctgtttttcctcatgaatgcagcagcacaaaatgctggttctttgtatctgctttcagggtggaaacctgtaacggtggtggggcagggctgggtgggcagagagggagtgctgctcccaccacacgagtcccttctccctgctttggctcctcaccagttgtcaggttatgattatagaatctagtcctactcagtgaaagaactttcatacatgtatgtgtaggacagcatgataaaattcccaagccagaccaaagtcaaggtgctttttatcactgtaggttggtgagtgggcgattcggaaactgggcctcgtgaagtatggagaaaaatatgctggtaactatagtggaggcaacaaacgcaagctctctacagccatggctttgatcggcgggcctcctgtggtgtttctggtgagtataactgtggatggaaaactgttgttctggcctgagtggaaaacatgactgttcaaaagtcctatatgtccagggctgttgtatgattggcttgtcttcccccagggacagcagagcaaccttggaaaagcagagggaagcttctcccttggcacacactggggtggctgtaccatgcctgcagatgctcccaaatagaggcactccaagcactttgtttcttagcgtgattgaggctggatatgtgatttgatctttctctggaacattctttctaatcatctttgtgttcattccctgaaaatgaagagtgtggacacagctttaaaatccccaaggtagcaactaggtcatagttccttacacacggatagatgaaaaacagatcagactgggaagtggcccttgaccttttttcttctgtagataagagcattgatgttattacgggaagaagcctttgaggcttttatgtattccacctcggtctggaatttgtttctgtaaggctaacagttgcaatatactagggtaatctgagtgagctggaattaaaaaaaaaaaggaatttcaccccaatcttatactgacttcaatagaggtttcagacaaaaagttgttttgtat

SEQ ID NO: 29 - DNA, 5138 bp, Homo sapiens
  misc_feature (1)...(5138): n = a, t, c, or g
ngccnngttnaaaangaaaatttnnnnnaaattnaannttannggngnnntttccccagaaaaaacnaaaangatttccncccnggggggncccccnantcnaaaaggccccncttntttgnggngagggaaagntttttttggaatttttaatttttggtcccccaaaacctattattgagaatttaattacataaaaaagtactcagaatatttgagtttcctgcatcaataagacatttataataatgaccttgtttacaaatgaatttgaaagttactctaattctttgattcatcaagaaataactagaatggcaagttaaaatttaagctgtttcaaagatgcttctgcatttaaaaacaaatttatctttgattttttttccccccagcaaataagacttattttattctaattacaggatgaacccaccacaggcatggatcccaaagcccggcggttcttgtggaattgtgccctaagtgttgtcaaggaggggagatcagtagtgcttacatctcataggtccgtagtaaagtcttgggttcctcactgtgggatgttttaactttccaagtagaatatgcgatcattttgtaaaaattagaaaatacagaaaagcaaagagtaaaacaattattacctgaaattatatatgcatattcttacaaaaatgcaagcccagtataaatactgctctttttcacttaatatattgtaaacattattccaagtcagtgcatttaggtgtcatttcttatagctggatagtattccattaggatatactcttatttaactattcccccttttgtagacatttggattatttccaacttgttcacaattgtaaacaccactacactgaacagcatcatccctatatccacatgtacttgtaacagaatacaattccctaggaagctggaatgctggaagtcatggtgatgttctcatggttacagagaatctctctaaaactaaaacctctttctgttttaccgcagtatggaagaatgtgaagctctttgcactaggatggcaatcatggtcaatggaaggttcaggtgccttggcagtgtccagcatctaaaaaataggtaataaagataatttctttgggatagtgcctagtgagaaggcttgatatttattcttttgtgagtatataaatggtgcctctaaaataaagggaaataaaactgagcaaaacagtatagtggaaagaatgagggctttgaagtccgaactgcattcaaattctgtctttaccatttactggttctgtgactcttgggcaagttacttaactactgtaagagttagtttccctggaagatctacctcctagctttgtgctatagatgaaatgaaaaaaatttacatgtgccagtactggtgagagcgcaagctttggagtcaaacacaaatgggtttgcatcctggccctaccaattatgagctctgagccatgggcaagtgactaactccctgggcctcagtttctctgtaacatctgtcagacttcatgggtccaggtgaggattaaaggagatcatgtatttacagcacatggcatggtgcttcacataaaataagtatttagtaaatgataactggttccttctctcagaaacttatttctgggcctgccaggggccgccctttttcatggcacaagttgggttcccagggttcagtattcttttaaatagttttctggagatcctccatttgggtattttttcctgctttcaggtttggagatggttatacaatagttgtacgaatagcagggtccaacccggacctgaagcctgtccaggatttctttggacttgcatttcctggaagtgttcyaaaagagaaacaccggaacatgctacaataccagcttccatcttcattatcttctctggccaggatattcagcatcctctcccagagcaaaaagcgactccacatagaagactactctgtttctcagacaacacttgaccaagtaagctttgagtgtcaaaacagatttacttctcagggtgtggattcctgccccgacactcccgcccataggtccaagagcagtttgtatcttgaattggtgcttgaattcctgatctactattcctagctatgctttttactaaacctctctgaacctgaaaagggagatgatgcctatgtactctataggattattgtgagaatttactgtaataataaccataaaaactaccatttagtgagcacctaccatgggccaggcattttacttggtgcctaatcctatttaaattagataaaaaagtaccaaataggtcctgacacttaagaagtactcagtaaatattttcttccctcttccctttaatcaagaccgtatgtgccaaagtaaatggatgactgagcagttggtgatgtaggggtggggggcgatatagaaagtcagtttttggccgggcgtggtggctcatgcctgtaatcccagcactttgggaggctgaggagcaggcagatcatgaggtcaggagatccagataatcctggccaacagggtgaaaccccgtctctactaaaaatacaaaaattagctgggcatggtggtgcgcacttgtagtcccagctacttgcgaggctgaggcaggagaattgctcgaacccaggaggtggaggttacagtgagccaaggtctcgccactgcactccagcctggggacagagcaagaccccatttcaaggggggaaaaaaagtctatttttaagttgttattgcttttttcaagtattcttccctccttcacacacagttttctagttaatccatttatgtaattctgtatgctcctacttgacctaatttcaacatctggaaaaatagaactagaataaagaatgagcaagttgagtggtatttataaaggtccatcttaatcttttaacaggtatttgtgaactttgccaaggaccaaagtgatgatgaccacttaaaagacctctcattacacaaaaaccagacagtagtggacgttgcagttctcacatcttttctacaggatgagaaagtgaaagaaagctatgtatgaagaatcctgttcatacggggtggctgaaagtaaagaggaactagactttcctttgcaccatgtgaagtgttgtggagaaaagagccagaagttgatgtgggaagaagtaaactggatactgtactgatactattcaatgcaatgcaattcaatgcaatgaaaacaaaattccattacaggggcagtgcctttgtagcctatgtcttgtatggctctcaagtgaaagacttgaatttagttttttacctatacctatgtgaaactctattatggaacccaatggacatatgggtttgaactcacacttttttttttttttttgttcctgtgtattctcattggggttgcaacaataattcatcaagtaatcatggccagcgattattgatcaaaatcaaaaggtaatgcacatcctcattcactaagccatgccatgcccaggagactggtttcccggtgacacatccattgctggcaatgagtgtgccagagttattagtgccaagtttttcagaaagtttgaagcaccatggtgtgtcatgctcacttttgtgaaagctgctctgctcagagtctatcaacattgaatatcagttgacagaatggtgccatgcgtggctaacatcctgctttgattccctctgataagctgttctggtggcagtaacatgcaacaaaaatgtgggtgtctccaggcacgggaaacttggttccattgttatattgtcctatgcttcgagccatgggtctacagggtcatccttatgagactcttaaatatacttagatcctggtaagaggcaaagaatcaacagccaaactgctggggctgcaactgctgaagccagggcatgggattaaagagattgtgcgttcaaacctagggaagcctgtgcccatttgtcctgactgtctgctaacatggtacactgcatctcaagatgtttatctgacacaagtgtattatttctggctttttgaattaatctagaaaatgaaaagatggagttgtattttgacaaaaatgtttgtactttttaatgttatttggaattttaagttctatcagtgacttctgaatccttagaatggcctctttgtagaaccctgtggtatagaggagtatggccactgcccactatttttattttcttatgtaagtttgcatatcagtcatgactagtgcctagaaagcaatgtgatggtcaggatctcatgacattatatttgagtttctttcagatcatttaggatactcttaatctcacttcatcaatcaaatattttttgagtgtatgctgtagctgaaagagtatgtacgtacgtataagactagagagatattaagtctcagtacacttcctgtgccatgttattcagctcactggtttacaaatataggttgtcttgtggttgtaggagcccactgtaacaatactgggcagcctttttttttttttttttaattgcaacaatgcaaaagccaagaaagtttaagggtcacaagtctaaacaatgaattcttcaacagggaaaacagctagcttgaaaacttgctgaaaaacacaacttgtgtttatggcatttagtaccttcaaataattggctttgcagatattggataccccattaaatctgacagtctcaaatttttcatctcttcaatcactagtcaagaaaaaatataaaaacaacaaatacttccatatggagcatttttcagagttttctaacccagtcttatttttctagtcagtaaacatttgtaaaaatactgtttcactaatacttactgttaactgtcttgagagaaaagaaaaatatgagagaactattgtttggggaagttcaagtgatctttcaatatcattactaacttcttccactttttccagaatttgaatattaacgctaaaggtgtaagacttcagatttcaaattaatctttctatattttttaaatttacagaatattatataacccactgctgaaaaagaaacaaatgattgttttagaagttaaaggtcaatattgattttaaaatattaag

SEQ ID NO: 30 - DNA, 20 bp, Homo sapiens
gtgttcctgcagagggcatg

SEQ ID NO: 31 - DNA, 20 bp, Homo sapiens
cacttccagtaacagctgac

SEQ ID NO: 32 - DNA, 21 bp, Homo sapiens
ctttgcgcatgtccttcatgc

SEQ ID NO: 33 - DNA, 21 bp, Homo sapiens
gacatcagccctcagcatctt

SEQ ID NO: 34 - DNA, 19 bp, Homo sapiens
caacaagccatgttccctc

SEQ ID NO: 35 - DNA, 18 bp, Homo sapiens
catgttccctcagccagc

SEQ ID NO: 36 - DNA, 19 bp, Homo sapiens
cagagctcacagcagggac

SEQ ID NO: 37 - protein, 21 aa, Homo sapiens
CSVRLSYPPYEQHECHFPNKA

SEQ ID NO: 38 - DNA, 14 bp, Homo sapiens
gcctgtgtgtcccc

SEQ ID NO: 39 - DNA, 14 bp, Homo sapiens
  misc_feature (1)...(14): n = t or c
gcctgtgngtcccc

SEQ ID NO: 40 - DNA, 45 bp, Homo sapiens
aagaagatgctgcctgtgtgtcccccaggggcaggggggctgcct

SEQ ID NO: 41 - protein, 15 aa, Homo sapiens
KKMLPVCPPGAGGLP

SEQ ID NO: 42 - protein, 15 aa, Mus musculus
KKMLPVCPPGAGGLP

SEQ ID NO: 43 - protein, 15 aa, Homo sapiens
KKMLPVRPPGAGGLP

SEQ ID NO: 44 - protein, 5 aa, Caenorhabditis elegans
LLGGS

SEQ ID NO: 45 - DNA, 45 bp, Homo sapiens
aagaagatgctgcctgtgcgtcccccaggggcaggggggctgcct

SEQ ID NO: 46 - DNA, 14 bp, Homo sapiens
gcctacttgcagga

SEQ ID NO: 47 - DNA, 14 bp, Homo sapiens
gcctacttgcggga

SEQ ID NO: 48 - DNA, 45 bp, Homo sapiens
tgggggggcttcgcctacttgcaggatgtggtggagcaggcaatc

SEQ ID NO: 49 - protein, 15 aa, Homo sapiens
WGGFAYLQDVVEQAI

SEQ ID NO: 50 - protein, 15 aa, Mus musculus
WGGFAYLQDVVEQAI

SEQ ID NO: 51 - protein, 15 aa, Homo sapiens
WGGFAYLRDVVEQAI

SEQ ID NO: 52 - protein, 12 aa, Caenorhabditis elegans
FMTVQRAVDVAI

SEQ ID NO: 53 - DNA, 45 bp, Homo sapiens
tgggggggcttcgcctacttgcgggatgtggtggagcaggcaatc

SEQ ID NO: 54 - DNA, 25 bp, Homo sapiens
  misc_feature (1)...(25): n is a, t, c, or g.
tcattcctcttgtnngcncngnncn

SEQ ID NO: 55 - DNA, 45 bp, Homo sapiens
agtagcctcattcctcttcttgtgagcgctggcctgctagtggtc

SEQ ID NO: 56 - protein, 15 aa, Homo sapiens
SSLIPLLVSAGLLVV

SEQ ID NO: 57 - protein, 15 aa, Mus musculus
SSLIPLLVSAGLLVV

SEQ ID NO: 58 - protein, 14 aa, Homo sapiens
SSLIPLVSAGLLVV

SEQ ID NO: 59 - protein, 15 aa, Caenorhabditis elegans
INYAKLTFAVIVLTI

SEQ ID NO: 60 - DNA, 42 bp, Homo sapiens
agtagcctcattcctcttgtgagcgctggcctgctagtggtc

SEQ ID NO: 61 - DNA, 25 bp, Homo sapiens
  misc_feature (1)...(25): n is a, t, c, or g.
tgatgaagatgananncngnngcga

SEQ ID NO: 62 - DNA, 36 bp, Homo sapiens
aatgatgaagatgaagatgtgaggcgggaaagacag

SEQ ID NO: 63 - protein, 12 aa, Homo sapiens
NDEDEDVRRERQ

SEQ ID NO: 64 - protein, 12 aa, Mus musculus
NDEDEDVRRERQ

SEQ ID NO: 65 - protein, 10 aa, Homo sapiens
NDEDVRRERQ

SEQ ID NO: 66 - protein, 15 aa, Caenorhabditis elegans
DERDVEDSDVIAEKS

SEQ ID NO: 67 - DNA, 30 bp, Homo sapiens
aatgatgaagatgtgaggcgggaaagacag

SEQ ID NO: 68 - DNA, 14 bp, Homo sapiens
agttgtacgaatag

SEQ ID NO: 69 - DNA, 14 bp, Homo sapiens
  misc_feature (1)...(14): n is t or c.
agttgtangaatag

SEQ ID NO: 70 - DNA, 20 bp, Homo sapiens
ggctggattagcagtcctca

SEQ ID NO: 71 - DNA, 20 bp, Homo sapiens
ggatttcccagatcccagtg

SEQ ID NO: 72 - DNA, 20 bp, Homo sapiens
gacagacttggcatgaagca

SEQ ID NO: 73 - DNA, 20 bp, Homo sapiens
gcacttggcagtcacttctg

SEQ ID NO: 74 - DNA, 20 bp, Homo sapiens
cgtttctccactgtcccatt

SEQ ID NO: 75 - DNA, 20 bp, Homo sapiens
acttcaaggacccagcttcc

SEQ ID NO: 76 - DNA, 24 bp, Homo sapiens
tcggtttcttgtttgttaaactca

SEQ ID NO: 77 - DNA, 20 bp, Homo sapiens
tcccaaggctttgagatgac

SEQ ID NO: 78 - DNA, 19 bp, Homo sapiens
ggctccaaagcccttgtaa

SEQ ID NO: 79 - DNA, 20 bp, Homo sapiens
gctgctgtgatggggtatct

SEQ ID NO: 80 - DNA, 25 bp, Homo sapiens
tttgtaaattttgtagtgctcctca

SEQ ID NO: 81 - DNA, 20 bp, Homo sapiens
tagtcagcccttgcctccta

SEQ ID NO: 82 - DNA, 20 bp, Homo sapiens
aaaggggcttggtaagggta

SEQ ID NO: 83 - DNA, 20 bp, Homo sapiens
gatgtggtgctccctctagc

SEQ ID NO: 84 - DNA, 20 bp, Homo sapiens
caagtgagtgcttgggattg

SEQ ID NO: 85 - DNA, 21 bp, Homo sapiens
gcaaattcaaatttctccagg

SEQ ID NO: 86 - DNA, 20 bp, Homo sapiens
tcaaggaggaaatggacctg

SEQ ID NO: 87 - DNA, 20 bp, Homo sapiens
ctgaaagttcaagcgcagtg

SEQ ID NO: 88 - DNA, 20 bp, Homo sapiens
tgcagactgaatggagcatc

SEQ ID NO: 89 - DNA, 20 bp, Homo sapiens
gccaggggacactgtattct

SEQ ID NO: 90 - DNA, 20 bp, Homo sapiens
aggtcctctgccttcactca

SEQ ID NO: 91 - DNA, 20 bp, Homo sapiens
ccagtgcttacccctgctaa

SEQ ID NO: 92 - DNA, 21 bp, Homo sapiens
cacacaacagagcttcttgga

SEQ ID NO: 93 - DNA, 20 bp, Homo sapiens
acctggaacaggtgtggtgt

SEQ ID NO: 94 - DNA, 21 bp, Homo sapiens
gggctaacatgccactcagta

SEQ ID NO: 95 - DNA, 20 bp, Homo sapiens
gtttgttgcagatggggaag

SEQ ID NO: 96 - DNA, 20 bp, Homo sapiens
caccagaagaaggagcatgg

SEQ ID NO: 97 - DNA, 20 bp, Homo sapiens
ctggactcgtagggatttgc

SEQ ID NO: 98 - DNA, 21 bp, Homo sapiens
gcctgtcacagagaaatgctt

SEQ ID NO: 99 - DNA, 21 bp, Homo sapiens
ttacggaatgatcctgtgctc

SEQ ID NO: 100 - DNA, 20 bp, Homo sapiens
agtcaggtttccggtcacac

SEQ ID NO: 101 - DNA, 22 bp, Homo sapiens
ccgttccttatatcctcaggtg

SEQ ID NO: 102 - DNA, 21 bp, Homo sapiens
ccttgtacacactcgcactga

SEQ ID NO: 103 - DNA, 20 bp, Homo sapiens
tgttgtccacaggttccaga

SEQ ID NO: 104 - DNA, 20 bp, Homo sapiens
tgaggtttatgggcatggtt

SEQ ID NO: 105 - DNA, 20 bp, Homo sapiens
atgtttttccttggctgtgc

SEQ ID NO: 106 - DNA, 20 bp, Homo sapiens
atctgccctttcttgtctga

SEQ ID NO: 107 - DNA, 20 bp, Homo sapiens
agggagctgcacagtggata

SEQ ID NO: 108 - DNA, 24 bp, Homo sapiens
tcactcccatatttcagaacttga

SEQ ID NO: 109 - DNA, 22 bp, Homo sapiens
tgtttattggaagatcggtgaa

SEQ ID NO: 110 - DNA, 25 bp, Homo sapiens
cgttagagactgaatctttgtcctg

SEQ ID NO: 111 - DNA, 20 bp, Homo sapiens
agtcctgccttccacagttg

SEQ ID NO: 112 - DNA, 21 bp, Homo sapiens
ggtagttacgtgttaggggca

SEQ ID NO: 113 - DNA, 21 bp, Homo sapiens
caggaacattaggccagattg

SEQ ID NO: 114 - DNA, 23 bp, Homo sapiens
catgtatgtgtaggacagcatga

SEQ ID NO: 115 - DNA, 21 bp, Homo sapiens
ctgtttcaaagatgcttctgc

SEQ ID NO: 116 - DNA, 20 bp, Homo sapiens
cctaggaagctggaatgctg

SEQ ID NO: 117 - DNA, 20 bp, Homo sapiens
gggttcccagggttcagtat

SEQ ID NO: 118 - DNA, 23 bp, Homo sapiens
cttgacctaatttcaacatctgg

SEQ ID NO: 119 - DNA, 20 bp, Homo sapiens
atccccaactcaaaaccaca

SEQ ID NO: 120 - DNA, 21 bp, Homo sapiens
aagtccaatttagcccacgtt

SEQ ID NO: 121 - DNA, 20 bp, Homo sapiens
ccagccattcaaaattctcc

SEQ ID NO: 122 - DNA, 20 bp, Homo sapiens
ggtgcaggtcaatttccaat

SEQ ID NO: 123 - DNA, 20 bp, Homo sapiens
ccccttcaccaccattacaa

SEQ ID NO: 124 - DNA, 20 bp, Homo sapiens
tgtccaaggaaaagcctcac

SEQ ID NO: 125 - DNA, 20 bp, Homo sapiens
aggacctcttgccagactca

SEQ ID NO: 126 - DNA, 20 bp, Homo sapiens
aggagatgacacaggccaag

SEQ ID NO: 127 - DNA, 20 bp, Homo sapiens
cgcacacctctgaagctacc

SEQ ID NO: 128 - DNA, 20 bp, Homo sapiens
acctcactcacacctgggaa

SEQ ID NO: 129 - DNA, 20 bp, Homo sapiens
gcctcctgcctgaaccttat

SEQ ID NO: 130 - DNA, 23 bp, Homo sapiens
caaaatcatgacaccaagttgag

SEQ ID NO: 131 - DNA, 20 bp, Homo sapiens
catgcacatgcacacacata

SEQ ID NO: 132 - DNA, 20 bp, Homo sapiens
ccttagcccgtgttgagcta

SEQ ID NO: 133 - DNA, 21 bp, Homo sapiens
tgcttttattcagggactcca

SEQ ID NO: 134 - DNA, 20 bp, Homo sapiens
cccatgcactgcagagattc

SEQ ID NO: 135 - DNA, 19 bp, Homo sapiens
aaggcaggagacatcgctt

SEQ ID NO: 136 - DNA, 20 bp, Homo sapiens
gggatcagcatggtttccta

SEQ ID NO: 137 - DNA, 20 bp, Homo sapiens
gcttaagtcccactcctccc

SEQ ID NO: 138 - DNA, 20 bp, Homo sapiens
attttcctccgcatgtgtgt

SEQ ID NO: 139 - DNA, 20 bp, Homo sapiens
tcacagaagcctagccatga

SEQ ID NO: 140 - DNA, 20 bp, Homo sapiens
aacagagcagggagatggtg

SEQ ID NO: 141 - DNA, 20 bp, Homo sapiens
tctgcacctctcctcctctg

SEQ ID NO: 142 - DNA, 20 bp, Homo sapiens
actggggccaacattaatca

SEQ ID NO: 143 - DNA, 20 bp, Homo sapiens
cttccccatctgcaacaaac

SEQ ID NO: 144 - DNA, 20 bp, Homo sapiens
gctaaaggccatccaaagaa

SEQ ID NO: 145 - DNA, 20 bp, Homo sapiens
tcaagtgcatctgggcataa

SEQ ID NO: 146 - DNA, 20 bp, Homo sapiens
tctgaagtccattcccttgg

SEQ ID NO: 147 - DNA, 20 bp, Homo sapiens
caatgtggcatgcagttgat

SEQ ID NO: 148 - DNA, 19 bp, Homo sapiens
gaagctaccagcccatcct

SEQ ID NO: 149 - DNA, 20 bp, Homo sapiens
catttcccccactgtttcag

SEQ ID NO: 150 - DNA, 20 bp, Homo sapiens
ccaaggctttcttcaatcca

SEQ ID NO: 151 - DNA, 20 bp, Homo sapiens
gatccgtttaacctgccaac

SEQ ID NO: 152 - DNA, 19 bp, Homo sapiens
atgcccctgccaactttac

SEQ ID NO: 153 - DNA, 20 bp, Homo sapiens
ctctgcagctgttcccctac

SEQ ID NO: 154 - DNA, 20 bp, Homo sapiens
tatcaatccatggccctgac

SEQ ID NO: 155 - DNA, 20 bp, Homo sapiens
agagtccctgccctccttct

SEQ ID NO: 156 - DNA, 20 bp, Homo sapiens
aaggcagtcagcagtgtcaa

SEQ ID NO: 157 - DNA, 20 bp, Homo sapiens
ggggaacatcctgtgcttag

SEQ ID NO: 158 - DNA, 20 bp, Homo sapiens
ccattggtgagtgtttccct

SEQ ID NO: 159 - DNA, 20 bp, Homo sapiens
agtcagcaaactgctgggtt

SEQ ID NO: 160 - DNA, 20 bp, Homo sapiens
attgctccatcctggcataa

SEQ ID NO: 161 - DNA, 23 bp, Homo sapiens
tcatggatgattttatgtgcttc

SEQ ID NO: 162 - DNA, 20 bp, Homo sapiens
gcgtgtggaaaagccataag

SEQ ID NO: 163 - DNA, 20 bp, Homo sapiens
gccaatcatacaacagccct

SEQ ID NO: 164 - DNA, 23 bp, Homo sapiens
tgatcgcatattctacttggaaa

SEQ ID NO: 165 - DNA, 22 bp, Homo sapiens
tccctttattttagaggcacca

SEQ ID NO: 166 - DNA, 21 bp, Homo sapiens
gatcaggaattcaagcaccaa

SEQ ID NO: 167 - DNA, 24 bp, Homo sapiens
tgggttccataatagagtttcaca

SEQ ID NO: 168 - DNA, 22 bp, Homo sapiens
tgtcagctgttactggaagtgg

SEQ ID NO: 169 - DNA, 22 bp, Homo sapiens
tgtcagctgctgctggaagtgg

SEQ ID NO: 170 - DNA, 21 bp, Homo sapiens
aggagctggccgaagccacaa

SEQ ID NO: 171 - DNA, 21 bp, Homo sapiens
aggagctggctgaagccacaa

SEQ ID NO: 172 - DNA, 21 bp, Homo sapiens
aatgatgccaccaaacaaatg

SEQ ID NO: 173 - DNA, 21 bp, Homo sapiens
aatgatgccatcaaacaaatg

SEQ ID NO: 174 - DNA, 21 bp, Homo sapiens
gaggtggctccgatgaccaca

SEQ ID NO: 175 - DNA, 21 bp, Homo sapiens
gaggtggctctgatgaccaca

SEQ ID NO: 176 - DNA, 21 bp, Homo sapiens
ttccttaacagaaatagtatc

SEQ ID NO: 177 - DNA, 21 bp, Homo sapiens
ttccttaacaaaaatagtatc

SEQ ID NO: 178 - DNA, 21 bp, Homo sapiens
ggaagtgttccaaaagagaaa

SEQ ID NO: 179 - DNA, 21 bp, Homo sapiens
ggaagtgttctaaaagagaaa

SEQ ID NO: 180 - DNA, 21 bp, Homo sapiens
agtaaagagggactagacttt

SEQ ID NO: 181 - DNA, 21 bp, Homo sapiens
agtaaagaggaactagacttt

SEQ ID NO: 182 - DNA, 21 bp, Homo sapiens
gcctacttgcaggatgtggtg

SEQ ID NO: 183 - DNA, 21 bp, Homo sapiens
gcctacttgcgggatgtggtg

SEQ ID NO: 184 - DNA, 23 bp, Homo sapiens
cctcattcctcttcttgtgagcg

SEQ ID NO: 185 - DNA, 20 bp, Homo sapiens
cctcattcctcttgtgagcg

SEQ ID NO: 186 - DNA, 21 bp, Homo sapiens
gcaggactacgtgggcttcac

SEQ ID NO: 187 - DNA, 21 bp, Homo sapiens
gcaggactacatgggcttcac

SEQ ID NO: 188 - DNA, 21 bp, Homo sapiens
aaaagtctaccgagatgggat

SEQ ID NO: 189 - DNA, 21 bp, Homo sapiens
aaaagtctactgagatgggat

SEQ ID NO: 190 - DNA, 21 bp, Homo sapiens
ggccagatcacctccttcctg

SEQ ID NO: 191 - DNA, 21 bp, Homo sapiens
ggccagatcatctccttcctg

SEQ ID NO: 192 - DNA, 21 bp, Homo sapiens
acacaccacatggatgaagcg

SEQ ID NO: 193 - DNA, 21 bp, Homo sapiens
acacaccacacggatgaagcg

SEQ ID NO: 194 - DNA, 21 bp, Homo sapiens
cctggaagaagtaagttaagt

SEQ ID NO: 195 - DNA, 21 bp, Homo sapiens
cctggaagaactaagttaagt

SEQ ID NO: 196 - DNA, 21 bp, Homo sapiens
gctgcctgtgtgtcccccagg

SEQ ID NO: 197 - DNA, 21 bp, Homo sapiens
gctgcctgtgcgtcccccagg

SEQ ID NO: 198 - DNA, 22 bp, Homo sapiens
tagccattatggaattactgct

SEQ ID NO: 199 - DNA, 21 bp, Homo sapiens
tagccattatcaattactgct

SEQ ID NO: 200 - DNA, 26 bp, Homo sapiens
gatgaagatgaagatgtgaggcggga

SEQ ID NO: 201 - DNA, 20 bp, Homo sapiens
gatgaagatgtgaggcggga

SEQ ID NO: 202 - DNA, 21 bp, Homo sapiens
aatagttgtacgaatagcagg

SEQ ID NO: 203 - DNA, 21 bp, Homo sapiens
aatagttgtatgaatagcagg

SEQ ID NO: 204 - DNA, 21 bp, Homo sapiens
acacgctgggggtgctggctg

SEQ ID NO: 205 - DNA, 21 bp, Homo sapiens
acacgctgggcgtgctggctg

SEQ ID NO: 206 - DNA, 20 bp, Homo sapiens
gaccagccacggcgtccctg

SEQ ID NO: 207 - DNA, 21 bp, Homo sapiens
gaccagccacgggcgtccctg

SEQ ID NO: 208 - DNA, 22 bp, Homo sapiens
cattttcttagaaaagagaggt

SEQ ID NO: 209 - DNA, 22 bp, Homo sapiens
cattttcttagagaagagaggt

SEQ ID NO: 210 - DNA, 21 bp, Homo sapiens
gaaaattagtatgtaaggaag

SEQ ID NO: 211 - DNA, 21 bp, Homo sapiens
gaaaattagtctgtaaggaag

SEQ ID NO: 212 - DNA, 25 bp, Homo sapiens
cctccgcctgccaggttcagcgatt

SEQ ID NO: 213 - DNA, 25 bp, Homo sapiens
cctccgcctgccgggttcagcgatt

SEQ ID NO: 214 - DNA, 25 bp, Homo sapiens
tatgtgctgaccatgggagcttgtt

SEQ ID NO: 215 - DNA, 25 bp, Homo sapiens
tatgtgctgaccgtgggagcttgtt

SEQ ID NO: 216 - DNA, 21 bp, Homo sapiens
gtgacacccaacggagtaggg

SEQ ID NO: 217 - DNA, 21 bp, Homo sapiens
gtgacacccagcggagtaggg

SEQ ID NO: 218 - DNA, 21 bp, Homo sapiens
agtatcccttgttcacgagaa

SEQ ID NO: 219 - DNA, 25 bp, Homo sapiens
agtatccctcccttgttcacgagaa

SEQ ID NO: 220 - DNA, 21 bp, Homo sapiens
ctgggttcctgtatcacaacc

SEQ ID NO: 221 - DNA, 21 bp, Homo sapiens
ctgggttcctatatcacaacc

SEQ ID NO: 222 - DNA, 21 bp, Homo sapiens
ggcctaccaagggagaaactg

SEQ ID NO: 223 - DNA, 21 bp, Homo sapiens
ggcctaccaaaggagaaactg

SEQ ID NO: 224 - DNA, 20 bp, Homo sapiens
tttaaagggggtgattagga

SEQ ID NO: 225 - DNA, 20 bp, Homo sapiens
tttaaaggggttgattagga

SEQ ID NO: 226 - DNA, 22 bp, Homo sapiens
gaagaaatttgtttttttgatt

SEQ ID NO: 227 - DNA, 22 bp, Homo sapiens
gaagaaatttttttttttgatt

SEQ ID NO: 228 - DNA, 21 bp, Homo sapiens
gcgggcatcccgagggagggg

SEQ ID NO: 229 - DNA, 21 bp, Homo sapiens
gcgggcatcctgagggagggg

SEQ ID NO: 230 - DNA, 21 bp, Homo sapiens
agggaggggggctgaagatca

SEQ ID NO: 231 - DNA, 21 bp, Homo sapiens
agggagggggactgaagatca

SEQ ID NO: 232 - DNA, 20 bp, Homo sapiens
aggagccaaacgctcattgt

SEQ ID NO: 233 - DNA, 21 bp, Homo sapiens
aggagccaaagcgctcattgt

SEQ ID NO: 234 - DNA, 21 bp, Homo sapiens
aagccactgtttttaaccagt

SEQ ID NO: 235 - DNA, 21 bp, Homo sapiens
aagccactgtatttaaccagt

SEQ ID NO: 236 - DNA, 21 bp, Homo sapiens
cgtgggcttcacactcaagat

SEQ ID NO: 237 - DNA, 21 bp, Homo sapiens
cgtgggcttcccactcaagat

SEQ ID NO: 238 - DNA, 21 bp, Homo sapiens
tcacactcaagatcttcgctg

SEQ ID NO: 239 - DNA, 21 bp, Homo sapiens
tcacactcaacatcttcgctg

SEQ ID NO: 240 - DNA, 21 bp, Homo sapiens
gcagcctcacccgctcttccc

SEQ ID NO: 241 - DNA, 21 bp, Homo sapiens
gcagcctcactcgctcttccc

SEQ ID NO: 242 - DNA, 21 bp, Homo sapiens
agaagagaatatcagaaatct

SEQ ID NO: 243 - DNA, 21 bp, Homo sapiens
agaagagaatgtcagaaatct

SEQ ID NO: 244 - DNA, 21 bp, Homo sapiens
gcgcagtgccctgtgtcctta

SEQ ID NO: 245 - DNA, 21 bp, Homo sapiens
gcgcagtgcgctgtgtcctta

SEQ ID NO: 246 - DNA, 21 bp, Homo sapiens
gatctaaggttgtcattctgg

SEQ ID NO: 247 - DNA, 21 bp, Homo sapiens
gatctaaggtggtcattctgg

SEQ ID NO: 248 - DNA, 23 bp, Homo sapiens
ctcttctgttagcacagaagaga

SEQ ID NO: 249 - DNA, 23 bp, Homo sapiens
ctcttctgttatcacagaagaga

SEQ ID NO: 250 - DNA, 21 bp, Homo sapiens
cattctagggatcatagccat

SEQ ID NO: 251 - DNA, 21 bp, Homo sapiens
cattctaggggtcatagccat

SEQ ID NO: 252 - DNA, 22 bp, Homo sapiens
aagtacagtgggaggaacagcg

SEQ ID NO: 253 - DNA, 22 bp, Homo sapiens
aagtacagtgtgaggaacagcg

SEQ ID NO: 254 - DNA, 22 bp, Homo sapiens
attcctaaaaaatagaaatgca

SEQ ID NO: 255 - DNA, 22 bp, Homo sapiens
attcctaaaaagtagaaatgca

SEQ ID NO: 256 - DNA, 21 bp, Homo sapiens
ggcccctgccttattattact

SEQ ID NO: 257 - DNA, 21 bp, Homo sapiens
ggcccctgccgtattattact

SEQ ID NO: 258 - DNA, 22 bp, Homo sapiens
tgagagaattacttgaacccgg

SEQ ID NO: 259 - DNA, 22 bp, Homo sapiens
tgagagaattgcttgaacccgg

SEQ ID NO: 260 - DNA, 21 bp, Homo sapiens
tttgctgaaacaatcactgac

SEQ ID NO: 261 - DNA, 21 bp, Homo sapiens
tttgctgaaataatcactgac

SEQ ID NO: 262 - DNA, 22 bp, Homo sapiens
aacctcagttccctcatctgtg

SEQ ID NO: 263 - DNA, 22 bp, Homo sapiens
aacctcagtttcctcatctgtg

SEQ ID NO: 264 - DNA, 21 bp, Homo sapiens
ctggacaccagaaataatgtc

SEQ ID NO: 265 - DNA, 21 bp, Homo sapiens
ctggacaccaaaaataatgtc

SEQ ID NO: 266 - DNA, 21 bp, Homo sapiens
tcctatgtgtcctccaccaat

SEQ ID NO: 267 - DNA, 21 bp, Homo sapiens
tcctatgtgtgctccaccaat

SEQ ID NO: 268 - DNA, 21 bp, Homo sapiens
aagaagtggcttgtattttgc

SEQ ID NO: 269 - DNA, 21 bp, Homo sapiens
aagaagtggcctgtattttgc

SEQ ID NO: 270 - DNA, 23 bp, Homo sapiens
aactgatttgattggtatagctg

SEQ ID NO: 271 - DNA, 23 bp, Homo sapiens
aactgatttggttggtatagctg

SEQ ID NO: 272 - DNA, 21 bp, Homo sapiens
cagggtccaacccggacctga

SEQ ID NO: 273 - DNA, 21 bp, Homo sapiens
cagggtccaatccggacctga

SEQ ID NO: 274 - DNA, 22 bp, Homo sapiens
ttgggaggctaaggcaggagaa

SEQ ID NO: 275 - DNA, 22 bp, Homo sapiens
ttgggaggctgaggcaggagaa

SEQ ID NO: 276 - DNA, 15 bp, Gallus gallus
accaggggaatctcc

SEQ ID NO: 277 - DNA, 15 bp, Gallus gallus
accagggaaatctcc

SEQ ID NO: 278 - DNA, 45 bp, Gallus gallus
cgctacccaacaccaggggaatctcctggtattgttggaaacttc

SEQ ID NO: 279 - protein, 15 aa, Homo sapiens
RYPTPGEAPGVVGNF

SEQ ID NO: 280 - protein, 15 aa, Mus musculus
RYPTPGEAPGVVGNF

SEQ ID NO: 281 - protein, 15 aa, Gallus gallus
RYPTPGESPGIVGNF

SEQ ID NO: 282 - protein, 15 aa, Gallus gallus
RYPTPGKSPGIVGNF

SEQ ID NO: 283 - DNA, 45 bp, Gallus gallus
cgctacccaacaccagggaaatctcctggtattgttggaaacttc

SEQ ID NO: 284 - DNA, 19 bp, Homo sapiens
gcgtcagggatggggacag

SEQ ID NO: 285 - DNA, 20 bp, Homo sapiens
gcgtcagggattggggacag

SEQ ID NO: 286 - DNA, 17 bp, Homo sapiens
ccacttcggtctccatg

SEQ ID NO: 287 - DNA, 17 bp, Homo sapiens
ccacttcgatctccatg

(19) United States
(12) Patent Application Publication
(10) Pub. No.: US 2005/0136421 A1
(43) Pub. Date: Jun. 23, 2005

(54) Methods and reagents for modulating cholesterol levels

(76) Inventors: Michael R. Hayden, Vancouver (CA); Angela R. Brooks-Wilson, Richmond (CA); Simon N. Pimstone, Vancouver (CA)

Correspondence Address: CARELLA, BYRNE, BAIN, GILFILLAN,;CECCHI, STEWART & OLSTEIN, 6 Becker Farm Road, Roseland, NJ, 07068, US

(21) Appl. No.: 10/818,279

(22) Filed: Apr. 5, 2004

Related U.S. Application Data
(63) Continuation of application No. 10/745,377, filed on Dec. 23, 2003.

(51) Int. Cl. C12Q 1/68

(52) U.S. Cl. 435/6

(57) ABSTRACT

The invention features ABC1 nucleic acids and polypeptides for the diagnosis and treatment of abnormal cholesterol regulation. The invention also features methods for identifying compounds for modulating cholesterol levels in an animal (e.g., a human).

This application claims priority from U.S. Provisional Application No. 60/124,702, filed Mar. 15, 1999, U.S. Provisional Application No. 60/138,048, filed Jun. 8, 1999, U.S. Provisional Application No. 60/139,600, filed Jun. 17, 1999, and U.S. Provisional Application No. 60/151,977, filed Sep. 1, 1999.

### BACKGROUND OF THE INVENTION

Low HDL cholesterol (HDL-C), or hypoalphalipoproteinemia, is a blood lipid abnormality which correlates with a high risk of cardiovascular disease (CVD), in particular coronary artery disease (CAD), but also cerebrovascular disease, coronary restenosis, and peripheral vascular disease. HDL, or ‘good cholesterol’ levels are influenced by both environmental and genetic factors.

Epidemiological studies have consistently demonstrated that plasma HDL-C) concentration is inversely related to the incidence of CAD. HDL-C levels are a strong graded and independent cardiovascular risk factor. Protective effects of an elevated HDL-C persist until 80 years of age. A low HDL-C is associated with an increased CAD risk even with normal (<5.2 mmol/l) total plasma cholesterol levels. Coronary disease risk is increased by 2% in men and 3% in women for every 1 mg/dL (0.026 mmol/l) reduction in HDL-C and in the majority of studies this relationship is statistically significant even after adjustment for other lipid and non-lipid risk factors. Decreased HDL-C levels are the most common lipoprotein abnormality seen in patients with premature CAD. Four percent of patients with premature CAD with have an isolated form of decreased HDL-C levels with no other lipoprotein abnormalities while 25% have low HDL levels with accompanying hypertriglyceridemia.

Even in the face of other dyslipidemias or secondary factors, HDL-C levels are important predictors of CAD. In a cohort of diabetics, those with isolated low HDL cholesterol had a 65% increased death rate compared to diabetics with normal HDL cholesterol levels (>0.9 mmol/l). Furthermore, it has been shown that even within high risk populations, such as those with familial hypercholesterolemia, HDL cholesterol level is an important predictor of CAD. Low HDL cholesterol levels thus constitute a major, independent, risk for CAD.

These findings have led to increased attention to HDL cholesterol levels as a focus for treatment, following the recommendations of the National Cholesterol Education Program. These guidelines suggest that HDL cholesterol values below 0.9 mmol/l confer a significant risk for men and women. As such, nearly half of patients with CAD would have low HDL cholesterol. It is therefore crucial that we obtain a better understanding of factors which contribute to this phenotype. In view of the fact that pharmacological intervention of low HDL cholesterol levels has so far proven unsatisfactory, it is also important to understand the factors that regulate these levels in the circulation as this understanding may reveal new therapeutic targets.

Absolute levels of HDL cholesterol may not always predict risk of CAD. In the case of CETP deficiency, individuals display an increased risk of developing CAD, despite increased HDL cholesterol levels. What seems to be important in this case is the functional activity of the reverse cholesterol transport pathway, the process by which intracellular cholesterol is trafficked out of the cell to acceptor proteins such as ApoAI or HDL. Other important genetic determinants of HDL cholesterol levels, and its inverse relation with CAD, may reside in the processes leading to HDL formation and intracellular cholesterol trafficking and efflux. To date, this process is poorly understood, however, and clearly not all of the components of this pathway have been identified. Thus, defects preventing proper HDL-mediated cholesterol efflux may be important predictors of CAD. Therefore it is critical to identify and understand novel genes involved in the intracellular cholesterol trafficking and efflux pathways.

HDL particles are central to the process of reverse cholesterol transport and thus to the maintenance of tissue cholesterol homeostasis. This process has multiple steps which include the binding of HDL to cell surface components, the acquisition of cholesterol by passive absorption, the esterification of this cholesterol by LCAT and the subsequent transfer of esterified cholesterol by CETP, to VLDL and chylomicron remnants for liver uptake. Each of these steps is known to impact the plasma concentration of HDL.

Changes in genes for ApoAI-CIII, lipoprotein lipase, CETP, hepatic lipase, and LCAT all contribute to determination of HDL-C levels in humans. One rare form of genetic HDL deficiency is Tangier disease (TD), diagnosed in approximately 40 patients world-wide, and associated with almost complete absence of HDL cholesterol (HDL-C) levels (listed in OMIM as an autosomal recessive trait (OMIM 205400)). These patients have very low HDL cholesterol and ApoAI levels, which have been ascribed to hypercatabolism of nascent HDL and ApoAI, due to a delayed acquisition of lipid and resulting failure of conversion to mature HDL. TD patients accumulate cholesterol esters in several tissues, resulting in characteristic features, such as enlarged yellow tonsils, hepatosplenomegaly, peripheral neuropathy, and cholesterol ester deposition in the rectal mucosa. Defective removal of cellular cholesterol and phospholipids by ApoAI as well as a marked deficiency in HDL mediated efflux of intracellular cholesterol has been demonstrated in TD fibroblasts. Even though this is a rare disorder, defining its molecular basis could identify pathways relevant for cholesterol regulation in the general population. The decreased availability of free cholesterol for efflux in the surface membranes of cells in Tangier Disease patients appears to be due to a defect in cellular lipid metabolism or trafficking. Approximately 45% of Tangier patients have signs of premature CAD, suggesting a strong link between decreased cholesterol efflux, low HDL cholesterol and CAD. As increased cholesterol is observed in the rectal mucosa of persons with TD, the molecular mechanism responsible for TD may also regulate cholesterol adsorption from the gastrointestinal (GI) tract.

A more common form of genetic HDL deficiency occurs in patients who have low plasma HDL cholesterol usually below the 5th percentile for age and sex (OMIM 10768), but an absence of clinical manifestations specific to Tangier disease (Marcil et al., Arterioscler. Thromb. Vasc. Biol. 19:159-169, 1999; Marcil et al., Arterioscler. Thromb. Vasc. Biol. 15:1015-1024, 1995). These patients have no obvious environmental factors associated with this lipid phenotype, and do not have severe hypertriglyceridemia nor have known causes of severe HDL deficiency (mutations in ApoAI, LCAT, or LPL deficiency) and are not diabetic. The pattern of inheritance of this condition is most consistent with a Mendelian dominant trait (OMIM 10768).

The development of drugs that regulate cholesterol metabolism has so far progressed slowly. Thus, there is a need for a better understanding of the genetic components of the cholesterol efflux pathway. Newly-discovered components can then serve as targets for drug design.

Low HDL levels are likely to be due to multiple genetic factors. The use of pharmacogenomics in the aid of designing treatment tailored to the patient makes it desirable to identify polymorphisms in components of the cholesterol efflux pathway. An understanding of the effect of these polymorphisms on protein function would allow for the design of a therapy that is optimal for the patient.

### SUMMARY OF THE INVENTION

In a first aspect, the invention features a substantially pure ABC1 polypeptide having ABC1 biological activity. Preferably, the ABC1 polypeptide is human ABC1 (e.g., one that includes amino acids 1 to 60 or amino acids 61 to 2261 of SEQ ID NO: 1). In one preferred embodiment, the ABC1 polypeptide includes amino acids 1 to 2261 of SEQ ID NO: 1.

Specifically excluded from the polypeptides of the invention are the polypeptide having the exact amino acid sequence as GenBank accession number CAA10005.1 and the nucleic acid having the exact sequence as AJ012376.1. Also excluded is protein having the exact amino acid sequence as GenBank accession number X75926.

In a related aspect, the invention features a substantially pure ABC1 polypeptide that includes amino acids 1 to 2261 of SEQ ID NO: 1.

In another aspect, the invention features a substantially pure nucleic acid molecule encoding an ABC1 polypeptide having ABC1 biological activity (e.g., a nucleic acid molecule that includes nucleotides 75 to 254 or nucleotides 255 to 6858 of SEQ ID NO: 2). In one preferred embodiment, the nucleic acid molecule includes nucleotides 75 to 6858 of SEQ ID NO: 2.

In a related aspect, the invention features an expression vector, a cell, or a non-human mammal that includes the nucleic acid molecule of the invention.

In yet another aspect, the invention features a substantially pure nucleic acid molecule that includes nucleotides 75 to 254 of SEQ ID NO: 2, nucleotides 255 to 6858 of SEQ ID NO: 2, or nucleotides 75 to 6858 of SEQ ID NO: 2.

In still another aspect, the invention features a substantially pure nucleic acid molecule that includes at least fifteen nucleotides corresponding to the 5′ or 3′ untranslated region from a human ABC1 gene. Preferably, the 3′ untranslated region includes nucleotides 7015-7860 of SEQ ID NO: 2.

In a related aspect, the invention features a substantially pure nucleic acid molecule that hybridizes at high stringency to a probe comprising nucleotides 7015-7860 of SEQ ID NO: 2.

In another aspect, the invention features a method of treating a human having low HDL cholesterol or a cardiovascular disease, including administering to the human an ABC1 polypeptide, or cholesterol-regulating fragment thereof, or a nucleic acid molecule encoding an ABC1 polypeptide, or cholesterol-regulating fragment thereof. In a preferred embodiment, the human has a low HDL cholesterol level relative to normal. Preferably, the ABC1 polypeptide is wild-type ABC1, or has a mutation that increases its stability or its biological activity. A preferred biological activity is regulation of cholesterol.

In a related aspect, the invention features a method of preventing or treating cardiovascular disease, including introducing into a human an expression vector comprising an ABC1 nucleic acid molecule operably linked to a promoter and encoding an ABC1 polypeptide having ABC1 biological activity.

In another related aspect, the invention features a method of preventing or ameliorating the effects of a disease-causing mutation in an ABC1 gene, including introducing into a human an expression vector comprising an ABC1 nucleic acid molecule operably linked to a promoter and encoding an ABC1 polypeptide having ABC1 biological activity.

In still another aspect, the invention features a method of treating or preventing cardiovascular disease, including administering to an animal (e.g., a human) a compound that mimes the activity of wild-type ABC1 or modulates the biological activity of ABC1.

One preferred cardiovascular disease that can be treated using the methods of the invention is coronary artery disease. Others include cerebrovascular disease and peripheral vascular disease.

The discovery that the ABC1 gene and protein are involved in cholesterol transport that affects serum HDL levels allows the ABC1 protein and gene to be used in a variety of diagnostic tests and assays for identification of HDL-increasing or CVD-inhibiting drugs. In one family of such assays, the ability of domains of the ABC1 protein to bind ATP is utilized; compounds that enhance this binding are potential HDL-increasing drugs. Similarly, the anion transport capabilities and membrane pore-forming functions in cell membranes can be used for drug screening.

ABC1 expression can also serve as a diagnostic tool for low HDL or CVD; determination of the genetic subtyping of the ABC1 gene sequence can be used to subtype low HDL individuals or families to determine whether the low HDL phenotype is related to ABC1 function. This diagnostic process can lead to the tailoring of drug treatments according to patient genotype (referred to as pharmacogenomics), including prediction of the patient's response (e.g., increased or decreased efficacy or undesired side effects upon administration of a compound or drug.

Antibodies to an ABC1 polypeptide can be used both as therapeutics and diagnostics. Antibodies are produced by immunologically challenging a B-cell-containing biological system, e.g., an animal such as a mouse, with an ABC1 polypeptide to stimulate production of anti-ABC1 protein by the B-cells, followed by isolation of the antibody from the biological system. Such antibodies can be used to measure ABC1 polypeptide in a biological sample such as serum, by contacting the sample with the antibody and then measuring immune complexes as a measure of the ABC1 polypeptide in the sample. Antibodies to ABC1 can also be used as therapeutics for the modulation of ABC1 biological activity.

Thus, in another aspect, the invention features a purified antibody that specifically binds to ABC1.

In yet another aspect, the invention features a method for determining whether a candidate compound modulates ABC1 biological activity, comprising: (a) providing an ABC1 polypeptide; (b) contacting the ABC1 polypeptide with the candidate compound; and (c) measuring ABC1 biological activity, wherein altered ABC1 biological activity, relative to an ABC1 polypeptide not contacted with the compound, indicates that the candidate compound modulates ABC1 biological activity. Preferably, the ABC1 polypeptide is in a cell or is in a cell-free assay system.

In still another aspect, the invention features a method for determining whether a candidate compound modulates ABC1 expression. The method includes (a) providing a nucleic acid molecule comprising an ABC1 promoter operably linked to a reporter gene; (b) contacting the nucleic acid molecule with the candidate compound; and (c) measuring reporter gene expression, wherein altered reporter gene expression, relative to a nucleic acid molecule not contacted with the compound, indicates that the candidate compound modulates ABC1 expression.

In another aspect, the invention features a method for determining whether candidate compound is useful for modulating cholesterol levels, the method including the steps of: (a) providing an ABC1 polypeptide; (b) contacting the polypeptide with the candidate compound; and (c) measuring binding of the ABC1 polypeptide, wherein binding of the ABC1 polypeptide indicates that the candidate compound is useful for modulating cholesterol levels.

In a related aspect, the invention features method for determining whether a candidate compound mimics ABC1 biological activity. The method includes (a) providing a cell that is not expressing an ABC1 polypeptide; (b) contacting the cell with the candidate compound; and (c) measuring ABC1 biological activity of the cell, wherein altered ABC1 biological activity, relative to a cell not contacted with the compound, indicates that the candidate compound modulates ABC1 biological activity. Preferably, the cell has an ABC1 null mutation. In one preferred embodiment, the cell is in a mouse or a chicken (e.g., a WHAM chicken) in which its ABC1 gene has been mutated.

In still another aspect, the invention features a method for determining whether a candidate compound is useful for the treatment of low HDL cholesterol. The method includes (a) providing an ABC transporter (e.g., ABC1); (b) contacting the transporter with the candidate compound; and (c) measuring ABC transporter biological activity, wherein increased ABC transporter biological activity, relative to a transporter not contacted with the compound, indicates that the candidate compound is useful for the treatment of low HDL cholesterol. Preferably the ABC transporter is in a cell or a cell free assay system.

In yet another aspect, the invention features a method for determining whether candidate compound is useful for modulating cholesterol levels. The method includes (a) providing a nucleic acid molecule comprising an ABC transporter promoter operably linked to a reporter gene; (b) contacting the nucleic acid molecule with the candidate compound; and (c) measuring expression of the reporter gene, wherein increased expression of the reporter gene, relative to a nucleic acid molecule not contacted with the compound, indicates that the candidate compound is useful for modulating cholesterol levels.

In still another aspect, the invention features a method for determining whether a candidate compound increases the stability or decreases the regulated catabolism of an ABC transporter polypeptide. The method includes (a) providing an ABC transporter polypeptide; (b) contacting the transporter with the candidate compound; and (c) measuring the half-life of the ABC transporter polypeptide, wherein an increase in the half-life, relative to a transporter not contacted with the compound, indicates that the candidate compound increases the stability or decreases the regulated catabolism of an ABC transporter polypeptide. Preferably the ABC transporter is in a cell or a cell free assay system.

In a preferred embodiment of the screening methods of the present invention, the cell is in an animal. The preferred ABC transporters are ABC1, ABC2, ABCR, and ABC8, and the preferred biological activity is transport of cholesterol (e.g., HDL cholesterol or LDL cholesterol) or interleukin-1, or is binding or hydrolysis of ATP by the ABC1 polypeptide.

Preferably, the ABC1 polypeptide used in the screening methods includes amino acids 1-60 of SEQ ID NO: 1. Alternatively, the ABC1 polypeptide can include a region encoded by a nucleotide sequence that hybridizes under high stringency conditions to nucleotides 75 to 254 of SEQ ID NO: 2.

In another aspect, the invention features a method for determining whether a patient has an increased risk for cardiovascular disease. The method includes determining whether an ABC1 gene of the patient has a mutation, wherein a mutation indicates that the patient has an increased risk for cardiovascular disease.

In related aspect, the invention features a method for determining whether a patient has an increased risk for cardiovascular disease. The method includes determining whether an ABC1 gene of the patient has a polymorphism, wherein a polymorphism indicates that the patient has an increased risk for cardiovascular disease.

In another aspect, the invention features a method for determining whether a patient has an increased risk for cardiovascular disease. The method includes measuring ABC1 biological activity in the patient, wherein increased or decreased levels in the ABC1 biological activity, relative to normal levels, indicates that the patient has an increased risk for cardiovascular disease.

In still another aspect, the invention features a method for determining whether a patient has an increased risk for cardiovascular disease. The method includes measuring ABC1 expression in the patient, wherein decreased levels in the ABC1 expression relative to normal levels, indicates that the patient has an increased risk for cardiovascular disease. Preferably, the ABC1 expression is determined by measuring levels of ABC1 polypeptide or ABC1 RNA.

In another aspect, the invention features a non-human mammal having a transgene comprising a nucleic acid molecule encoding a mutated ABC1 polypeptide. In one embodiment, the mutation is a dominant-negative mutation.

In a related aspect, the invention features a non-human mammal, having a transgene that includes a nucleic acid molecule encoding an ABC1 polypeptide having ABC1 biological activity.

In another related aspect, the invention features a cell from a non-human mammal having a transgene that includes a nucleic acid molecule encoding an ABC1 polypeptide having ABC1 biological activity.

In still another aspect, the invention features a method for determining whether a candidate compound decreases the inhibition of a dominant-negative ABC1 polypeptide. The method includes (a) providing a cell expressing a dominant-negative ABC1 polypeptide; (b) contacting the cell with the candidate compound; and (c) measuring ABC1 biological activity of the cell, wherein an increase in the ABC1 biological activity, relative to a cell not contacted with the compound, indicates that the candidate compound decreases the inhibition of a dominant-negative ABC1 polypeptide.

By “polypeptide” is meant any chain of more than two amino acids, regardless of post-translational modification such as glycosylation or phosphorylation.

By “substantially identical” is meant a polypeptide or nucleic acid exhibiting at least 50%, preferably 85%, more preferably 90%, and most preferably 95% identity to a reference amino acid or nucleic acid sequence. For polypeptides, the length of comparison sequences will generally be at least 16 amino acids, preferably at least 20 amino acids, more preferably at least 25 amino acids, and most preferably 35 amino acids. For nucleic acids, the length of comparison sequences will generally be at least 50 nucleotides, preferably at least 60 nucleotides, more preferably at least 75 nucleotides, and most preferably 110 nucleotides.

Sequence identity is typically measured using sequence analysis software with the default parameters specified therein (e.g., Sequence Analysis Software Package of the Genetics Computer Group, University of Wisconsin Biotechnology Center, 1710 University Avenue, Madison, Wis. 53705). This software program matches similar sequences by assigning degrees of homology to various substitutions, deletions, and other modifications. Conservative substitutions typically include substitutions within the following groups: glycine, alanine, valine, isoleucine, leucine; aspartic acid, glutamic acid, asparagine, glutamine; serine, threonine; lysine, arginine; and phenylalanine, tyrosine.

By “high stringency conditions” is meant hybridization in 2×SSC at 40_C with a DNA probe length of at least 40 nucleotides. For other definitions of high stringency conditions, see F. Ausubel et al., Current Protocols in Molecular Biology, pp. 6.3.1-6.3.6, John Wiley & Sons, New York, N.Y., 1994, hereby incorporated by reference.

By “substantially pure polypeptide” is meant a polypeptide that has been separated from the components that naturally accompany it. Typically, the polypeptide is substantially pure when it is at least 60%, by weight, free from the proteins and naturally-occurring organic molecules with which it is naturally associated. Preferably, the polypeptide is an ABC1 polypeptide that is at least 75%, more preferably at least 90%, and most preferably at least 99%, by weight, pure. A substantially pure ABC1 polypeptide may be obtained, for example, by extraction from a natural source (e.g., a pancreatic cell), by expression of a recombinant nucleic acid encoding a ABC1 polypeptide, or by chemically synthesizing the protein. Purity can be measured by any appropriate method, e.g., by column chromatography, polyacrylamide gel electrophoresis, or HPLC analysis.

A polypeptide is substantially free of naturally associated components when it is separated from those contaminants that accompany it in its natural state. Thus, a polypeptide which is chemically synthesized or produced in a cellular system different from the cell from which it naturally originates will be substantially free from its naturally associated components. Accordingly, substantially pure polypeptides include those which naturally occur in eukaryotic organisms but are synthesized in E. coli or other prokaryotes.

By “substantially pure nucleic acid” is meant nucleic acid that is free of the genes which, in the naturally-occurring genome of the organism from which the nucleic acid of the invention is derived, flank the nucleic acid. The term therefore includes, for example, a recombinant nucleic acid that is incorporated into a vector; into an autonomously replicating plasmid or virus; into the genomic nucleic acid of a prokaryote or a eukaryote cell; or that exists as a separate molecule (e.g., a cDNA or a genomic or cDNA fragment produced by PCR or restriction endonuclease digestion) independent of other sequences. It also includes a recombinant nucleic acid that is part of a hybrid gene encoding additional polypeptide sequence.

By “modulates” is meant increase or decrease. Preferably, a compound that modulates cholesterol levels (e.g., HDL-cholesterol levels, LDL-cholesterol levels, or total cholesterol levels), or ABC1 biological activity, expression, stability, or degradation does so by at least 10%, more preferably by at least 25%, and most preferably by at least 50%.

By “purified antibody” is meant antibody which is at least 60%, by weight, free from proteins and naturally occurring organic molecules with which it is naturally associated. Preferably, the preparation is at least 75%, more preferably 90%, and most preferably at least 99%, by weight, antibody. A purified antibody may be obtained, for example, by affinity chromatography using recombinantly-produced protein or conserved motif peptides and standard techniques.

By “specifically binds” is meant an antibody that recognizes and binds to, for example, a human ABC1 polypeptide but does not substantially recognize and bind to other non-ABC1 molecules in a sample, e.g., a biological sample, that naturally includes protein. A preferred antibody binds to the ABC1 polypeptide sequence of FIG. 9A (SEQ ID NO: 1).

By “polymorphism” is meant that a nucleotide or nucleotide region is characterized as occurring in several different forms. A “mutation” is a form of a polymorphism in which the expression level, stability, function, or biological activity of the encoded protein is substantially altered.

By “ABC transporter” or “ABC polypeptide” is meant any transporter that hydrolyzes ATP and transports a substance across a membrane. Preferably, an ABC transporter polypeptide includes an ATP Binding Cassette and a transmembrane region. Examples of ABC transporters include, but are not limited to, ABC1, ABC2, ABCR, and ABC8.

By “ABC1 polypeptide” is meant a polypeptide having substantial identity to an ABC1 polypeptide having the amino acid sequence of SEQ ID NO: 1.

By “ABC biological activity” or “ABC1 biological activity” is meant hydrolysis or binding of ATP, transport of a compound (e.g., cholesterol, interleukin-1) or ion across a membrane, or regulation of cholesterol or phospholipid levels (e.g., either by increasing or decreasing HDL-cholesterol or LDL-cholesterol levels).

The invention provides screening procedures for identifying therapeutic compounds (cholesterol-modulating or anti-CVD pharmaceuticals) which can be used in human patients. Compounds that modulate ABC biological activity (e.g., ABC1 biological activity) are considered useful in the invention, as are compounds that modulate ABC concentration, protein stability, regulated catabolism, or its ability to bind other proteins or factors. In general, the screening methods of the invention involve screening any number of compounds for therapeutically active agents by employing any number of in vitro or in vivo experimental systems. Exemplary methods useful for the identification of such compounds are detailed below.

The methods of the invention simplify the evaluation, identification and development of active agents for the treatment and prevention of low HDL and CVD. In general, the screening methods provide a facile means for selecting natural product extracts or compounds of interest from a large population which are further evaluated and condensed to a few active and selective materials. Constitutes of this pool are then purified and evaluated in the methods of the invention to determine their HDL-raising or anti-CVD activities or both.

Other features and advantages of the invention will be apparent from the following description of the preferred embodiments thereof, and from the claims.

### BRIEF DESCRIPTION OF THE DRAWINGS

FIGS. 1A and 1B are schematic illustrations showing two pedigrees with Tangier Disease, (TD-1 and TD-2). Square and circle symbols represent males and females, respectively. Diagonal lines are placed through the symbols of all deceased individuals. A shaded symbol on both alleles indicates the probands with Tangier Disease. Individuals with half shaded symbols have HDL-C levels at or below the 10th percentile for age and sex, while those with quarter shaded symbols have HDL-C between the 11th and 20th percentiles.

Each individual's ID number, age at the time of lipid measurement, triglyceride level and HDL cholesterol level followed by their percentile ranking for age and sex are listed below the pedigree symbol. Markers spanning the 9q31.1 region are displayed to the left of the pedigree. The affected allele is represented by the darkened bars which illustrate the mapping of the limits of the shared haplotype region as seen in FIG. 3. Parentheses connote inferred marker data, questions marks indicate unknown genotypes, and large arrows show the probands.

FIG. 1C shows ApoAI (10 μg/mL)-mediated cellular cholesterol efflux in control fibroblasts (n=5, normalized to 100%) and two subjects with Tangier disease (TD). Cells were ³H-cholesterol (0.2 □Ci/mL) labeled during growth and cholesterol (20 □g/mL) loaded in growth arrest. Cholesterol efflux is determined as ³H medium/(³H cell+³H medium)

FIGS. 2A-2D are schematic illustrations showing four French Canadian pedigrees with FHA (FHA-1 to -4). The notations are as in FIG. 1. Exclamation points on either side of a genotype (as noted in Families FHA-3 and FHA-4) are used when the marker data appears to be inconsistent due to potential microsatellite repeat expansions. A bar that becomes a single thin line suggests that the haplotype is indeterminate at that marker.

FIGS. 3A-3E are a schematic illustration showing a genetic and physical map of 9q31 spanning 35 cM. FIG. 3A: YACs from the region of 9q22-34 were identified and a YAC contig spanning this region was constructed. FIG. 3B: A total of 22 polymorphic CA microsatellite markers were mapped to the contig and used in haplotype analysis in TD-1 and TD-2. FIG. 3C: The mutant haplotypes for probands in TD-1 and -2 indicate a significant region of homozygosity in TD-2, while the proband in TD-1 has 2 different mutant haplotypes. The candidate region can be narrowed to the region of homozygosity for CA markers in proband 2. A critical crossover at D9S1690 in TD-1 (A)* also provides a centromeric boundary for the region containing the gene. Three candidate genes in this region (ABC1, LPA-R and RGS-3) are shown. FIG. 3D: Meiotic recombinations in the FHA families (A-H) refine the minimal critical region to 1.2 cM between D9S277 and D9S1866. The heterozygosity of the TD-2 proband at D9S127, which ends a continuous region of homozygosity in TD-2, further refines the region to less than 1 cM. This is the region to which ABC1 has been mapped. FIG. 3E: Isolated YAC DNA and selected markers from the region were used to probe high-density BAC grid filters, selecting BACs which via STS-content mapping produced an 800 Kb contig. Four BACs containing ABC1 were sequenced using high-throughput methods.

FIG. 4A shows sequence of one mutation in family TD-1. Patient III-01 is heterozygous for a T to C transition at nucleotide 4503 of the cDNA; the control is homozygous for T at this position. This mutation corresponds to a cysteine to arginine substitution in the ABC1 protein (C1477R).

FIG. 4B shows the amino acid sequence conservation of residue 1477 in mouse and human, but not a related C. elegans gene. A change from cysteine to arginine likely has an important effect on the protein secondary and tertiary structure, as noted by its negative scores in most substitution matrices (Schuler et al., A Practical Guide to the Analysis of Genes and Proteins, eds. Baxevanis, A.D. & Ouellette, B.F.F. 145:171,1998). The DNA sequences of the normal and mutant genes are shown above and below the amino acid sequences, respectively.

FIG. 4C shows the segregation of the T4503C mutation in TD-1. The presence of the T4503C mutation (+) was assayed by restriction enzyme digestion with Hgal, which cuts only the mutant (C) allele (Õ). Thus, in the absence of the mutation, only the 194 bp PCR product (amplified between ø and Ø) is observed, while in its presence the PCR product is cleaved into fragments of 134 bp and 60 bp. The proband (individual 111.01) was observed to be heterozygous for this mutation (as indicated by both the 194 bp and 134 bp bands), as were his daughter, father, and three paternal cousins. A fourth cousin and three of the father's siblings were not carriers of this mutation.

FIG. 4D shows Northern blot analysis with probes spanning the complete ABC1 gene reveal the expected ˜8 Kb transcript and, in addition, a ˜3.5 kb truncated transcript only seen in the proband TD-1 and not in TD-2 or control. This was detected by probes spanning exons 1-49 (a), 1-41 (b), 1-22 (c), and 23-29 (d), but not with probes spanning exons 30-41 (e) or 4249 (f).

FIG. 5A shows the sequence of the mutation in family TD-2. Patient IV-10 is homozygous for an A to G transition at nucleotide 1864 of the cDNA (SEQ ID NO: 2); the control is homozygous for A at this position. This mutation corresponds to a glutamine to arginine substitution in the ABC1 protein (Q597R).

FIG. 5B shows that the glutamine amino acid, which is mutated in the TD-2 proband, is conserved in human and mouse ABC1 as well as in an ABC orthologue from C. elegans, revealing the specific importance of this residue in the structure/function of this ABC protein in both worms and mammals. The DNA sequences of the normal and mutant proteins are shown above and below the amino acid sequences, respectively.

FIG. 5C shows the segregation of the A1864G mutation in TD-2. The presence of the A1864G mutation (indicated by +) was assayed by restriction enzyme digestion with Acil. The 360 bp PCR product has one invariant Acil recognition site (Õ), and a second one is created by the A1864G mutation. The wild-type allele is thus cleaved to fragments of 215 bp and 145 bp, while the mutant allele (G-allele) is cleaved to fragments of 185 bp, 145 bp and 30 bp. The proband (individual IV-10), the product of a consanguineous mating, was homozygous for the A1864G mutation (+/+), as evidenced by the presence of only the 185 bp and 145 bp bands, while four other family members for whom DNA was tested are heterozygous carriers of this mutation (both the 215 bp and 185 bp fragments were present). Two unaffected individuals (−/−), with only the 215 bp and 145 bp bands are shown for comparison.

FIG. 6A shows a sequence of the mutation in family FHA-1. Patient III-01 is heterozygous for a deletion of nucleotides 2151-2153 of the cDNA (SEQ ID NO: 2). This deletion was detected as a superimposed sequence starting at the first nucleotide after the deletion. This corresponds to deletion of leucine 693 in the ABC1 protein (SEQ ID NO: 1).

FIG. 6B is an alignment of the human and mouse wild-type amino acid sequences, showing that the human and mouse sequences are identical in the vicinity of L693. L693 is also conserved in C. elegans. This highly conserved residue lies within a predicted transmembrane domain. The DNA sequences of the normal and mutant proteins are shown above and below the amino acid sequences, respectively.

FIG. 6C shows segregation of the L693 mutation in FHA-1, as assayed by Earl restriction digestion. Two invariant Earl restriction sites (indicated by Õ) are present within the 297 bp PCR product located between the horizontal arrows (øØ) while a third site is present in the wild-type allele only. The presence of the mutant allele is thus distinguished by the presence of a 210 bp fragment (+), while the normal allele produces a 151 bp fragment (−). The proband of this family (III.01) is heterozygous for this mutation, as indicated by the presence of both the 210 and 151 bp bands.

FIG. 6D shows a sequence of the mutation in family FHA-3. Patient III-01 is heterozygous for a deletion of nucleotides 5752-5757 of the cDNA (SEQ ID NO: 2). This deletion was detected as a superimposed sequence starting at the first nucleotide after the deletion. This corresponds to deletion of glutamic acid 1893 and aspartic acid 1894 in the ABC1 protein (SEQ ID NO: 1).

FIG. 6E is an alignment of the human and mouse wild-type amino acid sequences, showing that the human and mouse sequences are identical in the vicinity of 5752-5757. This region is highly conserved in C. elegans. The DNA sequences of the normal and mutant proteins are shown above and below the amino acid sequences, respectively.

FIG. 6F shows a sequence of the mutation in family FHA-2. Patient III-01 is heterozygous for a for a C to T transition at nucleotide 6504 of the cDNA (SEQ ID NO: 2). This alteration converts an arginine at position 2144 of SEQ ID NO: 1 to a STOP codon, causing truncation of the last 118 amino acids of the ABC1 protein.

FIGS. 7A and 7B show cholesterol efflux from human skin fibroblasts treated with ABC1 antisense oligonucleotides. Fibroblasts from a control subject were labeled with ³H cholesterol (0.2 □Ci/mL) during growth for 48 hours and transfected with 500 nM ABC1 antisense AN-1 (5′-GCA GAG GGC ATG GCT TTA TTT G-3′; SEQ ID NO: 3) with 7.5 μg lipofectin for 4 hours. Following transfection, cells were cholesterol loaded (20 μg/mL) for 12 hours and allowed to equilibrate for 6 hours. Cells were either then harvested for total RNA and 10 □g was used for Northern blot analysis. Cholesterol efflux experiments were carried out as described herein. FIG. 7A: AN-1 was the oligonucleotide that resulted in a predictable decrease in ABC1 RNA transcript levels. FIG. 7B: A double antisense transfection method was used. In this method, cells were labeled and transfected with AN-1 as above, allowed to recover for 20 hours, cholesterol loaded for 24 hours, and then re-transfected with AN-1. Twenty hours after the second transfection, the cholesterol efflux as measured. A ˜50% decrease in ABC1 transcript levels was associated with a significant decrease in cholesterol efflux intermediate between that seen in wild-type and TD fibroblasts.

FIG. 7C shows show cholesterol efflux from human skin fibroblasts treated with antisense oligonucleotides directed to the region encoding the amino-terminal 60 amino acids. Note that the antisense oligonucleotide AN-6, which is directed to the previously unrecognized translation start site, produces a substantial decrease in cellular cholesterol efflux.

FIG. 8 is a schematic illustration showing predicted topology, mutations, and polymorphisms of ABC1 in Tangier disease and FHA. The two transmembrane and ATP binding domains are indicated. The locations of mutations are indicated by the arrows with the amino acid changes, which are predicted from the human ABC1 cDNA sequence. These mutations occur in different regions of the ABC1 protein.

FIG. 9A shows the amino acid sequence of the human ABC1 protein (SEQ ID NO: 1).

FIGS. 9B-9E show the nucleotide sequence of the human ABC1 cDNA (SEQ ID NO: 2).

FIG. 10 shows the 5′ and 3′ nucleotide sequences suitable for use as 5′ and 3′ PCR primers, respectively, for the amplification of the indicated ABC1 exon.

FIG. 11 shows a summary of alterations found in ABC1, including sequencing errors, mutations, and polymorphisms.

FIG. 12 shows a series of genomic contigs (SEQ ID NOS. 14-29) containing the ABC1 promoter (SEQ ID NO: 14), as well as exons 1-49 (and flanking intronic sequence) of ABC1. The exons (capitalized letters) are found in the contigs as follows: SEQ ID NO: 14—exon 1; SEQ ID NO: 15—exon 2; SEQ ID NO: 16—exon 3; SEQ ID NO: 17—exon 4; SEQ ID NO: 18—exon 5; SEQ ID NO: 19—exon 6; SEQ ID NO: 20—exons 7 and 8; SEQ ID NO: 21—exons 9 through 22; SEQ ID NO: 22—exons 23 through 28; SEQ ID NO: 23—exon 29; SEQ ID NO: 24—exons 30 and 31; SEQ ID NO: 25—exon 32; SEQ ID NO: 26—exons 33 through 36; SEQ ID NO: 27—exons 37 through 41; SEQ ID NO: 28—exons 42-45; SEQ ID NO: 29—exons 46-49.

FIG. 13 is a series of illustrations showing that the amino-terminal 60 amino acid region of ABC1 is protein-coding. Lysates of normal human fibroblasts were immunoblotted in parallel with a rabbit polyclonal antibody to amino acids 1-20 of human ABC1 (1); a rabbit polyclonal antibody to amino acids 1430-1449 of human ABC1 (2); and a mouse monoclonal antibody to amino acids 2236-2259 of human ABC1. The additional bands detected in lane 2 may be due to a lack of specificity of that antibody or the presence of degradation products of ABC1.

FIG. 14 is a schematic illustration showing that the WHAM chicken contains a non-conservative substitution (G265A) resulting in an amino acid change (E89K).

FIG. 15 is a schematic illustration showing that the mutation in the WHAM chicken is at an amino acid that is conserved among human, mouse, and chicken.

FIG. 16 show a summary of locations of consensus transcription factor binding sites in the human ABC1 promoter (nucleotides 1-8238 of SEQ ID NO: 14). The abbreviations are as follows: PPRE=peroxisome proliferator-activated receptor. SRE=steroid response element-binding protein site. ROR=RAR-related orphan receptor.

### DETAILED DESCRIPTION

Genes play a significant role influencing HDL levels. Tangier disease (TD) was the first reported genetic HDL deficiency. The molecular basis for TD is unknown, but has been mapped to 9q31 in three families. We have identified two additional probands and their families, and confirmed linkage and refined the locus to a limited genomic region. Mutations in the ABC1 gene accounting for all four alleles in these two families were detected. A more frequent cause of low HDL levels is a distinct disorder, familial HDL deficiency (FHA). On the basis of independent linkage, meiotic recombinants and disease associated haplotypes, FHA was localized to a small genomic region encompassing the ABC1 gene. A mutation in a conserved residue in ABC1 segregated with FHA. Antisense reduction of the ABC1 transcript in fibroblasts was associated with a significant decrease in cholesterol efflux.

Cholesterol is normally assembled with intracellular lipids and secreted, but in TD the process is diverted and cholesterol is degraded in lysosomes. This disturbance in intracellular trafficking of cholesterol results in an increase in intracellular cholesterol ester accumulation associated with morphological changes of lysosomes and the Golgi apparatus and cholesteryl ester storage in histiocytes, Schwann cells, smooth muscle cells, mast cells and fibroblasts.

The clinical and biochemical heterogeneity in patients with TD has led to the possibility that genetic heterogeneity may also underlie this disorder. Considering this, we initially performed linkage analysis on these two families of different ancestries (TD-1 is Dutch, TD-2 is British; Frohlich et al., Clin. Invest. Med. 10:377-382, 1987) and confirmed that the genetic mutations underlying TD in these families were localized to the same 9q31 region, to which a large family with TD had been assigned (Rust et al., Nature Genetics 20:96-98, 1998). Detailed haplotype analysis, together with the construction of a physical map, refined the localization of this gene. Mutations in the ABC1 gene were found in TD.

FHA is much more common than TD, although its precise frequency is not known. While TD has been described to date in only 40 families, we have identified more than 40 FHA families in the Netherlands and Quebec alone. After initial suggestions of linkage to 9q31, thirteen polymorphic markers spanning approximately 10 cM in this region were typed and demonstrated the highest LOD score at D9S277. Analysis of the homozygosity of markers in the TD-2 proband, who was expected to be homozygous for markers close to TD due to his parents' consanguinity, placed the TD gene distal to D95127. Combined genetic data from TD and FHA families pointed to the same genomic segment spanning approximately 1,000 kb between D9S127 and D9S1866. The ABC1 transporter gene was contained within the minimal genomic region. RT-PCR analysis in one family demonstrated a deletion of leucine at residue 693 (693) in the first transmembrane domain of ABC1, which segregated with the phenotype of HDL deficiency in this family.

ABC1 is part of the ATP-binding cassette (ABC transporter) superfamily, which is involved in energy-dependent transport of a wide variety of substrates across membranes (Dean et al., Curr. Opin. Gen. Dev. 5:779-785, 1995). These proteins have characteristic motifs conserved throughout evolution which distinguish this class of proteins from other ATP binding proteins. In humans these genes essentially encode two ATP binding segments and two transmembrane domains (Dean et al., Curr. Opin. Gen. Dev. 5:779-785, 1995). We have now shown that the ABC1 transporter is crucial for intracellular cholesterol transport.

We have demonstrated that reduction of the ABC1 transcript using oligonucleotide antisense approaches results in decreased efflux, clearly demonstrating the link between alterations in this gene and its functional effects. TD and FHA now join the growing list of genetic diseases due to defects in the ABC group of proteins including cystic fibrosis (Zielenski, et al., Annu. Rev. Genet. 29:777-807, 1995), adrenoleukodystrophy (Mosser et al., Nature 361: 726-730, 1993), Zellweger syndrome (Gartner et al., Nat. Genet. 1:23, 1992), progressive familial intrahepatic cholestatis (Bull et al., Nat. Genet. 18:219-224, 1998), and different eye disorders including Stargardt disease (Allikmets et al., Nat. Genet.15:236-246, 1997), autosomal recessive retinitis pigmentosa (Allikmets et al., Science 277:1805-1807, 1997), and cone-rod dystrophy (Cremers et al., Hum. Mol. Genet. 7:355-362,1998).

Patients with TD have been distinguished from patients with FHA on the basis that Tangier disease was an autosomal recessive disorder (OMIM 20540) while FHA is inherited as an autosomal dominant trait (OMIM 10768). Furthermore, patients with TD have obvious evidence for intracellular cholesterol accumulation which is not seen in FHA patients. It is now evident that heterozygotes for TD do have reduced HDL levels and that the same mechanisms underlie the HDL deficiency and cholesterol efflux defects seen in heterozygotes for TD as well as FHA. Furthermore, the more severe phenotype in TD represents loss of function from both alleles of the ABC1 gene.

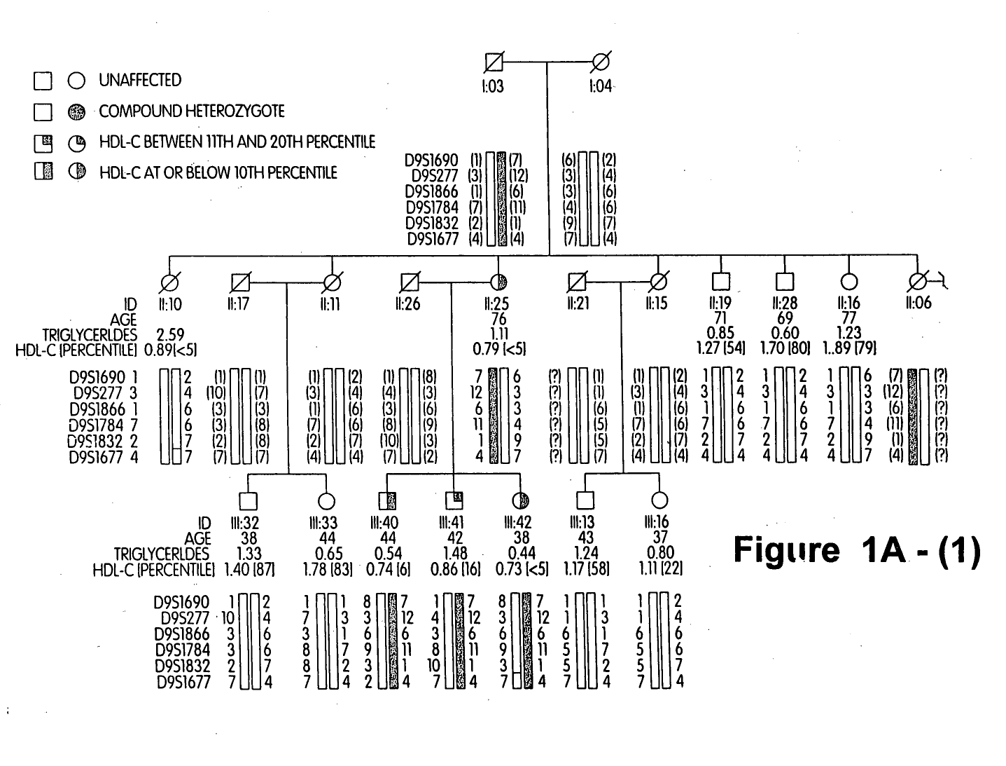

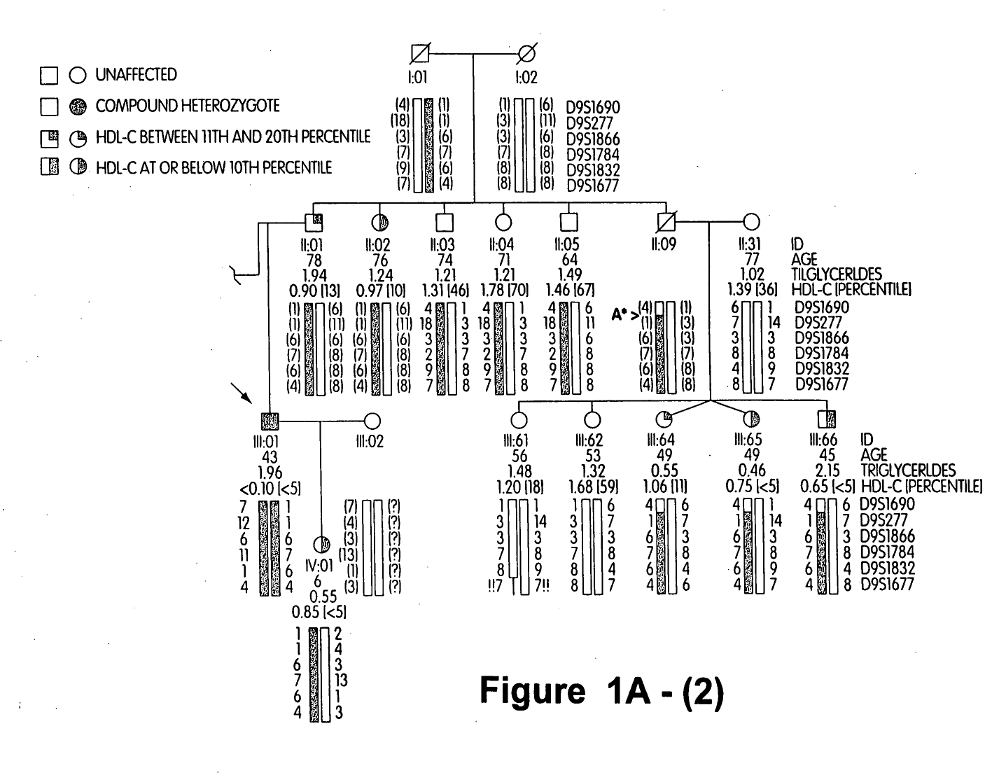

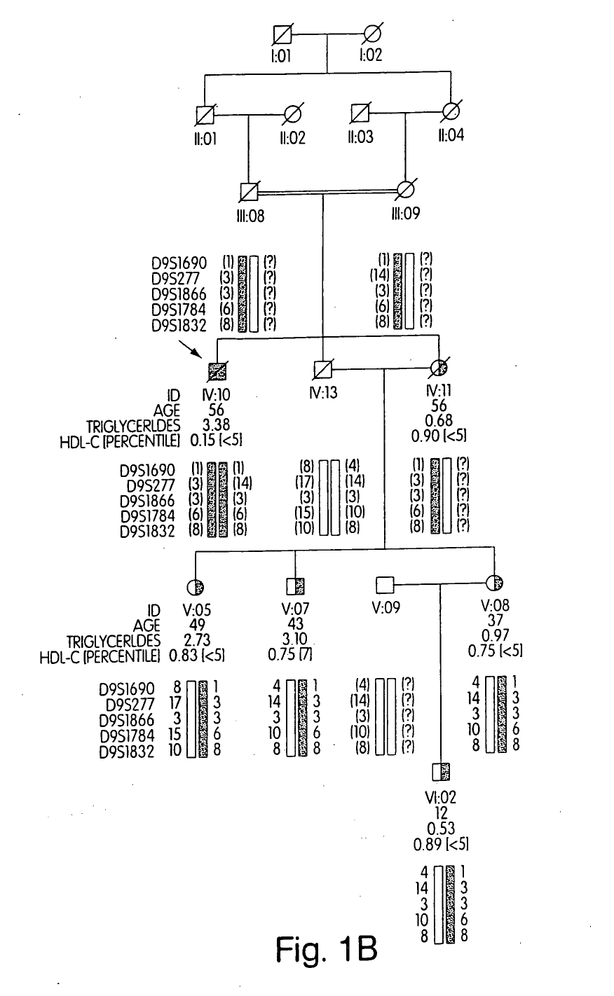

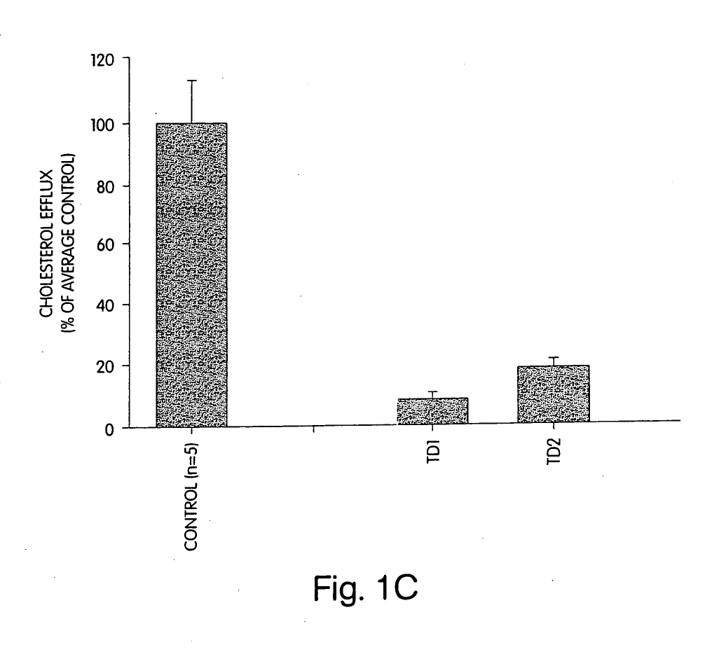

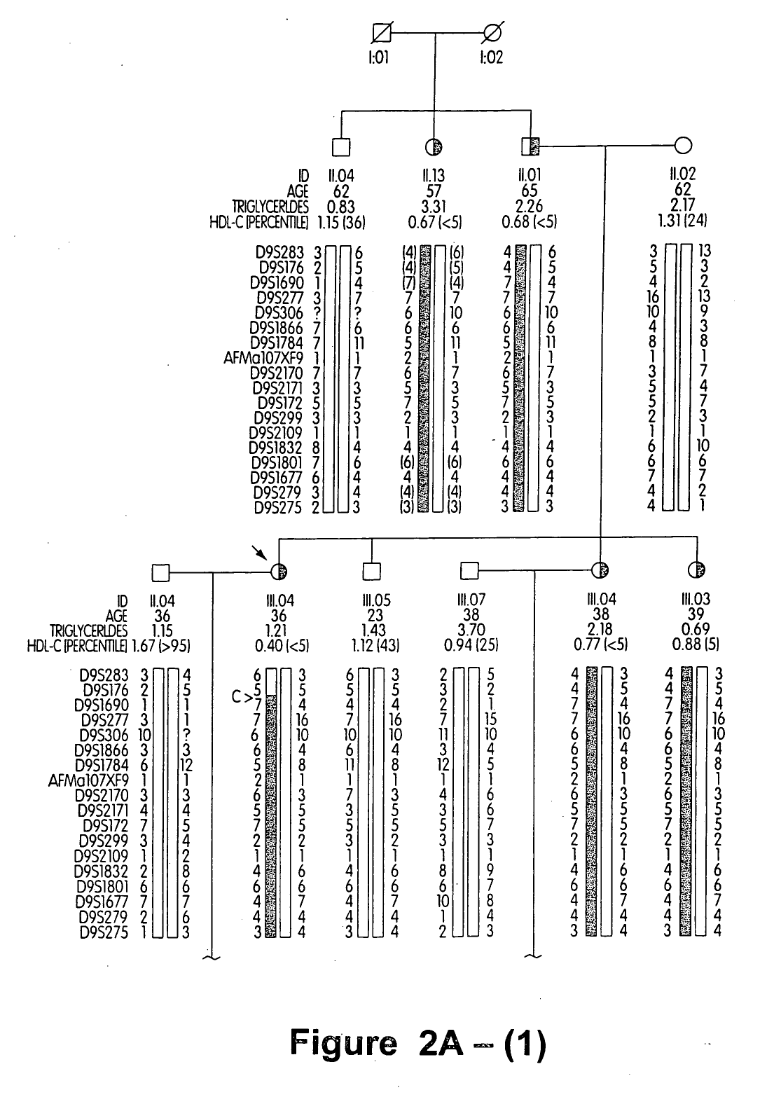

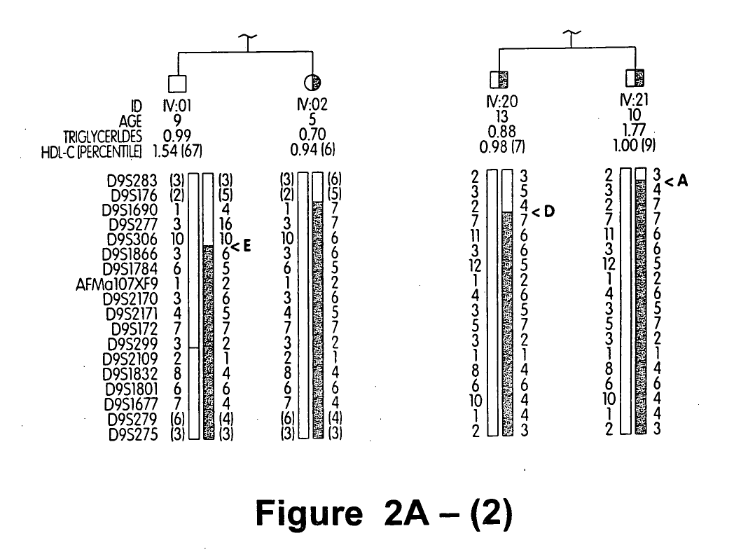

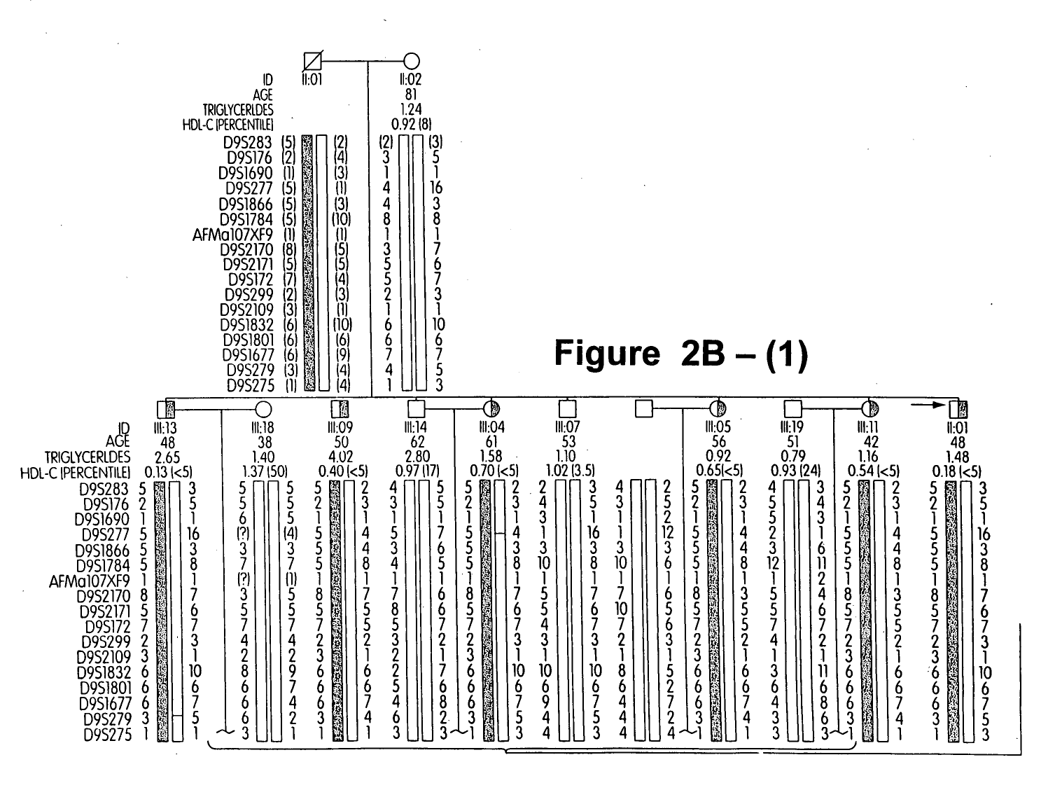

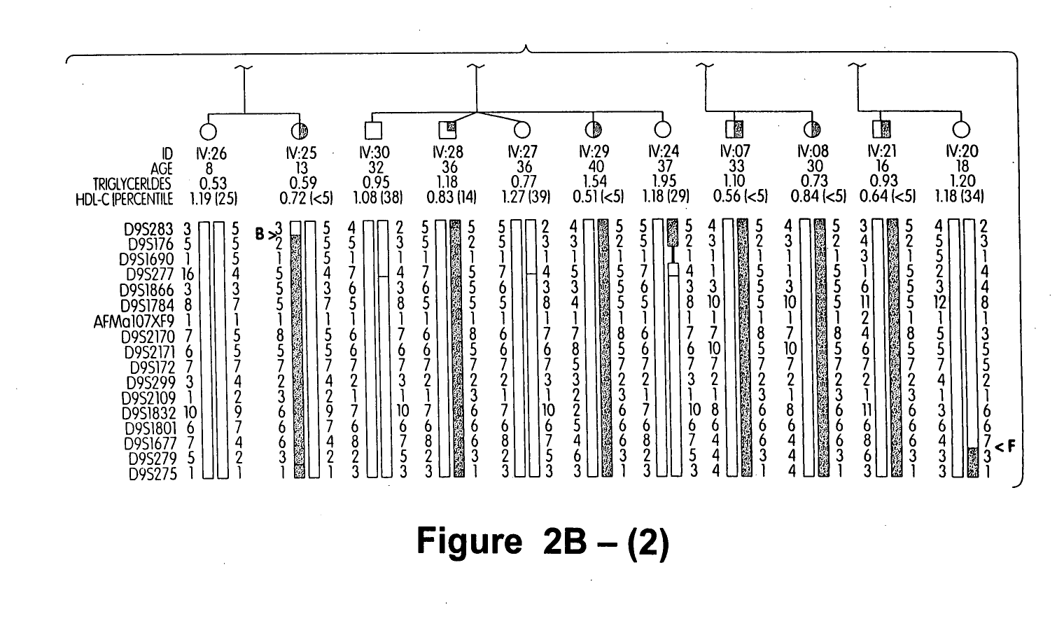

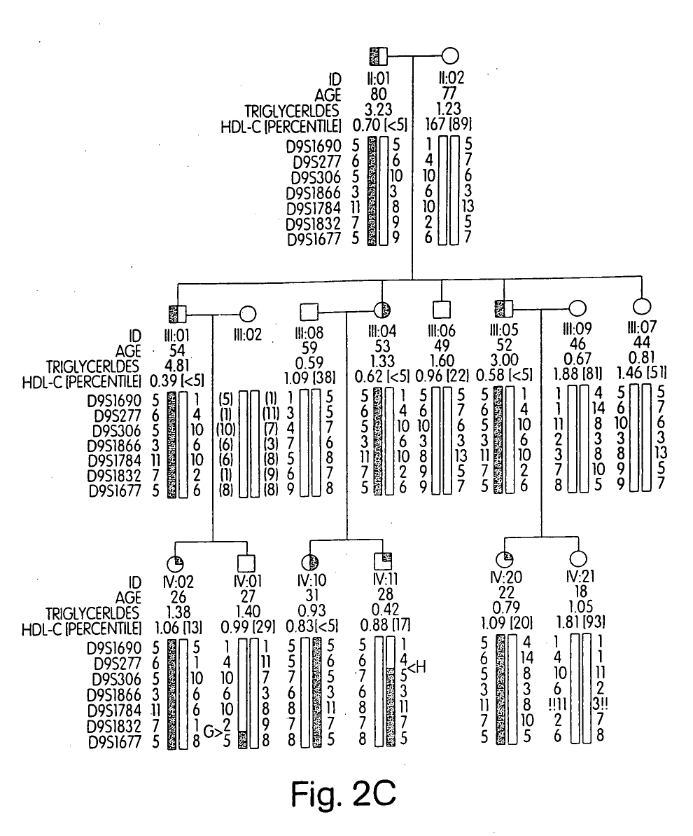

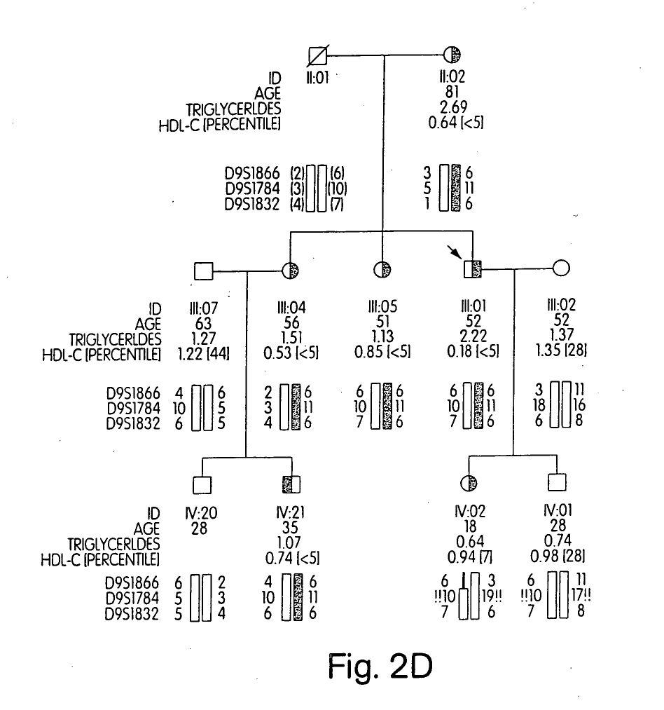

ABC1 is activated by protein kinases, presumably via phosphorylation, which also provides one explanation for the essential role of activation of protein kinase C in promoting cholesterol efflux (Drobnick et al., Arterioscler. Thromb. Vasc. Biol. 15: 1369-1377, 1995). Brefeldin, which inhibits trafficking between the endoplasmic reticulum and the Golgi, significantly inhibits cholesterol efflux, essentially reproducing the effect of mutations in ABC1, presumably through the inhibition of ABC1 biological activity. This finding has significance for the understanding of mechanisms leading to premature atherosclerosis. TD homozygotes develop premature coronary artery disease, as seen in the proband of TD-1 (III-01) who had evidence for coronary artery disease at 38 years. This is particular noteworthy as TD patients, in addition to exhibiting significantly reduced HDL, also have low LDL cholesterol, and yet they develop atherosclerosis despite this. This highlights the importance of HDL intracellular transport as an important mechanism in atherogenesis. There is significant evidence that heterozygotes for TD are also at increased risk for premature vascular disease (Schaefer et al., Ann. Int. Med. 93:261-266, 1980; Serfaty-Lacrosniere et al., Atherosclerosis 107:85-98, 1994). There is also preliminary evidence for premature atherosclerosis in some probands with FHA (FIG. 2B), e.g., the proband in FHA-2 (III-01) had a coronary artery bypass graft at 46 years while the proband in FHA-3 (FIG. 2C) had evidence for CAD around 50 years of age. The TD-1 proband had more severe efflux deficiency than the TD-2 proband (FIG. 1C). Interestingly, the TD-2 proband had no evidence for CAD by 62 when he died of unrelated causes, providing preliminary evidence for a relationship between the degree of cholesterol efflux (mediated in part by the nature of the mutation) and the likelihood of atherosclerosis.

The ABC1 gene plays a crucial role in cholesterol transport and, in particular, intracellular cholesterol trafficking in monocytes and fibroblasts. It also appears to play a significant role in other tissues such as the nervous system, GI tract, and the cornea. Completely defective intracellular cholesterol transport results in peripheral neuropathy, corneal opacities, and deposition of cholesterol esters in the rectal mucosa.

HDL deficiency is heterogeneous in nature. The delineation of the genetic basis of TD and FHA underlies the importance of this particular pathway in intracellular cholesterol transport, and its role in the pathogenesis of atherosclerosis. Unraveling of the molecular basis for TD and FHA defines a key step in a poorly defined pathway of cholesterol efflux from cells and could lead to new approaches to treatment of patients with HDL deficiency in the general population.

HDL has been implicated in numerous other biological processes, including but not limited to: prevention of lipoprotein oxidation; absorption of endotoxins; protection against Trypanosoma brucei infection; modulation of endothelial cells; and prevention of platelet aggregation (see Genest et al., J. Invest. Med. 47: 3142, 1999, hereby incorporated by reference). Any compound that modulates HDL levels may be useful in modulating one or more of the foregoing processes. The present discovery that ABC1 functions to regulate HDL levels links, for the first time, ABC1 with the foregoing processes.

The following examples are to illustrate the invention. They are not meant to limit the invention in any way.

Analysis of TD Families

Studies of Cholesterol Efflux

Both probands had evidence of marked deficiency of cholesterol efflux similar to that previously demonstrated in TD patients (FIG. 1C). TD-1 is of Dutch descent while TD-2 is of British descent.

Linkage Analysis and Establishment of a Physical Map

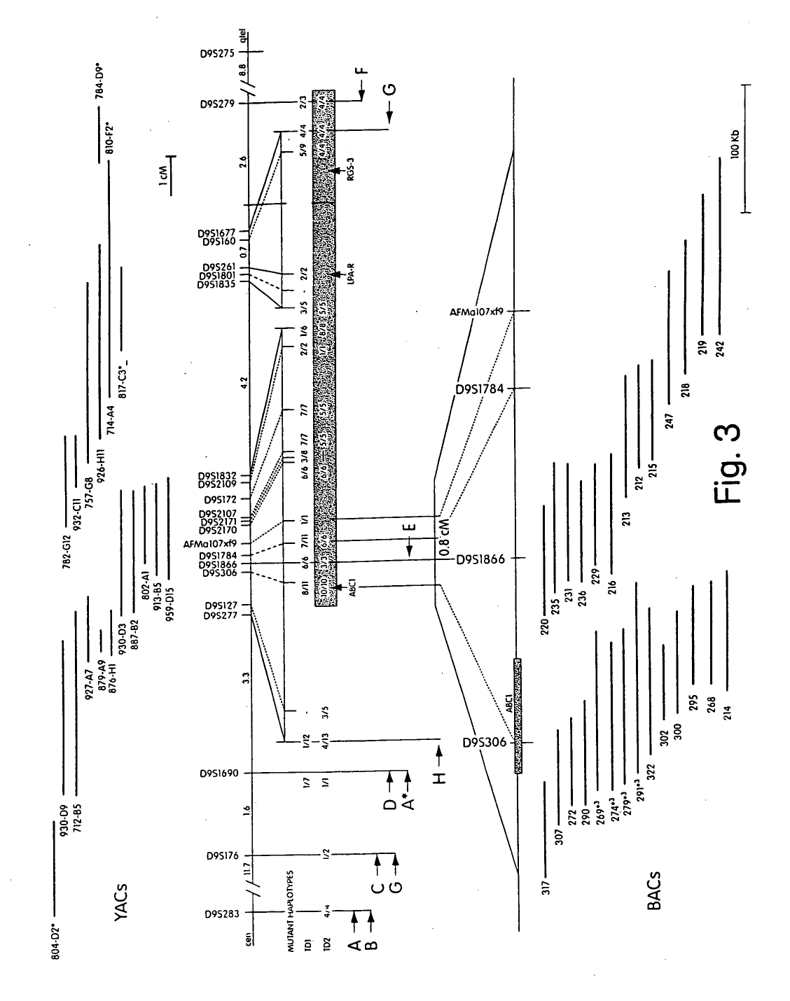

Multiple DNA markers were genotyped in the region of 9q31 to which linkage to TD had been described (Rust et al., Nat. Genet. 20, 96-98, 1998). Two point linkage analysis gave a maximal peak LOD score of 6.49 at D9S1832 (Table 1) with significant evidence of linkage to all markers in a ˜10 cM interval. Recombination with the most proximal marker, D9S1690 was seen in II-09 in Family TD-1 (A* in FIG. 3D) providing a centromeric boundary for the disease gene. Multipoint linkage analysis of these data did not increase the precision of the positioning of the disease trait locus.

A physical map spanning approximately 10 cM in this region was established with the development of a YAC contig (FIG. 3A). In addition, 22 other polymorphic multi-allelic markers which spanned this particular region were mapped to the contig (FIG. 3B) and a subset of these were used in construction of a haplotype for further analysis (FIGS. 1A and 1B; Table 2). The condensed haplotype in these families is shown in FIGS. 1A and 1B.

While the family of Dutch decent did not demonstrate any consanguinity, the proband in TD-2 was the offspring of a first-cousin consanguineous marriage (FIG. 1B). We postulated, therefore, that it was most likely that this proband would be homozygous for the mutation while the proband in the Dutch family was likely to be a compound heterozygote. The Dutch proband shows completely different mutation bearing haplotypes, supporting this hypothesis (FIG. 3C).

The TD-2 proband was homozygous for all markers tested (FIG. 1B) distal to D9S127 but was heterozygous at D9S127 and DNA markers centromeric to it (FIG. 3C). This suggested that the gene for TD was likely located to the genomic region telomeric of D9S127 and encompassed by the markers demonstrating homozygosity (FIG. 3B).

TABLE 1
Two Point Linkage Analysis of TD-1 and TD-2

| Marker Locus | LOD Score at recombination fraction | | | | | | |
|---|---|---|---|---|---|---|---|
| | 0 | 0.01 | 0.05 | 0.10 | 0.20 | 0.30 | 0.40 |
| D9S1690 | −infini | 4.25 | 4.52 | 4.26 | 3.39 | 2.30 | 1.07 |
| D9S277 | 6.22 | 6.11 | 5.67 | 5.10 | 3.90 | 2.60 | 1.17 |
| D9S1866 | 4.97 | 4.87 | 4.49 | 4.00 | 2.96 | 1.85 | 0.70 |
| D9S1784 | 5.50 | 5.40 | 5.00 | 4.47 | 3.36 | 2.17 | 0.92 |
| D9S1832 | 6.49 | 6.37 | 5.91 | 5.31 | 4.05 | 2.69 | 1.21 |
| D9S1677 | 4.60 | 4.51 | 4.18 | 3.76 | 2.88 | 1.93 | 0.93 |

Results of pairwise linkage analysis using MLINK. Values correspond to LOD score for linkage between the disease locus and a marker locus for specified values of the recombination fraction.

TABLE 2
Microsatellite markers used in this study

| | | | Number | Allele fraquency^{t} |
|---|---|---|---|---|
| Genetic Markers | Type | Heterozygocity | of alleles | size.bp (proportion |
| D9S283 | CA | 0.80 | 10 | 179(0.04); 181(0.34); 183(0.19); 185(0.20); |
| | | | | 189(0.05); 193(0.04); 197(0.07); 199(0.02); |
| | | | | 201(0.04); 203(0.04) |
| D9S176 | CA | 0.82 | 9 | 129(0.03); 131(0.06); 133(0.26); 135(0.12); |
| | | | | 137(0.25); 139(0.03); 141(0.01); 145(0.05); |
| | | | | 147(0.05) |
| D9S1690 | CA | 0.79 | 8 | 225(0.38); 227(0.14); 229(0.04); 231(0.12); |
| | | | | 233(0.05); 235(0.16); 237(0.05); 239(0.05) |
| D9S277 | CA | 0.89 | 15 | 167(0.07); 171(0.02); 173(0.15); 175(0.11); |
| | | | | 177(0.07); 179(0.04); 181(0.17); 183(0.06); |
| | | | | 185(0.02); 187(0.02); 189(0.13); 191(0.13); |
| | | | | 193(0.02); 197(0.00); 199(0.00) |
| D9S127 | CA | 0.72 | 6 | 149(0.11); 151(0.07); 153(0.25); 155(0.03); |
| | | | | 157(0.45); 159(0.06) |
| D9S306 | CA | 0.87 | 13 | 102(0.06); 104(0.01); 110(0.03); 112(0.08); |
| | | | | 114(0.16); 116(0.15); 118(0.11); 120(0.23); |
| | | | | 122(0.06); 124(0.06); 126(0.03); 134(0.02); |
| | | | | 136(0.01) |
| D9S1866 | CA | 0.62 | 11 | 248(0.06); 252(0.04); 254(0.01); 256(58); |
| | | | | 258(0.03); 260(0.06); 262(0.02); 264(0.12); |
| | | | | 266(0.06); 268(0.03); 270(0.01) |
| D9S1784 | CA | 0.86 | 15 | 174(0.10); 176(0.02); 178(0.00); 180(0.08); |
| | | | | 182(0.11); 184(0.22); 136(0.15); 158(0.06); |
| | | | | 190(0.04); 192(0.07); 194(0.08); 196(0.07); |
| | | | | 198(0.01); 200(0.01); 202(0.01) |
| AFMa107xf9 | CA | n.a. | n.a. | n.a. |
| D9S2170 | CA | n.a. | n.a. | n.a. |
| D9S2171 | CA | n.a. | n.a. | n.a. |
| D9S2107 | CA | 0.63 | 5 | n.a. |
| D9S172 | CA | 0.54 | 5 | 291(0.00); 297(0.05); 299(0.32); 303(0.62); |
| | | | | 305(0.02) |
| D9S2109 | CA | 0.51 | 3 | 1(0.42); 2(0.56); 3(0.02) |
| D9S1832 | CA | 0.88 | 12 | 161(0.04); 163(0.02); 167(0.02); 169(0.04); |
| | | | | 171(0.10); 173(0.09); 175(0.15); 177(0.28); |
| | | | | 179(0.19); 181(0.04); 183(0.01); 185(0.01) |
| D9S1835 | CA | 0.48 | 4 | 110(0.02); 112(0.23); 116(0.68); 118(0.07); |
| D9S1801 | CA | 0.77 | 10 | 166(0.10); 172(0.04); 174(0.02); 182(0.02); |
| | | | | 184(0.19); 186(0.40); 188(0.15); 190(0.04); |
| | | | | 192(0.02); 194(0.02) |
| D9S261 | CA | 0.63 | 7 | 90(0.02); 92(0.52); 94(0.02); 98(0.02); |
| | | | | 100(0.10); 102(0.04); 104(0.08) |
| D9S160 | CA | 0.62 | 6 | 136(0.25); 138(0.53); 140(0.01); 142(0.12); |
| | | | | 144(0.00); 146(0.07) |
| D9S1677 | CA | 0.81 | 10 | 251(0.27); 257(0.27); 259(0.07); 261(0.09); |
| | | | | 263(0.27); 265(0.14); 267(0.02); 267(0.02); |
| | | | | 271(0.04); 273(0.02) |
| D9S279 | CA | 0.78 | 6 | 244(0.09); 246(0.18); 248(0.29); 250(0.29); |
| | | | | 252(0.07); 254(0.09) |
| D9S275 | CA | 0.62 | 4 | 190(0.31); 196(0.07); 198(0.52); 200(0.09); |

^{t}In a Caucasian population of French Canadian or French descent (J. Weissenbach, Personal Communication 1993).
n.a. = not assessed

These polymorphic microsatellite markers were used for DNA typing in the region of 9q31 seen in FIG. 3. The majority come from the last version of the Généthon human linkage map. The frquency of heterozygosity, the number of alleles as well as the allele frequency of each marker are presented.

Mutation Detection

Based on the defect in intracellular cholesterol transport in patients with TD, we reviewed the EST database for genes in this region which might be relevant to playing a role in this process. One gene that we reviewed as a candidate was the lysophosphatidic acid (LPA) receptor (EDG2) which mapped near D9S1801 (FIG. 3C). This receptor binds LPA and stimulates phospholipase-C (PLC), and is expressed in fibroblasts. It has previously been shown that the coordinate regulation of PLC that is necessary for normal HDL3 mediated cholesterol efflux is impaired in TD (Walter et al., J. Clin. Invest. 98:2315-2323, 1996). Therefore this gene represented an excellent candidate for the TD gene. Detailed assessment of this gene, using Northern blot and RT-PCR and sequencing analysis, revealed no changes segregating with the mutant phenotype in this family, in all likelihood excluding this gene as the cause for TD. Polymorphisms were detected, however, in the RT-PCR product, indicating expression of transcripts from both alleles.

The second candidate gene (RGS3) encodes a member of a family regulating G protein signaling which could also be involved in influencing cholesterol efflux (Mendez et al., Trans. Assoc. Amer. Phys. 104:48-53, 1991). This gene mapped 0.7 cM telomeric to the LPA-receptor (FIG. 3C), and is expressed in fibroblasts. It was assessed by exon-specific amplification, as its genomic organization was published (Chatterjee et al., Genomics 45:429433, 1997). No significant sequence changes were detected.

The ABC1 transporter gene had previously been mapped to 9q31, but its precise physical location had not been determined (Luciani et al., Genomics 21:150-159, 1994). The ABC1 gene is a member of the ATP binding cassette transporters which represents a super family of highly conserved proteins involved in membrane transport of diverse substrates including amino acids, peptides, vitamins and steroid hormones (Luciani et al., Genomics 21:150-159, 1994; Dean et al., Curr. Opin. Gen. Dev. 5:779-785, 1995). Primers to the 3′ UTR of this gene mapped to YACs spanning D9S306 (887-B2 and 930-D3) compatible with it being a strong candidate for TD. We initiated large scale genomic sequencing of BACs spanning approximately 800 kb around marker D9S306 (BACs 269, 274, 279 and 291) (FIG. 3E). The ABC1 gene was revealed encompassing 49 exons and a minimum of 75 Kb of genomic sequence. In view of the potential function of a gene in this family as a cholesterol transporter, its expression in fibroblasts and localization to the minimal genomic segment underlying TD, we formally assessed ABC1 as a candidate.

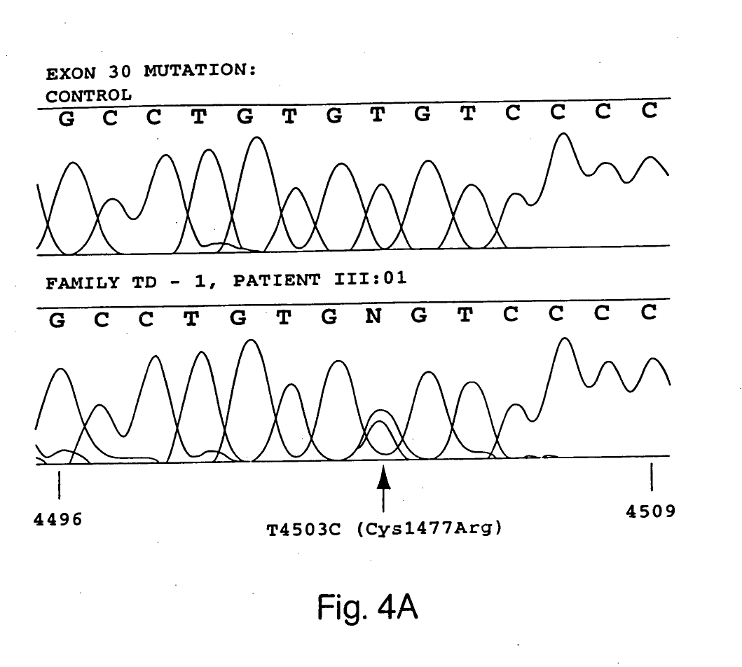

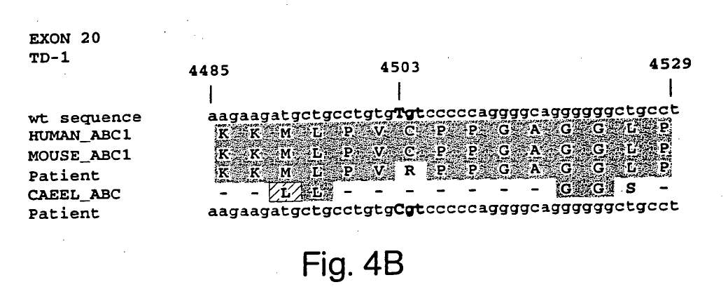

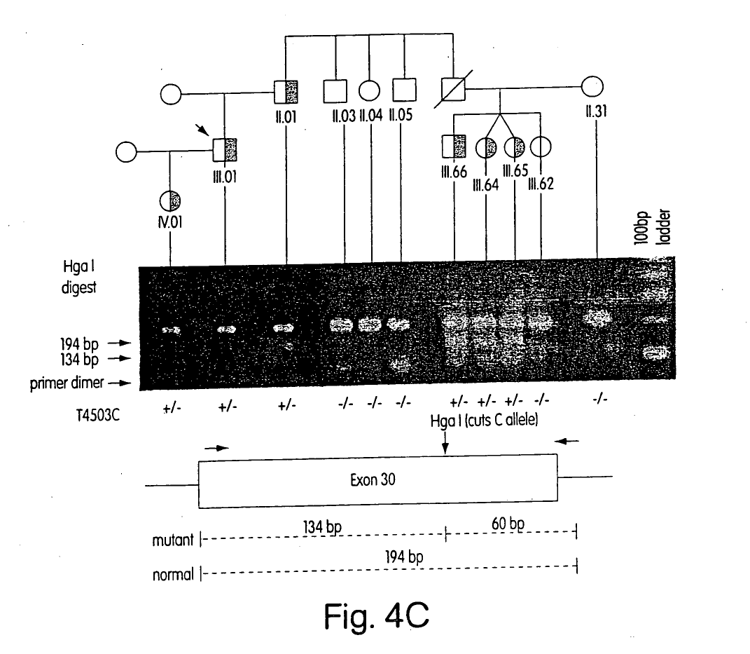

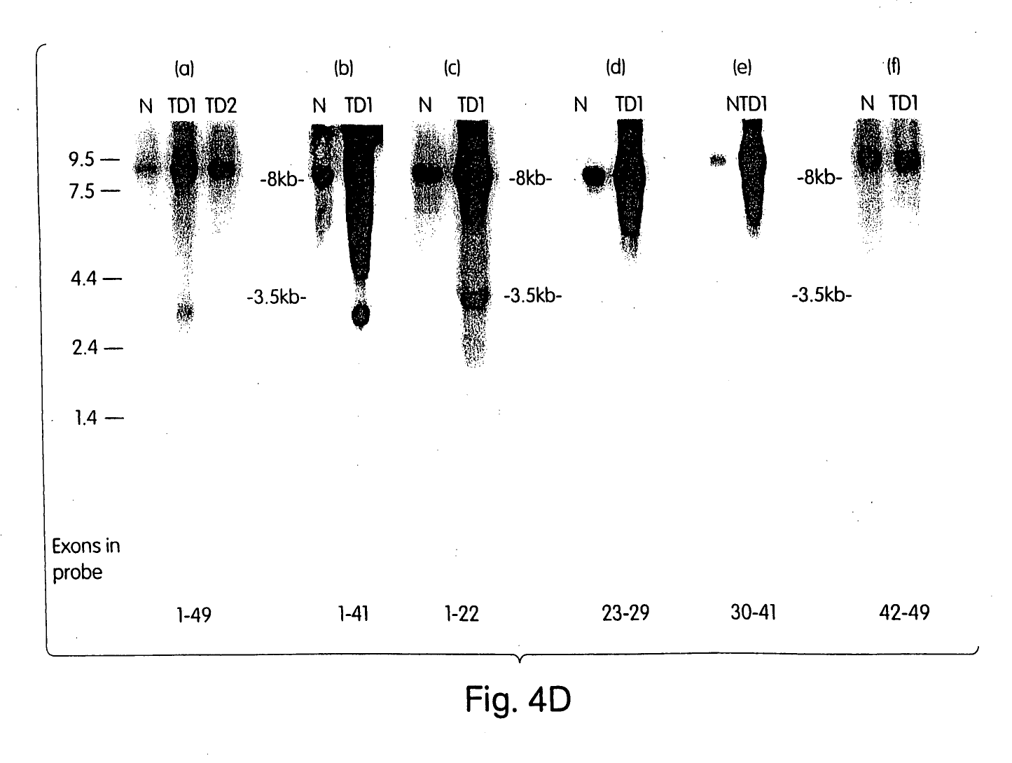

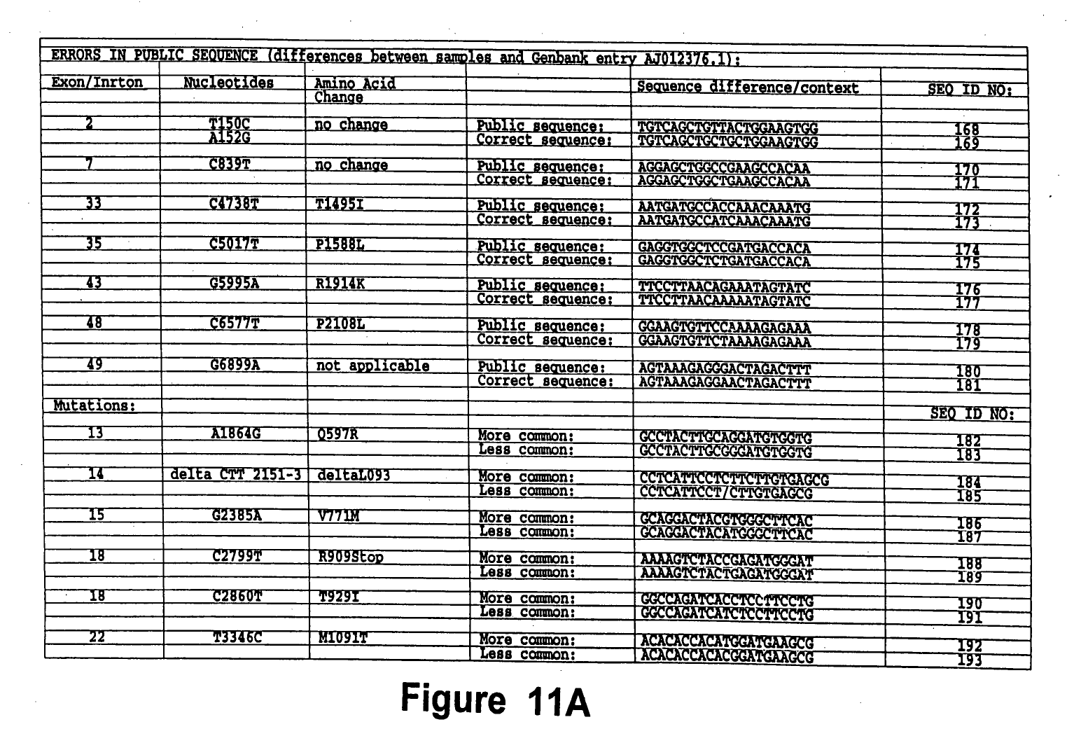

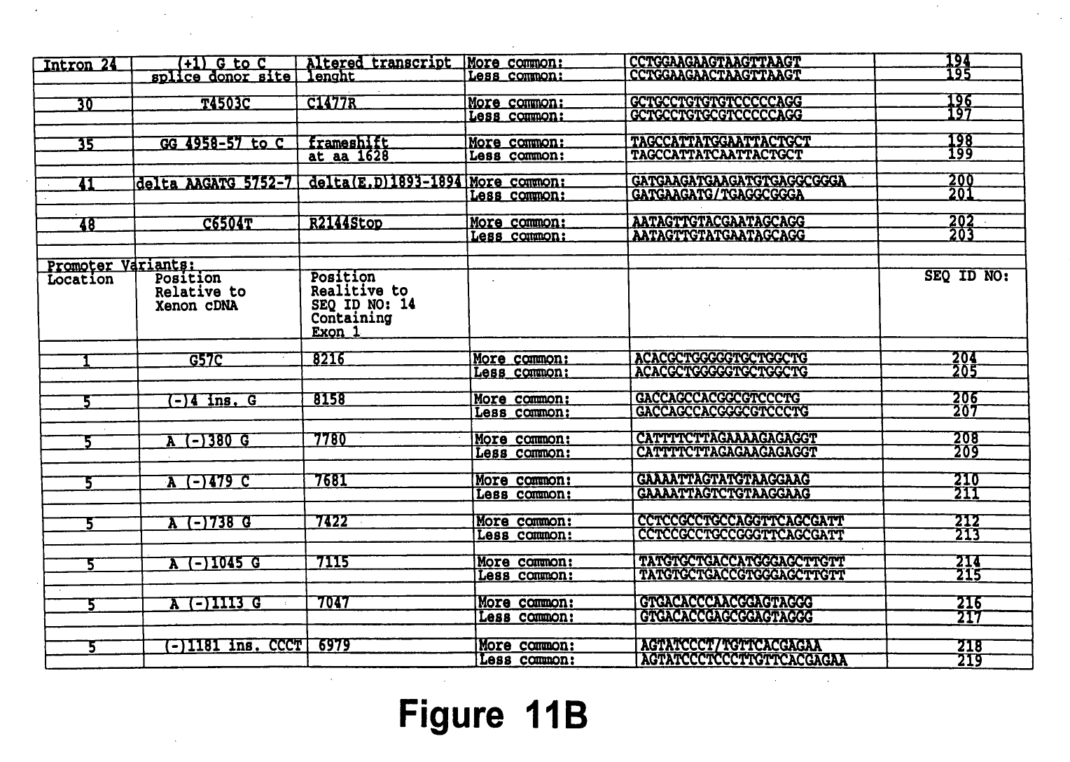

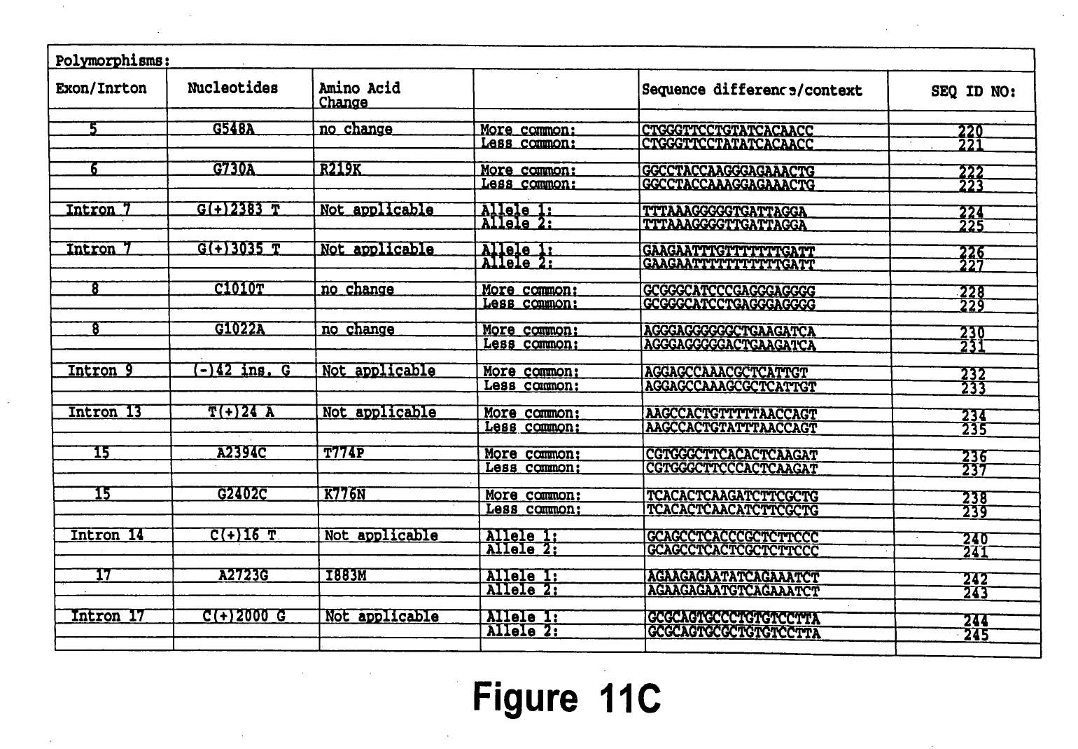

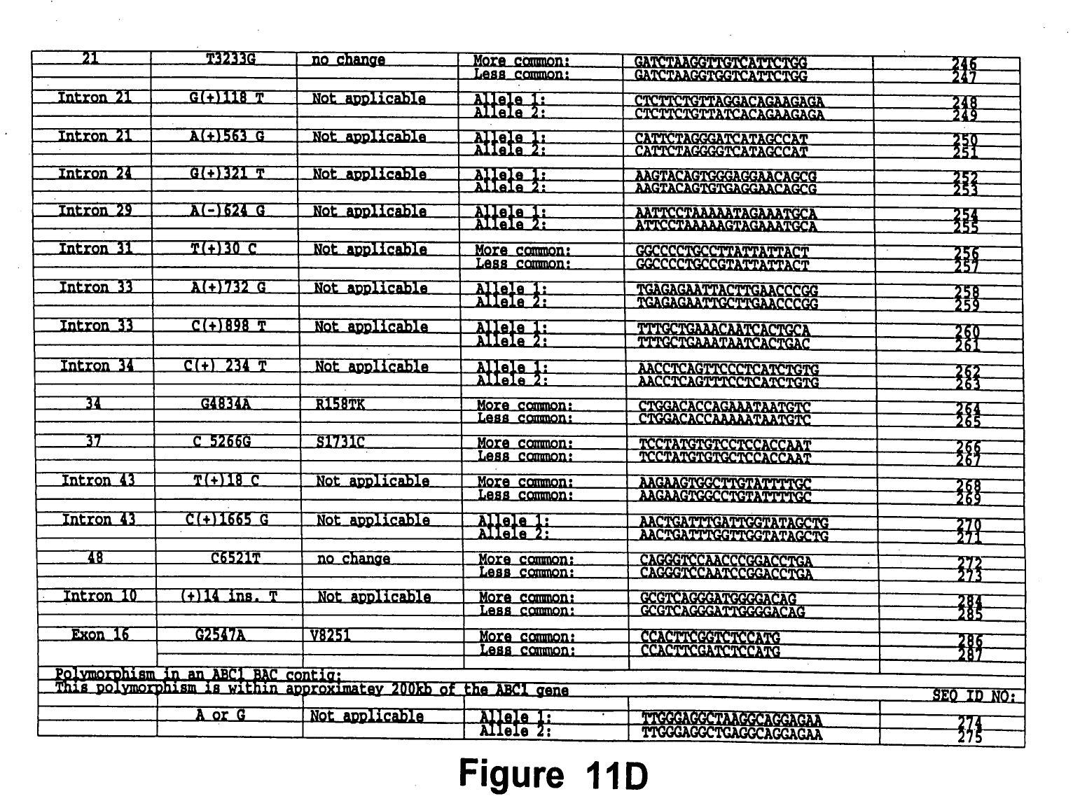

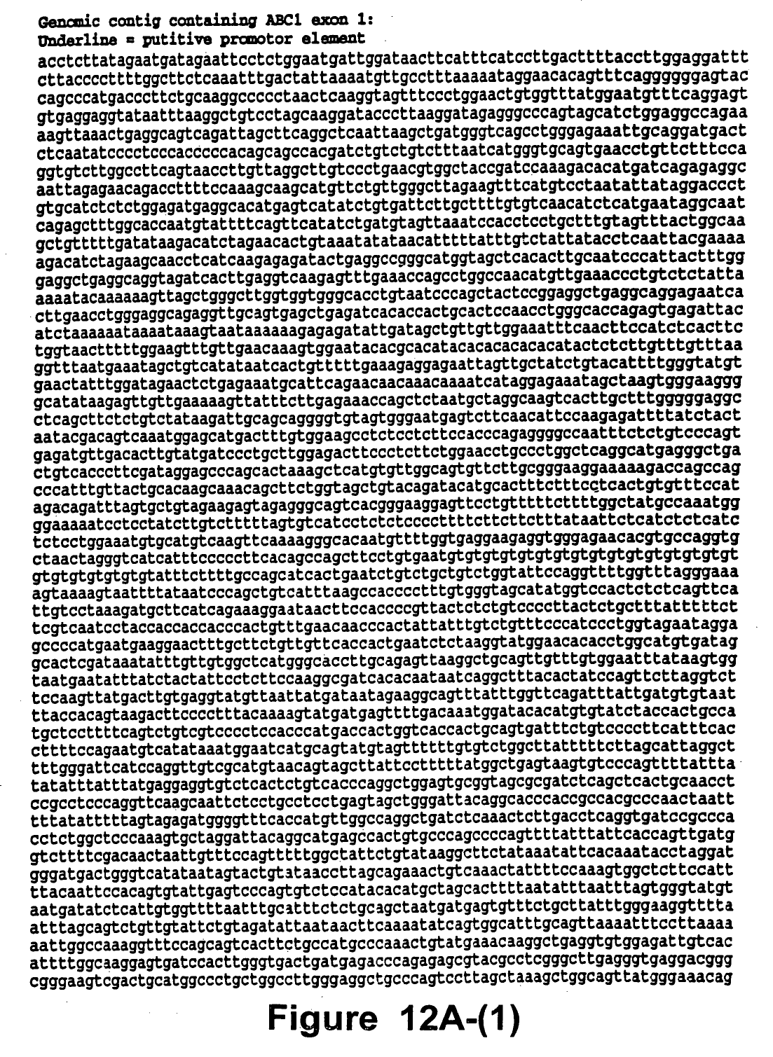

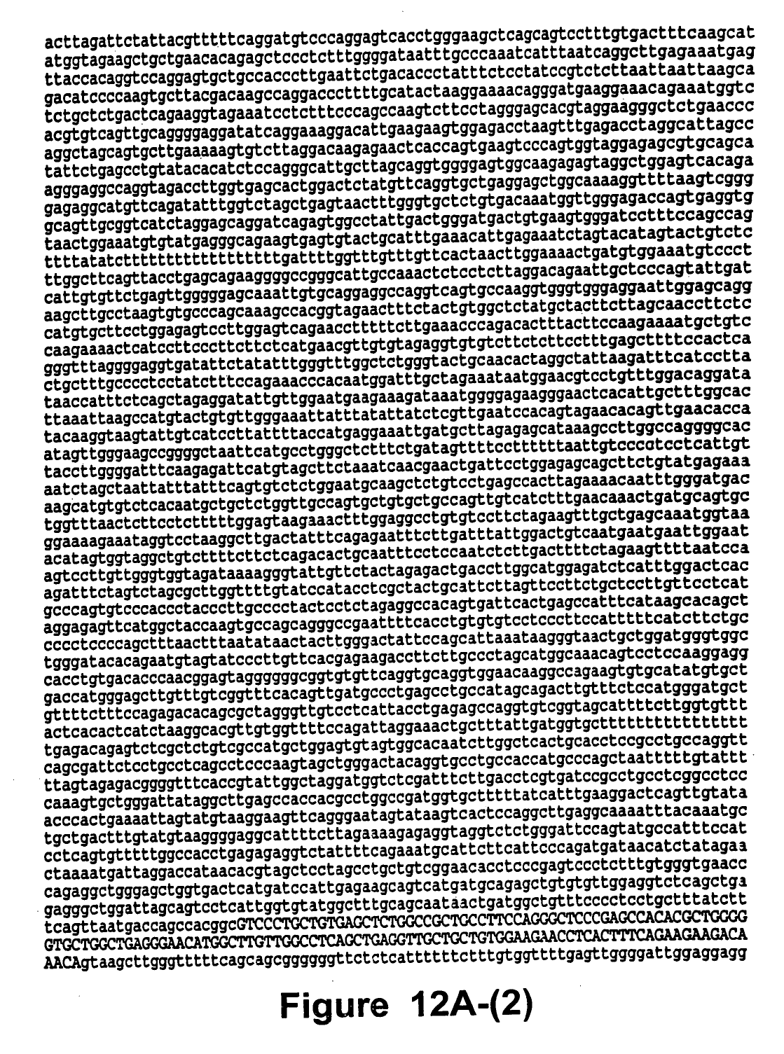

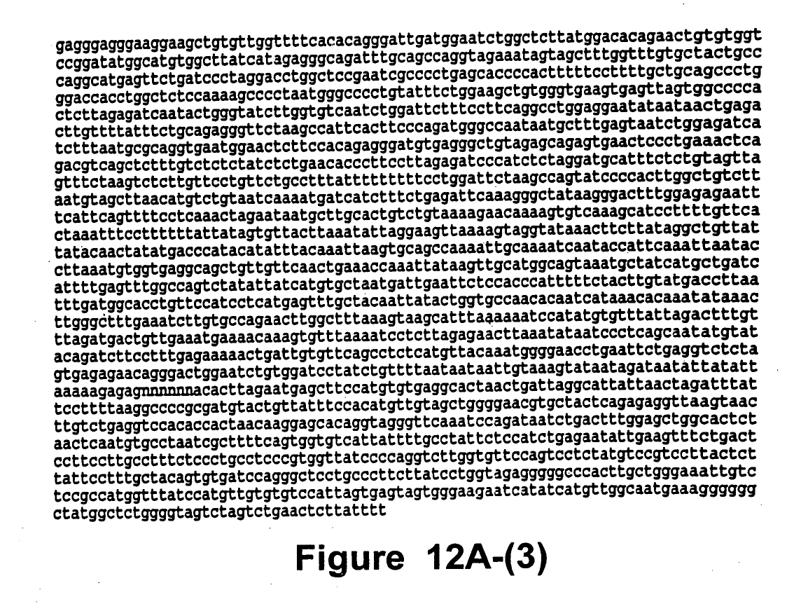

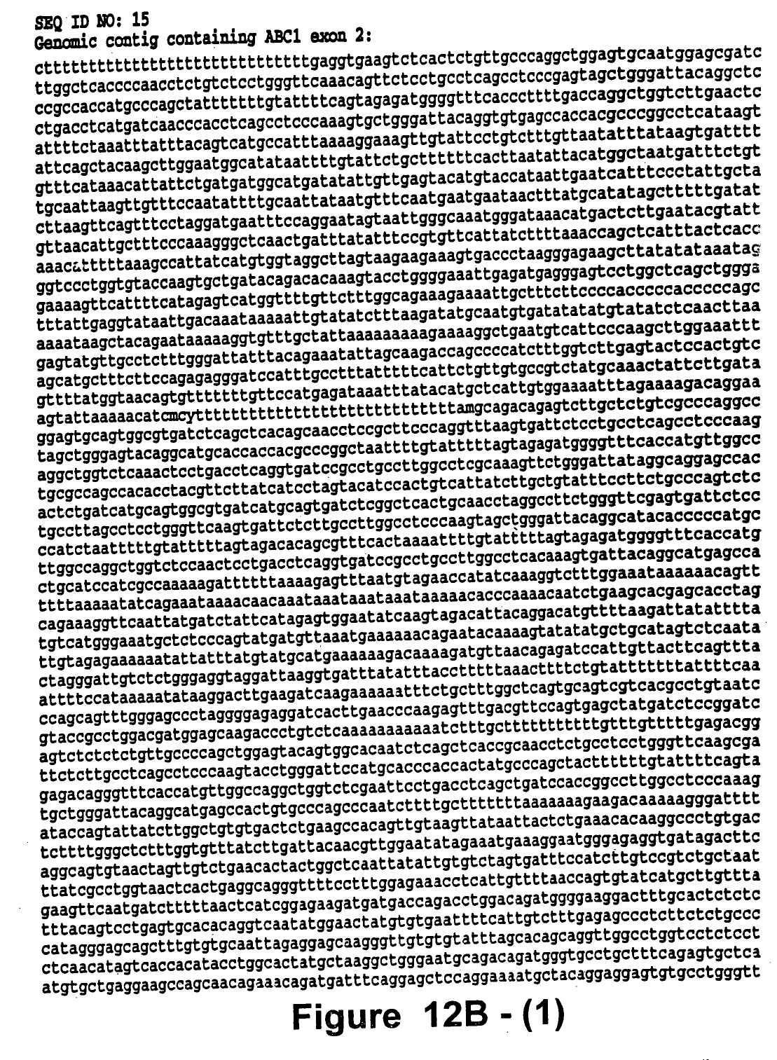

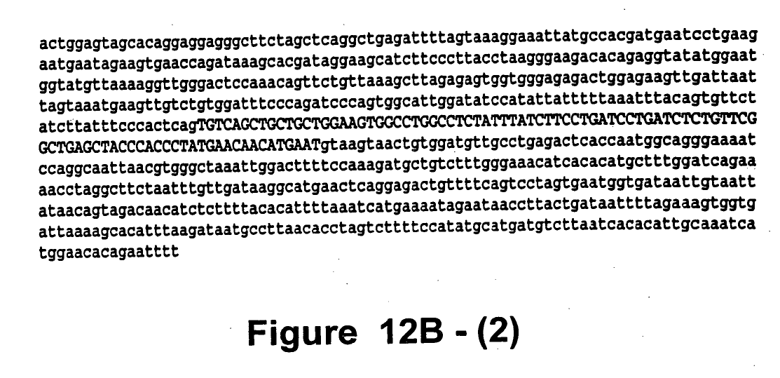

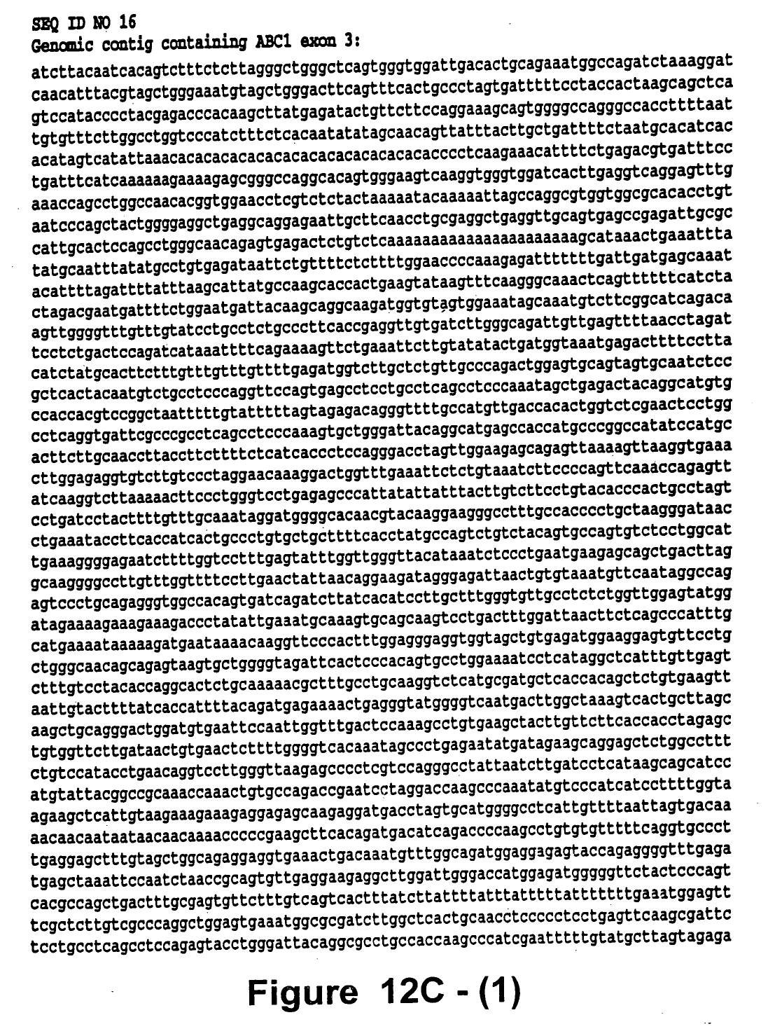

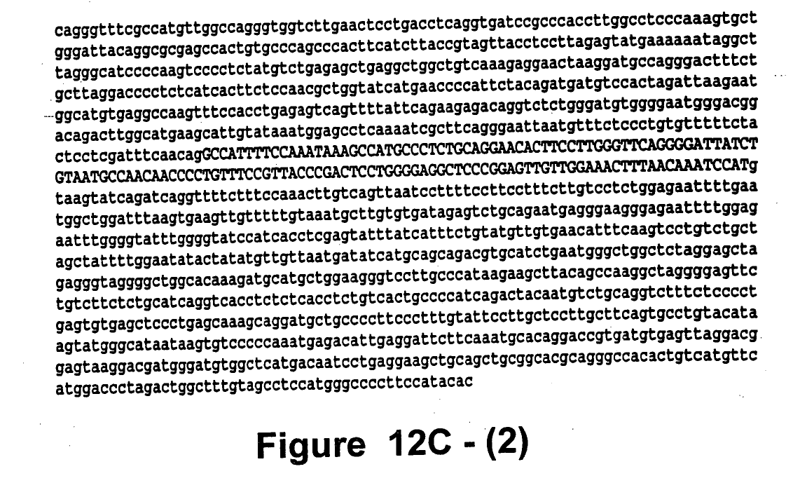

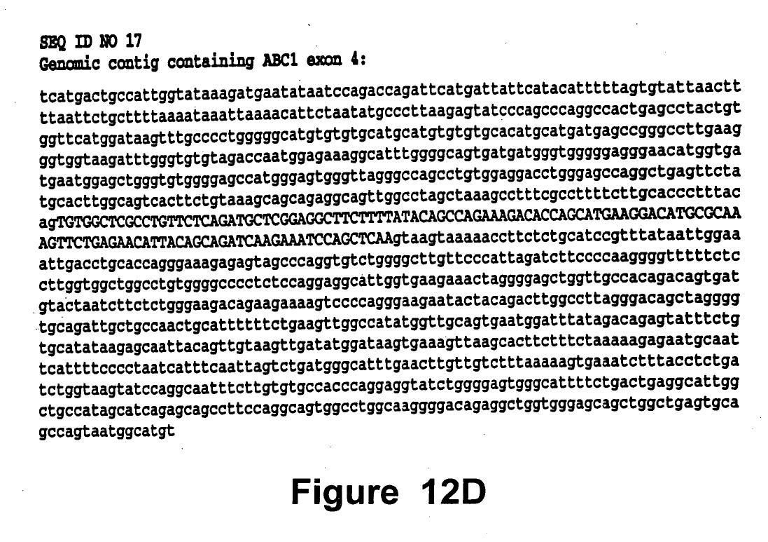

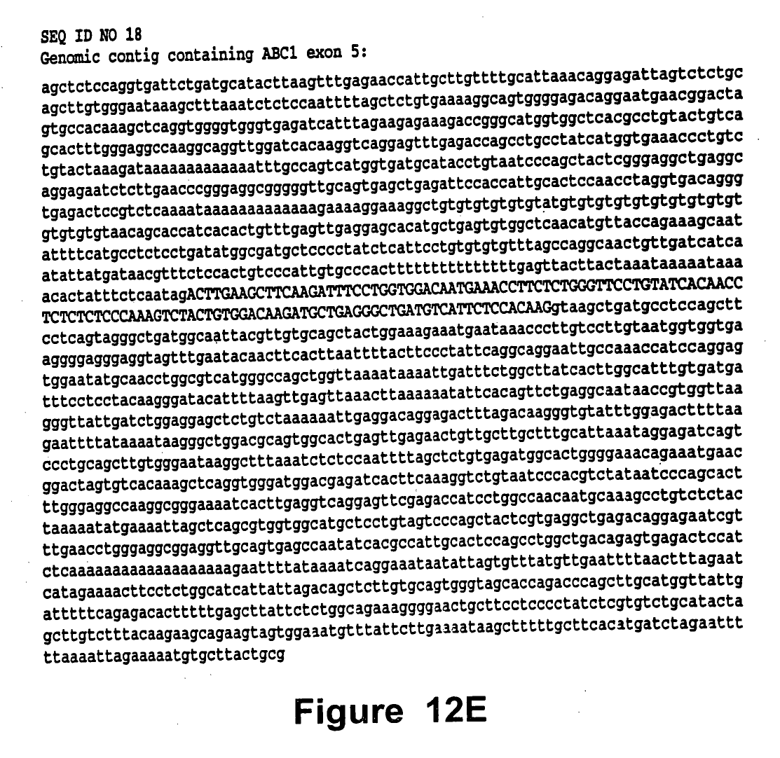

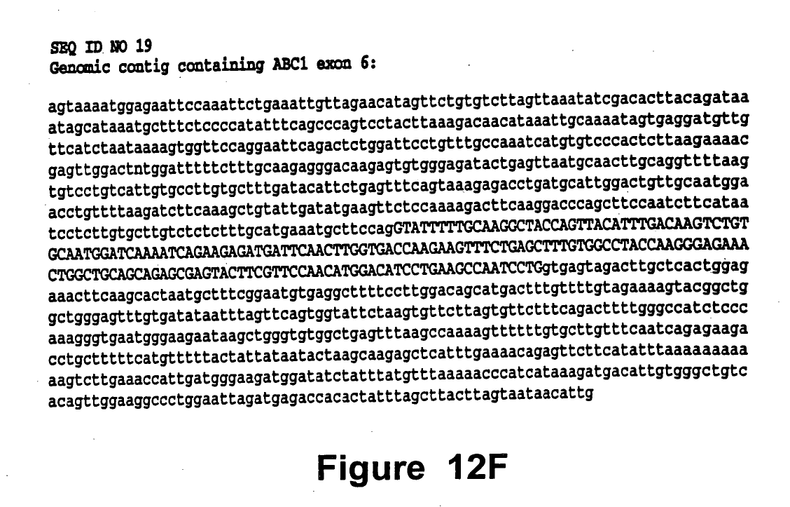

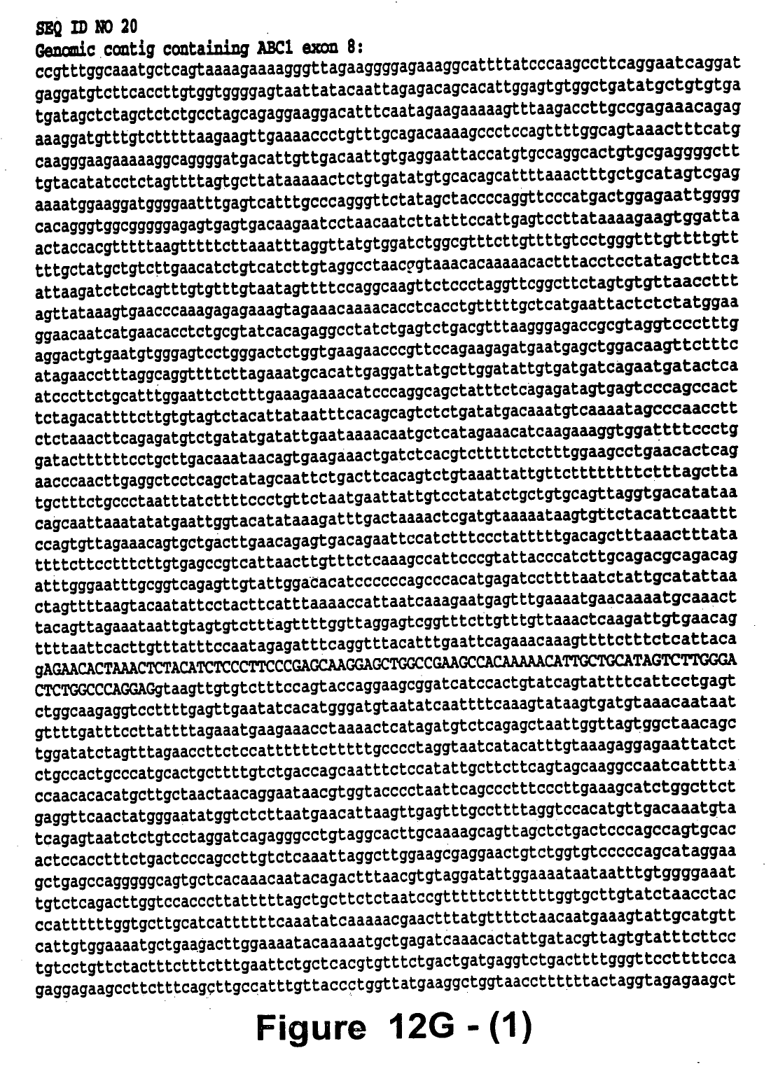

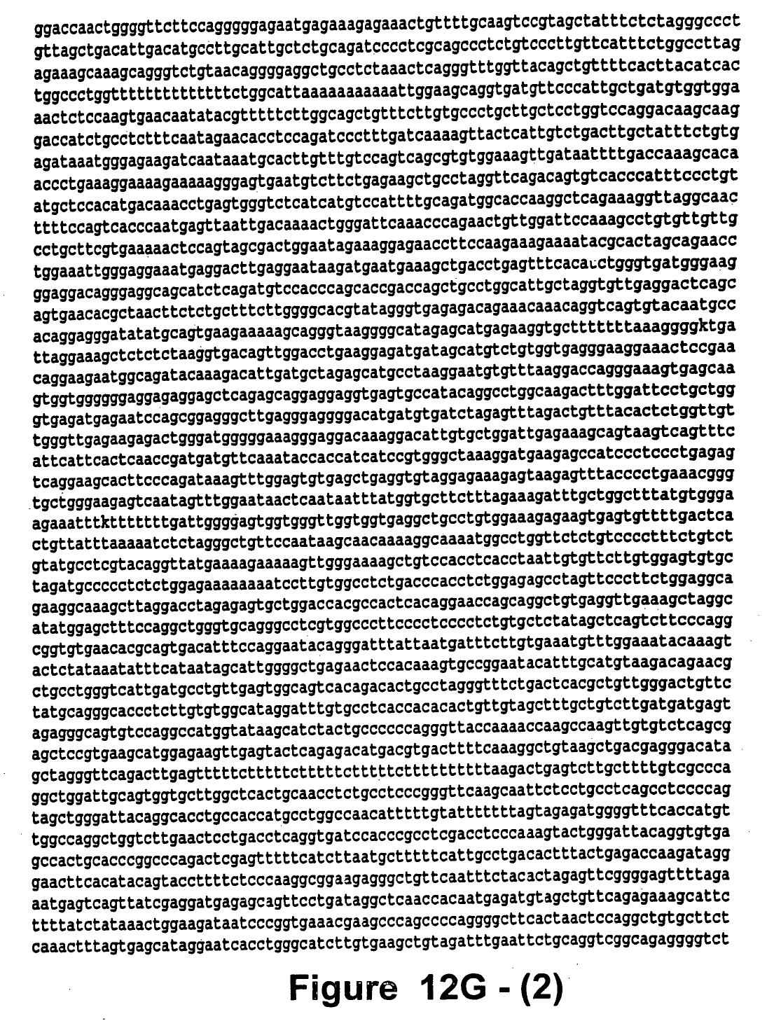

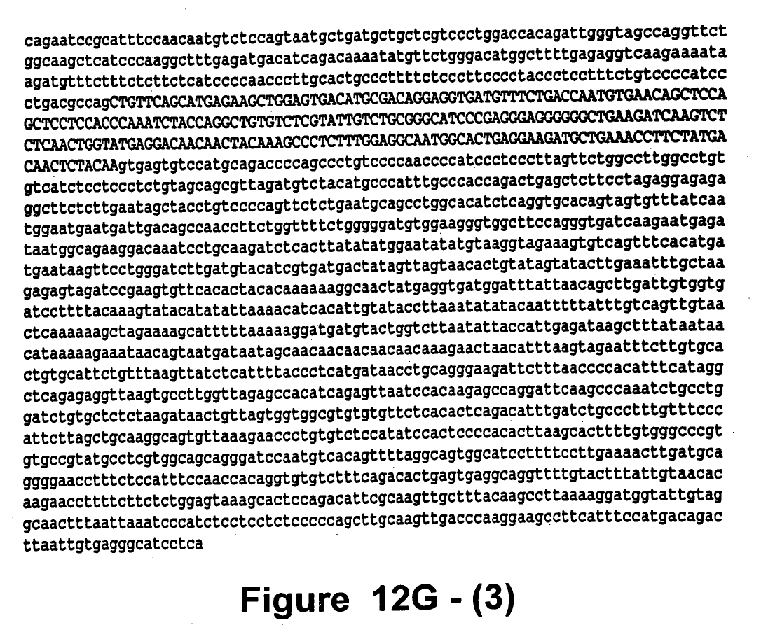

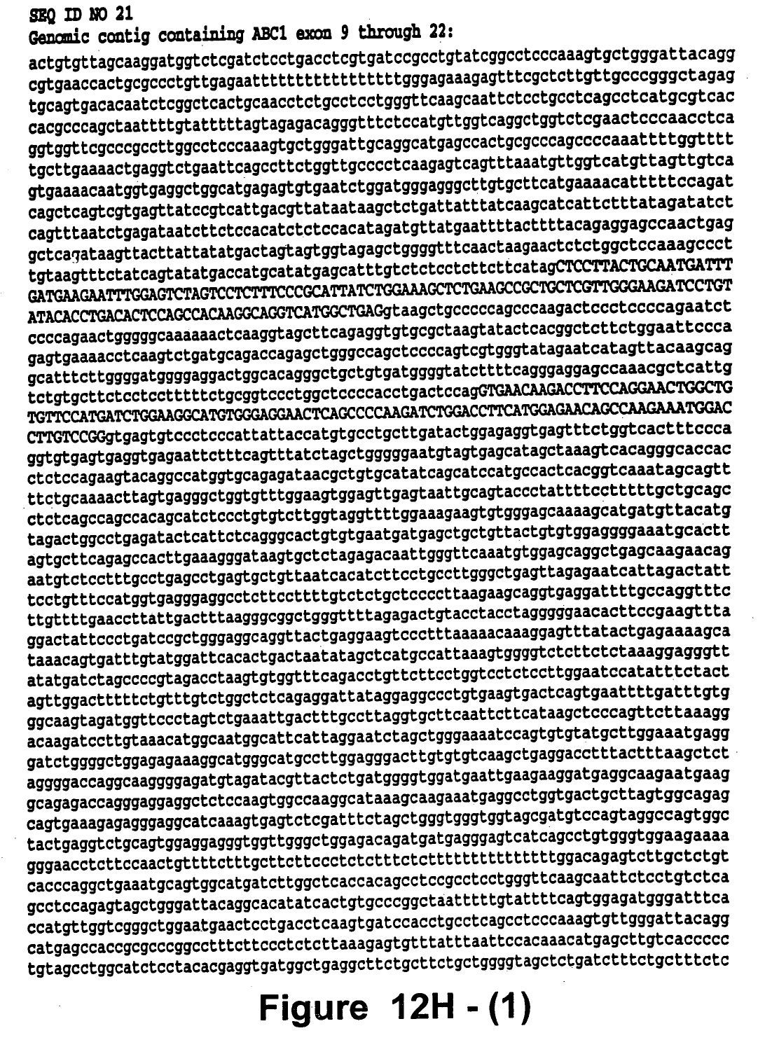

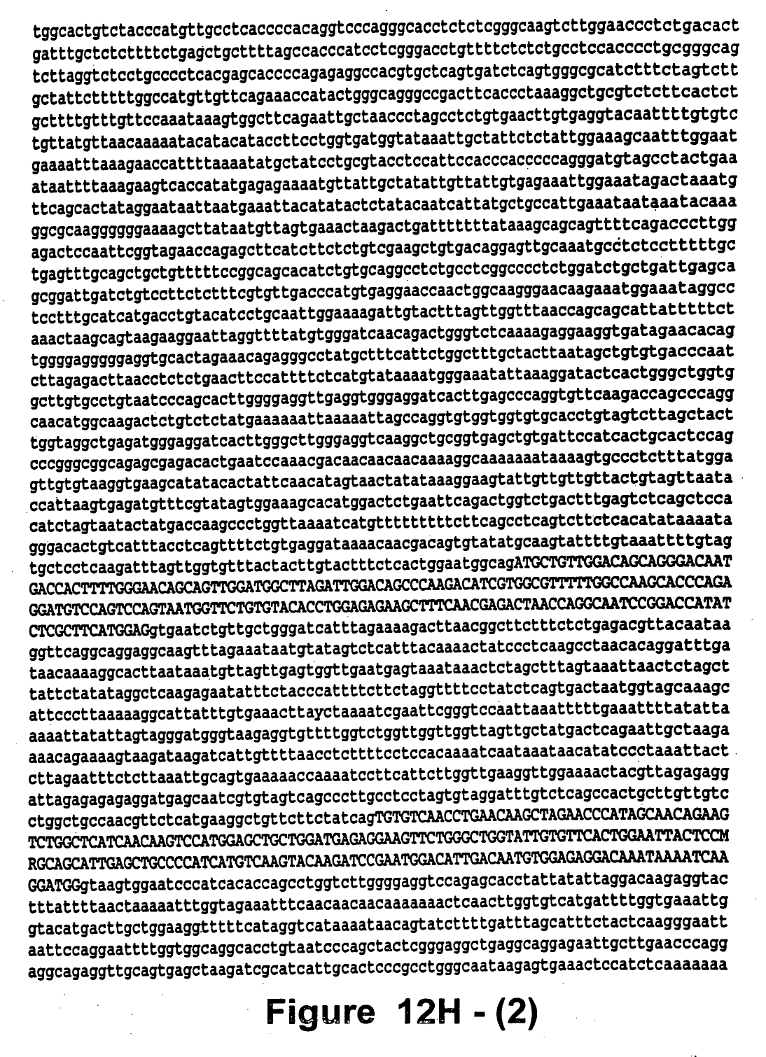

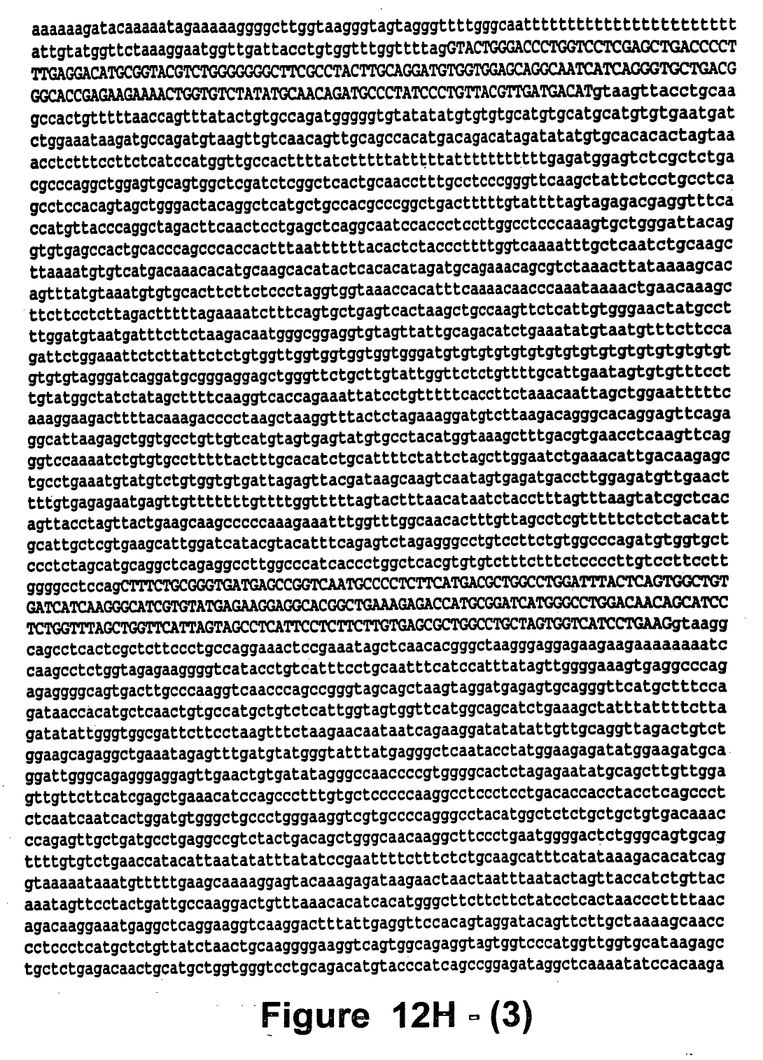

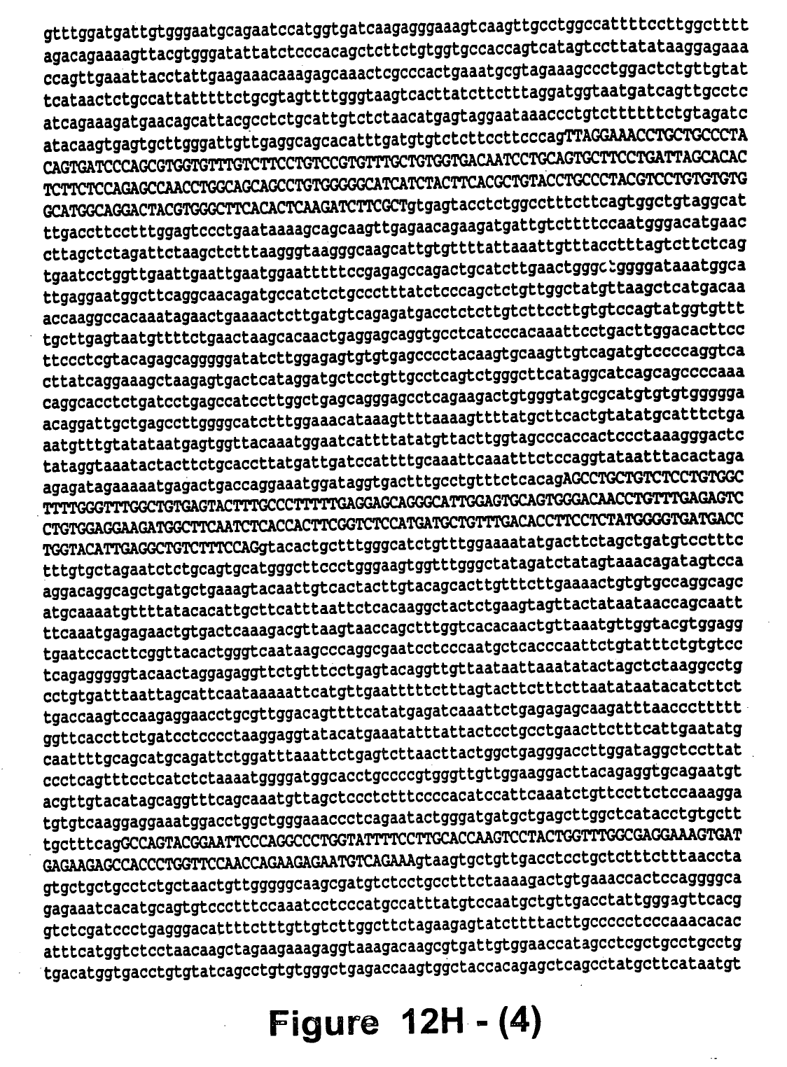

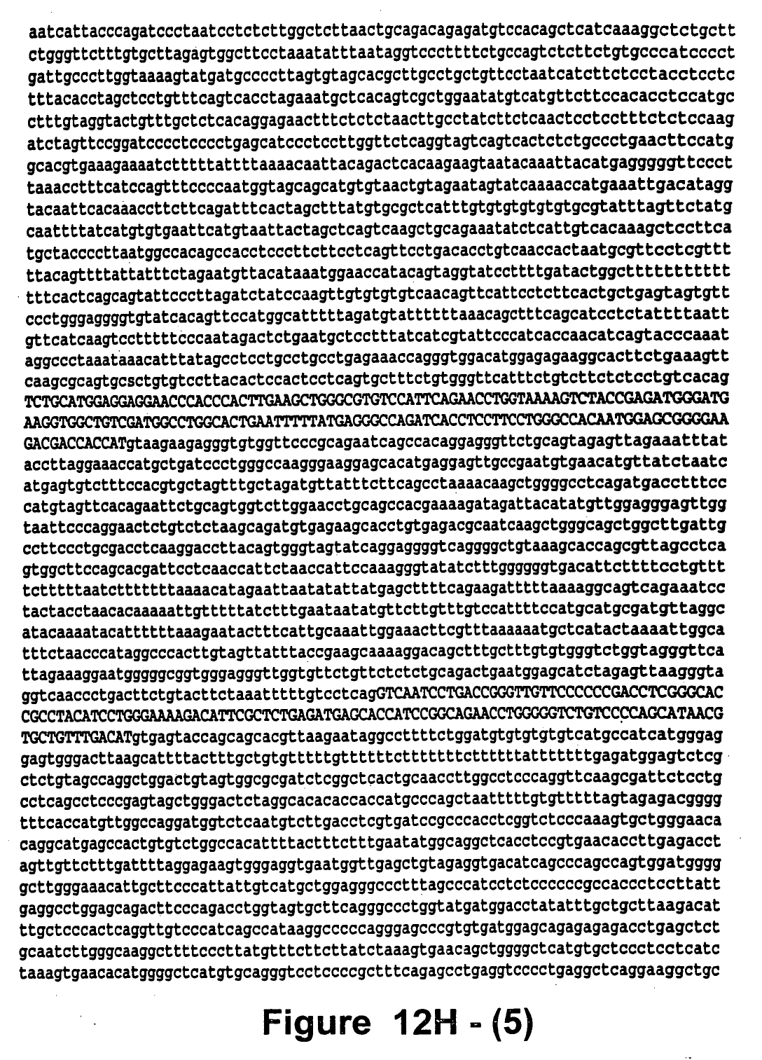

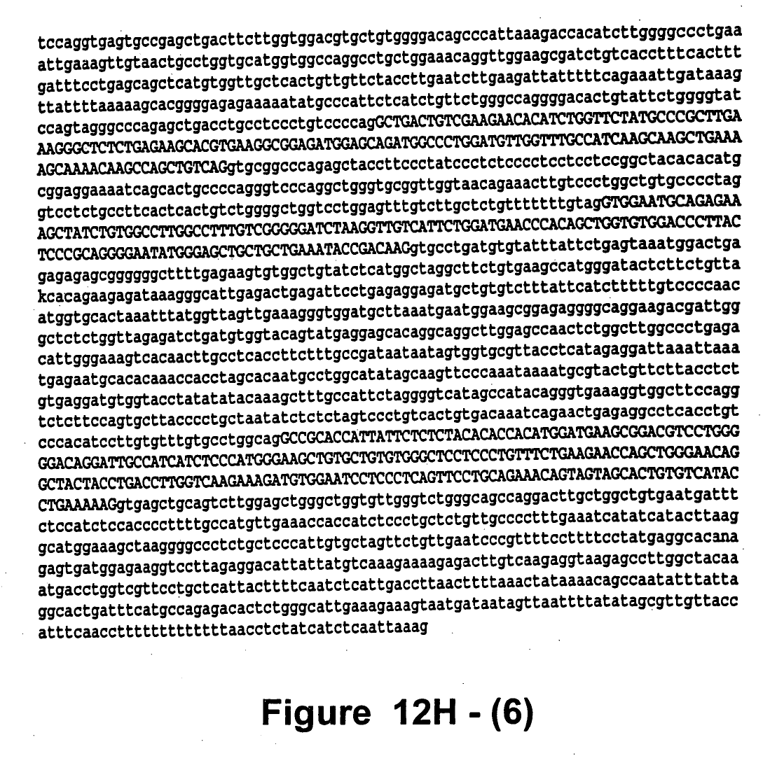

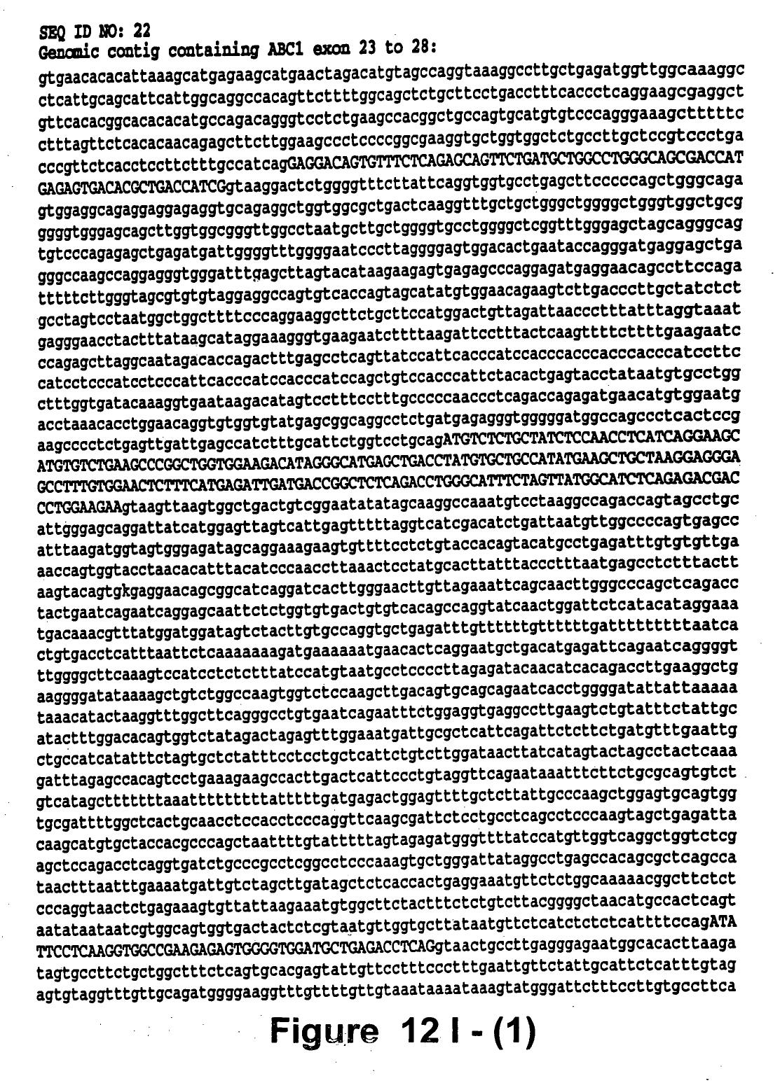

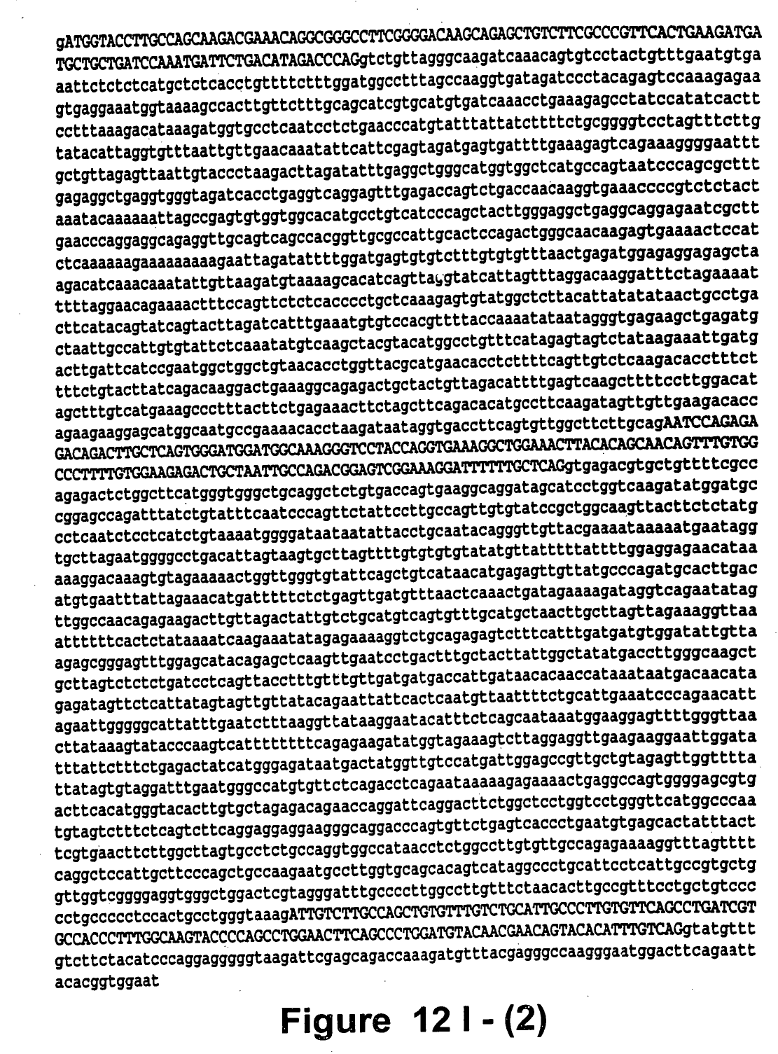

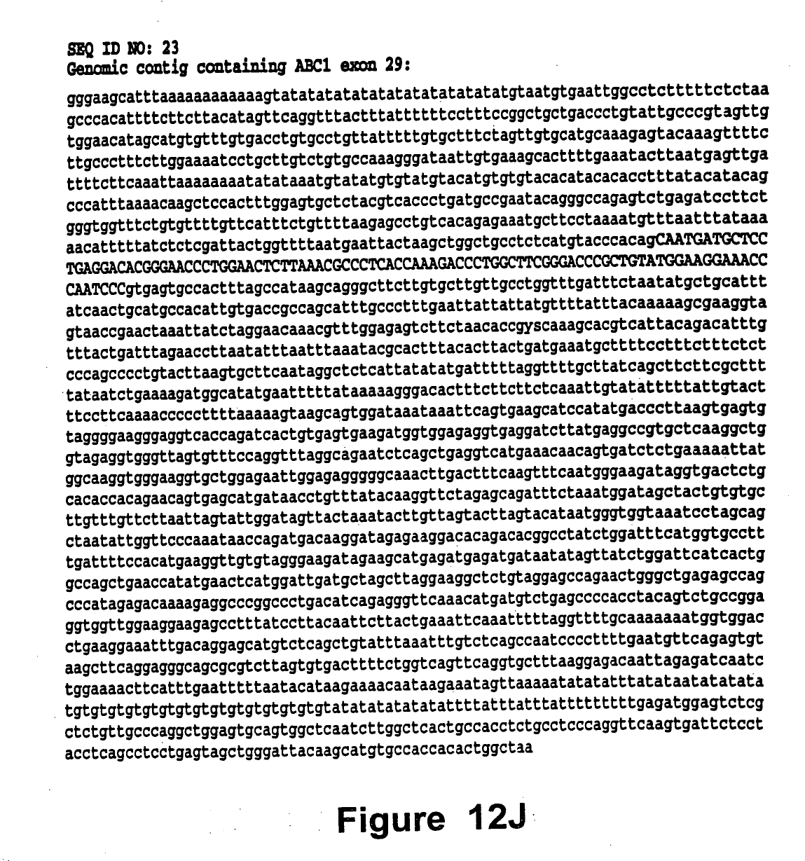

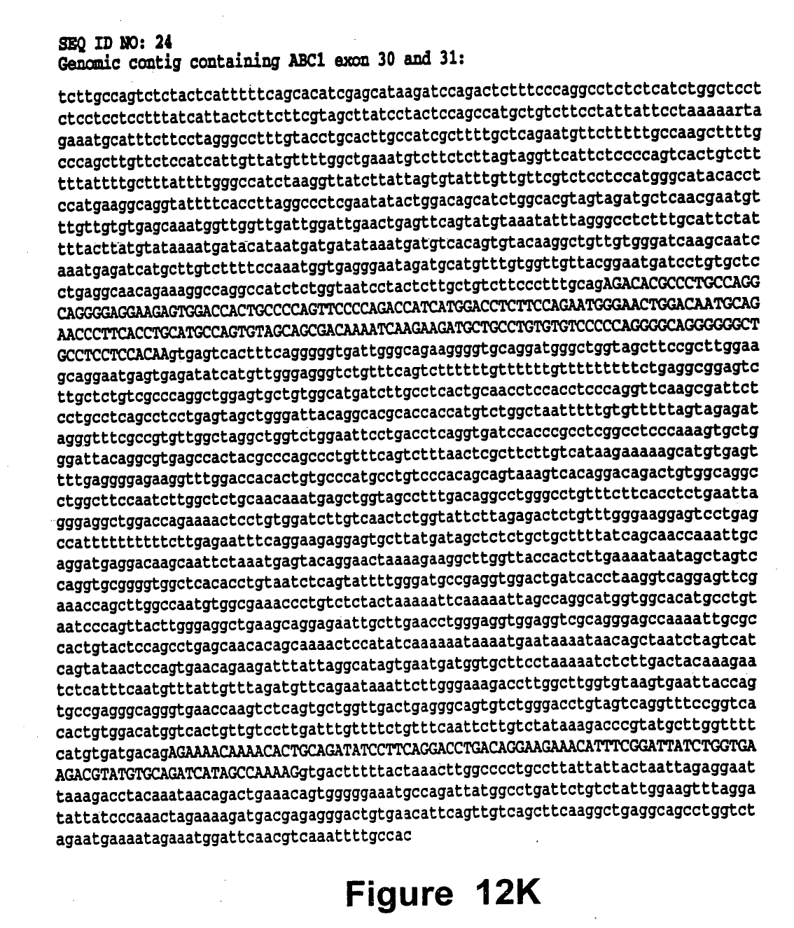

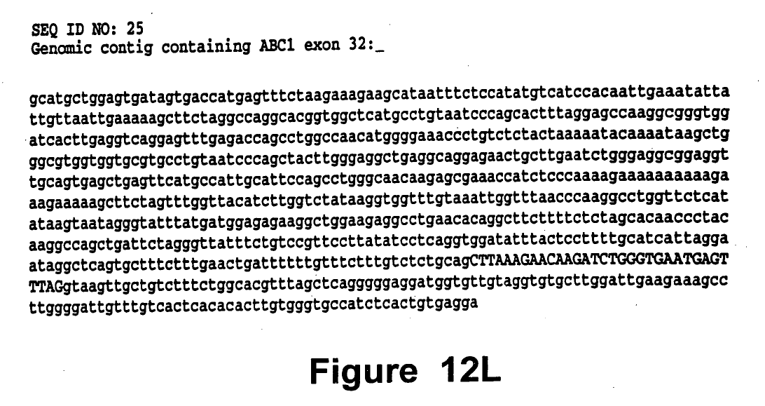

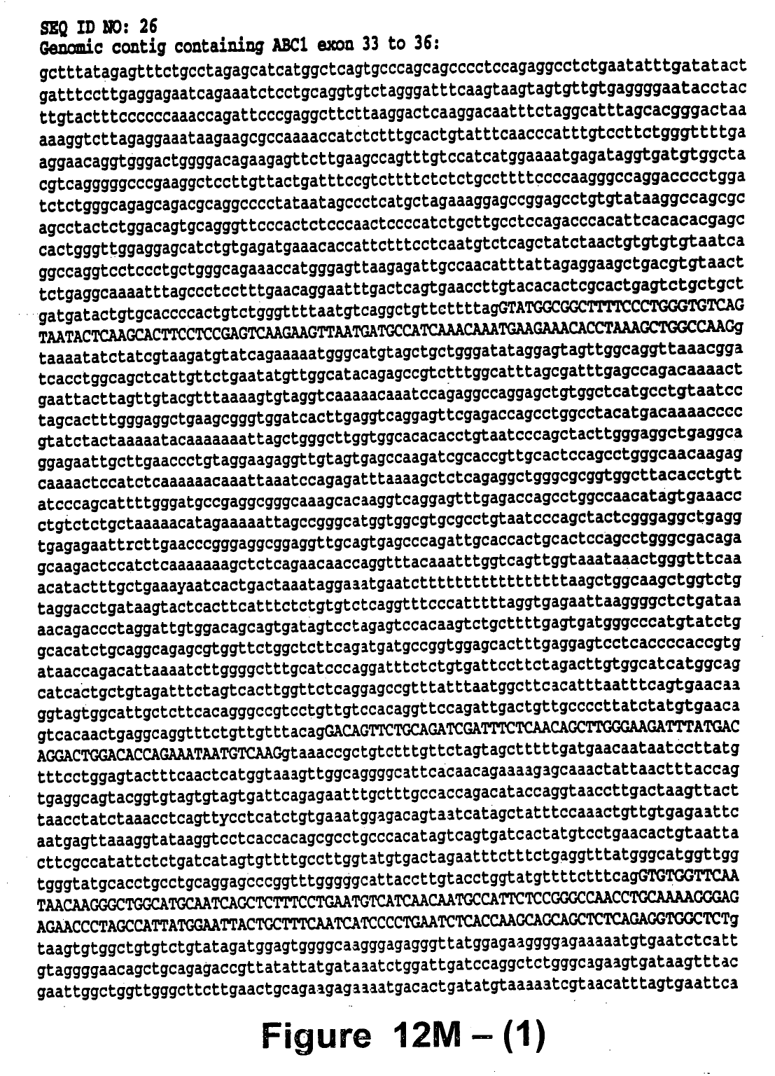

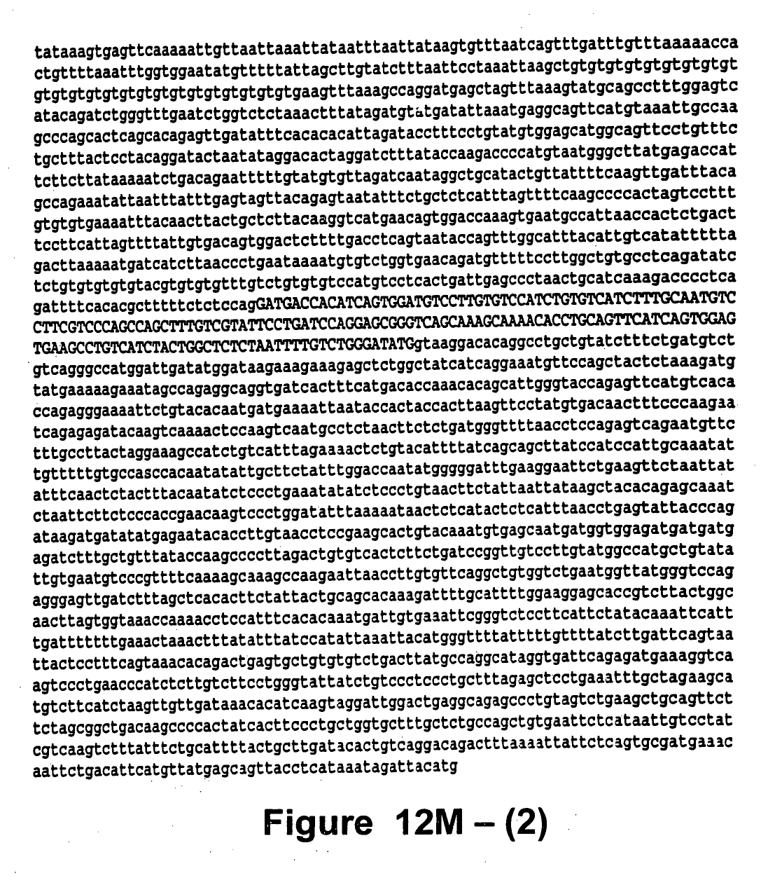

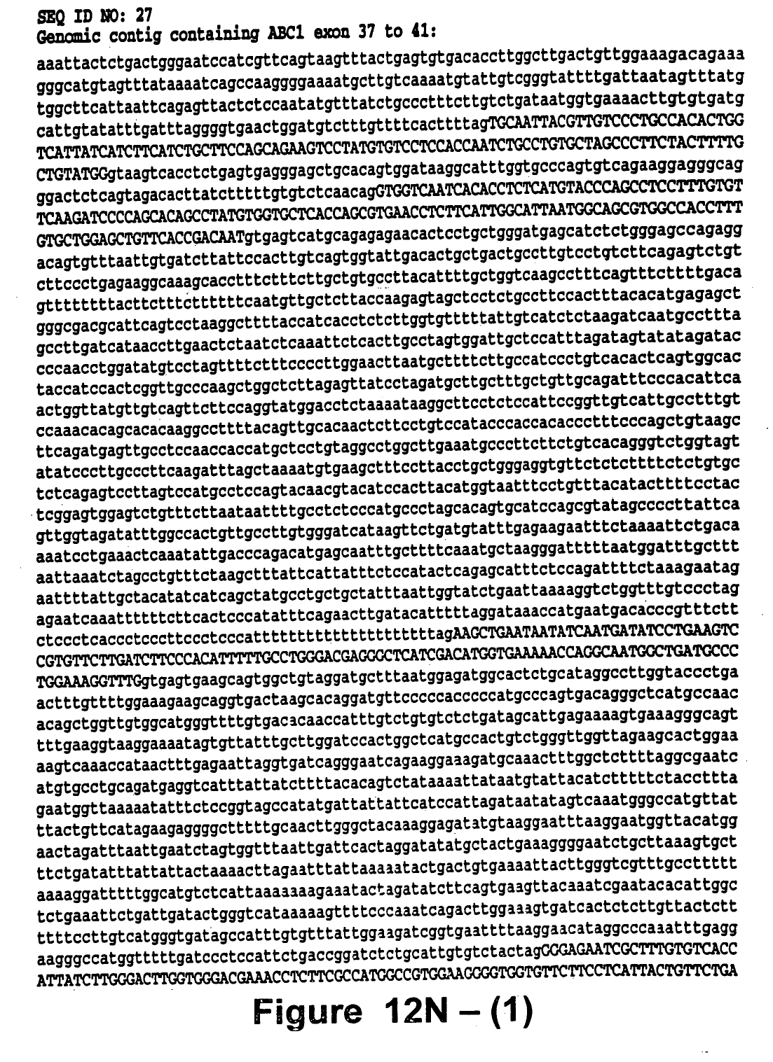

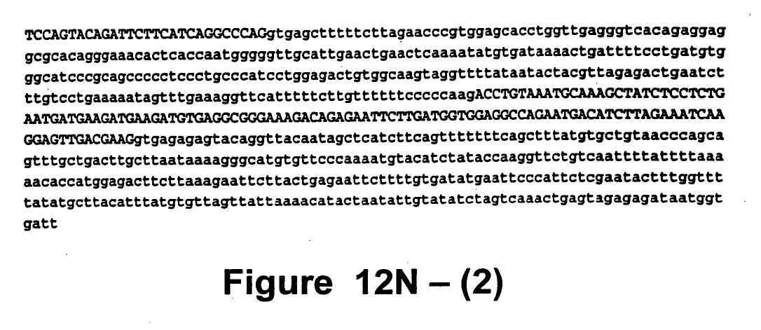

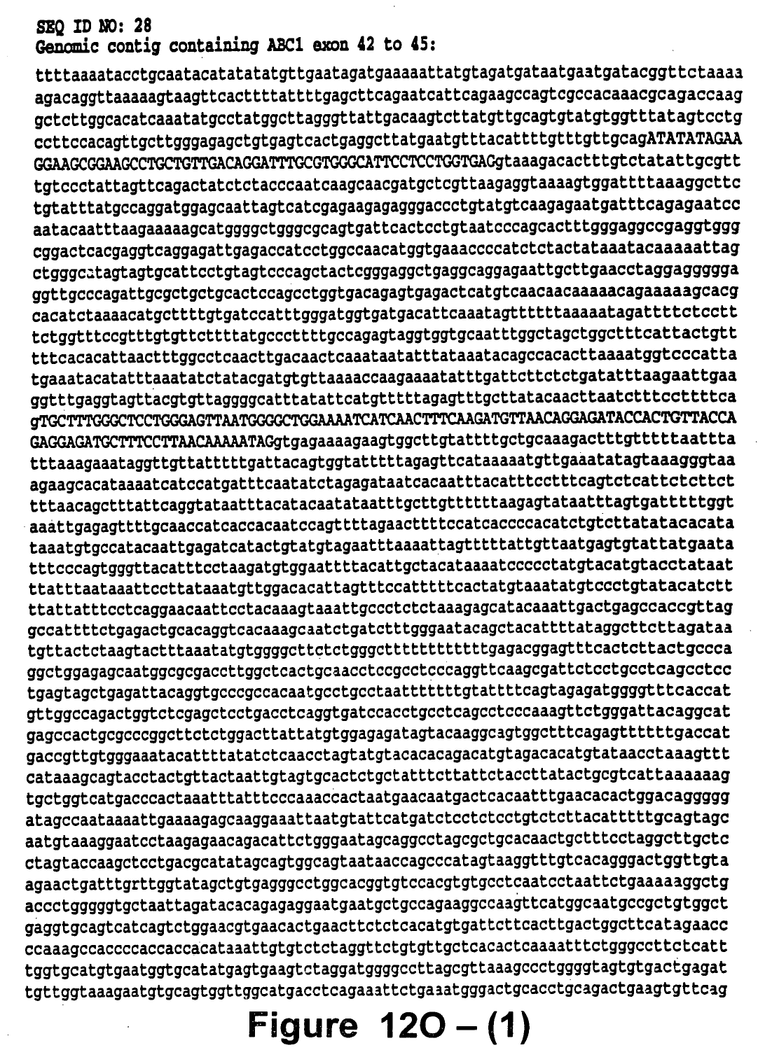

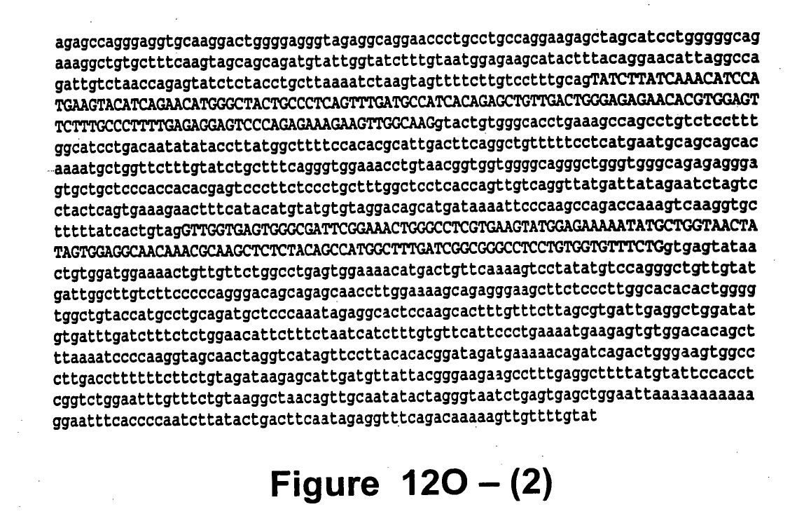

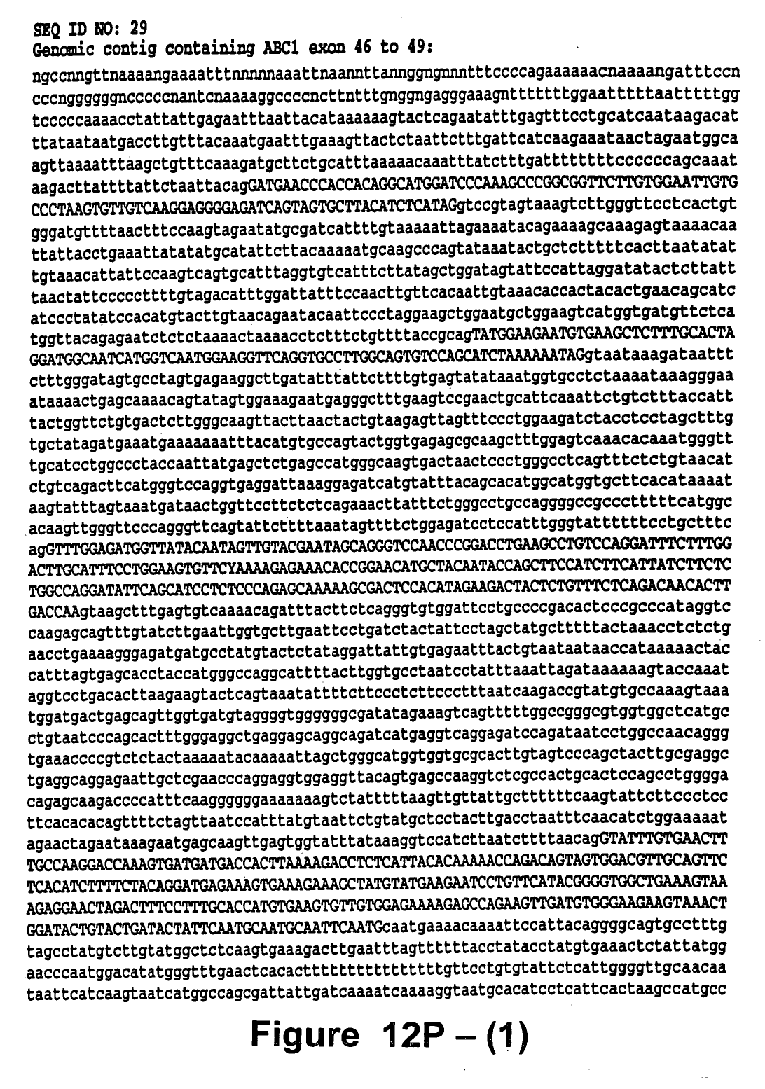

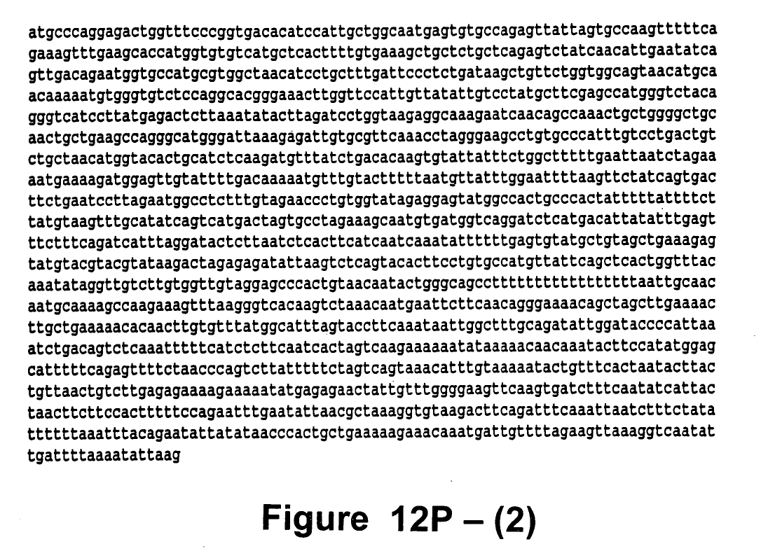

Patient and control total fibroblast RNA was used in Northern blot analysis and RT-PCR and sequence analyses. RT-PCR and sequence analysis of TD-1 revealed a heterozygous T to C substitution (FIG. 4A) in the TD-1 proband, which would result in a substitution of arginine for cysteine at a conserved residue between mouse and man (FIG. 4B). This mutation, confirmed by sequencing exon 30 of the ABC1 gene, exhibited complete segregation with the phenotype on one side of this family (FIG. 4C). This substitution creates a Hgal site, allowing for RFLP analysis of amplified genomic DNA and confirmation of the mutation (FIG. 4C). The point mutation in exon 30 was not seen on over 200 normal chromosomes from unaffected persons of Dutch decent, and 250 chromosomes of Western European decent, indicating it is unlikely to be a polymorphism. Northern blot analysis of fibroblast RNA from this patient, using a cDNA encompassing exons 1 to 49 of the gene, revealed a normal sized ˜8 Kb transcript and a truncated mutant transcript which was not visible in control RNA or in RNA from other patients with HDL deficiency (FIG. 4D). Additionally, Northern blot analysis using clones encompassing discrete regions of the cDNA revealed that the mutant transcript was detected with a cDNA compassing exons 1 to 49 (a), 1 to 41 (b), 1 to 22 (c), much more faintly with a probe spanning exon 23 to 29 (d) and not seen with probes encompassing exons 30 to 42 (e), but not seen with cDNA fragment spanning exons 30 to 49 (f). This was repeated on multiple filters with control RNA, RNA from other patients with HDL deficiency and the other TD proband, and only in TD-1 was the truncated transcript observed. Sequence analysis of the coding region did not reveal an alteration in sequence that could account for this finding. Furthermore, DNA analysis by Southern blot did not reveal any major rearrangements. Completion of exon sequencing in genomic DNA showed that this mutation was a G to C transversion at position (+1) of intron 24, (FIG. 11) affecting a splice donor site and causing aberrant splicing.

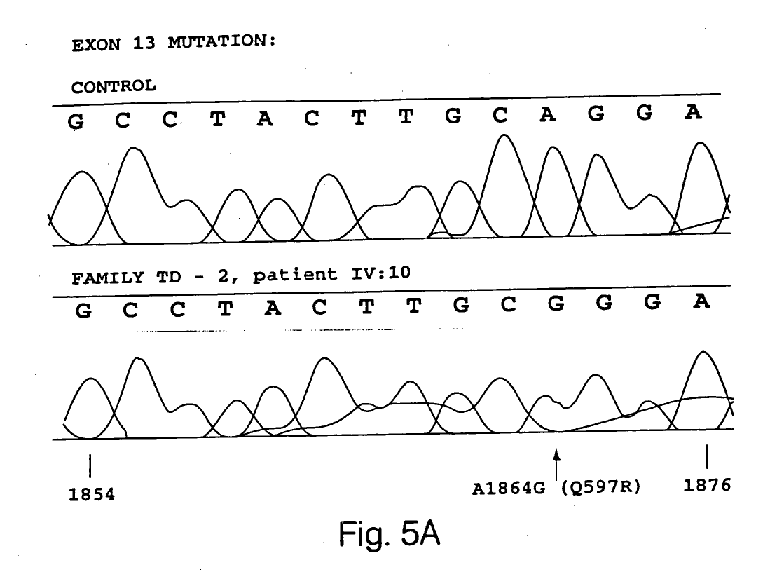

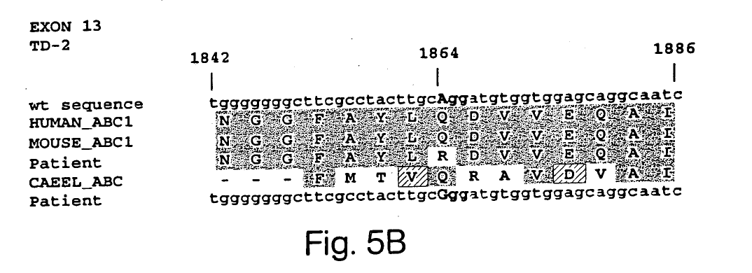

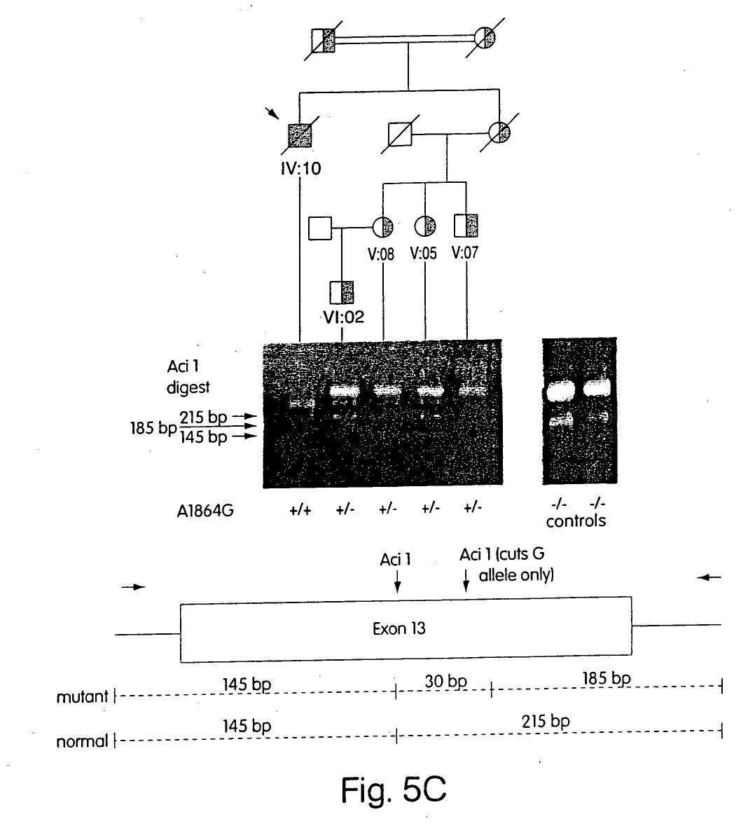

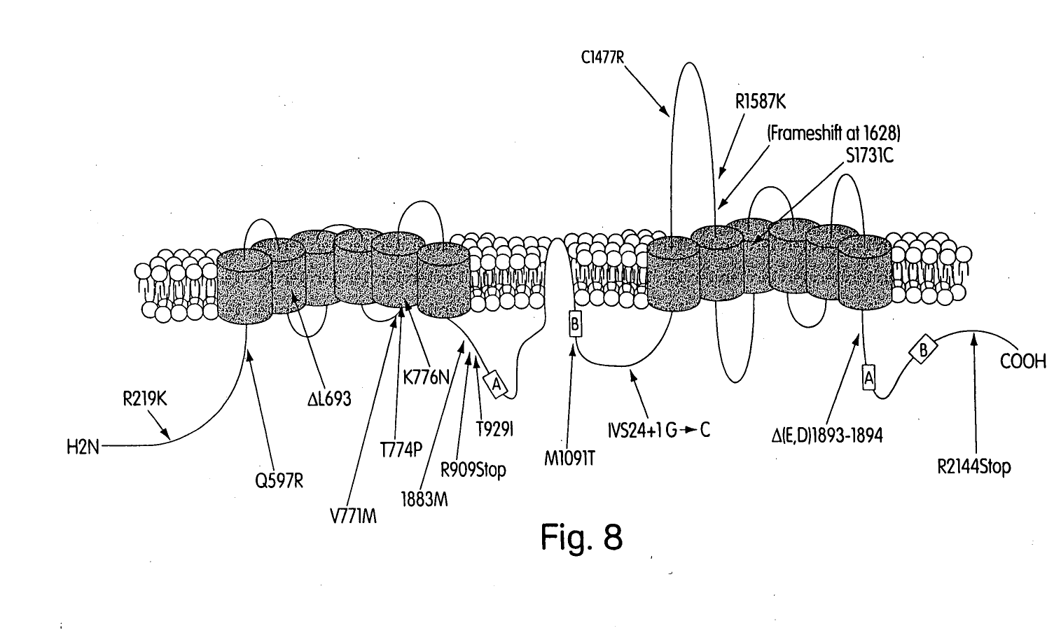

RT-PCR analysis of fibroblast RNA encoding the ABC1 gene from the proband in TD-2 (FIG. 1B) revealed a homozygous nucleotide change of A to G at nucleotide 1864 of SEQ ID NO: 2 in exon 13 (FIG. 5A), resulting in a substitution of arginine for glutamine at residue 597 of SEQ ID NO: 1 (FIG. 5B), occurring just proximal to the first predicted transmembrane domain of ABC1 (FIG. 8) at a residue conserved in mouse and as well as a C. elegans homolog. This mutation creates a second Acil site within exon 13. Segregation analysis of the mutation in this family revealed complete concordance between the mutation and the low HDL phenotype as predicted (FIG. 5C). The proband in TD-2 is homozygous for this mutation, consistent with our expectation of a disease causing mutation in this consanguineous family.

Analysis of FHA Families

Linkage Analysis and Refinement of the Minimal Genomic Region Containing the Gene for FHA

Data from microsatellite typing of individual family members from the four pedigrees of French Canadian origin were analyzed (FIG. 2). A maximum LOD score of 9.67 at a recombination fraction of 0.0 was detected at D9S277 on chromosome 9q31 (FIG. 3; Table 3). Thereafter, 22 markers were typed in a region spanning 10 cM around this locus in these families (FIGS. 2 and 3). The frequency for these markers were estimated from a sample of unrelated and unaffected subjects of French ancestry (Table 2).

TABLE 3
Two Point Linkage Analysis of FHA

| Marker Locus | LOD Score at recombination fraction | | | | | | |
|---|---|---|---|---|---|---|---|
| | 0 | 0.01 | 0.05 | 0.10 | 0.20 | 0.30 | 0.40 |
| D9S283 | −infini | −2.57 | 0.51 | 1.48 | 1.84 | 1.48 | 0.76 |
| D9S176 | −infini | 1.42 | 3.07 | 3.39 | 3.05 | 2.22 | 1.12 |
| D9S1690 | −infini | 3.11 | 4.04 | 4.04 | 3.33 | 2.24 | 0.96 |
| D9S277 | 9.67 | 9.51 | 8.89 | 8.06 | 6.29 | 4.30 | 2.10 |
| D9S306 | 5.60 | 5.51 | 5.13 | 4.62 | 3.55 | 2.36 | 1.11 |
| D9S1866 | −infini | 7.24 | 7.35 | 6.87 | 5.50 | 3.82 | 1.91 |
| D9S1784 | −infini | 8.85 | 7.76 | 9.03 | 7.09 | 4.78 | 2.25 |
| D9S172 | −infini | 2.63 | 3.00 | 2.87 | 2.26 | 1.50 | 0.67 |
| D9S1832 | −infini | 5.20 | 5.97 | 5.75 | 4.59 | 3.02 | 1.30 |
| D9S1801 | 0.14 | 0.13 | 0.11 | 0.09 | 0.06 | 0.03 | 0,.01 |
| D9S1677 | −infini | 7.83 | 7.90 | 7.38 | 5.90 | 4.08 | 2.01 |
| D9S279 | −infini | 3.43 | 3.80 | 3.66 | 3.01 | 2.12 | 1.05 |
| D9S275 | −infini | 2.57 | 2.98 | 2.91 | 2.41 | 1.69 | 0.81 |

Results of pairwise linkage analysis using MLINK. Values correspond to LOD score for linkage between the disease locus and a marker locus for specified values of the recombination fraction.

TD and FHA have thus far been deemed distinct with separate clinical and biochemical characteristics. Even though the genes for these disorders mapped to the same region, it was uncertain whether FHA and TD were due to mutations in the same gene or, alternatively, due to mutations in genes in a similar region.

Refinement of the region containing the gene for FHA was possible by examining haplotype sharing and identification of critical recombination events (FIG. 2). Seven separate meiotic recombination events were seen in these families (“A” through “G” in FIGS. 2 and 3), clearly indicating that the minimal genomic region containing the potential disease gene was a region of approximately 4.4 cM genomic DNA spanned by marker D9S1690 and D9S1866 (FIGS. 2 and 3). This region is consistent with the results of two point linkage analysis which revealed maximal LOD scores with markers D9S277 and D9S306 and essentially excluded the region centromeric to D9S1690 or telomeric to D9S1866. An 8^{th} meiotic recombination event (“H” in FIG. 3) further refined the FHA region to distal to D9S277.

As described herein, the ABC1 gene mapped within this interval. The overlapping genetic data strongly suggested that FHA may in fact be allelic to TD. Utilization of sets of genetic data from FHA and TD provided a telomeric boundary at D9S1866 (meiotic recombinant) (FIG. 3D) and a centromeric marker at D9S127 based on the homozygosity data of TD-2. This refined the locus to approximately 1 Mb between D9S127 and D9S1866. The ABC1 gene mapped within this minimal region (FIG. 3E).

Mutation Detection in FHA

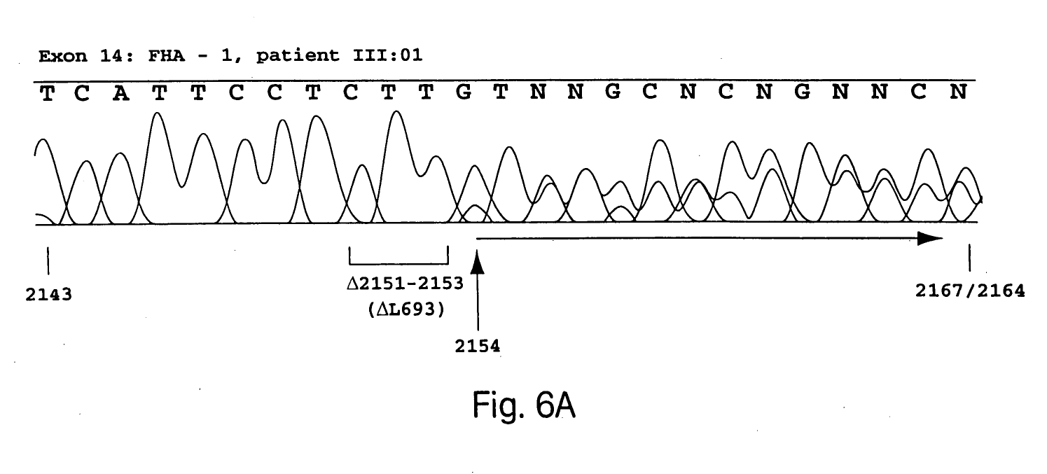

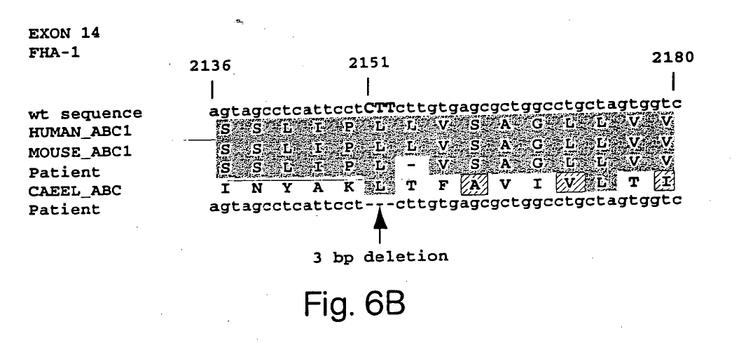

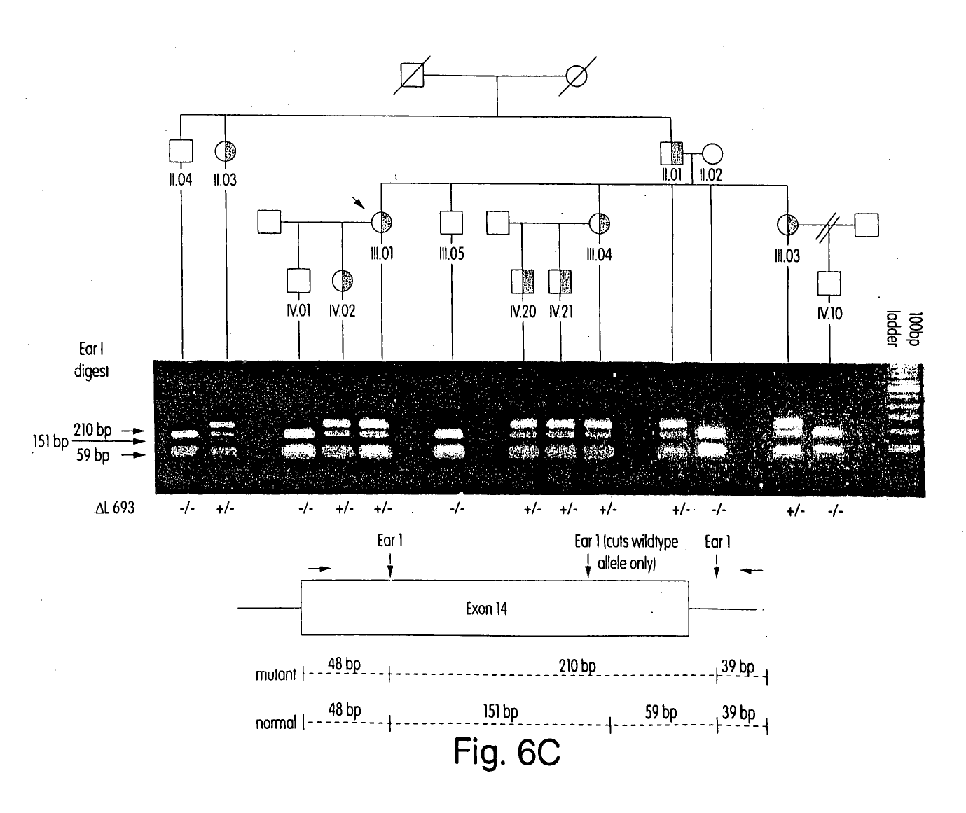

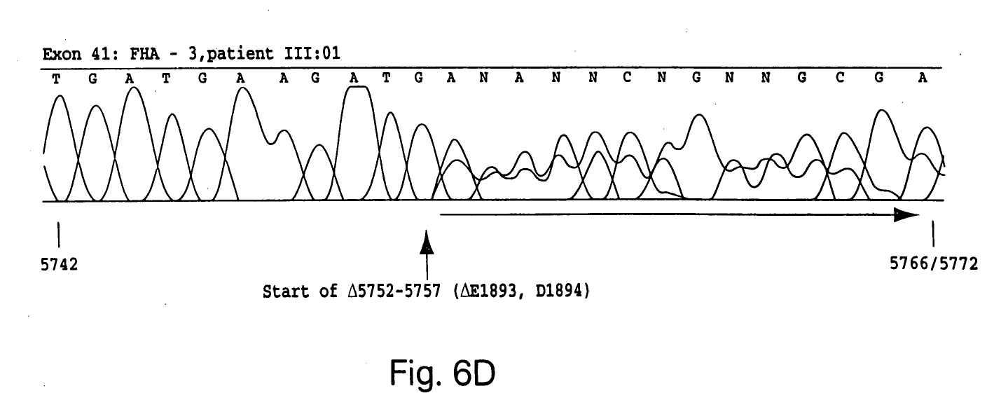

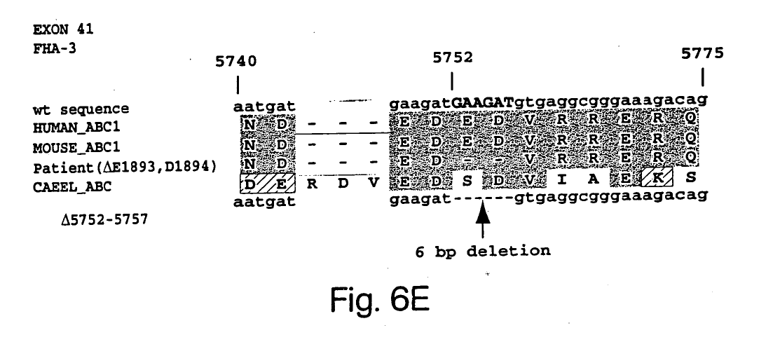

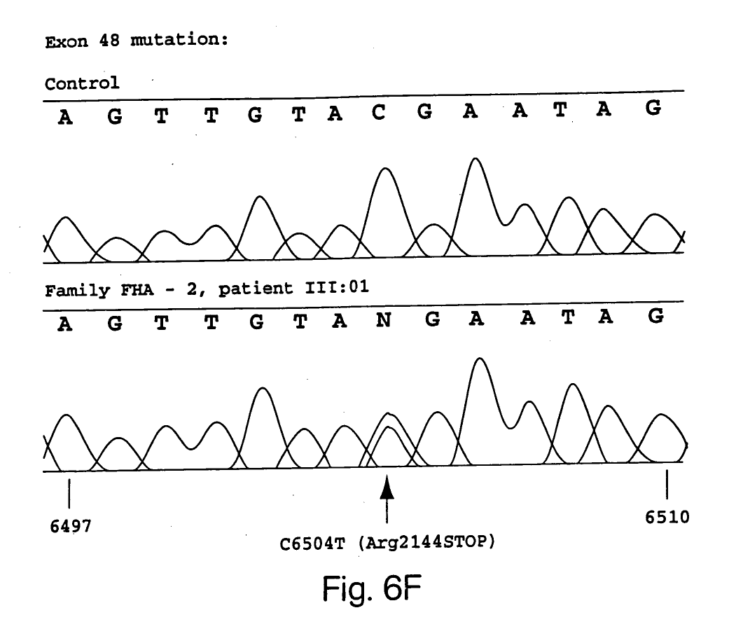

Mutation assessment of the ABC1 gene was undertaken in FHA-1 (FIG. 2A). Using primers that spanned overlapping segments of the mRNA we performed RT-PCR analysis and subjected these fragments to mutational analysis. A deletion of three nucleotides is evident in the RT-PCR sequence of FHA-1 III.01 (FIG. 6A), resulting in a loss of nucleotides 2151-2153 of SEQ ID NO: 2 and deletion of a leucine (L693) at amino acid position 693 of SEQ ID NO: 1 (FIG. 6A). This leucine is conserved in mouse and C. elegans (FIG. 6B). The alteration was detected in the RT-PCR products as well as in genomic sequence from exon 14 specific amplification. This mutation results in a loss of an Earl restriction site. Analysis of genomic DNA from the family indicated that the mutation segregated completely with the phenotype of HDL deficiency. The loss of the Earl site results in a larger fragment being remaining in persons heterozygous for this mutation (FIG. 6C). This mutation maps to the first putative transmembrane domain of ABC1 (FIG. 8) and was not seen in 130 chromosomes from persons of French Canadian descent nor seen in over 400 chromosomes from persons of other Western European ancestry.

A mutation has also been found in patient genomic DNA in pedigree FHA-3 from Quebec. The alteration, a 6 bp deletion of nucleotides 5752-5757 of SEQ ID NO: 2 within exon 41, results in a deletion of amino acids 1893 (Glu) and 1894 (Asp) of SEQ ID NO: 1. The deletion was detected as a double, superimposed, sequence starting from the point of the deletion (FIG. 6D), and was detected in sequence reads in both directions. The deletion can be detected on 3% agarose or 10% polyacrylamide gels, and segregates with disease in FHA-3. It was not seen in 128 normal chromosomes of French-Canadian origin or in 434 other control chromosomes. Amino acids 1893 and 1894 are in a region of the ABC1 protein that is conserved between human, mouse, and C. elegans (FIG. 6E), implying that it is of functional importance.

An additional mutation has been found in patient genomic DNA in pedigree FHA-2 from Quebec (FIG. 6F). The alteration, a C to T transition at position 6504 of SEQ ID NO: 2, converts an arginine at position 2144 of SEQ ID NO: 1 to a STOP codon, causing truncation of the last 118 amino acids of the ABC1 protein. This alteration segregates with disease in family FHA-2.

A summary of all mutations and polymorphisms found in ABC1 is shown in FIG. 11. Each variant indicated as a mutation segregates with low HDL in its family, and was not seen in several hundred control chromosomes.

Functional Relationship Between Changes in ABC1 Transcript Levels and Cholesterol Efflux

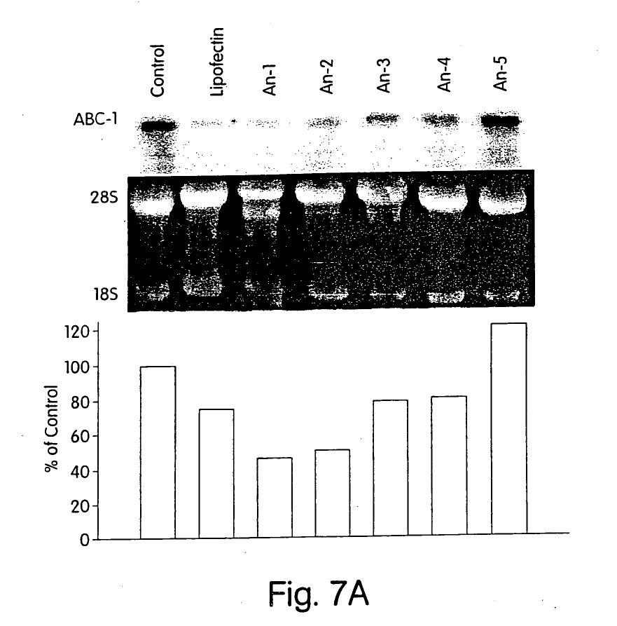

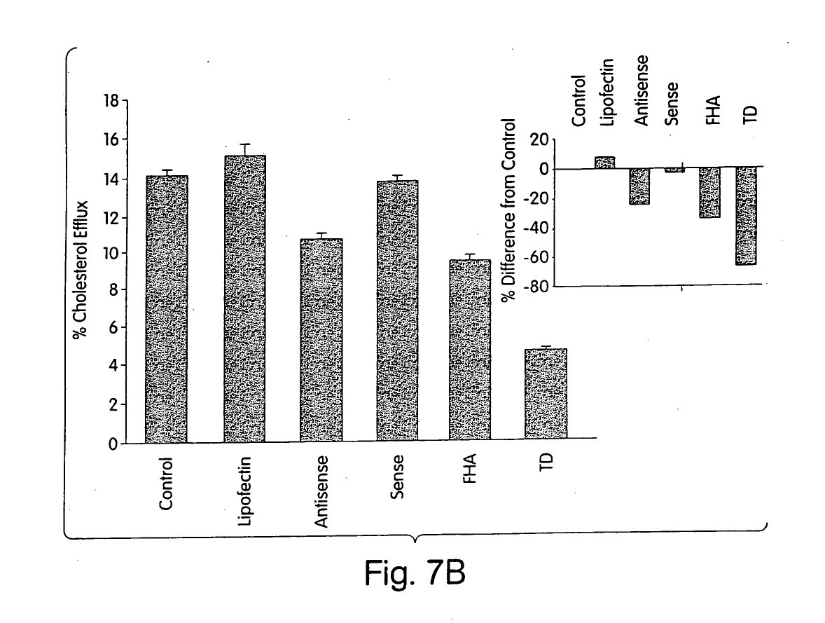

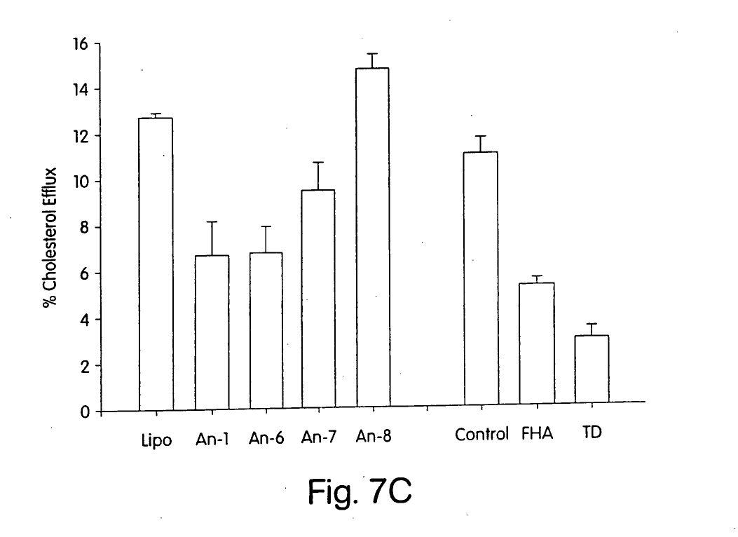

Antisense approaches were undertaken to decrease the ABC1 transcript and assess the effect of alteration of the transcript on intracellular cholesterol transport. The use of antisense primers to the 5′ end of ABC1 clearly resulted in a decrease to approximately 50% of normal RNA levels (FIG. 7A). This would be expected to mimic in part the loss of function due to mutations on one allele, similar to that seen in heterozygotes for TD and patients with FHA. Importantly, reduction in the mRNA for the ABC1 gene resulted in a significant reduction in cellular cholesterol efflux (FIG. 7B), further establishing the role of this protein in reverse cholesterol transport and providing evidence that the mutations detected are likely to constitute loss of function mutations. Furthermore, these data support the functional importance of the first 60 amino acids of the protein. Antisense oligonucleotide AN-6 is directed to the novel start codon 5′ to the one indicated in AJ012376.1; this antisense oligonucleotide effectively suppresses efflux.

The above-described results were obtained using the following materials and methods.

Patient Selection

The probands in TD families had previously been diagnosed as suffering from TD based on clinical and biochemical data. Study subjects with FHA were selected from the Cardiology Clinic of the Clinical Research Institute of Montreal. The main criterion was an HDL-C level <5th percentile for age and gender, with a plasma concentration of triglycerides <95th percentile in the proband and a first-degree relative with the same lipid abnormality. In addition, the patients did not have diabetes.

Biochemical Studies

Blood was withdrawn in EDTA-containing tubes for plasma lipid, lipoprotein cholesterol, ApoAI, and triglyceride analyses, as well as storage at. −80° C. Leukocytes were isolated from the buffy coat for DNA extraction.

Lipoprotein measurement was performed on fresh plasma as described elsewhere (Rogler et al., Arterioscler. Thromb. Vasc. Biol. 15:683-690, 1995). The laboratory participates and meets the criteria of the Lipid Research Program Standardization Program. Lipids, cholesterol and triglyceride levels were determined in total plasma and plasma at density d<1.006 g/mL (obtained after preparative ultracentrifugation) before and after precipitation with dextran manganese. Apolipoprotein measurement was performed by nephelometry for ApoB and ApoAI.

Linkage Analysis

Linkage between the trait locus and microsatellite loci was analyzed using the FASTLINK version (4.0 P). FASTLINK/MLINK was used for two-point linkage analysis assuming an autosomal dominant trait with complete penetrance. In FHA and TD heterozygotes, the phenotype was HDL deficiency <5th percentile for age and sex. The disease allele frequency was estimated to be 0.005. Marker allele frequencies were estimated from the genotypes of the founders in the pedigrees using NEWPREP. Multipoint linkage analysis was carried out using FASTLINK/LINKMAP.

Genomic Clone Assembly and Physical Map Construction of the 9q31 Region

Using the Whitehead Institute/MIT Center for Genome Research map as a reference, the genetic markers of interest at 9q31 were identified within YAC contigs. Additional markers that mapped to the approximate 9q31 interval from public databases and the literature were then assayed against the YAC clones by PCR and hybridization analysis. The order of markers was based on their presence or absence in the anchored YAC contigs and later in the BAC contig. Based on the haplotype analysis, the region between D9S277 and D9S306 was targeted for higher resolution physical mapping studies using bacterial artificial chromosomes (BACs). BACs within the region of interest were isolated by hybridization of DNA marker probes and whole YACs to high-density filters containing clones from the RPCI-11 human BAC library (FIG. 3).

Sequence Retrieval and Alignment

The human ABC1 mRNA sequence was retrieved from GenBank using the Entrez nucleotide query (Baxevanis et al., A Practical Guide to the Analysis of Genes and Proteins, eds. Baxevanis, A. D. & Ouellette, B. F. F. 98:120, 1998) as GenBank accession number AJ012376.1. The version of the protein sequence we used as wild-type (normal) was CAA10005.1.

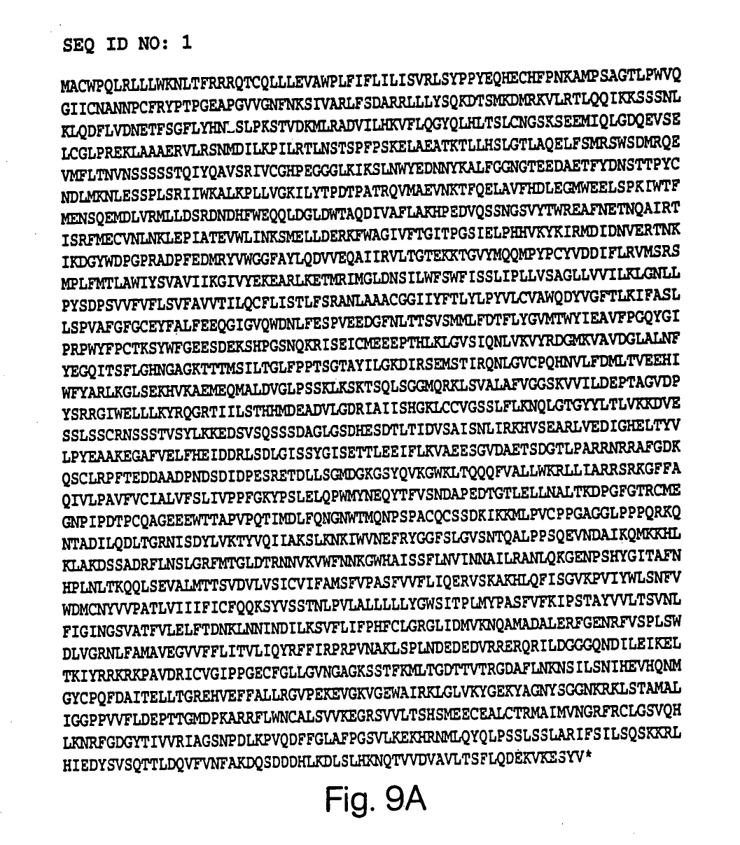

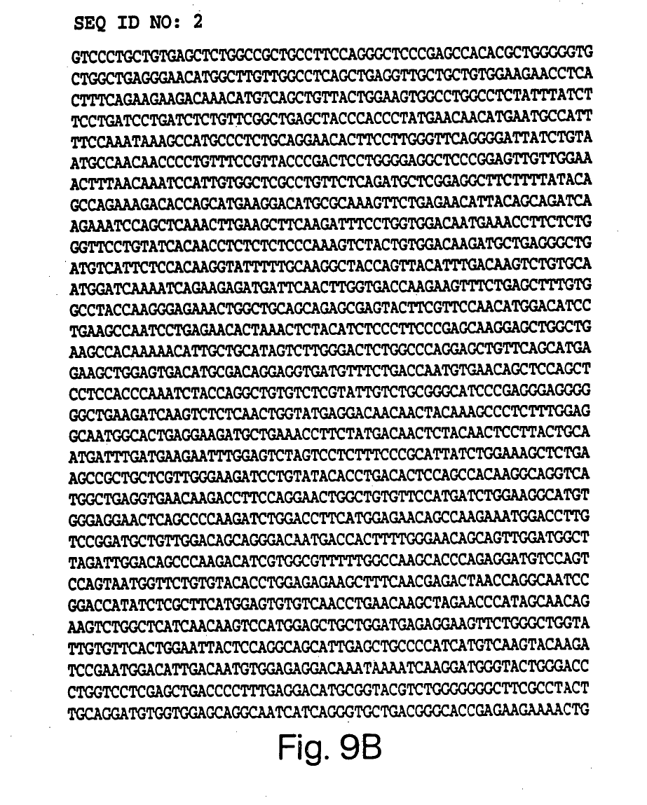

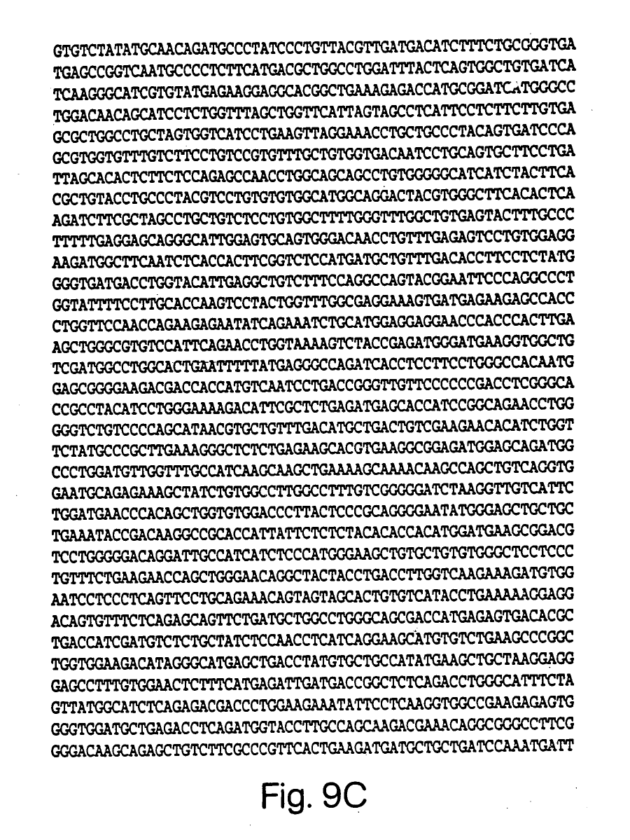

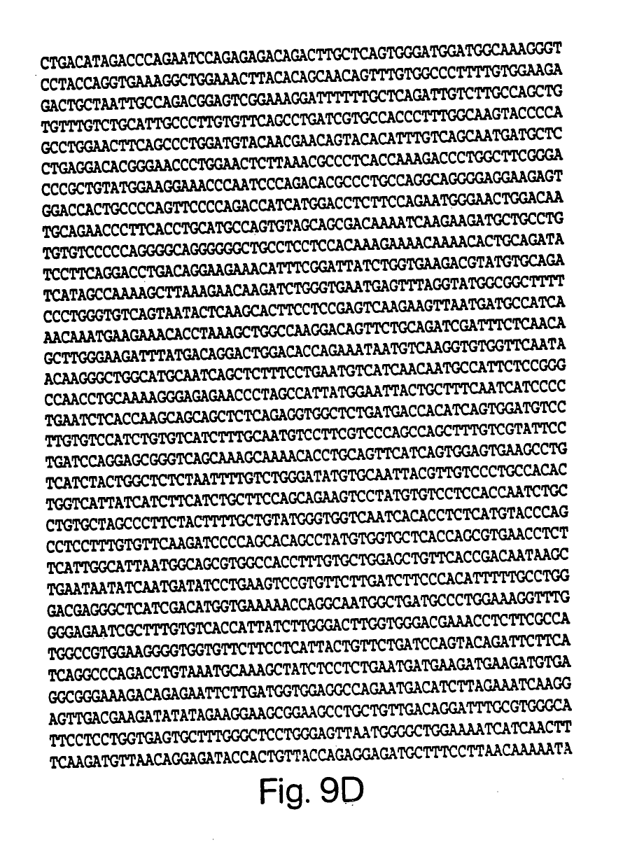

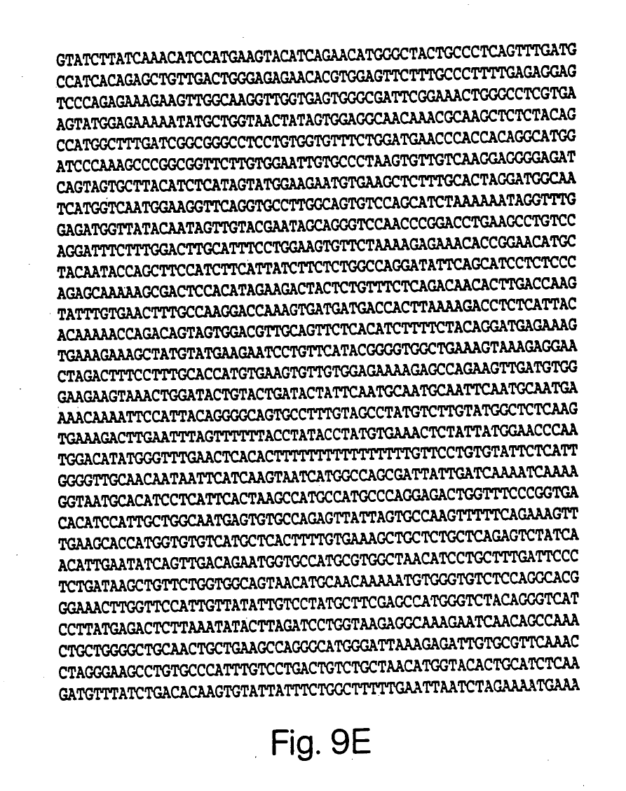

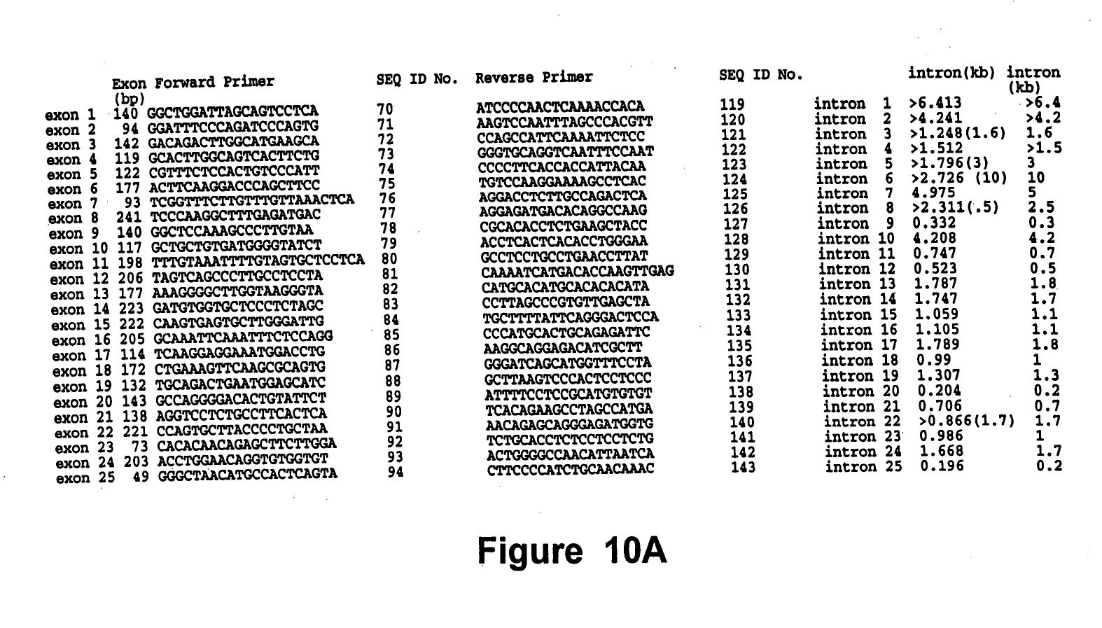

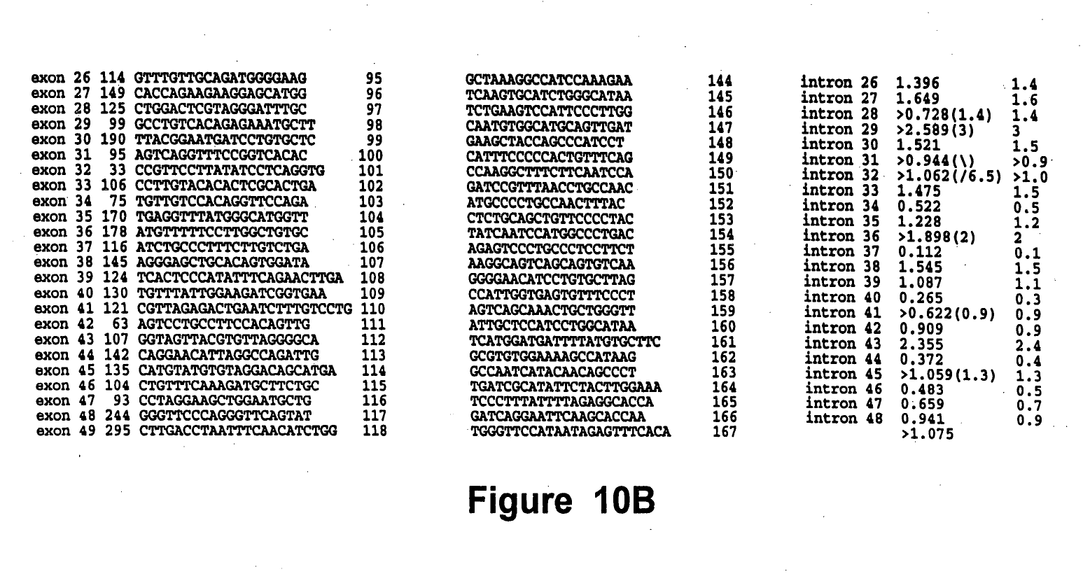

We identified an additional 60 amino acids in-frame with the previously-believed start methionine (FIG. 9A). Bioinformatic analysis of the additional amino acids indicates the presence of a short stretch of basic amino acid residues, followed by a hydrophobic stretch, then several polar residues. This may represent a leader sequence, or another transmembrane or membrane-associated region of the ABC1 protein. In order to differentiate among the foregoing possibilities, antibodies directed to the region of amino acids 1-60 are raised against and used to determine the physical relationship of amino acids 1-60 in relation to the cell membrane. Other standard methods can also be employed, including, for example, expression of fusion proteins and cell fractionation.

We also identified six errors in the previously-reported nucleotide sequence (at positions 839, 4738, 5017, 5995, 6557, and 6899 of SEQ ID NO: 2; FIG. 11). Hence, the sequence of the ABC1 polypeptide of FIG. 9A differs from CAA10005.1 as follows: Thr_Ile at position 1554; Pro_Leu at position 1642; Arg_Lys at position 1973; and Pro_Leu at position 2167. We also identified 5′ and 3′ UTR sequence (FIGS. 9B-9E).

The mouse ABC1 sequence used has accession number X75926. It is very likely that this mouse sequence is incomplete, as it lacks the additional 60 amino acids described herein for human ABC1.

Version 1.7 of ClustalW was used for multiple sequence alignments with BOXSHADE for graphical enhancement (www.isrec.isb-sib.ch:8080/software/BOX_form.html) with the default parameter. A Caenorhabditis elegans ABC1 orthologue was identified with BLAST (version 2.08) using CAA1005.1 (see above) as a query, with the default parameter except for doing an organism filter for C. elegans. The selected protein sequence has accession version number MC69223.1 with a score of 375, and an E value of 103.

Genomic DNA Sequencing

BAC DNA was extracted from bacterial cultures using NucleoBond Plasmid Maxi Kits (Clontech, Palo Alto, Calif.). For DNA sequencing, a sublibrary was first constructed from each of the BAC DNAs (Rowen et al., Automated DNA Sequencing and Analysis, eds. Adams, M. D., Fields, C. & Venter, J. C., 1994). In brief, the BAC DNA was isolated and randomly sheared by nebulization. The sheared DNA was then size fractionated by agarose gel electrophoresis and fragments above 2 kb were collected, treated with Mung Bean nuclease followed by T4 DNA polymerase and klenow enzyme to ensure blunt-ends, and cloned into Smal-cut M13mp19. Random clones were sequenced with an ABI373 or 377 sequencer and fluorescently labeled primers (Applied BioSystems, Foster City, Calif.). DNAStar software was used for gel trace analysis and contig assembly. All DNA sequences were examined against available public databases primarily using BLASTn with RepeatMasker (University of Washington).

Reverse Transcription (RT)-PCR Amplification and Sequence Analysis

Total RNA was isolated from the cultured fibroblasts of TD and FHA patients, and reverse transcribed with a CDS primer containing oligo d(T)18 using 250 units of SuperScript II reverse transcriptase (Life Technologies, Inc., Rockville, Md.) as described (Zhang et al., J. Biol. Chem. 27:1776-1783, 1996). cDNA was amplified with Taq DNA polymerase using primers derived from the published human ABC1 cDNA sequence (Luciani et al., Genomics 21:150-159, 1994). Six sets of primer pairs were designed to amplify each cDNA sample, generating six DNA fragments which are sequentially overlapped covering 135 to 7014 bp of the full-length human ABC1 cDNA. The nucleotides are numbered according to the order of the published human cDNA sequence (AJ012376.1). Primer pairs (1): 135-158 (f) and 1183-1199 (r); (2): 1080-1107 (f) and 2247-2273 (r); (3): 2171-2197 (f) and 3376-3404 (r); (4): 3323-3353 (f) and 45874617 (r); (5) 45154539 (f) and 5782-5811 (r); (6): 5742-5769 (f) and 6985-7014 (r). RT-PCR products were purified by Qiagen spin columns. Sequencing was carried out in a Model 373A Automated DNA sequencer (Applied Biosystems) using Taq di-deoxy terminator cycle sequencing and Big Dye Kits according to the manufacturer's protocol.

Northem Blot Analysis

Northern transfer and hybridizations were performed essentially as described (Zhang et al., J. Biol. Chem. 27:1776-1783, 1996). Briefly, 20 μg of total fibroblast RNA samples were resolved by electrophoresis in a denaturing agarose (1.2%; w/v) gel in the presence of 7% formaldehyde, and transferred to nylon membranes. The filters were probed with ³²P-labeled human ABC1 cDNA as indicated. Pre-hybridization and hybridizations were carried out in an ExpressHyb solution (ClonTech) at 68° C. according to the manufacturer's protocol.

Detection of the Mutations in TD

Genotyping for the T4503C and A1864G variants was performed by PCR amplification of exon 30 followed by restriction digestion with Hgal and amplification of exon 13 followed by digestion with Acil, respectively. PCR was carried out in a total volume of 50 μL with 1.5 mM MgCl₂, 187.5 nM of each dNTP, 2.5U Taq polymerase and 15 pmol of each primer (forward primer in exon 30: 5′-CTG CCA GGC AGG GGA GGA AGA GTG-3′ (SEQ ID NO: 4); reverse primer spanning the junction of exon 30 and intron 30: 5′-GAA AGT GAC TCA CTT GTG GAG GA-3′ (SEQ ID NO: 5); forward primer in intron 12: 5′-AAA GGG GCT TGG TAA GGG TA-3′ (SEQ ID NO: 6); reverse in intron 13: 5′-CAT GCA CAT GCA CAC ACA TA -3′ (SEQ ID NO: 7)). Following an initial denaturation of 3 minutes at 95° C., 35 cycles consisting of 95° C. 10 seconds, 58° C. 30 seconds, 72° C. 30 seconds were performed, with a final extension of 10 minutes at 72° C. For detection of the T4503C mutation, 15 μL of exon 30 PCR product was incubated with 4 U Hgal in a total volume of 25 μL, for 2 hours at 37° C., and the resulting fragments were separated on a 1.5% agarose gel. The presence of the T4503C mutation creates a restriction site for Hgal, and thus the 194 bp PCR product will be cut into fragments of 134 and 60 bp in the presence of the T4503C variant, but not in its absence. For detection of the A1864G mutation, 15 μL of exon 13 PCR products were digested with 8 U Acil for three hours at 37° C. Products were separated on 2% agarose gels. The presence of the A1864G mutation creates a second Acil site within the PCR product. Thus, the 360 bp PCR product is cleaved into fragments of 215 bp and 145 bp on the wild-type allele, but 185 bp, 145 bp and 30 bp on the mutant allele.

Detection of Mutation in FHA

Genotyping for the 693 variant was performed by PCR amplification of exon 14 followed by restriction enzyme digestion with Earl. PCR was carried out in a total volume of 80 μL with 1.5 mM MgCl₂, 187.5 nM of each dNTP, 2.5 U Taq polymerase and 20 pmol of each primer (forward primer in exon 14: 5′-CTT TCT GCG GGT GAT GAG CCG GTC AAT-3′ (SEQ ID NO: 8); reverse primer in intron 14: 5′-CCT TAG CCC GTG TTG AGC TA-3′ (SEQ ID NO: 9)). Following an initial denaturation of 3 minutes at 95° C., 35 cycles consisting of 95° C. 10 seconds, 55° C. 30 seconds, 72° C. 30 seconds were performed, with a final extension of 10 minutes at 72° C. Twenty microliters of PCR product was incubated with 4 U Earl in a total volume of 25 μL, for two hours at 37° C., and the fragments were separated on a 2% agarose gel. The presence of the 693 mutation destroys a restriction site for Earl, and thus the 297 bp PCR product will be cut into fragments of 151 bp, 59 bp, 48 bp and 39 bp in the presence of a wild-type allele, but only fragments of 210 bp, 48 bp and 39 bp in the presence of the deletion.

A 6 bp deletion encompassing nucleotides 5752-5757 (inclusive), was detected in exon 41 in the proband of family FHA-3 by genomic sequencing using primers located within the introns flanking this exon. Genotyping of this mutation in family FHA-3 and controls was carried out by PCR with forward (5′-CCT GTA MT GCA MG CTA TCT CCT CT-3′ (SEQ ID NO: 10)) and reverse primers (5′-CGT CM CTC CTT GAT TTC TM GAT GT (SEQ ID NO: 11)) located near the 5′ and 3′ ends of exon 41, respectively. Each PCR was carried out as for the genotyping of the 693 variant, but with annealing temperature of 58° C. Twenty microliters of PCR product was resolved on 3% agarose or 10% acrylamide gels. The wild type allele was detected as a 117 bp band and the mutant allele as a 111 bp band upon staining with ethidium bromide.

A C to T transition was detected at nucleotide 6504 in genomic DNA of the proband of family FHA-2. It was detectable as a double C and T peak in the genomic sequence of exon 48 of this individual, who is heterozygous for the alteration. This mutation, which creates a STOP codon that results in truncation of the last 118 amino acids of the ABC1 protein, also destroys an Rsal restriction site that is present in the wild type sequence. Genotyping of this mutation in family FHA-2 and controls was carried out by PCR with forward (5′-GGG TTC CCA GGG TTC AGT AT-3′) (SEQ ID NO: 12)) and reverse (5′-GAT CAG GM TTC MG CAC CAA-3′) (SEQ ID NO: 13)) primers directed to the intronic sequences flanking exon 48. PCR was done as for the 693 variant. Fifteen microliters of PCR product was digested with 5 Units of Rsal at 37° C. for two hours and the digestion products resolved on 1.5% agarose gels. The mutant allele is detected as an uncut 436 bp band. The normal sequence is cut by Rsal to produce 332 and 104 bp bands.

Cell Culture

Skin fibroblast cultures were established from 3.0 mm punch biopsies of the forearm of FHD patients and healthy control subjects as described (Marcil et al., Arterioscler. Thromb. Vasc. Biol. 19:159-169, 1999).

Cellular Cholesterol Labeling and Loading

The protocol for cellular cholesterol efflux experiments was described in detail elsewhere (Marcil et al., Arterioscler. Thromb. Vasc. Biol. 19:159-169, 1999). The cells were ³H-cholesterol labeled during growth and free cholesterol loaded in growth arrest.

Cholesterol Efflux Studies

Efflux studies were carried out from 0 to 24 hours in the presence of purified ApoAI (10 μg protein/mL medium). Efflux was determined as a percent of free cholesterol in the medium after the cells were incubated for specified periods of time. All experiments were performed in triplicate, in the presence of cells from one control subject and the cells from the study subjects to be examined. All results showing an efflux defect were confirmed at least three times.

Oligonucleotide Synthesis

Eight phosphorothioate deoxyoligonucleotides complementary to various regions of the human ABC1 cDNA sequence were obtained from GIBCO BRL. The oligonucleotides were purified by HPLC. The sequences of the antisense oligonucleotides and their location are listed. One skilled in the art will recognize that other ABC1 antisense sequences can also be produced and tested for their ability to decrease ABC1-mediated cholesterol regulation.

| | | | |
|---|---|---|---|
| Name | Sequence (5′-3′) | mRNA target | % control |

| | | | | |
|---|---|---|---|---|
| AN-1 | GCAGAGGGCATGGCTTTATTTG | (SEQ ID NO: 3) | AUG codon | 46 |
| AN-2 | GTGTTCCTGCAGAGGGCATG | (SEQ ID NO: 30) | AUG codon | 50 |
| AN-3 | CACTTCCAGTAACAGCTGAC | (SEQ ID NO: 31) | 5′-Untranslated | 79 |
| AN-4 | CTTTGCGCATGTCCTTCATGC | (SEQ ID NO: 32) | Coding | 80 |
| AN-5 | GACATCAGCCCTCAGCATCTT | (SEQ ID NO: 33) | Coding | 120 |
| AN-6: | CAACAAGCCATGTTCCCTC | (SEQ ID NO: 34) | Coding | |
| AN-7: | CATGTTCCCTCAGCCAGC | (SEQ ID NO: 35) | Coding | |
| AN-8: | CAGAGCTCACAGCAGGGA C | (SEQ ID NO: 36) | Coding | |

Cell Transfection With Antisense Oligonucleotides

Cells were grown in 35 mm culture dishes until 80% confluent, then washed once with DMEM medium (serum and antibiotics free). One milliliter of DMEM (serum and antibiotics free) containing 500 nM antisense oligonucleotides and 5 μg/ml or 7.5 μg/ml of lipofectin (GIBCO BRL) were added to each well according to the manufacturer's protocol. The cells were incubated at 37° C. for 4 hours, and then the medium was replaced by DMEM containing 10% FCS. Twenty-four hours after the transfection, the total cell RNA was isolated. Ten micrograms of total RNA was resolved on a 1% of agarose-formaldehyde gel and transferred to nylon membrane. The blot was hybridized with γ-³²P dCTP labeled human ABC1 cDNA overnight at 68° C. The membrane was subsequently exposed to x-ray film. The hybridizing bands were scanned by optical densitometry and standard to 28S ribosome RNA.

Cholesterol Efflux With Anti-ABC1 Oligonucleotides

Human skin fibroblasts were plated in 6-well plates. The cells were labeled with ³H-cholesterol (0.2 μCi/ml) in DMEM with 10% FBS for two days when the cell reached 50% confluence. The cells were then transfected with the antisense ABC1 oligonucleotides at 500 nM in DMEM (serum and antibiotic free) with 7.5 μg/ml Lipofectin (GIBCO BRL) according to the manufacturer's protocol. Following the transfection, and the cells were loaded with nonlipoprotein (20 μg/ml) for 12 hours in DMEM containing 2 mg/ml BSA without serum. The cellular cholesterol pools were then allowed to equilibrate for 6 hours in DMEM-BSA. The cholesterol efflux mediated by ApoAI (10μg/ml, in DMEM-BSA) were then carried out which is 48 hours after transfection.

Radiolabeled cholesterol released into the medium is expressed as a percentage of total ³H-cholesterol per well (medium+cell ). Results are the mean +/−SD of triplicate dishes.

Determination of Genomic Structure of the ABC1 Gene

Most splice junction sequences were determined from genomic sequence generated from BAC clones spanning the ABC1 gene. More than 160 kb of genomic sequence were generated. Genomic sequences were aligned with cDNA sequences to identify intron/exon boundaries. In some cases, long distance PCR between adjacent exons was used to amplify intron/exon boundary sequences using amplification primers designed according to the cDNA sequence.

Functionality of the Newly-Discovered 60 Amino Acids at the N-Terminus

Antisense Experiments

Phosphorothioate antisense oligonucleotides were designed to be complementary to the regions of the cDNA near newly discovered translation start site. AN-6 and AN-7 both overlap the initiator methionine codon; this site is in the middle of oligonucleotide AN-6. AN-8 is complementary to the very 5′ end of the ABC1 cDNA. Antisense oligonucleotide AN-1 is complementary to the region of the ABC1 cDNA corresponding to the site identified as the ABC1 initiator methionine in AJ012376. FIG. 7C shows that antisense oligonucleotide AN-6 interferes with cellular cholesterol efflux in normal fibroblasts to the same extent as does antisense oligonucleotide AN-1. Transfection with either of these antisense oligonucleotides results in a decrease in cellular cholesterol efflux almost as severe as that seen in FHA cells. In general, antisense oligonucleotides complementary to coding sequences, especially near the 5′ end of a gene's coding sequence, are expected to be more effective in decreasing the effective amount of transcript than are oligonucleotides directed to more 3′ sequences or to non-coding sequences. The observation that AN-6 depresses cellular cholesterol efflux as effectively as AN-1 implies that both of these oligonucleotides are complementary to ABC1 coding sequences, and that the amino terminal 60 amino acids are likely to be contained in ABC1 protein. In contrast, the ineffectiveness of AN-8 shows that it is likely to be outside the protein coding region of the transcript, as predicted by presence of an in-frame stop codon between the initiator methionine and the region targeted by AN-8.

Antibody Experiments

Polyclonal and monoclonal antibodies have been generated using peptides corresponding to discrete portions of the ABC1 amino acid sequence. One of these, 20-amino acid peptide #2 (Pep2: CSVRLSYPPYEQHECHFPNKA (SEQ ID NO: 37), in which the N-terminal cysteine was added to facilitate conjugation of the peptide) corresponds to a protein sequence within the 60 amino-terminal amino acids of the newly-discovered ABC1 protein sequence. The peptide was coupled to the KLH carrier protein and 300 □g injected at three intervals into two Balb/c mice over a four week period. The spleen was harvested from the mouse with the highest ELISA-determined immune response to free peptide, and the cells fused to NS-1 myeloma cells by standard monoclonal antibody generation methods. Positive hybridomas were selected first by ELISA and then further characterized by western blotting using cultured primary human fibroblasts. Monoclonal cell lines producing a high antibody titre and specifically recognizing the 245 kD human ABC1 protein were saved. The same size ABC1 protein product was detected by antibodies directed to four other discrete regions of the same protein. The 245 kD band could be eliminated in competition experiments with appropriate free peptide, indicating that it represents ABC1 protein (FIG. 13).

The foregoing experiments indicate that ABC1 protein is detected not only by antibodies corresponding to amino acid sequences within the previously-described ABC1 amino acid sequence, but also by the Pep2 monoclonal antibody that recognizes an epitope within the newly-discovered N-terminal 60 amino acids. The N-terminal 60 amino acid region is therefore coding, and is part of the ABC1 protein.

The epitope recognized by the Pep2 monoclonal antibody is also conserved among human, mouse, and chicken. Liver tissues from these three species employed in a Western blot produced an ABC1 band of 245 kD when probed with the Pep2 monoclonal antibody. This indicates that the 60 amino acid N-terminal sequence is part of the ABC1 coding sequence in humans, mice, and chickens. Presence of this region is therefore evolutionarily conserved and likely to be of important functional significance for the ABC1 protein.

Bioinformatic Analyses of ABC1 Protein Sequences

Transmembrane prediction programs indicate 13 transmembrane (TM) regions, the first one being between amino acids 26 and 42 (psort.nibb.ac.jp:8800/psort/helpwww2.ealom). The tentative number of TM regions for the threshold 0.5 is 13. (INTEGRAL Likelihood=−7.75 Transmembrane 26-42). The other 12 TM range in value between −0.64 and −12 (full results below). It is therefore very likely that the newly-discovered 60 amino acids contain a TM domain, and that the amino end of ABC1 may be on the opposite side of the membrane than originally thought.

ALOM: TM region allocation
Init position for calculation: 1
Tentative number of TMs for the threshold 0.5: 13
INTEGRAL Likelihood=−7.75 Transmembrane 26-42
INTEGRAL Likelihood=−3.98 Transmembrane 640-656
INTEGRAL Likelihood=−8.70 Transmembrane 690-706
INTEGRAL Likelihood=−9.61 Transmembrane 717-733
INTEGRAL Likelihood=−1.44 Transmembrane 749-765
INTEGRAL Likelihood=−0.64 Transmembrane 771-787
INTEGRAL Likelihood=−1.28 Transmembrane 1041-1057
INTEGRAL Likelihood=−12.79 Transmembrane 1351-1367
INTEGRAL Likelihood=−8.60 Transmembrane 1661-1677
INTEGRAL Likelihood=−6.79 Transmembrane 1708-1724
INTEGRAL Likelihood=−3.40 Transmembrane 1737-1753
INTEGRAL Likelihood=−1.49 Transmembrane 1775-1791
INTEGRAL Likelihood=−8.39 Transmembrane 1854-1870
PERIPHERAL Likelihood=0.69 (at 1643)
ALOM score: −12.79 (number of TMSS: 13)

There does not appear to be an obvious cleaved peptide, so this first 60 amino acid residues are not likely to be cleaved, and are therefore not specifically a signal/targeting sequence. No other signals (e.g., for targeting to specific organelles) are apparent.

Agonists and Antagonists

Useful therapeutic compounds include those which modulate the expression, activity, or stability of ABC1. To isolate such compounds, ABC1 expression, biological activity, or regulated catabolism is measured following the addition of candidate compounds to a culture medium of ABC1-expressing cells. Alternatively, the candidate compounds may be directly administered to animals (for example mice, pigs, or chickens) and used to screen for their effects on ABC1 expression.

In addition its role in the regulation of cholesterol, ABC1 also participates in other biological processes for which the development of ABC1 modulators would be useful. In one example, ABC1 transports interleukin-1β (IL-1β) across the cell membrane and out of cells. IL-1β is a precursor of the inflammatory response and, as such, inhibitors or antagonists of ABC1 expression or biological activity may be useful in the treatment of any inflammatory disorders, including but not limited to rheumatoid arthritis, systemic lupus erythematosis (SLE), hypo- or hyper-thyroidism, inflammatory bowel disease, and diabetes mellitus. In another example, ABC1 expressed in macrophages has been shown to be engaged in the engulfment and clearance of dead cells. The ability of macrophages to ingest these apoptotic bodies is impaired after antibody-mediated blockade of ABC1. Accordingly, compounds that modulate ABC1 expression, stability, or biological activity would be useful for the treatment of these disorders.

ABC1 expression is measured, for example, by standard Northern blot analysis using an ABC1 nucleic acid sequence (or fragment thereof as a hybridization probe, or by Western blot using an anti-ABC1 antibody and standard techniques. The level of ABC1 expression in the presence of the candidate molecule is compared to the level measured for the same cells, in the same culture medium, or in a parallel set of test animals, but in the absence of the candidate molecule. ABC1 activity can also be measured using the cholesterol efflux assay.

Transcriptional Regulation of ABC1 Expression

ABC1 mRNA is increased approximately 8-fold upon cholesterol loading. This increase is likely controlled at the transcriptional level. Using the promoter sequence described herein, one can identify transcription factors that bind to the promoter by performing, for example, gel shift assays, DNAse protection assays, or in vitro or in vivo reporter gene-based assays. The identified transcription factors are themselves drug targets. In the case of ABC1, drug compounds that act through modulation of transcription of ABC1 could be used for HDL modulation, atherosclerosis prevention, and the treatment of cardiovascular disease. For example, using a compound to inhibit a transcription factor that represses ABC1 would be expected to result in up-regulation of ABC1 and, therefore, HDL levels. In another example, a compound that increases transcription factor expression or activity would also increase ABC1 expression and HDL levels.

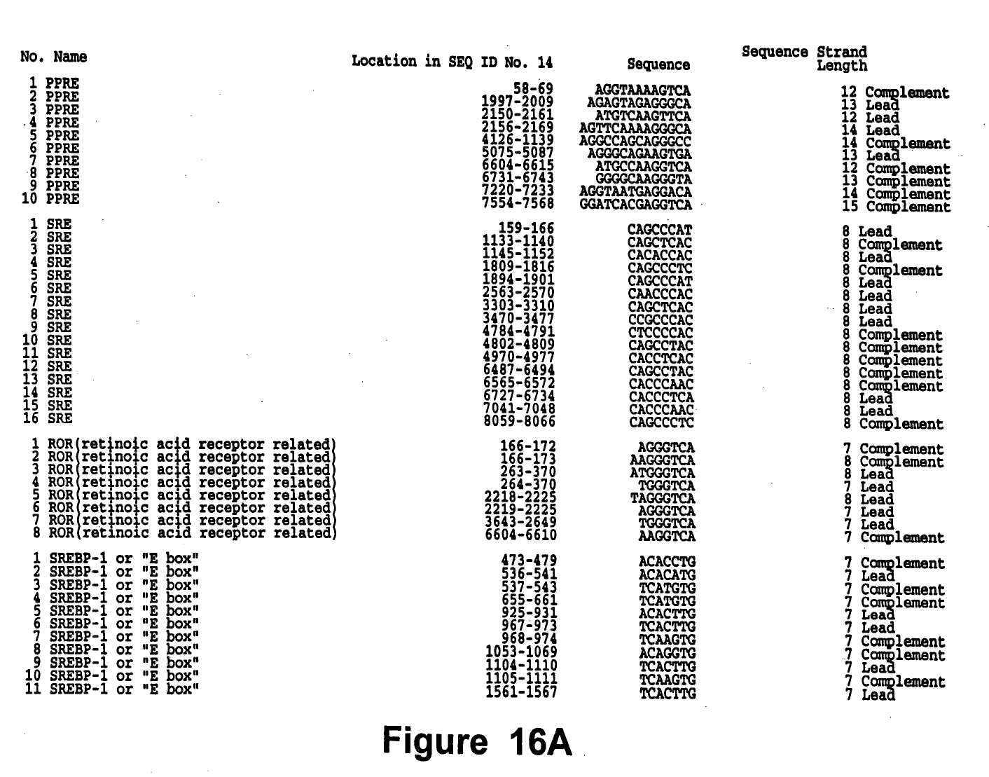

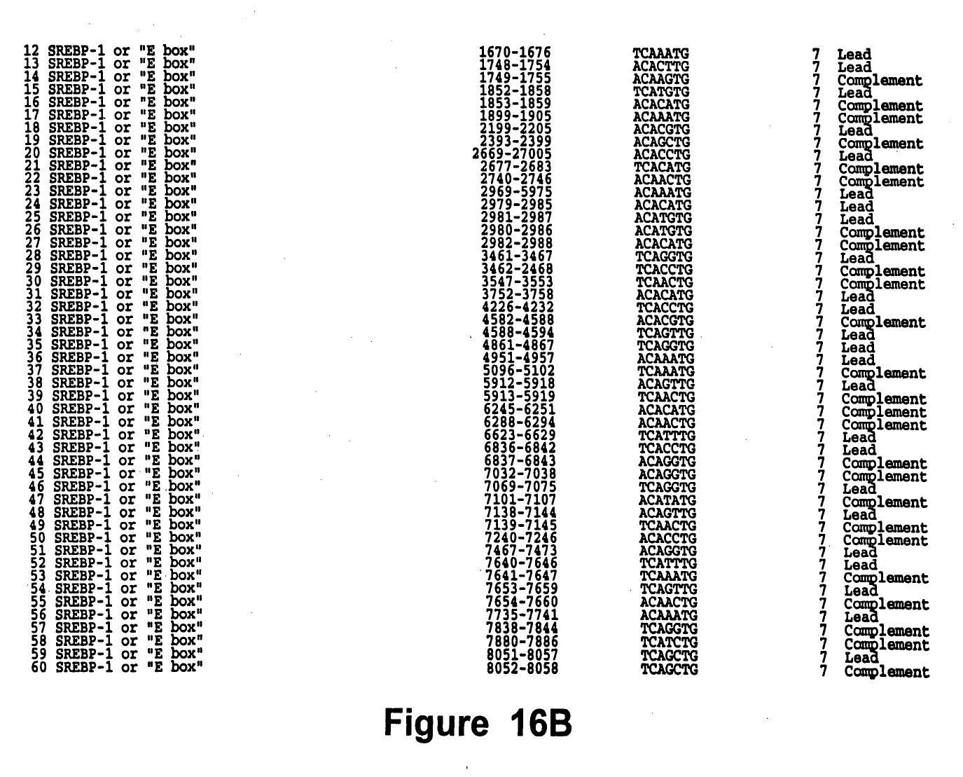

Transcription factors known to regulate other genes in the regulation of apolipoprotein genes or other cholesterol- or lipid-regulating genes are of particular relevance. Such factors include, but are not limited to, the steroid response element binding proteins (SREBP-1 and SREBP-2), the PPAR (peroxisomal proliferation-activated receptor) transcription factors. Several consensus sites for certain elements are present in the sequenced region 5′ to the ABC1 gene (FIG. 16) and are likely to modulate ABC1 expression. For example, PPARs may alter transcription of ABC1 by mechanisms including heterodimerization with retinoid X receptors (RXRs) and then binding to specific proliferator response elements (PPREs). Examples of such PPARs include PPARα, β, γ and δ. These distinct PPARs have been shown to have transcriptional regulatory effects on different genes. PPARα is expressed mainly in liver, whereas PPARγ is expressed in predominantly in adipocytes. Both PPARα and PPARγ are found in coronary and carotid artery atherosclerotic plaques and in endothelial cells, smooth muscle cells, monocytes and monocyte-derived macrophages. Activation of PPAR□ results in altered lipoprotein metabolism through PPARα's effect on genes such as lipoprotein lipase (LPL), apolipoprotein CIII (apo CIII) and apolipoprotein AI (apo AI) and AII (apo AII). PPAR□ activation results in overexpression of LPL and apoA-I and apoA-II, but inhibits the expression of apo CIII. PPAR□ activation also inhibits inflammation, stimulates lipid oxidation and increases the hepatic uptake and esterification of free fatty acids (FFA's). PPAR□ and PPAR□ activation may inhibit nitric oxide (NO) synthase in macrophages and prevent interleukin-1 (IL-1) induced expression of IL-6 and cyclo-oxygenase-2 (COX-2) and thrombin induced endothelin-1 expression secondary to negative transcriptional regulation of NF-KB and activation of protein-1 signaling pathway. It has also been shown that PPAR□ induces apoptosis in monocyte-derived macrophages through the inhibition of NF-KB activity.

Activation of PPARα can be achieved by compounds such as fibrates, β-estradiol, arachidonic acid derivatives, WY-14,643 and LTB4 or 8(s)HETE. PPARγ activation can be achieved through compounds such as thiozolidinedione antidiabetic drugs, 9-HODE and 13-HODE. Additional compounds such as nicotinic acid or HMG CoA reductase inhibitors may also alter the activity of PPARs.

Compounds which alter activity of any of the PPARs (e.g., PPARα or PPARγ) may have an effect on ABC1 expression and thereby could affect HDL levels, atherosclerosis and risk of CAD. PPARs are also regulated by fatty acids (including modified fatty acids such as 3 thia fatty acids), leukotrienes such as leukotriene B4 and prostaglandin J2, which is a natural activator/ligand for PPARγ. Drugs that modulate PPARs may therefore have an important effect on modulating lipid levels (including HDL and triglyceride levels) and altering CAD risk. This effect could be achieved through the modulation of ABC1 gene expression. Drugs may also effect ABC1 gene expression and thereby HDL levels, by an indirect effect on PPARs via other transcriptional factors such as adipocyte differentiation and determination factor-1 (ADD-1) and sterol regulatory element binding protein-1 and 2 (SREBP-1 and 2). Drugs with combined PPARα and PPARγ agonist activity or PPARα and PPARγ agonists given in combination for example, may increase HDL levels even more.

A PPAR binding site (PPRE element) is found 5′ to the ABC1 gene (nucleotides 2150 to 2169 of SEQ ID NO: 14). Like the PPRE elements found in the C-ACS, HD, CYP4A6 and ApoA-I genes, this PPRE site is a trimer related to the PPRE consensus sequence. Partly because of its similarity in the number and arrangement of repeats in this PPAR binding site, this element in particular is very likely to be of physiological relevance to the regulation of the ABC1 gene.

Additional transcription factors which may also have an effect in modulating ABC1 gene expression and thereby HDL levels, atherosclerosis and CAD risk include; REV-ERBα, SREBP-1 & 2, ADD-1, EBPα, CREB binding protein, P300, HNF 4, RAR, LXR, and RORα. Additional degenerate binding sites for these factors can be found through examination of the sequence in SEQ ID NO: 14.

Additional Utility of ABC1 Polypeptides, Nucleic Acids, and Modulators

ABC1 may act as a transporter of toxic proteins or protein fragments (e.g., APP) out of cells. Thus, ABC1 agonists/upregulators may be useful in the treatment of other disease areas, including Alzheimer's disease, Niemann-Pick disease, and Huntington's disease.

ABC transporters have been shown to increase the uptake of long chain fatty acids from the cytosol to peroxisomes and, moreover, to play a role in □-oxidation of very long chain fatty acids. Importantly, in x-linked adrenoleukodystrophy (ALD), fatty acid metabolism is abnormal, due to defects in the peroxisomal ABC transporter. Any agent that upregulates ABC transporter expression or biological activity may therefor be useful for the treatment of ALD or any other lipid disorder.

ABC1 is expressed in macrophages and is required for engulfment of cells undergoing programmed cell death. The apoptotic process itself, and its regulation, have important implications for disorders such as cancer, one mechanism of which is failure of cells to undergo cell death appropriately. ABC1 may facilitate apoptosis, and as such may represent an intervention point for cancer treatment. Increasing ABC1 expression or activity or otherwise up-regulating ABC1 by any method may constitute a treatment for cancer by increasing apoptosis and thus potentially decreasing the aberrant cellular proliferation characterized by this disease. Conversely, down-regulation of ABC1 by any method may provide opportunity for decreasing apoptosis and allowing increased proliferation of cells in conditions where cell growth is limited. Such disorders include but are not limited to neurodeficiencies and neurodegeneration, and growth disorders. ABC1 could, therefore, potentially be used as a method for identification of compounds for use in the treatment of cancer, or in the treatment of degenerative disorders.

Agents that have been shown to inhibit ABC1 include, for example, the anti-diabetic agents glibenclamide and glyburide, flufenamic acid, diphenylamine-2-carbonic acid, sulfobromophthalein, and DIDS.

Agents that upregulate ABC1 expression or biological activity include but are not limited to protein kinase A, protein kinase C, vanadate, okadaic acid, and IBMX1.

Those in the art will recognize that other compounds can also modulate ABC1 biological activity, and these compounds are also in the spirit of the invention.

Drug Screens Based on the ABC1 Gene or Protein

The ABC1 protein and gene can be used in screening assays for identification of compounds which modulate its activity and may be potential drugs to regulate cholesterol levels. Useful ABC1 proteins include wild-type and mutant ABC1 proteins or protein fragments, in a recombinant form or endogenously expressed. Drug screens to identify compounds acting on the ABC1 expression product may employ any functional feature of the protein. In one example, the phosphorylation state or other post-translational modification is monitored as a measure of ABC1 biological activity. ABC1 has ATP binding sites, and thus assays may wholly or in part test the ability of ABC1 to bind ATP or to exhibit ATPase activity. ABC1, by analogy to similar proteins, is thought to be able to form a channel-like structure; drug screening assays could be based upon assaying for the ability of the protein to form a channel, or upon the ability to transport cholesterol or another molecule, or based upon the ability of other proteins bound by or regulated by ABC1 to form a channel. Alternatively, phospholipid or lipid transport can also be used as measures of ABC1 biological activity.

There is evidence that, in addition to its role as a regulator of cholesterol levels, ABC1 also transports anions. Functional assays could be based upon this property, and could employ drug screening technology such as (but not limited to) the ability of various dyes to change color in response to changes in specific ion concentrations in such assays can be performed in vesicles such as liposomes, or adapted to use whole cells.

Drug screening assays can also be based upon the ability of ABC1 or other ABC transporters to interact with other proteins. Such interacting proteins can be identified by a variety of methods known in the art, including, for example, radioimmunoprecipitation, co-immunoprecipitation, co-purification, and yeast two-hybrid screening. Such interactions can be further assayed by means including but not limited to fluorescence polarization or scintillation proximity methods. Drug screens can also be based upon functions of the ABC1 protein deduced upon X-ray crystallography of the protein and comparison of its 3-D structure to that of proteins with known functions. Such a crystal structure has been determined for the prokaryotic ABC family member HisP, histidine permease. Drug screens can be based upon a function or feature apparent upon creation of a transgenic or knockout mouse, or upon overexpression of the protein or protein fragment in mammalian cells in vitro. Moreover, expression of mammalian (e.g., human) ABC1 in yeast or C. elegans allows for screening of candidate compounds in wild-type and mutant backgrounds, as well as screens for mutations that enhance or suppress an ABC1-dependent phenotype. Modifier screens can also be performed in ABC1 transgenic or knock-out mice.

Additionally, drug screening assays can also be based upon ABC1 functions deduced upon antisense interference with the gene function. Intracellular localization of ABC1, or effects which occur upon a change in intracellular localization of the protein, can also be used as an assay for drug screening. Immunocytochemical methods will be used to determine the exact location of the ABC1 protein.

Human and rodent ABC1 protein can be used as an antigen to raise antibodies, including monoclonal antibodies. Such antibodies will be useful for a wide variety of purposes, including but not limited to functional studies and the development of drug screening assays and diagnostics. Monitoring the influence of agents (e.g., drugs, compounds) on the expression or biological activity of ABC1 can be applied not only in basic drug screening, but also in clinical trials. For example, the effectiveness of an agent determined by a screening assay as described herein to increase ABC1 gene expression, protein levels, or biological activity can be monitored in clinical trails of subjects exhibiting altered ABC1 gene expression, protein levels, or biological activity. Alternatively, the effectiveness of an agent determined by a screening assay to modulate ABC1 gene expression, protein levels, or biological activity can be monitored in clinical trails of subjects exhibiting decreased altered gene expression, protein levels, or biological activity. In such clinical trials, the expression or activity of ABC1 and, preferably, other genes that have been implicated in, for example, cardiovascular disease can be used to ascertain the effectiveness of a particular drug.

For example, and not by way of limitation, genes, including ABC1, that are modulated in cells by treatment with an agent (e.g., compound, drug or small molecule) that modulates ABC1 biological activity (e.g., identified in a screening assay as described herein) can be identified. Thus, to study the effect of agents on cholesterol levels or cardiovascular disease, for example, in a clinical trial, cells can be isolated and RNA prepared and analyzed for the levels of expression of ABC1 and other genes implicated in the disorder. The levels of gene expression can be quantified by Northern blot analysis or RT-PCR, or, alternatively, by measuring the amount of protein produced, by one of a number of methods known in the art, or by measuring the levels of biological activity of ABC1 or other genes. In this way, the gene expression can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of an ABC1 protein, mRNA, or genomic DNA in the preadministration sample; (iii) obtaining one or more post-administration samples from the subject; (iv) detecting the level of expression or activity of the ABC1 protein, mRNA, or genomic DNA in the post-administration samples; (v) comparing the level of expression or activity of the ABC1 protein, mRNA, or genomic DNA in the pre-administration sample with the ABC1 protein, mRNA, or genomic DNA in the post administration sample or samples; and (vi) altering the administration of the agent to the subject accordingly. For example, increased administration of the agent may be desirable to increase the expression or activity of ABC1 to higher levels than detected, i.e., to increase the effectiveness of the agent. Alternatively, decreased administration of the agent may be desirable to decrease expression or activity of ABC1 to lower levels than detected.

The ABC1 gene or a fragment thereof can be used as a tool to express the protein in an appropriate cell in vitro or in vivo (gene therapy), or can be cloned into expression vectors which can be used to produce large enough amounts of ABC1 protein to use in in vitro assays for drug screening. Expression systems which may be employed include baculovirus, herpes virus, adenovirus, adeno-associated virus, bacterial systems, and eucaryotic systems such as CHO cells. Naked DNA and DNA-liposome complexes can also be used.

Assays of ABC1 activity includes binding to intracellular interacting proteins; interaction with a protein that up-regulates ABC1 activity; interaction with HDL particles or constituents; interaction with other proteins which facilitate interaction with HDL or its constituents; and measurement of cholesterol efflux. Furthermore, assays may be based upon the molecular dynamics of macromolecules, metabolites and ions by means of fluorescent-protein biosensors. Alternatively, the effect of candidate modulators on expression or activity may be measured at the level of ABC1 protein production using the same general approach in combination with standard immunological detection techniques, such as Western blotting or immunoprecipitation with an ABC1-specific antibody. Again, useful cholesterol-regulating or anti-CVD therapeutic modulators are identified as those which produce an change in ABC1 polypeptide production. Agonists may also affect ABC1 activity without any effect on expression level.

Candidate modulators may be purified (or substantially purified) molecules or may be one component of a mixture of compounds (e.g., an extract or supernatant obtained from cells). In a mixed compound assay, ABC1 expression is tested against progressively smaller subsets of the candidate compound pool (e.g., produced by standard purification techniques, e.g., HPLC or FPLC; Ausubel et al.) until a single compound or minimal compound mixture is demonstrated to modulate ABC1 expression.

Agonists, antagonists, or mimetics found to be effective at modulating the level of cellular ABC1 expression or activity may be confirmed as useful in animal models (for example, mice, pigs, rabbits, or chickens). For example, the compound may ameliorate the low HDL levels of mouse or chicken hypoalphalipoproteinemias.

A compound that promotes an increase in ABC1 expression or activity is considered particularly useful in the invention; such a molecule may be used, for example, as a therapeutic to increase the level or activity of native, cellular ABC1 and thereby treat a low HDL condition in an animal (for example, a human).

One method for increasing ABC biological activity is to increase the stabilization of the ABC protein or to prevent its degradation. Thus, it would be useful to identify mutations in an ABC polypeptide (e.g., ABC1) that lead to increased protein stability. These mutations can be incorporated into any protein therapy or gene therapy undertaken for the treatment of low HDL-C or any other condition resulting from loss of ABC1 biological activity. Similarly, compounds that increase the stability of a wild-type ABC polypeptide or decrease its catabolism may also be useful for the treatment of low HDL-C or any other condition resulting from loss of ABC1 biological activity. Such mutations and compounds can be identified using the methods described herein.

In one example, cells expressing an ABC polypeptide having a mutation are transiently metabolically labeled during translation and the half-life of the ABC polypeptide is determined using standard techniques. Mutations that increase the half-life of an ABC polypeptide are ones that increase ABC protein stability. These mutations can then be assessed for ABC biological activity. They can also be used to identify proteins that affect the stability of ABC1 mRNA or protein. One can then assay for compounds that act on these factors or on the ability of these factors to bind ABC1.

In another example, cells expressing wild-type ABC polypeptide are transiently metabolically labeled during translation, contacted with a candidate compounds, and the half-life of the ABC polypeptide is determined using standard techniques. Compounds that increase the half-life of an ABC polypeptide are useful compounds in the present invention.

If desired, treatment with an agonist of the invention may be combined with any other HDL-raising or anti-CVD therapies.

It is understood that, while ABC1 is the preferred ABC transporter for the drug screens described herein, other ABC transporters can also be used. The replacement of ABC1 with another ABC transporter is possible because it is likely that ABC transporter family members, such as ABC2, ABCR, or ABC8 will have a similar mechanism of regulation.

Exemplary assays are described in greater detail below.

Protein-Based Assays

ABC1 polypeptide (purified or unpurified) can be used in an assay to determine its ability to bind another protein (including, but not limited to, proteins found to specifically interact with ABC1). The effect of a compound on that binding is then determined.

Protein Interaction Assays

ABC1 protein (or a polypeptide fragment thereof or an epitope-tagged form or fragment thereof) is harvested from a suitable source (e.g., from a prokaryotic expression system, eukaryotic cells, a cell-free system, or by immunoprecipitation from ABC1-expressing cells). The ABC1 polypeptide is then bound to a suitable support (e.g., nitrocellulose or an antibody or a metal agarose column in the case of, for example, a his-tagged form of ABC1). Binding to the support is preferably done under conditions that allow proteins associated with ABC1 polypeptide to remain associated with it. Such conditions may include use of buffers that minimize interference with protein-protein interactions. The binding step can be done in the presence and absence of compounds being tested for their ability to interfere with interactions between ABC1 and other molecules. If desired, other proteins (e.g., a cell lysate) are added, and allowed time to associate with the ABC polypeptide. The immobilized ABC1 polypeptide is then washed to remove proteins or other cell constituents that may be non-specifically associated with it the polypeptide or the support. The immobilized ABC1 polypeptide is then dissociated from its support, and so that proteins bound to it are released (for example, by heating), or, alternatively, associated proteins are released from ABC1 without releasing the ABC1 polypeptide from the support. The released proteins and other cell constituents can be analyzed, for example, by SDS-PAGE gel electrophoresis, Western blotting and detection with specific antibodies, phosphoamino acid analysis, protease digestion, protein sequencing, or isoelectric focusing. Normal and mutant forms of ABC1 can be employed in these assays to gain additional information about which part of ABC1 a given factor is binding to. In addition, when incompletely purified polypeptide is employed, comparison of the normal and muatant forms of the protein can be used to help distinguish true binding proteins.

The foregoing assay can be performed using a purified or semipurified protein or other molecule that is known to interact with ABC1. This assay may include the following steps.

1. Harvest ABC1 protein and couple a suitable fluorescent label to it;

2. Label an interacting protein (or other molecule) with a second, different fluorescent label. Use dyes that will produce different quenching patterns when they are in close proximity to each other vs. when they are physically separate (i.e., dyes that quench each other when they are close together but fluoresce when they are not in close proximity);

3. Expose the interacting molecule to the immobilized ABC1 in the presence or absence of a compound being tested for its ability to interfere with an interaction between the two; and

4. Collect fluorescent readout data.

Another assay is includes Fluorescent Resonance Energy Transfer (FRET) assay. This assay can be performed as follows.

1. Provide ABC1 protein or a suitable polypeptide fragment thereof and couple a suitable FRET donor (e.g., nitro-benzoxadiazole (NBD)) to it;

2. Label an interacting protein (or other molecule) with a FRET acceptor (e.g., rhodamine);

3. Expose the acceptor-labeled interacting molecule to the donor-labeled ABC1 in the presence or absence of a compound being tested for its ability to interfere with an interaction between the two; and

4. Measure fluorescence resonance energy transfer.

Quenching and FRET assays are related. Either one can be applied in a given case, depending on which pair of fluorophores is used in the assay.

Membrane Permeability Assay

The ABC1 protein can also be tested for its effects on membrane permeability. For example, beyond its putative ability to translocate lipids, ABC1 might affect the permeability of membranes to ions. Other related membrane proteins, most notably the cystic fibrosis transmembrane conductance regulator and the sulfonylurea receptor, are associated with and regulate ion channels.

ABC1 or a fragment of ABC1 is incorporated into a synthetic vesicle, or, alternatively, is expressed in a cell and vesicles or other cell sub-structures containing ABC1 are isolated. The ABC1-containing vesicles or cells are loaded with a reporter molecule (such as a fluorescent ion indicator whose fluorescent properties change when it binds a particular ion) that can detect ions (to observe outward movement), or alternatively, the external medium is loaded with such a molecule (to observe inward movement). A molecule which exhibits differential properties when it is inside the vesicle compared to when it is outside the vesicle is preferred. For example, a molecule that has quenching properties when it is at high concentration but not when it is at another low concentration would be suitable. The movement of the charged molecule (either its ability to move or the kinetics of its movement) in the presence or absence of a compound being tested for its ability to affect this process can be determined.

In another assay, membrane permeability is determined electro-physiologically by measuring ionic influx or efflux mediated by or modulated by ABC1 by standard electrophysiological techniques. A suitable control (e.g., TD cells or a cell line with very low endogenous ABC1 expression) can be used as a control in the assay to determine if the effect observed is specific to cells expressing ABC1.

In still another assay, uptake of radioactive isotopes into or out of a vesicle can be measured. The vesicles are separated from the extravesicular medium and the radioactivity in the vesicles and in the medium is quantitated and compared.

Nucleic Acid-Based Assays

ABC1 nucleic acid may be used in an assay based on the binding of factors necessary for ABC1 gene transcription. The association between the ABC1 DNA and the binding factor may be assessed by means of any system that discriminates between protein-bound and non-protein-bound DNA (e.g., a gel retardation assay). The effect of a compound on the binding of a factor to ABC1 DNA is assessed by means of such an assay. In addition to in vitro binding assays, in vivo assays in which the regulatory regions of the ABC1 gene are linked to reporter genes can also be performed.

Assays Measuring ABC1 Stability

A cell-based or cell-free system can be used to screen for compounds based on their effect on the half-life of ABC1 mRNA or ABC1 protein. The assay may employ labeled mRNA or protein. Alternatively, ABC1 mRNA may be detected by means of specifically hybridizing probes or a quantitative PCR assay. Protein can be quantitated, for example, by fluorescent antibody-based methods.

In Vitro mRNA Stability Assay

1. Isolate or produce, by in vitro transcription, a suitable quantity of ABC1 mRNA;

2. Label the ABC1 mRNA;

3. Expose aliquots of the mRNA to a cell lysate in the presence or absence of a compound being tested for its ability to modulate ABC1 mRNA stability;

4. Assess intactness of the remaining mRNA at suitable time points.

In Vitro Protein Stability Assay

1. Express a suitable amount of ABC1 protein;

2. Label the protein;

3. Expose aliquots of the labeled protein to a cell lysate in the presence or absence of a compound being tested for its ability to modulate ABC1 protein stability;

4. Assess intactness of the remaining protein at suitable time points

In Vivo mRNA or Protein Stability Assay

1. Incubate cells expressing ABC1 mRNA or protein with a tracer (radiolabeled ribonucleotide or radiolabeled amino acid, respectively) for a very brief time period (e.g., five minutes) in the presence or absence of a compound being tested for its effect on mRNA or protein stability;

2. Incubate with unlabeled ribonucleotide or amino acid; and

3. Quantitate the ABC1 mRNA or protein radioactivity at time intervals beginning with the start of step 2 and extending to the time when the radioactivity in ABC1 mRNA or protein has declined by approximately 80%. It is preferable to separate the intact or mostly intact mRNA or protein from its radioactive breakdown products by a means such as gel electrophoresis in order to quantitate the mRNA or protein.

Assays Measuring Inhibition of Dominant Negative Activity

Mutant ABC1 polypeptides are likely to have dominant negative activity (i.e., activity that interferes with wild-type ABC1 function). An assay for a compound that can interfere with such a mutant may be based on any method of quantitating normal ABC1 activity in the presence of the mutant. For example, normal ABC1 facilitates cholesterol efflux, and a dominant negative mutant would interfere with this effect. The ability of a compound to counteract the effect of a dominant negative mutant may be based on cellular cholesterol efflux, or on any other normal activity of the wild-type ABC1 that was inhibitable by the mutant.

Assays Measuring Phosphorylation

The effect of a compound on ABC1 phosphorylation can be assayed by methods that quantitate phosphates on proteins or that assess the phosphorylation state of a specific residue of a ABC1. Such methods include but are not limited to ³²P labelling and immunoprecipitation, detection with antiphosphoamino acid antibodies (e.g., antiphosphoserine antibodies), phosphoamino acid analysis on 2-dimensional TLC plates, and protease digestion fingerprinting of proteins followed by detection of ³²P-labeled fragments.

Assays Measuring Other Post-Translational Modifications

The effect of a compound on the post-translational modification of ABC1 is based on any method capable of quantitating that particular modification. For example, effects of compounds on glycosylation may be assayed by treating ABC1 with glycosylase and quantitating the amount and nature of carbohydrate released.

Assays Measuring ATP Binding

The ability of ABC1 to bind ATP provides another assay to screen for compounds that affect ABC1. ATP binding can be quantitated as follows.

1. Provide ABC1 protein at an appropriate level of purity and reconstitute it in a lipid vesicle;

2. Expose the vesicle to a labeled but non-hydrolyzable ATP analog (such as gamma ³⁵S-ATP) in the presence or absence of compounds being tested for their effect on ATP binding. Note that azido-ATP analogs can be used to allow covalent attachment of the azido-ATP to protein (by means of U.V. light), and permit easier quantitation of the amount of ATP bound to the protein.

3. Quantitate the amount of ATP analog associated with ABC1

Assays Measuring ATPase Activity

Quantitation of the ATPase activity of ABC1 can also be assayed for the effect of compounds on ABC1. This is preferably performed in a cell-free assay so as to separate ABC1 from the many other ATPases in the cell. An ATPase assay may be performed in the presence or absence of membranes, and with or without integration of ABC1 protein into a membrane. If performed in a vesicle-based assay, the ATP hydrolysis products produced or the ATP hydrolyzed may be measured within or outside of the vesicles, or both. Such an assay may be based on disappearance of ATP or appearance of ATP hydrolysis products.

For high-throughput screening, a coupled ATPase assay is preferable. For example, a reaction mixture containing pyruvate kinase and lactate dehydrogenase can be used. The mixture includes phosphoenolpyruvate (PEP), nicotinamide adenine dinucleotide (NAD+), and ATP. The ATPase activity of ABC1 generates ADP from ATP. The ADP is then converted back to ATP as part of the pyruvate kinase reaction. The product, pyruvate, is then converted to lactate. The latter reaction generates a colored quinone (NADH) from a colorless substrate (NAD+), and the entire reaction can be monitored by detection of the color change upon formation of NADH. Since ADP is limiting for the pyruvate kinase reaction, this coupled system precisely monitors the ATPase activity of ABC1.

Assays Measuring Cholesterol Efflux

A transport-based assay can be performed in vivo or in vitro. For example, the assay may be based on any part of the reverse cholesterol transport process that is readily re-created in culture, such as cholesterol or phospholipid efflux. Alternatively, the assay may be based on net cholesterol transport in a whole organism, as assessed by means of a labeled substance (such as cholesterol).

For high throughput, fluorescent lipids can be used to measure ABC1-catalyzed lipid efflux. For phospholipids, a fluorescent precursor, C6-NBD-phosphatidic acid, can be used. This lipid is taken up by cells and dephosphorylated by phosphatidic acid phosphohydrolase. The product, NBD-diglyceride, is then a precursor for synthesis of glycerophospholipids like phosphatidylcholine. The efflux of NBD-phosphatidylcholine can be monitored by detecting fluorescence resonance energy transfer (FRET) of the NBD to a suitable acceptor in the cell culture medium. This acceptor can be rhodamine-labeled phosphatidylethanolamine, a phospholipid that is not readily taken up by cells. The use of short-chain precursors obviates the requirement for the phospholipid transfer protein in the media. For cholesterol, NBD-cholesterol ester can be reconstituted into LDL. The LDL can efficiently deliver this lipid to cells via the LDL receptor pathway. The NBD-cholesterol esters are hydrolyzed in the lysosomes, resulting in NBD-cholesterol that can now be transported back to the plasma membrane and efflux from the cell. The efflux can be monitored by the aforementioned FRET assay in which NBD transfers its fluorescence resonance energy to the rhodamine-phosphatidylethanoline acceptor.

Animal Model Systems

Compounds identified as having activity in any of the above-described assays are subsequently screened in any available animal model system, including, but not limited to, pigs, rabbits, and WHAM chickens. Test compounds are administered to these animals according to standard methods. Test compounds may also be tested in mice bearing mutations in the ABC1 gene. Additionally, compounds may be screened for their ability to enhance an interaction between ABC1 and any HDL particle constituent such as ApoAI, ApoAII, orApoE.

The Cholesterol Efflux Assay as a Drug Screen

The cholesterol efflux assay measures the ability of cells to transfer cholesterol to an extracellular acceptor molecule and is dependent on ABC1 function. In this procedure, cells are loaded with radiolabeled cholesterol by any of several biochemical pathways (Marcil et al., Arterioscler. Thromb. Vasc. Biol. 19:159-169, 1999). Cholesterol efflux is then measured after incubation for various times (typically 0 to 24 hours) in the presence of HDL3 or purified ApoAI. Cholesterol efflux is determined as the percentage of total cholesterol in the culture medium after various times of incubation. ABC1 expression levels and/or biological activity are associated with increased efflux while decreased levels of ABC1 are associated with decreased cholesterol efflux.

This assay can be readily adapted to the format used for drug screening, which may consist of a multi-well (e.g., 96-well) format. Modification of the assay to optimize it for drug screening would include scaling down and streamlining the procedure, modifying the labeling method, using a different cholesterol acceptor, altering the incubation time, and changing the method of calculating cholesterol efflux. In all these cases, the cholesterol efflux assay remains conceptually the same, though experimental modifications may be made. A transgenic mouse overexpressing ABC1 would be expected to have higher than normal HDL levels.

Knock-Out Mouse Model

An animal, such as a mouse, that has had one or both ABC1 alleles inactivated (e.g., by homologous recombination) is likely to have low HDL-C levels, and thus is a preferred animal model for screening for compounds that raise HDL-C levels. Such an animal can be produced using standard techniques. In addition to the initial screening of test compounds, the animals having mutant ABC1 genes are useful for further testing of efficacy and safety of drugs or agents first identified using one of the other screening methods described herein. Cells taken from the animal and placed in culture can also be exposed to test compounds. HDL-C levels can be measured using standard techniques, such as those described herein.

WHAM Chickens: An Animal Model for Low HDL Cholesterol

Wisconsin Hypo-Alpha Mutant (WHAM) chickens arose by spontaneous mutation in a closed flock. Mutant chickens came to attention through their a Z-linked white shank and white beak phenotype referred to as ‘recessive white skin’ (McGibbon, 1981) and were subsequently found to have a profound deficiency of HDL (Poernama et al., 1990).

This chicken low HDL locus (Y) is Z-Iinked, or sex-linked. (In birds, females are ZW and males are ZZ). Genetic mapping placed the Y locus on the long arm of the Z chromosome (Bitgood, 1985), proximal to the ID locus (Bitgood, 1988). Examination of current public mapping data for the chicken genome mapping project, ChickMap (maintained by the Roslin Institute; http://www.ri.bbsrc.ac.uk/ chickmap/ChickMapHomePage.html) showed that a region of synteny with human chromosome 9 lies on the long arm of the chicken Z chromosome (Zq) proximal to the ID locus. Evidence for this region of synteny is the location of the chicken aldolase B locus (ALDOB) within this region. The human ALDOB locus maps to chromosome 9q22.3 (The Genome Database, http://gdbwww.gdb.org/), not far from the location of human ABC1. This comparison of maps showed that the chicken Zq region near chicken ALDOB and the human 9q region near human ALDOB represent a region of synteny between human and chicken.

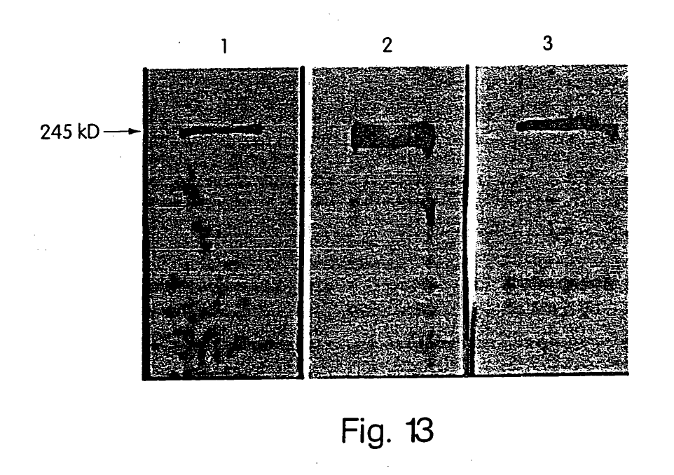

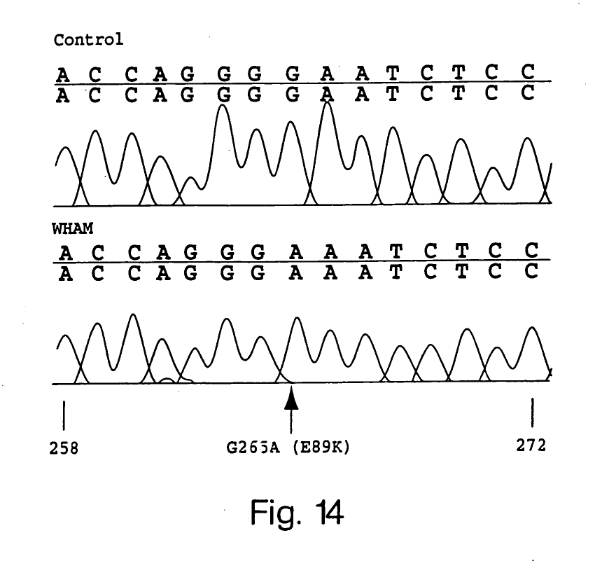

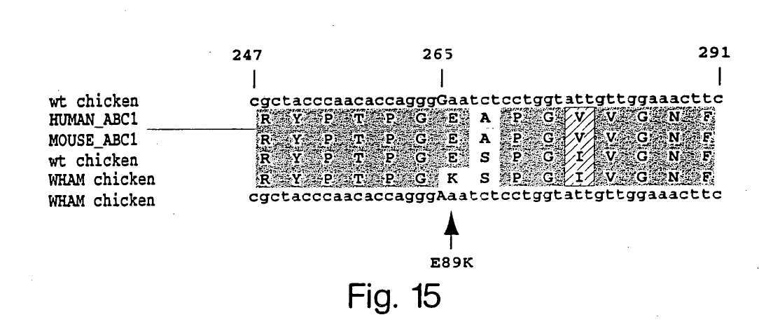

Since a low HDL locus maps to the 9q location in humans and to the Zq region in chickens, these low HDL loci are most probably located within the syntenic region. Thus we predicted that ABC1 is mutated in WHAM chickens. In support of this, we have identified an E_K mutation at a position that corresponds to amino acid 89 of human ABC1 (FIGS. 14 and 15). This non-conservative substitution is at a position that is conserved among human, mouse, and chicken, indicating that it is in a region of the protein likely to be of functional importance.

Discovery of the WHAM mutation in the amino-terminal portion of the ABC1 protein also establishes the importance of the amino-terminal region. This region may be critical because of association with other proteins required to carry out cholesterol efflux or related tasks. It may be an important regulatory region (there is a phosphorylation site for casein kinase near the mutated residue), or it may help to dictate a precise topological relationship with cellular membranes (the N-terminal 60 amino acid region contains a putative membrane-spanning or membrane-associated segment).

The amino-terminal region of the protein (up to the first 6-TM region at approximately amino acid 639) is an ideal tool for screening factors that affect ABC1 activity. It can be expressed as a truncated protein in ABC1 wild type cells in order to test for interference of the normal ABC1 function by the truncated protein. If the fragment acts in a dominant negative way, it could be used in immunoprecipitations to identify proteins that it may be competing away from the normal endogenous protein.

The C-terminus also lends itself to such experiments, as do the intracellular portions of the molecule, expressed as fragments or tagged or fusion proteins, in the absence of transmembrane regions.

Since it is possible that there are several genes in the human genome which affect cholesterol efflux, it is important to establish that any animal model to be used for a human genetic disease represents the homologous locus in that animal, and not a different locus with a similar function. The evidence above establishes that the chicken Y locus and the human chromosome 9 low HDL locus are homologous. WHAM chickens are therefore an important animal model for the identification of drugs that modulate cholesterol efflux.

The WHAM chickens' HDL deficiency syndrome is not, however, associated with an increased susceptibility to atherosclerosis in chickens. This probably reflects the shorter lifespan of the chicken rather than an inherent difference in the function of the chicken ABC1 gene compared to the human gene. We propose the WHAM chicken as a model for human low HDL for the development and testing of drugs to raise HDL in humans. Such a model could be employed in several forms, through the use of cells or other derivatives of these chickens, or by the use of the chickens themselves in tests of drug effectiveness, toxicity, and other drug development purposes.

Therapy

Compounds of the invention, including but not limited to, ABC1 polypeptides, ABC1 nucleic acids, other ABC transporters, and any therapeutic agent that modulates biological activity or expression of ABC1 identified using any of the methods disclosed herein, may be administered with a pharmaceutically-acceptable diluent, carrier, or excipient, in unit dosage form. Conventional pharmaceutical practice may be employed to provide suitable formulations or compositions to administer such compositions to patients. Although intravenous administration is preferred, any appropriate route of administration may be employed, for example, perenteral, subcutaneous, intramuscular, intracranial, intraorbital, ophthalmic, intraventricular, intracapsular, intraspinal, intracisternal, intraperitoneal, intranasal, aerosol, or oral administration. Therapeutic formulations may be in the form of liquid solutions or suspension; for oral administration, formulations may be in the form of tablets or capsules; and for intranasal formulations, in the form of powders, nasal drops, or aerosols.

Methods well known in the art for making formulations are found in, for example, Remington: The Science and Practice of Pharmacy, (19th ed.) ed. A.R. Gennaro AR., 1995, Mack Publishing Company, Easton, Pa. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for agonists of the invention include ethylenevinyl acetate copolymer particles, osmotic pumps, implantable infusion systems, and liposomes. Formulations for inhalation may contain excipients, or example, lactose, or may be aqueous solutions containing, for example, polyoxyethylene-9-lauryl ether, glycocholate and deoxycholate, or may be oily solutions for administration in the form of nasal drops, or as a gel.

Compounds

In general, novel drugs for the treatment of aberrant cholesterol levels and/or CVD are identified from large libraries of both natural product or synthetic (or semi-synthetic) extracts or chemical libraries according to methods known in the art. Those skilled in the field or drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their HDL-raising and anti-CVD activities should be employed whenever possible.

When a crude extract is found to have cholesterol-modulating or anti-CVD activities or both, further fractionation of the positive lead extract is necessary to isolate chemical constituent responsible for the observed effect. Thus, the goal of the extraction, fractionation, and purification process is the careful characterization and identification of a chemical entity within the crude extract having cholesterol-modulating or anti-CVD activities. The same in vivo and in vitro assays described herein for the detection of activities in mixtures of compounds can be used to purify the active component and to test derivatives thereof. Methods of fractionation and purification of such heterogeneous extracts are known in the art. If desired, compounds shown to be useful agents for the treatment of pathogenicity are chemically modified according to methods known in the art. Compounds identified as being of therapeutic value are subsequently analyzed using any standard animal model of diabetes or obesity known in the art.

It is understood that compounds that modulate activity of proteins that modulate or are modulated by ABC1 are useful compounds for modulating cholesterol levels. Exemplary compounds are provided herein; others are known in the art.

Compounds that are structurally related to cholesterol, or that mimic ApoAI or a related apolipoprotein, and increase ABC1 biological activity are particularly useful compounds in the invention. Other compounds, known to act on the MDR protein, can also be used or derivatized and assayed for their ability to increase ABC1 biological activity. Exemplary MDR modulators are PSC833, bromocriptine, and cyclosporin A.

Screening Patients Having Low HDL-C

ABC1 expression, biological activity, and mutational analysis can each serve as a diagnostic tool for low HDL; thus determination of the genetic subtyping of the ABC1 gene sequence can be used to subtype low HDL individuals or families to determine whether the low HDL phenotype is related to ABC1 function. This diagnostic process can lead to the tailoring of drug treatments according to patient genotype, including prediction of side effects upon administration of HDL increasing drugs (referred to herein as pharmacogenomics). Pharmacogenomics allows for the selection of agents (e.g., drugs) for therapeutic or prophylactic treatment of an individual based on the genotype of the individual (e.g., the genotype of the individual is examined to determine the ability of the individual to respond to a particular agent).

Agents, or modulators which have a stimulatory or inhibitory effect on ABC1 biological activity or gene expression can be administered to individuals to treat disorders (e.g., cardiovascular disease or low HDL cholesterol) associated with aberrant ABC1 activity. In conjunction with such treatment, the pharmacogenomics (i.e., the study of the relationship between an individual's genotype and that individual's response to a foreign compound or drug) of the individual may be considered. Differences in efficacy of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of ABC1 protein, expression of ABC1 nucleic acid, or mutation content of ABC1 genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons (Eichelbaum, M., Clin. Exp. Pharmacol. Physiol., 23:983-985, 1996; Linder, M. W., Clin. Chem., 43:254-266, 1997). In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). Altered drug action may occur in a patient having a polymorphism (e.g., an single nucleotide polymorphism or SNP) in promoter, intronic, or exonic sequences of ABC1. Thus by determining the presence and prevalence of polymorphisms allow for prediction of a patient's response to a particular therapeutic agent. In particular, polymorphisms in the promoter region may be critical in determining the risk of HDL deficiency and CVD.

In addition to the mutations in the ABC1 gene described herein, we have detected polymorphisms in the human ABC1 gene (FIG. 11). These polymorphisms are located in promoter, intronic, and exonic sequence of ABC1. Using standard methods, such as direct sequencing, PCR, SSCP, or any other polymorphism-detection system, one could easily ascertain whether these polymorphisms are present in a patient prior to the establishment of a drug treatment regimen for a patient having low HDL, cardiovascular disease, or any other ABC1-mediated condition. It is possible that some these polymorphisms are, in fact, weak mutations. Individuals harboring such mutations may have an increased risk for cardiovascular disease; thus, these polymorphisms may also be useful in diagnostic assays.

Association Studies of ABC1 Gene Variants and HDL Levels or Cardiovascular Disease

The following polymorphisms have been examined for their effect on cholesterol regulation and the predisposition for the development of cardiovascular disease.

Substitution of G for A at nucleotide −1045 [G(−1045)A]. This variant is in complete linkage disequilibrium with the variant at −738 in the individuals we have sequenced, and thus any potential phenotypic effects currently attributed to the variant at −738 may at least in part be due to changes at this site.

Substitution of G for A at nucleotide −738 [G(−738)A]. This variant has been found at very high frequencies in populations selected for low HDL cholesterol or premature coronary artery disease.

Insertion of a G nucleotide at position −4 [G ins (−4)]. This variant has been associated with less coronary artery disease in its carriers than in non-carriers.

Substitution of a C for G at nucleotide −57 [G(−57)C]. This variant is in complete linkage disequilibrium with the variant at −4 in the individuals we have sequenced, and thus the phenotypic effects currently attributed to the variant at −4 may at least in part be due to changes at this site.

Substitution of A for G at nucleotide 730 (R219K). We have found carriers to have significantly less cardiovascular disease.

Substitution of C for T at nucleotide 1270 (V399A). Within the French Canadian population, this variant has only been found in individuals from the low HDL population. It has also been seen in individuals with low HDL or premature coronary artery disease in individuals of Dutch ancestry.

Substitution of A for G at nucleotide 2385 (V771M). This variant has been found at an increased frequency in a Dutch population selected for low HDL and at an increased frequency in a population selected for premature coronary artery disease compared to a control Dutch population, indicating carriers of this variant may have reduced HDL and an increased susceptibility to coronary artery disease.

Substitution of C for A at nucleotide 2394 (T774P). This variant has been seen at lower frequencies in populations with coronary artery disease or low HDL than in individuals without.

Substitution of C for G at nucleotide 2402 (K776N). This variant has been found at a significantly lower frequency (0.56% vs. 2.91%, p=0.02) in a coronary artery disease population vs. a control population of similar Dutch background.

Substitution of C for G at nucleotide 3590 (E1172D). This variant is seen at lower frequencies in individuals with low HDL and in some populations with premature coronary artery disease.

Substitution of A for G at nucleotide 4384 (R1587K). This variant has been found at decreased frequencies in the ⅓ of individuals with the highest HDL levels in our large Dutch coronary artery disease population (p=0.036), at increased frequencies in those with HDL cholesterol <0.9 mmol/L (p<0.0001) and at decreased frequencies in the cohorts with HDL cholesterol >1.4 mmol/L in both this population (p=0.02) and the Dutch control population (p=0.003).

Substitution of G for C at nucleotide 5266 (S1731C). Two FHA individuals who have this variant on the other allele have much lower HDL cholesterol (0.155±0.025) than the FHA individuals in the family who do not have this variant on the other allele (0.64±0.14, p=0.0009). This variant has also been found in one general population French Canadian control with HDL at the 8th percentile (0.92) and one French Canadian individual from a population selected for low HDL and coronary disease (0.72).

Substitution of G for A at nucleotide −1113 [A(−1113)G]. This variant has been seen at varying frequencies in populations distinguished by their HDL levels.

Additional polymorphisms that may be associated with altered risk for cardiovascular disease or altered cholesterol levels are as follows:

Substitution of G for A at nucleotide 2723 (1883M). This variant has been seen at a much higher frequency in individuals of Dutch ancestry with premature coronary artery disease.

Insertion of 4 nucleotides (CCCT) at position −1181.

Substitution of C for A at nucleotide −479 (linkage disequilibrium with −518).

Substitution of G for A at nucleotide −380.

### OTHER EMBODIMENTS

All publications mentioned in this specification are herein incorporated by reference to the same extent as if each independent publication was specifically and individually indicated to be incorporated by reference.

While the invention has been described in connection with specific embodiments thereof, it will be understood that it is capable of further modifications. This application is intended to cover any variations, uses, or adaptations following, in general, the principles of the invention and including such departures from the present disclosure within known or customary practice within the art to which the invention pertains and may be applied to the essential features hereinbefore set forth.

1. A method of determining risk of developing a cardiovascular disease in a mammal, comprising determining the presence of a polymorphism in the amino acid sequence of an ABCA1 polypeptide in a mammal wherein said ABCA1 polymorphism indicates risk of developing a cardiovascular disease.

2. The method of claim 1, wherein said polymorphism is in one of residues 1 to 60 of the amino acid sequence of said ABCA1 polypeptide.

3. The method of claim 1 wherein said mammal is a human being.

4. The method of claim 3, wherein said polymorphism is in the amino acid sequence of SEQ ID NO: 1.

5. The method of claim 4, wherein said polymorphism is selected from the group R219K, K776N and S1731C.

6. The method of claim 1, wherein more than one polymorphism in said ABCA1 protein is determined.

7. The method of claim 1, wherein said polymorphism is determined in the gene encoding said ABCA1 protein.

8. The method of claim 7, wherein said polymorphism in the gene is heterozygous.

9. The method of claim 7, wherein said polymorphism in the gene is homozygous.

10. The method of claim 4, wherein said cardiovascular disease is a member of the group consisting of coronary artery disease (CAD), cerebrovascular disease, coronary restenosis, atherosclerosis and peripheral vascular disease.

11. The method of claim 10, wherein said cardiovascular disease is coronary artery disease (CAD).

12. The method of claim 10 wherein said cardiovascular disease is atherosclerosis.

13. A method of determining risk of developing low plasma HDL levels in a mammal, comprising determining the presence of a polymorphism in the amino acid sequence of an ABCA1 polypeptide in a mammal wherein said ABCA1 polymorphism indicates risk of developing low HDL levels.

14. The method of claim 13, wherein said polymorphism is in one of residues 1 to 60 of the amino acid sequence of said ABCA1 polypeptide.

15. The method of claim 13, wherein said mammal is a human patient.

16. The method of claim 15, wherein said polymorphism is in the amino acid sequence of SEQ ID NO: 1.

17. The method of claim 16, wherein said polymorphism is selected from the group R219K, K776N and S1731C.

18. The method of claim 13, wherein said determination results in a finding of more than one polymorphism in said ABCA1 protein.

19. The method of claim 13, wherein said polymorphism is determined in the gene encoding said ABCA1 protein.

20. The method of claim 19, wherein said polymorphism in the gene is heterozygous.

21. The method of claim 19, wherein said polymorphism in the gene is homozygous.

22. A method of determining risk of developing elevated plasma triglyceride levels in a mammal, comprising determining the presence of a polymorphism in the amino acid sequence of an ABCA1 polypeptide in a mammal wherein the presence of said ABCA1 polymorphism indicates risk of developing elevated triglyceride levels.

23. The method of claim 22, wherein said polymorphism is in one of residues 1 to 60 of the amino acid sequence of said ABCA1 polypeptide.

24. The method of claim 22, wherein said mammal is a human patient.

25. The method of claim 24, wherein said polymorphism is in the amino acid sequence of SEQ ID NO: 1.

26. The method of claim 25, wherein said ABCA1 polymorphism is selected from the group R219K, K776N and S1731C.

27. The method of claim 22, wherein more than one polymorphism in said ABCA1 protein.

28. The method of claim 22, wherein said polymorphism is determined in the gene encoding said ABCA1 protein.

29. The method of claim 28, wherein said polymorphism in the gene is heterozygous.

30. The method of claim 28, wherein said polymorphism in the gene is homozygous.
